(19) United States
(12) Patent Application Publication
(10) Pub. No.: US 2026/0209363 A1
(43) Pub. Date: Jul. 23, 2026

(54) A POLYPEPTIDE COMPLEX WHOSE ANTIGEN-BINDING ACTIVITY VARIES DEPENDING ON THE CONCENTRATION OF A PLASMA PROTEIN

(71) Applicant: CHUGAI SEIYAKU KABUSHIKI KAISHA, Tokyo (JP)

(72) Inventors: Naoka HIRONIWA, Singapore (SG); Yuki NOGUCHI, Yokohama City, Kanagawa (JP); Kazuki SATO, Yokohama City, Kanagawa (JP); Kumiko MURAOKA, Yokohama City, Kanagawa (JP); Shu FENG, Singapore (SG); Shinya ISHII, Yokohama City, Kanagawa (JP); Tomoyuki IGAWA, Tokyo (JP); Shun SHIMIZU, Yokohama City, Kanagawa (JP)

(21) Appl. No.: 19/141,546

(22) PCT Filed: Dec. 22, 2023
(86) PCT No.: PCT/JP2023/046096

§ 371 Date: Jun. 20, 2025

(30) Foreign Application Priority Data
Dec. 23, 2022 (JP) 2022-206130

(51) Int. Cl. C07K 16/28

(52) U.S. Cl. CPC C07K 16/2866; C07K 2317/31; C07K 2317/33; C07K 2317/76; C07K 2317/92; C07K 2317/94

(57) ABSTRACT

The present invention relates to a polypeptide complex comprising a first antigen-binding portion capable of specifically binding to a plasma protein and a second antigen-binding portion capable of binding to a target antigen other than a plasma protein, wherein the first antigen-binding portion and the second antigen-binding portion are directly linked without the mediation of a linker, or are linked via a non-cleavable linker consisting of not more than four amino acid residues. The polypeptide of the present invention does not bind to the target antigen in the presence of the plasma protein but binds to the target antigen in the absence of the plasma protein.

### TECHNICAL FIELD

The present invention relates to polypeptide complexes whose antigen-binding activity varies depending on the concentration of a plasma protein, pharmaceutical compositions comprising the polypeptide complexes, and such. Furthermore, the present invention relates to production methods and screening methods for the polypeptide complexes.

### BACKGROUND ART

Monoclonal antibodies have vast therapeutic potential for treatment of neurological or central nervous system (CNS) diseases, and monoclonal antibodies and such targeting endogenous active intracerebral proteins for treatment of CNS diseases are being developed. Generally, therapeutic monoclonal antibodies exert their activity by binding to target antigens. Therefore, when target antigens are expressed in both the disease site (CNS) and normal tissues (non-CNS), systemic administration of therapeutic monoclonal antibodies could give rise to unfavorable effects in the normal tissues (non-CNS).

In the field of cancer therapy, protease-dependent antigen-binding molecules are known as molecules that act specifically at the cancer lesion site. Protease-dependent antigen-binding molecules are composed of a peptide that inhibits an antigen-binding site, a protease cleavage site, and a target antigen-binding site, and prior to treatment with protease, binding to a target antigen by the target antigen-binding site is inhibited by a peptide that inhibits the antigen-binding site. When the protease-dependent antigen-binding molecule migrates to the cancer lesion site, the protease cleavage site of the molecule is cleaved by proteases whose expression level is elevated specifically at the cancer lesion site. As a result, the peptide that inhibits the target antigen-binding site is dissociated from the protease-dependent antigen-binding molecule, the target antigen-binding site becomes exposed, and binding takes place with the target antigen. As the peptide that inhibits an antigen-binding site, peptides that bind to the target antigen-binding site, peptides that bind to the plasma protein albumin, and such are known (see for example WO2019222282 (PTL 1) and WO2013192546 (PTL 2)). In both cases, the protease cleavage site is an essential component. If a protease cleavage site is absent and cleavage by a protease does not take place, the peptide that inhibits antigen-binding will not dissociate, and therefore, binding of the target antigen-binding site to the target antigen will not be restored.

Contrary to the above, if use of tissue-specific or disease site-specific proteases is difficult, use of protease-dependent antigen-binding molecules is difficult. Furthermore, when using protease-dependent antigen-binding molecules, since cleavage by proteases is irreversible, when molecules that have been cleaved by proteases are redistributed throughout the body, they may generate side effects by acting on normal tissues. In CNS disease therapy, even if the therapeutic molecules reach the disease site (CNS) in one instance, their redistribution throughout the body cannot be avoided, along with excretion of the cerebral tissue interstitial fluid and the cerebrospinal fluid (CSF); therefore, as the therapeutic molecules migrate from CNS to non-CNS, binding to the target antigen has to be decreased again. However, therapeutic molecules that do not act systemically in normal tissues (non-CNS) and the blood, and can exert their actions only at the disease site (CNS) are not known.

### CITATION LIST

### Patent Literature

[PTL 1] WO2019222282
[PTL 2] WO2013192546
[PTL 3] WO2012004384

### Non-Patent Literature

[NPL 1] Comput Struct Biotechnol J. 2013; 6:e201303009
[NPL 2] J Biol Chem. 2002 Sep. 20; 277(38):35035-43

### SUMMARY OF INVENTION

### Technical Problem

The present invention was achieved in view of the above circumstances. An objective of the present invention is to provide pharmaceutical compositions that are useful for treating CNS diseases and active ingredients thereof. In addition, an objective is to provide screening methods and production methods for the pharmaceutical compositions and the active ingredients.

### Solution to Problem

The present inventors conducted dedicated studies to achieve the above-described objectives, and discovered polypeptide complexes whose target antigen-binding activity varies with the plasma protein concentration. The present inventors also created screening methods and production methods for the polypeptide complexes, and thereby completed the present invention.

A polypeptide complex of the present invention comprises a first antigen-binding portion capable of specifically binding to a plasma protein and a second antigen-binding portion capable of binding to a target antigen other than a plasma protein. Regarding the polypeptide complexes of the present invention, in the presence of plasma protein (or in the presence of plasma protein at a high concentration), the first antigen-binding portion binds to a plasma protein, thereby restricting the binding of the second antigen-binding portion to the target antigen; whereas, in the absence of plasma protein (or in the presence of plasma protein at a low concentration), binding of the second antigen-binding portion to the target antigen is not restricted. Moreover, surprisingly, the polypeptide complexes of the present invention regain their binding to the target antigen by migrating from sites where the plasma protein is present (or where the plasma protein is present at a high concentration) to sites where the plasma protein is absent (or where the plasma protein is present at a low concentration), and the complexes do not require dissociation of the first antigen-binding portion by protease cleavage. Such polypeptide complexes or pharmaceutical compositions comprising the polypeptide complexes are expected to be useful for treating CNS diseases.

The present invention is based on these findings and, in one non-limiting specific embodiment, encompasses the following:

[A1] A polypeptide complex comprising a first antigen-binding portion capable of specifically binding to a plasma protein and a second antigen-binding portion capable of binding to a target antigen,
wherein the target antigen is not a plasma protein, and
wherein the first antigen-binding portion is linked with the second antigen-binding portion without a linker.

[A2] The polypeptide complex of [A1], which does not bind to the target antigen when bound to the plasma protein.

[A3] The polypeptide complex of [A1], wherein the binding activity of the complex to the target antigen in the presence of the plasma protein is lower than the binding activity of the complex to the target antigen in the absence of the plasma protein.

[A4] The polypeptide complex of [A1], wherein the binding activity of the complex to the target antigen in the presence of a first concentration of the plasma protein is different from the binding activity of the complex to the target antigen in the presence of a second concentration of the plasma protein.

[A5] The polypeptide complex of [A1], wherein the binding activity of the complex to the target antigen in a human plasma sample is lower than the binding activity of the complex to the target antigen in the absence of a human plasma protein.

[A6] The polypeptide complex of [A1], wherein the binding activity of the complex to the target antigen in a human plasma sample is lower than the binding activity of the complex to the target antigen in a human cerebrospinal fluid (CSF) sample.

[A7] The polypeptide complex of [A1], wherein the binding activity of the complex to the target antigen in the presence of 50 mg/ml of the plasma protein is lower than the binding activity of the complex to the target antigen in the presence of 0.25 mg/ml of the plasma protein.

[A8] The polypeptide complex of [A1], wherein the binding activity of the complex to the target antigen in the presence of about 20, 25, 30, 35, 40, 45, 50, 55, 60, 65, 70, 75 or 80 mg/mL of the plasma protein is lower than the binding activity of the complex to the target antigen in the presence of about 0.05, 0.10, 0.15, 0.20, 0.25, 0.30, 0.35, 0.40, 0.45 or 0.50 mg/mL of the plasma protein.

[A9] The polypeptide complex of [A1], wherein the KD value of the complex for the target antigen in the presence of 50 mg/ml of the plasma protein is 5 times or more, 10 times or more, 15 times or more, 20 times or more, 25 times or more, 30 times or more, or 35 times or more the KD value of the complex for the target antigen in the presence of 0.25 mg/ml of the plasma protein.

[A10] The polypeptide complex of [A1], wherein the KD value of the complex for the target antigen in the presence of about 20, 25, 30, 35, 40, 45, 50, 55, 60, 65, 70, 75 or 80 mg/mL of the plasma protein is 5 times or more, 10 times or more, 15 times or more, 20 times or more, 25 times or more, 30 times or more, or 35 times or more the KD value of the complex for the target antigen in the presence of about 0.05, 0.10, 0.15, 0.20, 0.25, 0.30, 0.35, 0.40, 0.45 or 0.50 mg/mL of the plasma protein.

[A11] The polypeptide complex of any one of [A1] to [A10], wherein the plasma protein is albumin.

[A12] The polypeptide complex of [A11], wherein the albumin is human albumin.

[A13] The polypeptide complex of any one of [A1] to [A12], which comprises two or more first antigen-binding portions and one or more second antigen-binding portions,
wherein one of the second antigen-binding portions is linked with two of the first antigen-binding portions without a linker.

[A14] The polypeptide complex of any one of [A1] to [A12], which comprises one or more first antigen-binding portions and one or more second antigen-binding portions,
wherein one of the second antigen-binding portions is linked with one of the first antigen-binding portions without a linker.

[A15] The polypeptide complex of any one of [A1] to [A14], wherein the second antigen-binding portion comprises a Fab or a scFv.

[A16] The polypeptide complex of any one of [A1] to [A15], wherein the second antigen-binding portion comprises a Fab,
wherein the N-terminal amino acid of the heavy chain variable region and/or light chain variable region of the Fab is linked with the C-terminal amino acid of the first antigen-binding portion without a linker.

[A17] The polypeptide complex of any one of [A1] to [A13], [A15], and [A16], which comprises two first antigen-binding portions and one second antigen-binding portion,
wherein the second antigen-binding portion comprises a Fab,
wherein each N-terminal amino acid of the heavy chain variable region and light chain variable region of the Fab is linked with each C-terminal amino acid of the first antigen-binding portions without a linker.

[A18] The polypeptide complex of any one of [A1] to [A13], [A15], and [A16], which comprises four first antigen-binding portions and two second antigen-binding portions,
wherein each of the second antigen-binding portions comprise a Fab,
wherein each N-terminal amino acid of the heavy chain variable region and light chain variable region of each Fab is linked with each C-terminal amino acid of the first antigen-binding portions without a linker.

[A19] The polypeptide complex of any one of [A1] to [A12] and [A14] to [A16], which comprises one first antigen-binding portion and one second antigen-binding portion,
wherein the second antigen-binding portion comprises a Fab,
wherein the N-terminal amino acid of the heavy chain variable region of the Fab is linked with the C-terminal amino acid of the first antigen-binding portion without a linker.

[A20] The polypeptide complex of any one of [A1] to [A12] and [A14] to [A16], which comprises one first antigen-binding portion and one second antigen-binding portion,
wherein the second antigen-binding portion comprises a Fab,
wherein the N-terminal amino acid of the light chain variable region of the Fab is linked with the C-terminal amino acid of the first antigen-binding portion without a linker.

[A21] The polypeptide complex of any one of [A1] to [A12] and [A14] to [A16], which comprises two first antigen-binding portions and two second antigen-binding portions,
wherein each of the second antigen-binding portions comprise a Fab,
wherein the N-terminal amino acid of the heavy chain variable region of each Fab is linked with each C-terminal amino acid of the first antigen-binding portions without a linker.

[A22] The polypeptide complex of any one of [A1] to [A12] and [A14] to [A16], which comprises two first antigen-binding portions and two second antigen-binding portions,
wherein each of the second antigen-binding portions comprise a Fab,
wherein the N-terminal amino acid of the light chain variable region of each Fab is linked with each C-terminal amino acid of the first antigen-binding portions without a linker.

[A23] The polypeptide complex of any one of [A1] to [A22], wherein the first antigen-binding portion is a Fab, a scFv, a VHH, a VH single domain, a VL single domain, or a peptide.

[A24] The polypeptide complex of any one of [A1] to [A23], wherein the first antigen-binding portion is a peptide comprising the amino acid sequence of SEQ ID NO: 3 or SEQ ID NO: 39.

[A25] The polypeptide complex of any one of [A1] to [A23], wherein the first antigen-binding portion is a peptide comprising the amino acid sequence of:

(SEQ ID NO: 40)
LA[X3]AK[X6][X7]AN[X10]ELD[X14]YGVSDFYKRLI[X26]
KAKTVEGVEALK[X39][X40]IL[X43][X44]LP,

wherein:
[X3] is selected from E, S, Q, or C;
[X6] is selected from E, S, or C;
[X7] is selected from A or S;
[X10] is selected from A, S, or R;
[X14] is selected from A, S, C, or K;
[X26] is selected from D or E;
[X39] is selected from D or E;
[X40] is selected from A or E;
[X43] is selected from A or K;
[X44] is selected from A, S, or E;
L at position 45 is present or absent; and
P at position 46 is present or absent.

[A26] The polypeptide complex of any one of [A1] to [A23], wherein the first antigen-binding portion comprises a peptide having a sequence identity of at least 95%, 96%, 97%, 98%, or 99% with the amino acid sequence of SEQ ID NO: 4.

[A27] The polypeptide complex of any one of [A1] to [A23], wherein the first antigen-binding portion is a peptide comprising the amino acid sequence of SEQ ID NO: 4.

[A28] The polypeptide complex of any one of [A1] to [A23], wherein the first antigen-binding portion comprises a streptococcal scaffold protein which optionally binds to albumin.

[A29] The polypeptide complex of any one of [A1] to [A23] and [A28], wherein the first antigen-binding portion comprises an albumin-binding domain derived from streptococcus strain G148 protein G, optionally wherein the albumin-binding domain comprises 3 alpha-helices.

[A30] The polypeptide complex of any one of [A1] to [A23], [A28], and [A29], wherein the first antigen-binding portion is a non-immunoglobulin-derived affinity protein.

[A31] The polypeptide complex of any one of [A1] to [A30], which further comprises an antibody Fc region.

[A32] The polypeptide complex of any one of [A1] to [A31], which further comprises an antibody Fc region, wherein the second antigen-binding portion is a Fab, wherein the N-terminal amino acid of the antibody Fc region is linked with the C-terminal amino acid of the heavy chain of the Fab.

[A33] The polypeptide complex of [A31] or [A32], wherein the antibody Fc region is a variant Fc region.

[A34] The polypeptide complex of any one of [A1] to [A33], which further comprises a Fab capable of binding to a human transferrin receptor.

[A35] A pharmaceutical composition comprising the polypeptide complex of any one of [A1] to [A34], and a pharmaceutically acceptable carrier.

[A36] An isolated nucleic acid encoding the polypeptide complex of any one of [A1] to [A34].

[A37] A vector comprising the nucleic acid of [A36].

[A38] A host cell comprising the vector of [A37].

[A39] A method for producing the polypeptide complex of any one of [A1] to [A34], comprising culturing the host cell of [A38].

[A40] A method for detecting and/or targeting an antigen in a central nervous system (CNS), comprising systemically administering the polypeptide complex of any one of [A1] to [A34] to a subject.

[A41] The method of [A40], wherein the systemic administration is intravenous administration or subcutaneous administration.

[B1] A polypeptide complex comprising a first antigen-binding portion capable of specifically binding to a plasma protein and a second antigen-binding portion capable of binding to a target antigen,
wherein the target antigen is not a plasma protein, and
wherein the first antigen-binding portion is linked with the second antigen-binding portion via a non-cleavable linker.

[B2] The polypeptide complex of [B1], wherein the non-cleavable linker is a peptide of 4 amino acid residues or less.

[B3] The polypeptide complex of [B1], wherein the non-cleavable linker is a peptide of 3 amino acid residues or less.

[B4] The polypeptide complex of [B1], wherein the non-cleavable linker is a peptide of 1 amino acid residue.

[B5] The polypeptide complex of any one of [B1] to [B4], which does not bind to the target antigen when bound to the plasma protein.

[B6] The polypeptide complex of any one of [B1] to [B4], wherein the binding activity of the complex to the target antigen in the presence of the plasma protein is lower than the binding activity of the complex to the target antigen in the absence of the plasma protein.

[B7] The polypeptide complex of any one of [B1] to [B4], wherein the binding activity of the complex to the target antigen in the presence of a first concentration of the plasma protein is different from the binding activity of the complex to the target antigen in the presence of a second concentration of the plasma protein.

[B8] The polypeptide complex of any one of [B1] to [B4], wherein the binding activity of the complex to the target antigen in a human plasma sample is lower than the binding activity of the complex to the target antigen in the absence of a human plasma protein.

[B9] The polypeptide complex of any one of [B1] to [B4], wherein the binding activity of the complex to the target antigen in a human plasma sample is lower than the binding activity of the complex to the target antigen in a human cerebrospinal fluid (CSF) sample.

[B10] The polypeptide complex of any one of [B1] to [B4], wherein the binding activity of the complex to the target antigen in the presence of 50 mg/ml of the plasma protein is lower than the binding activity of the complex to the target antigen in the presence of 0.25 mg/ml of the plasma protein.

[B11] The polypeptide complex of any one of [B1] to [B4], wherein the binding activity of the complex to the target antigen in the presence of about 20, 25, 30, 35, 40, 45, 50, 55, 60, 65, 70, 75 or 80 mg/mL of the plasma protein is lower than the binding activity of the complex to the target antigen in the presence of about 0.05, 0.10, 0.15, 0.20, 0.25, 0.30, 0.35, 0.40, 0.45 or 0.50 mg/mL of the plasma protein.

[B12] The polypeptide complex of any one of [B1] to [B4], wherein the KD value of the complex for the target antigen in the presence of 50 mg/ml of the plasma protein is 5 times or more, 10 times or more, 15 times or more, 20 times or more, 25 times or more, 30 times or more, or 35 times or more the KD value of the complex for the target antigen in the presence of 0.25 mg/ml of the plasma protein.

[B13] The polypeptide complex of any one of [B1] to [B4], wherein the KD value of the complex for the target antigen in the presence of about 20, 25, 30, 35, 40, 45, 50, 55, 60, 65, 70, 75 or 80 mg/mL of the plasma protein is 5 times or more, 10 times or more, 15 times or more, 20 times or more, 25 times or more, 30 times or more, or 35 times or more the KD value of the complex for the target antigen in the presence of about 0.05, 0.10, 0.15, 0.20, 0.25, 0.30, 0.35, 0.40, 0.45 or 0.50 mg/mL of the plasma protein.

[B14] The polypeptide complex of any one of [B1] to [B13], wherein the plasma protein is albumin.

[B15] The polypeptide complex of [B14], wherein the albumin is human albumin.

[B16] The polypeptide complex of any one of [B1] to [B15], which comprises two or more first antigen-binding portions and one or more second antigen-binding portions,
wherein one of the second antigen-binding portions is linked with two of the first antigen-binding portions via a non-cleavable linker.

[B17] The polypeptide complex of any one of [B1] to [B15], which comprises one or more first antigen-binding portions and one or more second antigen-binding portions,
wherein one of the second antigen-binding portions is linked with one of the first antigen-binding portions via a non-cleavable linker.

[B18] The polypeptide complex of any one of [B1] to [B17], wherein the second antigen-binding portion comprises a Fab or a scFv.

[B19] The polypeptide complex of any one of [B1] to [B18], wherein the second antigen-binding portion comprises a Fab,
wherein the N-terminal amino acid of the heavy chain variable region and/or light chain variable region of the Fab is linked with the C-terminal amino acid of the first antigen-binding portion via a non-cleavable linker.

[B20] The polypeptide complex of any one of [B1] to [B16], [B18], and [B19], which comprises two first antigen-binding portions and one second antigen-binding portion,
wherein the second antigen-binding portion comprises a Fab,
wherein each N-terminal amino acid of the heavy chain variable region and light chain variable region of the Fab is linked with each C-terminal amino acid of the first antigen-binding portions via a non-cleavable linker.

[B21] The polypeptide complex of any one of [B1] to [B16], [B18], and [B19], which comprises four first antigen-binding portions and two second antigen-binding portions,
wherein each of the second antigen-binding portions comprise a Fab,
wherein each N-terminal amino acid of the heavy chain variable region and light chain variable region of each Fab is linked with each C-terminal amino acid of the first antigen-binding portions via a non-cleavable linker.

[B22] The polypeptide complex of any one of [B1] to [B15] and [B17] to [B19], which comprises one first antigen-binding portion and one second antigen-binding portion,
wherein the second antigen-binding portion comprises a Fab,
wherein the N-terminal amino acid of the heavy chain variable region of the Fab is linked with the C-terminal amino acid of the first antigen-binding portion via a non-cleavable linker.

[B23] The polypeptide complex of any one of [B1] to [B15] and [B17] to [B19], which comprises one first antigen-binding portion and one second antigen-binding portion,
wherein the second antigen-binding portion comprises a Fab,
wherein the N-terminal amino acid of the light chain variable region of the Fab is linked with the C-terminal amino acid of the first antigen-binding portion via a non-cleavable linker.

[B24] The polypeptide complex of any one of [B1] to [B15] and [B17] to [B19], which comprises two first antigen-binding portions and two second antigen-binding portions,
wherein each of the second antigen-binding portions comprise a Fab,
wherein the N-terminal amino acid of the heavy chain variable region of each Fab is linked with each C-terminal amino acid of the first antigen-binding portions via a non-cleavable linker.

[B25] The polypeptide complex of any one of [B1] to [B15] and [B17] to [B19], which comprises two first antigen-binding portions and two second antigen-binding portions,
wherein each of the second antigen-binding portions comprise a Fab,
wherein the N-terminal amino acid of the light chain variable region of each Fab is linked with each C-terminal amino acid of the first antigen-binding portions via a non-cleavable linker.

[B26] The polypeptide complex of any one of [B1] to [B25], wherein the first antigen-binding portion is a Fab, a scFv, a VHH, a VH single domain, a VL single domain, or a peptide.

[B27] The polypeptide complex of any one of [B1] to [B26], wherein the first antigen-binding portion is a peptide comprising the amino acid sequence of SEQ ID NO: 3 or SEQ ID NO: 39.

[B28] The polypeptide complex of any one of [B1] to [B26], wherein the first antigen-binding portion is a peptide comprising the amino acid sequence of:

(SEQ ID NO: 40)
LA[X3]AK[X6][X7]AN[X10]ELD[X14]YGVSDFYKRLI[X26]
KAKTVEGVEALK[X39][X40]IL[X43][X44]LP,

wherein:
[X3] is selected from E, S, Q, or C;
[X6] is selected from E, S, or C;
[X7] is selected from A or S;
[X10] is selected from A, S, or R;
[X14] is selected from A, S, C, or K;
[X26] is selected from D or E;
[X39] is selected from D or E;
[X40] is selected from A or E;
[X43] is selected from A or K;
[X44] is selected from A, S, or E;
L at position 45 is present or absent; and
P at position 46 is present or absent.

[B29] The polypeptide complex of any one of [B1] to [B26], wherein the first antigen-binding portion comprises a peptide having a sequence identity of at least 95%, 96%, 97%, 98%, or 99% with the amino acid sequence of SEQ ID NO: 4.

[B30] The polypeptide complex of any one of [B1] to [B26], wherein the first antigen-binding portion is a peptide comprising the amino acid sequence of SEQ ID NO: 4.

[B31] The polypeptide complex of any one of [B1] to [B26], wherein the first antigen-binding portion comprises a streptococcal scaffold protein which optionally binds to albumin.

[B32] The polypeptide complex of any one of [B1] to [B26] and [B31], wherein the first antigen-binding portion comprises an albumin-binding domain derived from streptococcus strain G148 protein G, optionally wherein the albumin-binding domain comprises 3 alpha-helices.

[B33] The polypeptide complex of any one of [B1] to [B26], [B31] and [B32], wherein the first antigen-binding portion is a non-immunoglobulin-derived affinity protein. [B34] The polypeptide complex of any one of [B1] to [B33], which further comprises an antibody Fc region.

[B35] The polypeptide complex of any one of [B1] to [B34], which further comprises an antibody Fc region, wherein the second antigen-binding portion is a Fab, wherein the N-terminal amino acid of the antibody Fc region is linked with the C-terminal amino acid of the heavy chain of the Fab.

[B36] The polypeptide complex of [B29] or [B35], wherein the antibody Fc region is a variant Fc region.

[B37] The polypeptide complex of any one of [B1] to [B36], which further comprises a Fab capable of binding to a human transferrin receptor.

[B38] A pharmaceutical composition comprising the polypeptide complex of any one of [B1] to [B37], and a pharmaceutically acceptable carrier.

[B39] An isolated nucleic acid encoding the polypeptide complex of any one of [B1] to [B37].

[B40] A vector comprising the nucleic acid of [B39].

[B41] A host cell comprising the vector of [B40].

[B42] A method for producing the polypeptide complex of any one of [B1] to [B37], comprising culturing the host cell of [B41].

[B43] A method for detecting and/or targeting an antigen in a central nervous system (CNS), comprising systemically administering the polypeptide complex of any one of [B1] to [B37] to a subject.

[B44] The method of [B43], wherein the systemic administration is intravenous administration or subcutaneous administration.

In one non-limiting specific embodiment, the present invention encompasses the following:

[C1] A polypeptide complex comprising a first polypeptide chain and a second polypeptide chain,
wherein the first polypeptide chain comprises, in order from the N-terminus, a plasma protein-binding portion, a heavy chain variable region (VH), and a heavy chain constant region CH1 domain (CH1), and
   the second polypeptide chain comprises, in order from the N-terminus, a plasma protein-binding portion, a light chain variable region (VL), and a light chain constant region (CL),
wherein the VH and the VL form a binding portion to a target antigen,
wherein the target antigen is not a plasma protein.

[C2] A polypeptide complex comprising a first polypeptide chain and a second polypeptide chain,
wherein the first polypeptide chain comprises, in order from the N-terminus, a plasma protein-binding portion, a heavy chain variable region (VH), and a heavy chain constant region CH1 domain (CH1), and
   the second polypeptide chain comprises, in order from the N-terminus, a light chain variable region (VL) and a light chain constant region (CL),
wherein the VH and the VL form a binding portion to a target antigen,
wherein the target antigen is not a plasma protein.

[C3] A polypeptide complex comprising a first polypeptide chain and a second polypeptide chain,
wherein the first polypeptide chain comprises, in order from the N-terminus, a heavy chain variable region (VH) and a heavy chain constant region CH1 domain (CH1), and
the second polypeptide chain comprises, in order from the N-terminus, a plasma protein-binding portion, a light chain variable region (VL), and a light chain constant region (CL),
wherein the VH and the VL form a target antigen-binding portion,
wherein the target antigen is not a plasma protein.

[C4] The polypeptide complex of any one of [C1] to [C3], wherein the N-terminal amino acid of the VH and/or the VL is linked with the C-terminal amino acid of the plasma protein-binding portion without a linker.

[C5] The polypeptide complex of any one of [C1] to [C3], wherein the N-terminal amino acid of the VH and/or the VL is linked with the C-terminal amino acid of the plasma protein-binding portion via a non-cleavable linker.

[C6] The polypeptide complex of [C5], wherein the non-cleavable linker is a peptide of 4 amino acid residues or less.

[C7] The polypeptide complex of [C5], wherein the non-cleavable linker is a peptide of 3 amino acid residues or less.

[C8] The polypeptide complex of [C5], wherein the non-cleavable linker is a peptide of 1 amino acid residue.

[C9] The polypeptide complex of any one of [C1] to [C8], which further comprises an antibody Fc region.

[C10] The polypeptide complex of [C9], wherein the N-terminal amino acid of the antibody Fc region is linked with the C-terminal amino acid of the CH1.

[C11] The polypeptide complex of [C9] or [C10], wherein the antibody Fc region is a variant Fc region.

[C12] The polypeptide complex of any one of [C1] to [C11], which further comprises a Fab capable of binding to a human transferrin receptor.

[C13] The polypeptide complex of any one of [C1] to [C12], wherein the binding activity of the complex to the target antigen in the presence of the plasma protein is lower than the binding activity of the complex to the target antigen in the absence of the plasma protein.

[C14] The polypeptide complex of any one of [C1] to [C12], wherein the binding activity of the complex to the target antigen in the presence of a first concentration of the plasma protein is different from the binding activity of the complex to the target antigen in the presence of a second concentration of the plasma protein.

[C15] The polypeptide complex of any one of [C1] to [C12], wherein the binding activity of the complex to the target antigen in a human plasma sample is lower than the binding activity of the complex to the target antigen in the absence of a human plasma protein.

[C16] The polypeptide complex of any one of [C1] to [C12], wherein the binding activity of the complex to the target antigen in a human plasma sample is lower than the binding activity of the complex to the target antigen in a human cerebrospinal fluid (CSF) sample.

[C17] The polypeptide complex of any one of [C1] to [C12], wherein the binding activity of the complex to the target antigen in the presence of 50 mg/ml of the plasma protein is lower than the binding activity of the complex to the target antigen in the presence of 0.25 mg/ml of the plasma protein.

[C18] The polypeptide complex of any one of [C1] to [C12], wherein the binding activity of the complex to the target antigen in the presence of about 20, 25, 30, 35, 40, 45, 50, 55, 60, 65, 70, 75 or 80 mg/mL of the plasma protein is lower than the binding activity of the complex to the target antigen in the presence of about 0.05, 0.10, 0.15, 0.20, 0.25, 0.30, 0.35, 0.40, 0.45 or 0.50 mg/mL of the plasma protein.

[C19] The polypeptide complex of any one of [C1] to [C12], wherein the KD value of the complex for the target antigen in the presence of 50 mg/ml of the plasma protein is 5 times or more, 10 times or more, 15 times or more, 20 times or more, 25 times or more, 30 times or more, or 35 times or more the KD value of the complex for the target antigen in the presence of 0.25 mg/ml of the plasma protein.

[C20] The polypeptide complex of any one of [C1] to [C12], wherein the KD value of the complex for the target antigen in the presence of about 20, 25, 30, 35, 40, 45, 50, 55, 60, 65, 70, 75 or 80 mg/mL of the plasma protein is 5 times or more, 10 times or more, 15 times or more, 20 times or more, 25 times or more, 30 times or more, or 35 times or more the KD value of the complex for the target antigen in the presence of about 0.05, 0.10, 0.15, 0.20, 0.25, 0.30, 0.35, 0.40, 0.45 or 0.50 mg/mL of the plasma protein.

[C21] The polypeptide complex of any one of [C1] to [C20], wherein the plasma protein-binding portion is a Fab, a scFv, a VHH, a VH single domain, a VL single domain, or a peptide.

[C22] The polypeptide complex of any one of [C1] to [C21], wherein the plasma protein-binding portion is a peptide comprising the amino acid sequence of SEQ ID NO: 3 or SEQ ID NO: 39.

[C23] The polypeptide complex of any one of [C1] to [C21], wherein the plasma protein-binding portion is a peptide comprising the amino acid sequence of:

(SEQ ID NO: 40)
LA[X3]AK[X6][X7]AN[X10]ELD[X14]YGVSDFYKRLI[X26]
KAKTVEGVEALK[X39][X40]IL[X43][X44]LP,

wherein:
[X3] is selected from E, S, Q, or C;
[X6] is selected from E, S, or C;
[X7] is selected from A or S;
[X10] is selected from A, S, or R;
[X14] is selected from A, S, C, or K;
[X26] is selected from D or E;
[X39] is selected from D or E;
[X40] is selected from A or E;
[X43] is selected from A or K;
[X44] is selected from A, S, or E;
L at position 45 is present or absent; and
P at position 46 is present or absent.

[C24] The polypeptide complex of any one of [C1] to [C21], wherein the plasma protein-binding portion comprises a peptide having a sequence identity of at least 95%, 96%, 97%, 98%, or 99% with the amino acid sequence of SEQ ID NO: 4.

[C25] The polypeptide complex of any one of [C1] to [C21], wherein the plasma protein-binding portion is a peptide comprising the amino acid sequence of SEQ ID NO: 4.

[C26] The polypeptide complex of any one of [C1] to [C21], wherein the first antigen-binding portion comprises a streptococcal scaffold protein which optionally binds to albumin.

[C27] The polypeptide complex of any one of [C1] to [C21] and [C26], wherein the first antigen-binding portion comprises an albumin-binding domain derived from streptococcus strain G148 protein G, optionally wherein the albumin-binding domain comprises 3 alpha-helices.

[C28] The polypeptide complex of any one of [C1] to [C21], [C26] and [C27], wherein the first antigen-binding portion is a non-immunoglobulin-derived affinity protein.

[C29] The polypeptide complex of any one of [C1] to [C28], wherein the plasma protein is albumin.

[C30] The polypeptide complex of [C29], wherein the albumin is human albumin.

[C31] A pharmaceutical composition comprising the polypeptide complex of any one of [C1] to [C30], and a pharmaceutically acceptable carrier.

[C32] An isolated nucleic acid encoding the polypeptide complex of any one of [C1] to [C30].

[C33] A vector comprising the nucleic acid of [C32].

[C34] A host cell comprising the vector of [C33].

[C35] A method for producing the polypeptide complex of any one of [C1] to [C30], comprising culturing the host cell of [C34].

Furthermore, in one non-limiting specific embodiment, the present invention encompasses the following:

[D1] A method for producing a polypeptide complex whose binding activity to a target antigen varies with the concentration of a plasma protein, comprising:
(a) measuring the binding activity of a polypeptide complex to the target antigen both in the presence of the plasma protein and in the absence of the plasma protein,
(b) selecting a polypeptide complex whose binding activity to the target antigen is lower in the presence of the plasma protein than in the absence of the plasma protein,
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

[D2] A method for producing a polypeptide complex whose binding activity to a target antigen varies with the concentration of a plasma protein, comprising:
(a) measuring the binding activity of a polypeptide complex to the target antigen both in the presence of a first concentration of the plasma protein and in the presence of a second concentration of the plasma protein,
(b) selecting a polypeptide complex whose binding activity to the target antigen is different between in the presence of the first concentration of the plasma protein and in the presence of the second concentration of the plasma protein,
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

[D3] A method for producing a polypeptide complex whose binding activity to a target antigen varies with the concentration of a plasma protein, comprising:

(a) measuring the binding activity of a polypeptide complex to the target antigen both in a human plasma sample and in the absence of a human plasma protein,
(b) selecting a polypeptide complex whose binding activity to the target antigen is lower in the human plasma sample than in the absence of the plasma protein,
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

[D4] A method for producing a polypeptide complex whose binding activity to a target antigen varies with the concentration of a plasma protein, comprising:
(a) measuring the binding activity of a polypeptide complex to the target antigen both in a human plasma sample and in a human cerebrospinal fluid (CSF) sample,
(b) selecting a polypeptide complex whose binding activity to the target antigen is lower in the human plasma sample than in the human cerebrospinal fluid (CSF) sample,
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

[D5] A method for producing a polypeptide complex whose binding activity to a target antigen varies with the concentration of a plasma protein, comprising:
(a) measuring the binding activity of a polypeptide complex to the target antigen both in the presence of 50 mg/ml of the plasma protein and in the presence of 0.25 mg/ml of the plasma protein,
(b) selecting a polypeptide complex whose binding activity to the target antigen is lower in the presence of 50 mg/ml of the plasma protein than in the presence of 0.25 mg/ml of the plasma protein,
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

[D6] A method for producing a polypeptide complex whose binding activity to a target antigen varies with the concentration of a plasma protein, comprising:
(a) measuring the binding activity of a polypeptide complex to the target antigen both in the presence of about 20, 25, 30, 35, 40, 45, 50, 55, 60, 65, 70, 75 or 80 mg/mL of the plasma protein and in the presence of about 0.05, 0.10, 0.15, 0.20, 0.25, 0.30, 0.35, 0.40, 0.45 or 0.50 mg/mL of the plasma protein,
(b) selecting a polypeptide complex whose binding activity to the target antigen is lower in the presence of about 20, 25, 30, 35, 40, 45, 50, 55, 60, 65, 70, 75 or 80 mg/mL of the plasma protein than in the presence of about 0.05, 0.10, 0.15, 0.20, 0.25, 0.30, 0.35, 0.40, 0.45 or 0.50 mg/mL of the plasma protein,
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

[D7] A method for producing a polypeptide complex whose binding activity to a target antigen varies with the concentration of a plasma protein, comprising:
(a) measuring the binding activity of a polypeptide complex to the target antigen both in the presence of 50 mg/ml of the plasma protein and in the presence of 0.25 mg/ml of the plasma protein,
(b) selecting a polypeptide complex whose KD value for the target antigen in the presence of 50 mg/ml of the plasma protein is 5 times or more, 10 times or more, 15 times or more, 20 times or more, 25 times or more, 30 times or more, or 35 times or more the KD value of the complex for the target antigen in the presence of 0.25 mg/ml of the plasma protein.
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

[D8] A method for producing a polypeptide complex whose binding activity to a target antigen varies with the concentration of a plasma protein, comprising:
(a) measuring the binding activity of a polypeptide complex to the target antigen both in the presence of about 20, 25, 30, 35, 40, 45, 50, 55, 60, 65, 70, 75 or 80 mg/mL of the plasma protein and in the presence of about 0.05, 0.10, 0.15, 0.20, 0.25, 0.30, 0.35, 0.40, 0.45 or 0.50 mg/mL of the plasma protein,
(b) selecting a polypeptide complex whose KD value for the target antigen in the presence of about 20, 25, 30, 35, 40, 45, 50, 55, 60, 65, 70, 75 or 80 mg/mL of the plasma protein is 5 times or more, 10 times or more, 15 times or more, 20 times or more, 25 times or more, 30 times or more, or 35 times or more the KD value of the complex for the target antigen in the presence of about 0.05, 0.10, 0.15, 0.20, 0.25, 0.30, 0.35, 0.40, 0.45 or 0.50 mg/mL of the plasma protein.
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

[D9] The method of any one of [D1] to [D8], wherein, in the polypeptide complex, the first antigen-binding portion is linked with the second antigen-binding portion without a linker.

[D10] The method of any one of [D1] to [D8], wherein, in the polypeptide complex, the first antigen-binding portion is linked with the second antigen-binding portion via a non-cleavable linker.

[D11] The method of [D10], wherein the non-cleavable linker is a peptide of 4 amino acid residues or less.

[D12] The method of [D10], wherein the non-cleavable linker is a peptide of 3 amino acid residues or less.

[D13] The method of [D10], wherein the non-cleavable linker is a peptide of 1 amino acid residue.

[D14] The method of any one of [D1] to [D13], wherein the plasma protein is albumin.

[D15] The method of [D14], wherein the albumin is human albumin.

[D16] The method of any one of [D1] to [D15], wherein the target antigen is not a plasma protein.

[D17] The method of any one of [D1] to [D16], which further comprises, after step (d), recovering the polypeptide complex from the cell or culture medium thereof.

Furthermore, in one non-limiting specific embodiment, the present invention encompasses the following:

[E1] A method for producing a polypeptide complex whose binding activity to a target antigen varies with the concentration of a plasma protein, comprising: culturing a cell comprising a polynucleotide encoding the polypeptide complex, wherein:
(a) the polypeptide complex comprises a first antigen-binding portion capable of specifically binding to the plasma protein and a second antigen-binding portion capable of binding to the target antigen, and
(b) the binding activity of the polypeptide complex to the target antigen is lower in the presence of the plasma protein than in the absence of the plasma protein.

[E2] A method for producing a polypeptide complex whose binding activity to a target antigen varies with the concentration of a plasma protein, comprising: culturing a cell comprising a polynucleotide encoding the polypeptide complex, wherein:
(a) the polypeptide complex comprises a first antigen-binding portion capable of specifically binding to the plasma protein and a second antigen-binding portion capable of binding to the target antigen, and
(b) the binding activity of the polypeptide complex to the target antigen is different between in the presence of a first concentration of the plasma protein and in the presence of a second concentration of the plasma protein.

[E3] A method for producing a polypeptide complex whose binding activity to a target antigen varies with the concentration of a plasma protein, comprising:
culturing a cell comprising a polynucleotide encoding the polypeptide complex,
wherein:
   (a) the polypeptide complex comprises a first antigen-binding portion capable of specifically binding to the plasma protein and a second antigen-binding portion capable of binding to the target antigen, and
   (b) the binding activity of the polypeptide complex to the target antigen is lower in a human plasma sample than in the absence of the plasma protein.

[E4] A method for producing a polypeptide complex whose binding activity to a target antigen varies with the concentration of a plasma protein, comprising:
culturing a cell comprising a polynucleotide encoding the polypeptide complex,
wherein:
   (a) the polypeptide complex comprises a first antigen-binding portion capable of specifically binding to the plasma protein and a second antigen-binding portion capable of binding to the target antigen, and
   (b) the binding activity of the polypeptide complex to the target antigen is lower in a human plasma sample than in a human cerebrospinal fluid (CSF) sample.

[E5] A method for producing a polypeptide complex whose binding activity to a target antigen varies with the concentration of a plasma protein, comprising:
culturing a cell comprising a polynucleotide encoding the polypeptide complex,
wherein:
   (a) the polypeptide complex comprises a first antigen-binding portion capable of specifically binding to the plasma protein and a second antigen-binding portion capable of binding to the target antigen, and
   (b) the binding activity of the polypeptide complex to the target antigen is lower in the presence of 50 mg/ml of the plasma protein than in the presence of 0.25 mg/ml of the plasma protein.

[E6] A method for producing a polypeptide complex whose binding activity to a target antigen varies with the concentration of a plasma protein, comprising:
culturing a cell comprising a polynucleotide encoding the polypeptide complex,
wherein:
   (a) the polypeptide complex comprises a first antigen-binding portion capable of specifically binding to the plasma protein and a second antigen-binding portion capable of binding to the target antigen, and
   (b) the binding activity of the polypeptide complex to the target antigen is lower in the presence of about 20, 25, 30, 35, 40, 45, 50, 55, 60, 65, 70, 75 or 80 mg/mL of the plasma protein than in the presence of about 0.05, 0.10, 0.15, 0.20, 0.25, 0.30, 0.35, 0.40, 0.45 or 0.50 mg/mL of the plasma protein.

[E7] A method for producing a polypeptide complex whose binding activity to a target antigen varies with the concentration of a plasma protein, comprising:
culturing a cell comprising a polynucleotide encoding the polypeptide complex,
wherein:
   (a) the polypeptide complex comprises a first antigen-binding portion capable of specifically binding to the plasma protein and a second antigen-binding portion capable of binding to the target antigen, and
   (b) the KD value of the polypeptide complex for the target antigen in the presence of 50 mg/ml of the plasma protein is 5 times or more, 10 times or more, 15 times or more, 20 times or more, 25 times or more, 30 times or more, or 35 times or more the KD value of the polypeptide complex for the target antigen in the presence of 0.25 mg/ml of the plasma protein.

[E8] A method for producing a polypeptide complex whose binding activity to a target antigen varies with the concentration of a plasma protein, comprising:
culturing a cell comprising a polynucleotide encoding the polypeptide complex,
wherein:
   (a) the polypeptide complex comprises a first antigen-binding portion capable of specifically binding to the plasma protein and a second antigen-binding portion capable of binding to the target antigen, and
   (b) the KD value of the polypeptide complex for the target antigen in the presence of about 20, 25, 30, 35, 40, 45, 50, 55, 60, 65, 70, 75 or 80 mg/mL of the plasma protein is 5 times or more, 10 times or more, 15 times or more, 20 times or more, 25 times or more, 30 times or more, or 35 times or more the KD value of the polypeptide complex for the target antigen in the presence of about 0.05, 0.10, 0.15, 0.20, 0.25, 0.30, 0.35, 0.40, 0.45 or 0.50 mg/mL of the plasma protein.

[E9] The method of any one of [E1] to [E8], wherein, in the polypeptide complex, the first antigen-binding portion is linked with the second antigen-binding portion without a linker.

[E10] The method of any one of [E1] to [E8], wherein, in the polypeptide complex, the first antigen-binding portion is linked with the second antigen-binding portion via a non-cleavable linker.

[E11] The method of [E10], wherein the non-cleavable linker is a peptide of 4 amino acid residues or less.

[E12] The method of [E10], wherein the non-cleavable linker is a peptide of 3 amino acid residues or less.

[E13] The method of [E10], wherein the non-cleavable linker is a peptide of 1 amino acid residue.

[E14] The method of any one of [E1] to [E13], wherein the plasma protein is albumin.

[E15] The method of [E14], wherein the albumin is human albumin.

[E16] The method of any one of [E1] to [E15], wherein the target antigen is not a plasma protein.

[E17] The method of any one of [E1] to [E16], which further comprises recovering the polypeptide complex from the cell or culture medium thereof.

Furthermore, in one non-limiting specific embodiment, the present invention encompasses the following:

[DE1] The method of any one of [D1] to [D17] and [E1] to [E17], wherein the polypeptide complex comprises two or more first antigen-binding portions and one or more second antigen-binding portions,
wherein one of the second antigen-binding portions is linked with two of the first antigen-binding portions.

[DE2] The method of any one of [D1] to [D17] and [E1] to [E17], wherein the polypeptide complex comprises one or more first antigen-binding portions and one or more second antigen-binding portions,
wherein one of the second antigen-binding portions is linked with one of the first antigen-binding portions.

[DE3] The method of any one of [D1] to [D17] and [E1] to [E17], wherein the second antigen-binding portion comprises a Fab or a scFv.

[DE4] The method of any one of [D1] to [D17] and [E1] to [E17], wherein the second antigen-binding portion comprises a Fab,
wherein the N-terminal amino acid of the heavy chain variable region and/or light chain variable region of the Fab is linked with the C-terminal amino acid of the first antigen-binding portion.

[DE5] The method of any one of [D1] to [D17] and [E1] to [E17], wherein the polypeptide complex comprises two first antigen-binding portions and one second antigen-binding portion,
wherein the second antigen-binding portion comprises a Fab,
wherein each N-terminal amino acid of the heavy chain variable region and light chain variable region of the Fab is linked with each C-terminal amino acid of the first antigen-binding portions.

[DE6] The method of any one of [D1] to [D17] and [E1] to [E17], wherein the polypeptide complex comprises four first antigen-binding portions and two second antigen-binding portions,
wherein each of the second antigen-binding portions comprise a Fab,
wherein each N-terminal amino acid of the heavy chain variable region and light chain variable region of each Fab is linked with each C-terminal amino acid of the first antigen-binding portions.

[DE7] The method of any one of [D1] to [D17] and [E1] to [E17], wherein the polypeptide complex comprises one first antigen-binding portion and one second antigen-binding portion,
wherein the second antigen-binding portion comprises a Fab,
wherein the N-terminal amino acid of the heavy chain variable region of the Fab is linked with the C-terminal amino acid of the first antigen-binding portion.

[DE8] The method of any one of [D1] to [D17] and [E1] to [E17], wherein the polypeptide complex comprises one first antigen-binding portion and one second antigen-binding portion,
wherein the second antigen-binding portion comprises a Fab,
wherein the N-terminal amino acid of the light chain variable region of the Fab is linked with the C-terminal amino acid of the first antigen-binding portion.

[DE9] The method of any one of [D1] to [D17] and [E1] to [E17], wherein the polypeptide complex comprises two first antigen-binding portions and two second antigen-binding portions,
wherein each of the second antigen-binding portions comprise a Fab,
wherein the N-terminal amino acid of the heavy chain variable region of each Fab is linked with each C-terminal amino acid of the first antigen-binding portions.

[DE10] The method of any one of [D1] to [D17] and [E1] to [E17], wherein the polypeptide complex comprises two first antigen-binding portions and two second antigen-binding portions,
wherein each of the second antigen-binding portions comprise a Fab,
wherein the N-terminal amino acid of the light chain variable region of each Fab is linked with each C-terminal amino acid of the first antigen-binding portions.

[DE11] The method of any one of [D1] to [D17], [E1] to [E17] and [DE1] to [DE10], wherein the first antigen-binding portion is a Fab, a scFv, a VHH, a VH single domain, a VL single domain, or a peptide.

[DE12] The method of any one of [D1] to [D17], [E1] to [E17] and [DE1] to [DE11], wherein the first antigen-binding portion is a peptide comprising the amino acid sequence of SEQ ID NO: 3 or SEQ ID NO: 39.

[DE13] The method of any one of [D1] to [D17], [E1] to [E17] and [DE1] to [DE11], wherein the first antigen-binding portion is a peptide comprising the amino acid sequence of:

(SEQ ID NO: 40)
LA[X3]AK[X6][X7]AN[X10]ELD[X14]YGVSDFYKRLI[X26]
KAKTVEGVEALK[X39][X40]IL[X43][X44]LP,

wherein:
[X3] is selected from E, S, Q, or C;
[X6] is selected from E, S, or C;
[X7] is selected from A or S;
[X10] is selected from A, S, or R;
[X14] is selected from A, S, C, or K;
[X26] is selected from D or E;
[X39] is selected from D or E;
[X40] is selected from A or E;
[X43] is selected from A or K;
[X44] is selected from A, S, or E;
L at position 45 is present or absent; and
P at position 46 is present or absent.

[DE14] The method of any one of [D1] to [D17], [E1] to [E17] and [DE1] to [DE11], wherein the first antigen-binding portion comprises a peptide having a sequence identity of at least 95%, 96%, 97%, 98%, or 99% with the amino acid sequence of SEQ ID NO: 4.

[DE15] The method of any one of [D1] to [D17], [E1] to [E17] and [DE1] to [DE11], wherein the first antigen-binding portion is a peptide comprising the amino acid sequence of SEQ ID NO: 4.

[DE16] The polypeptide complex of any one of [D1] to [D17], [E1] to [E17] and [DE1] to [DE11], wherein the first antigen-binding portion comprises a streptococcal scaffold protein which optionally binds to albumin.

[DE17] The polypeptide complex of any one of [D1] to [D17], [E1] to [E17], [DE1] to [DE11] and [DE16], wherein the first antigen-binding portion comprises an albumin-binding domain derived from streptococcus strain G148 protein G, optionally wherein the albumin-binding domain comprises 3 alpha-helices.

[DE18] The polypeptide complex of any one of [D1] to [D17], [E1] to [E17], [DE1] to [DE11], [DE16] and [DE17], wherein the first antigen-binding portion is a non-immunoglobulin-derived affinity protein.

[DE19] The method of any one of [D1] to [D17], [E1] to [E17] and [DE1] to [DE18], wherein the polypeptide complex further comprises an antibody Fc region.

[DE20] The method of any one of [D1] to [D17], [E1] to [E17] and [DE1] to [DE19], wherein the polypeptide complex further comprises an antibody Fc region, and the second antigen-binding portion is a Fab, wherein the N-terminal amino acid of the antibody Fc region is linked to the C-terminal amino acid of the heavy chain of the Fab.

[DE21] The method of [DE19] or [DE20], wherein the antibody Fc region is a variant Fc region.

[DE22] The method of any one of [D1] to [D17], [E1] to [E17] and [DE1] to [DE21], wherein the polypeptide complex further comprises a Fab capable of binding to a human transferrin receptor.

Furthermore, in one non-limiting specific embodiment, the present invention encompasses the following:

[F1] A method of screening for a polypeptide complex whose binding activity to a target antigen varies with the concentration of a plasma protein, comprising:
(a) contacting a polypeptide complex with the target antigen both in the presence of the plasma protein and in the absence of the plasma protein,
(b) measuring the binding activity of the polypeptide complex to the target antigen both in the presence of the plasma protein and in the absence of the plasma protein,
(c) selecting a polypeptide complex whose binding activity to the target antigen is lower in the presence of the plasma protein than in the absence of the plasma protein,
wherein the polypeptide complex comprises a first antigen-binding portion capable of specifically binding to the plasma protein and a second antigen-binding portion capable of binding to the target antigen.

[F2] A method of screening for a polypeptide complex whose binding activity to a target antigen varies with the concentration of a plasma protein, comprising:
(a) contacting a polypeptide complex with the target antigen both in the presence of a first concentration of the plasma protein and in the presence of a second concentration of the plasma protein,
(b) measuring the binding activity of the polypeptide complex to the target antigen both in the presence of the first concentration of the plasma protein and in the presence of the second concentration of the plasma protein, and
(c) selecting a polypeptide complex whose binding activity to the target antigen is different between in the presence of the first concentration of the plasma protein and in the presence of the second concentration of the plasma protein,
wherein the polypeptide complex comprises a first antigen-binding portion capable of specifically binding to the plasma protein and a second antigen-binding portion capable of binding to the target antigen.

[F3] A method of screening for a polypeptide complex whose binding activity to a target antigen varies with the concentration of a plasma protein, comprising:
(a) contacting a polypeptide complex with the target antigen both in a human plasma sample and in the absence of a human plasma protein,
(b) measuring the binding activity of the polypeptide complex to the target antigen both in the human plasma sample and in the absence of the human plasma protein, and
(c) selecting a polypeptide complex whose binding activity to the target antigen is lower in the human plasma sample than in the absence of the plasma protein,
wherein the polypeptide complex comprises a first antigen-binding portion capable of specifically binding to the plasma protein and a second antigen-binding portion capable of binding to the target antigen.

[F4] A method of screening for a polypeptide complex whose binding activity to a target antigen varies with the concentration of a plasma protein, comprising:
(a) contacting a polypeptide complex with the target antigen both in a human plasma sample and in a human cerebrospinal fluid (CSF) sample,
(b) measuring the binding activity of the polypeptide complex to the target antigen both in the human plasma sample and in the human cerebrospinal fluid (CSF) sample, and
(c) selecting a polypeptide complex whose binding activity to the target antigen is lower in the human plasma sample than in the human cerebrospinal fluid (CSF) sample,
wherein the polypeptide complex comprises a first antigen-binding portion capable of specifically binding to the plasma protein and a second antigen-binding portion capable of binding to the target antigen.

[F5] A method of screening for a polypeptide complex whose binding activity to a target antigen varies with the concentration of a plasma protein, comprising:
(a) contacting a polypeptide complex with the target antigen both in the presence of 50 mg/ml of the plasma protein and in the presence of 0.25 mg/ml of the plasma protein,
(b) measuring the binding activity of the polypeptide complex to the target antigen both in the presence of 50 mg/ml of the plasma protein and in the presence of 0.25 mg/ml of the plasma protein, and
(c) selecting a polypeptide complex whose binding activity to the target antigen is lower in the presence of 50 mg/ml of the plasma protein than in the presence of 0.25 mg/ml of the plasma protein,
wherein the polypeptide complex comprises a first antigen-binding portion capable of specifically binding to the plasma protein and a second antigen-binding portion capable of binding to the target antigen.

[F6] A method of screening for a polypeptide complex whose binding activity to a target antigen varies with the concentration of a plasma protein, comprising:
(a) contacting a polypeptide complex with the target antigen both in the presence of about 20, 25, 30, 35, 40, 45, 50, 55, 60, 65, 70, 75 or 80 mg/mL of the plasma protein and in the presence of about 0.05, 0.10, 0.15, 0.20, 0.25, 0.30, 0.35, 0.40, 0.45 or 0.50 mg/mL of the plasma protein,
(b) measuring the binding activity of the polypeptide complex to the target antigen both in the presence of about 20, 25, 30, 35, 40, 45, 50, 55, 60, 65, 70, 75 or 80 mg/mL of the plasma protein and in the presence of about 0.05, 0.10, 0.15, 0.20, 0.25, 0.30, 0.35, 0.40, 0.45 or 0.50 mg/mL of the plasma protein, and
(c) selecting a polypeptide complex whose binding activity to the target antigen is lower in the presence of about 20, 25, 30, 35, 40, 45, 50, 55, 60, 65, 70, 75 or 80 mg/mL of the plasma protein than in the presence of about 0.05, 0.10, 0.15, 0.20, 0.25, 0.30, 0.35, 0.40, 0.45 or 0.50 mg/mL of the plasma protein,
wherein the polypeptide complex comprises a first antigen-binding portion capable of specifically binding to the plasma protein and a second antigen-binding portion capable of binding to the target antigen.

[F7] A method of screening for a polypeptide complex whose binding activity to a target antigen varies with the concentration of a plasma protein, comprising:
(a) contacting a polypeptide complex with the target antigen both in the presence of 50 mg/ml of the plasma protein and in the presence of 0.25 mg/ml of the plasma protein,
(b) measuring the binding activity of the polypeptide complex to the target antigen both in the presence of 50 mg/ml of the plasma protein and in the presence of 0.25 mg/ml of the plasma protein, and
(c) selecting a polypeptide complex whose KD value for the target antigen in the presence of 50 mg/ml of the plasma protein is 5 times or more, 10 times or more, 15 times or more, 20 times or more, 25 times or more, 30 times or more, or 35 times or more the KD value of the complex for the target antigen in the presence of 0.25 mg/ml of the plasma protein,
wherein the polypeptide complex comprises a first antigen-binding portion capable of specifically binding to the plasma protein and a second antigen-binding portion capable of binding to the target antigen.

[F8] A method of screening for a polypeptide complex whose binding activity to a target antigen varies with the concentration of a plasma protein, comprising:
(a) contacting a polypeptide complex with the target antigen both in the presence of about 20, 25, 30, 35, 40, 45, 50, 55, 60, 65, 70, 75 or 80 mg/mL of the plasma protein and in the presence of about 0.05, 0.10, 0.15, 0.20, 0.25, 0.30, 0.35, 0.40, 0.45 or 0.50 mg/mL of the plasma protein,
(b) measuring the binding activity of the polypeptide complex to the target antigen both in the presence of about 20, 25, 30, 35, 40, 45, 50, 55, 60, 65, 70, 75 or 80 mg/mL of the plasma protein and in the presence of about 0.05, 0.10, 0.15, 0.20, 0.25, 0.30, 0.35, 0.40, 0.45 or 0.50 mg/mL of the plasma protein, and
(c) selecting a polypeptide complex whose KD value for the target antigen in the presence of about 20, 25, 30, 35, 40, 45, 50, 55, 60, 65, 70, 75 or 80 mg/mL of the plasma protein is 5 times or more, 10 times or more, 15 times or more, 20 times or more, 25 times or more, 30 times or more, or 35 times or more the KD value of the complex for the target antigen in the presence of about 0.05, 0.10, 0.15, 0.20, 0.25, 0.30, 0.35, 0.40, 0.45 or 0.50 mg/mL of the plasma protein,
wherein the polypeptide complex comprises a first antigen-binding portion capable of specifically binding to the plasma protein and a second antigen-binding portion capable of binding to the target antigen.

[F9] The method of any one of [F1] to [E8], which further comprises, after step (c), selecting a polypeptide complex showing specific binding activity to the plasma protein.

[F10] The method of any one of [F1] to [E8], wherein, in the polypeptide complex, the first antigen-binding portion is linked with the second antigen-binding portion without a linker.

[F11] The method of any one of [F1] to [E8], wherein, in the polypeptide complex, the first antigen-binding portion is linked with the second antigen-binding portion via a non-cleavable linker.

[F12] The method of [F11], wherein the non-cleavable linker is a peptide of 4 amino acid residues or less.

[F13] The method of [F11], wherein the non-cleavable linker is a peptide of 3 amino acid residues or less.

[F14] The method of [F11], wherein the non-cleavable linker is a peptide of 1 amino acid residue.

[F15] The method of any one of [F1] to [E14], wherein the plasma protein is albumin.

[F16] The method of [F15], wherein the albumin is human albumin.

[F17] The method of any one of [F1] to [E16], wherein the target antigen is not a plasma protein.

### Effects of the Invention

The present invention provides a polypeptide complex (plasma protein switch molecule) whose binding activity to a target antigen varies depending on the concentration of a plasma protein. The polypeptide complexes of the present invention do not bind (or have low binding activity) to the target antigen in the presence of plasma protein (or in the presence of plasma protein at a high concentration), but can bind (or have high binding activity) to the target antigen in the absence of plasma protein (or in the presence of plasma protein at a low concentration).

The concentration of plasma protein is high in systemic blood and low in CNS. Therefore, polypeptide complexes of the present invention do not exert medicinal effect in systemic blood where the plasma protein concentration is high, but can exhibit medicinal effect in the CNS where plasma protein concentration is low. Furthermore, even after the polypeptide complexes of the present invention reach the CNS in one instance and become capable of exerting medicinal effect, when the complexes return to systemic blood where the plasma protein concentration is high, their binding to the target antigen decreases again. This is expected to improve medicinal effect in the CNS and decrease side-effects. In one embodiment, the plasma protein is preferably albumin.

Specifically, the polypeptide complexes of the present invention are expected to have the following effects.
The polypeptide complexes of the present invention can exert drug activity by not binding to the target antigen in non-CNS tissues and binding to the target antigen only in the CNS.
The polypeptide complexes of the present invention do not require a linker that can be cleaved by a protease; therefore, they can be used for treating diseases in CNS tissues where proteases specific for the disease site are not expressed. Furthermore, when protease-dependent antigen-binding molecules are used, since cleavage by a protease is irreversible, when molecules after protease cleavage redistribute throughout the body, they may cause side effects by acting on normal tissues. On the other hand, by using polypeptide complexes of the present invention, systemic side effects (which are actions “on-target but in the nontargeted tissues”) can be reduced and high medicinal effect can be achieved.
In the polypeptide complexes of the present invention, the plasma protein-binding portion and the target antigen-binding portion are connected without the mediation of a linker, or by the mediation of a very short non-cleavable linker. Accordingly, increase in immunogenicity accompanying the addition of a long linker, and such can be avoided, and the complexes are suitable for developing therapeutic antibodies.

### BRIEF DESCRIPTION OF DRAWINGS

FIG. 1 is a figure for showing the concept of regulating binding to the target antigen using a plasma protein-binding portion.

FIG. 2 is a figure for showing the concept of regulating binding to the target antigen using a plasma protein-binding portion.

FIG. 3 is a graph showing the amount of complex formed between an anti-IL6R antibody and human soluble IL-6R and the amounts of the complexes formed between various anti-IL-6R albumin switch molecules and human soluble IL-6R in mouse plasma.

FIG. 4 is a graph showing (i) the amount of complex formed between an anti-IL-6R antibody and human soluble IL-6R and the amounts of the complexes formed between various anti-IL-6R albumin switch molecules and human soluble IL-6R in human or rat plasma, and (ii) the amount of complex formed between an anti-IL-6R antibody and monkey soluble IL-6R and the amounts of the complexes formed between various anti-IL-6R albumin switch molecules and monkey soluble IL-6R in monkey plasma.

FIG. 5 is a graph showing the amount of complex formed between an anti-IL-6R antibody and human soluble IL-6R and the amounts of the complexes formed between anti-IL-6R albumin switch molecules (an antibody formed by adding ABD035 or G418) and human soluble IL-6R in mouse plasma.

FIG. 6 depicts binding histograms of antibodies when IC17, the anti-IL6R antibody (H54//IC17), or the anti-IL6R albumin switch molecule (ABD094(H/L)-H54//IC17) was reacted with IL6R-CHO cells at 4 degrees C. for 60 minutes: (a) MSA-free medium, (b) medium containing 50 mg/mL of MSA, and (c) mouse plasma. The X-axis indicates the fluorescence intensity and the Y-axis indicates the cell count number.

FIG. 7 is a graph showing the plasma antibody concentration-time profiles obtained when sIL6R was IV-administered 20 minutes after IV-administration of the anti-IL-6R antibody or the anti-IL-6R albumin switch molecule.

FIG. 8 is a graph showing the plasma sIL6R concentration-time profiles obtained when sIL6R was IV-administered 20 minutes after IV-administration of the anti-IL-6R antibody or the anti-IL-6R albumin switch molecule.

FIG. 9 depicts graphs showing the plasma antibody concentration-time profiles obtained when sIL6R was IV-administered 20 minutes after IV-administration of the anti-IL-6R antibody or the anti-IL-6R albumin switch molecule at (a) 0.2 mg/kg or (b) 1 mg/kg, respectively.

FIG. 10 depicts graphs showing the plasma sIL6R concentration-time profiles obtained when sIL6R was IV-administered 20 minutes after IV-administration of the anti-IL-6R antibody or the anti-IL-6R albumin switch molecule at (a) 0.2 mg/kg or (b) 1 mg/kg, respectively.

FIG. 11 depicts graphs showing the concentrations of total sIL6R in plasma at 5 minutes after sIL6R administration when sIL6R was IV-administered 20 minutes after IV-administration of the anti-IL-6R antibody or the anti-IL-6R albumin switch molecule at (a) 0.2 mg/kg or (b) 1 mg/kg, respectively.

FIG. 12 depicts graphs showing the concentrations of free sIL6R in plasma at 5 minutes after sIL6R administration when sIL6R was IV-administered 20 minutes after IV-administration of the anti-IL-6R antibody or the anti-IL-6R albumin switch molecule at (a) 0.2 mg/kg or (b) 1 mg/kg, respectively.

FIG. 13 depicts graphs showing the concentrations of bound sIL6R in plasma at 5 minutes after sIL6R administration when sIL6R was IV-administered 20 minutes after IV-administration of the anti-IL-6R antibody or the anti-IL-6R albumin switch molecule at (a) 0.2 mg/kg or (b) 1 mg/kg, respectively.

FIG. 14 is a graph showing the CSF total antibody concentration-time profiles obtained when sIL6R was administered to the lateral cerebroventricle 10 minutes after lateral cerebroventricular administration of the anti-IL-6R antibody or the anti-IL-6R albumin switch molecule.

FIG. 15 is a graph showing the CSF total sIL6R concentration-time profiles obtained when sIL6R was administered to the lateral cerebroventricle 10 minutes after lateral cerebroventricular administration of the anti-IL-6R antibody or the anti-IL-6R albumin switch molecule.

FIG. 16 depicts graphs showing the concentration of total antibodies in the CSF at (a) 10 minutes after and (b) 90 minutes after sIL6R administration, when sIL6R was administered to the lateral cerebroventricle 10 minutes after lateral cerebroventricular administration of the anti-IL-6R antibody or the anti-IL-6R albumin switch molecule.

FIG. 17 depicts graphs showing the concentration of total sIL6R in the CSF at (a) 10 minutes after and (b) 90 minutes after sIL6R administration, when sIL6R was administered to the lateral cerebroventricle 10 minutes after lateral cerebroventricular administration of the anti-IL-6R antibody or the anti-IL-6R albumin switch molecule.

FIG. 18 is a graph showing the concentration of free sIL6R in the CSF at 90 minutes after sIL6R administration when sIL6R was administered to the lateral cerebroventricle 10 minutes after lateral cerebroventricular administration of the anti-IL-6R antibody or the anti-IL-6R albumin switch molecule.

FIG. 19 depicts graphs showing the concentration of bound sIL6R in the CSF at (a) 10 minutes after and (b) 90 minutes after sIL6R administration when sIL6R was administered to the lateral cerebroventricle 10 minutes after lateral cerebroventricular administration of the anti-IL-6R antibody or the anti-IL-6R albumin switch molecule.

FIG. 20 is a graph showing the amount of complex formed between an anti-IL-6R antibody and human soluble IL-6R and the amounts of complexes formed between various anti-IL-6R albumin switch molecules (with/without linkers) and human soluble IL-6R in mouse plasma.

FIG. 21 is a graph showing the plasma sIL6R concentration-time profiles obtained when sIL6R was IV-administered 20 minutes after IV-administration of the anti-IL-6R antibody or the anti-IL-6R albumin switch molecule.

FIG. 22 is a graph showing the plasma sIL6R concentration-time profiles obtained when sIL6R was IV-administered 20 minutes after IV-administration of the anti-IL6R antibody or the anti-IL6R albumin switch molecule. Each point shows the mean+/−standard deviation (n=3).

FIG. 23 is a graph showing the amount of complex formed between an anti-mouse MOG antibody and mouse soluble MOG and the amounts of complexes formed between various anti-mouse MOG albumin switch molecules (with/without linkers) and mouse soluble MOG in mouse plasma.

FIG. 24 shows binding histograms of antibodies obtained when IC17, the anti-MOG antibody (MOG//IC17), or the anti-MOG albumin switch molecule (ABD094 (H/L)-MOG//IC17) was reacted with mouse brain cell suspension at 4 degrees C. for 60 minutes, and the fluorescence intensity of the antibodies in the oligodendrocyte fraction was measured by FACS: (a) MSA-free DPBS, (b) DPBS containing MSA at 50 mg/mL, and (c) mouse plasma. The X axis shows the antibody fluorescence intensity and the Y axis shows the cell count number.

FIG. 25 is a graph showing the plasma antibody concentration-time profiles obtained when MOG was IV-administered 20 minutes after IV-administration of the anti-MOG antibody or the anti-MOG albumin switch molecule. Time indicates the time since MOG administration.

FIG. 26 is a graph showing the plasma antigen concentration-time profiles obtained when MOG was IV-administered 20 minutes after IV-administration of the anti-MOG antibody or the anti-MOG albumin switch molecule. Time indicates the time since MOG administration.

FIG. 27 is a graph showing the plasma antibody concentration-time profiles obtained when the anti-MOG antibody or the anti-MOG albumin switch molecule was IV-administered.

FIG. 28 is a graph showing the brain antibody concentration-time profiles obtained when the anti-MOG antibody or the anti-MOG albumin switch molecule was IV-administered.

FIG. 29 is a graph showing the plasma antigen (sMOG) concentration-time profiles obtained when the anti-MOG antibody or the anti-MOG albumin switch molecules with various linker lengths was IV-administered.

### DESCRIPTION OF EMBODIMENTS

### Polypeptide Complexes

In one embodiment, the present invention relates to polypeptide complexes comprising a first antigen-binding portion that can specifically bind to a plasma protein (herein, also referred to as “plasma protein-binding portion”) and a second antigen-binding portion that can bind to a target antigen (herein, also referred to as “target antigen-binding portion”). In one embodiment, the first antigen-binding portion and the second antigen-binding portion are linked without the mediation of a linker, or by the mediation of a very short non-cleavable linker. In one embodiment, polypeptide complexes of the present invention are fusion polypeptides in which the first antigen-binding portion and the second antigen-binding portion are directly fused. In one embodiment, polypeptide complexes of the present invention are “plasma protein switch molecules” whose binding activity to a target antigen varies depending on the concentration of plasma protein. While various plasma proteins may be used, when using albumin, for example, the polypeptide complexes of the present invention are “albumin switch molecules” whose binding activity to a target antigen varies depending on the concentration of albumin.

In the state where the plasma protein-binding portion of a polypeptide complex of the present invention is bound to a plasma protein, the complex has decreased binding activity to a target antigen compared to the state where the plasma protein-binding portion is not bound to the plasma protein. In one embodiment, polypeptide complexes of the present invention cannot bind to target antigens in the state where the plasma protein-binding portion is bound to a plasma protein. When the plasma protein dissociates from the plasma protein-binding portion, polypeptide complexes of the present invention can restore their binding activity to the target antigen.

Antibodies whose binding activity to a target antigen varies in a protease-dependent manner (protease-dependent antigen-binding antibodies) are known as antibodies whose binding to therapeutic protein target antigens is regulated. A protease-dependent antigen-binding antibody is composed of a peptide that inhibits an antigen-binding site, a protease cleavage site, and a target antigen-binding site. In a protease-dependent antigen-binding antibody, its antigen-binding site acquires target antigen-binding activity when its protease cleavage site is cleaved by a protease (e.g., WO2019222282 and WO2013192546). As seen for protease-dependent antigen-binding antibodies, when regulating binding to a target antigen by masking the antigen-binding site of a therapeutic protein using another peptide or such, a protease cleavage site was considered to be an essential component. However, polypeptide complexes of the present invention may not comprise a protease cleavage site (including a protease-cleavable linker or a peptide linker). The finding that binding of a polypeptide complex to a target antigen can be regulated without the use of a protease cleavage site was surprising.

In one embodiment, polypeptide complexes of the present invention can comprise a linker not cleaved by proteases and such (non-cleavable linker) between the plasma protein-binding portion and the target antigen-binding portion. In one embodiment, the non-cleavable linker in the present disclosure may be a linker that does not comprise a sequence that may be cleaved by a protease. In one embodiment, the non-cleavable linker may be a non-cleavable peptide linker. Therefore, in the polypeptide complexes of the present invention, the plasma protein-binding portion and the target antigen-binding portion may be linked via a non-cleavable linker or a non-cleavable peptide linker.

There are cases where a protein is produced by fusing a plurality of antigen-binding portions with the objective of providing a therapeutic protein with a plurality of antigen specificities. In many of such fusion proteins, multiple antigen-binding portions are linked using linkers or peptide linkers. While there are various reasons for using linkers or peptide linkers, linkers having a certain length may be used to avoid interference among the multiple antigen-binding portions or to avoid the influence of one antigen-binding portion on the antigen-binding activity of another antigen-binding portion. For example, there are reports that in DVD-Ig produced by linking to the N terminus of the variable region of a certain antibody, the variable region of another antibody, its antigen-binding activity is affected by the length of the linker. Specifically, when using a short linker (5 or 6 amino acid residues), the binding affinity of the inner antigen binding site was shown to be decreased compared to when using a long linker (12 or 13 amino acid residues) (Jakob, C. G., et al., Structure reveals function of the dual variable domain immunoglobulin (DVD-Ig™) molecule. MAbs. 2013 May-June; 5(3):358-63). As described, when linking a plurality of antigen-binding portions, use of linkers, selection of their binding sites, selection of linker types and lengths, and such are not simple processes, and to obtain therapeutic proteins having the desired properties these processes require trial and error. In view of such common knowledge in the art, it was surprisingly revealed that polypeptide complexes having the desired properties can be obtained by linking the plasma protein-binding portion and the target antigen-binding portion without the mediation of a linker or a peptide linker. In particular, it was an unexpected finding that the target antigen-binding activity of the target antigen-binding portion is maintained (in the absence of plasma protein or under low plasma protein concentration) even without the use of a linker or a peptide linker, or even when a very short linker or peptide linker is used.

In one embodiment, polypeptide complexes of the present invention may comprise between the plasma protein-binding portion and the target antigen-binding portion, a non-cleavable linker of not more than 4 amino acid residues, not more than 3 amino acid residues, not more than 2 amino acid residues, or one amino acid residue. Preferably, the non-cleavable linker has 3 or fewer amino acid residues. More preferably, the polypeptide complexes of the present invention may comprise a non-cleavable linker of one amino acid residue, or no linker or peptide linker at all, between the plasma protein-binding portion and the target antigen-binding portion. Most preferably, the polypeptide complexes of the present invention may not comprise any linker or peptide linker between the plasma protein-binding portion and the target antigen-binding portion. In other words, in the polypeptide complexes of the present invention, the plasma protein-binding portion and the target antigen-binding portion may be linked without the mediation of a linker or a peptide linker. Specifically, in one embodiment, the present invention relates to a polypeptide complex comprising a first antigen-binding portion capable of specifically binding to a plasma protein (plasma protein-binding portion) and a second antigen-binding portion capable of binding to a target antigen (target antigen-binding portion), wherein the first antigen-binding portion and the second antigen-binding portion are linked via a linker consisting of 0 to 4 amino acid residues that is not cleaved by a protease.

The above phrase “are linked via a linker consisting of 0 to 4 amino acid residues” can be rephrased as “have an insertion of an amino acid sequence of 0 to 4 amino acid residues”. Alternatively, the above phrase “are linked via a linker consisting of 0 to 4 amino acid residues” can be rephrased as “are linked without the mediation of a linker, or linked via a linker consisting of 1 to 4 amino acid residues”. More specifically, in one embodiment, polypeptide complexes of the present invention have an insertion of an amino acid sequence of not more than 4 amino acid residues, not more than 3 amino acid residues, not more than 2 amino acid residues, or one amino acid residue between the plasma protein-binding portion and the target antigen-binding portion. Preferably, the inserted amino acid sequence has 3 or less amino acid residues. More preferably, the inserted amino acid sequence has one amino acid residue, or no amino acid sequence is inserted at all.

Examples of the non-cleavable linker include glycine linkers (G)n (where n is 1, 2, 3, or 4), glycine-serine linkers (for example, (GS)n (where n is 1 or 2), and (GGGS: SEQ ID NO: 42)), glycine-alanine linkers, alanine-serine linkers, and other linkers well-known in the art. Examples of flexible linkers consisting of glycine-serine linkers include, but are not limited to, the following:
Ser
Gly (corresponding to N10 in the Examples of the present application)
Gly Ser (GS) (corresponding to N11 in the Examples of the present application) Ser Gly (SG)
Gly Gly Ser (GGS) (corresponding to N12 in the Examples of the present application)
Gly Ser Gly (GSG)
Ser Gly Gly (SGG)
Gly Ser Ser (GSS)
Ser Ser Gly (SSG)
Ser Gly Ser (SGS)
Gly Gly Gly Ser (GGGS, SEQ ID NO: 42) (corresponding to N13 in the Examples of the present application)

```
  (GGSG, SEQ ID NO: 43)
Gly Gly Ser Gly
  (GSGG, SEQ ID NO: 44)
Gly Ser Gly Gly
  (SGGG, SEQ ID NO: 45)
Ser Gly Gly Gly
  (GSSG, SEQ ID NO: 46)
Gly Ser Ser Gly
```

Preferably, the non-cleavable linker is Gly, Gly Ser (GS), Gly Gly Ser (GGS), or Gly Gly Gly Ser (GGGS, SEQ ID NO: 42). More preferably, the non-cleavable linker is Gly, Gly Ser (GS), or Gly Gly Ser (GGS). Most preferably, the non-cleavable linker is Gly.

As described above, in conventional fusion polypeptides, linkers have been used with the objective of preventing interference among multiple antigen-binding portions, or preventing one antigen-binding portion from affecting the antigen-binding activity of another antigen-binding portion. In the present invention the phrases “without the mediation of a linker” and “not comprising a linker” mean that a peptide sequence is not inserted between the first antigen-binding portion and the second antigen-binding portion under such intended purposes. Therefore, if glycine, serine, alanine, and such often observed in peptide linkers are included at the C terminus of the first antigen-binding portion and/or the N terminus of the second antigen-binding portion, and if this results in generation of a moiety having a linker-like sequence when the C-terminal amino acid residue of the first antigen-binding portion and the N-terminal amino acid residue of the second antigen-binding portion are linked, polypeptide complexes carrying such a moiety will be included in the polypeptide complexes in which the first antigen-binding portion and the second antigen-binding portion are linked without the mediation of a linker.

### First Antigen-Binding Portions (Plasma Protein-Binding Portions)

Examples of a first antigen-binding portion (plasma protein-binding portion) of the present invention include small molecule compounds having specific binding activity for plasma proteins, peptides having specific binding activity for plasma proteins, protein domains that bind to plasma proteins, or fragments thereof, and their origin is not particularly limited.

Plasma proteins of the present invention are not particularly limited as long as they are proteins present at higher concentration in the brain tissue interstitial fluid or the cerebrospinal fluid (CSF) than in the plasma. Examples of the ratio of concentration in the plasma to concentration in the brain tissue interstitial fluid or the cerebrospinal fluid (CSF) are not particularly limited, but include 10-fold or more, preferably 50-fold or more, and more preferably 100-fold or more. Examples of plasma proteins in the present invention include albumin, cystinylated albumin, IgG, beta-Trace (prostaglandin D synthase), Transthyretin, transferrin, alpha1-Antitrypsin, Apolipoprotein A, gamma-Trace (cystatin-C), Orosomucoid, and Haemopexin, but are not limited thereto. Furthermore, there is no limit on the biological species from which the plasma proteins originate. Examples of plasma proteins of the present invention include plasma proteins derived from humans, rabbits, mice, rats, monkeys, bovines, and such. These plasma proteins can be obtained readily by techniques known to those skilled in the art. Alternatively, they may be purchased from suppliers. First antigen-binding portions having specific binding activity toward plasma proteins derived from humans, rabbits, mice, rats, monkeys, bovines, and such may be used for the polypeptide complexes of the present invention.

Examples of a plasma protein-binding portion of the present invention include small molecule compounds, peptides, protein domains, and fragments thereof. Furthermore, the plasma protein-binding portion may be, an antibody fragment such as Fab, scFv, domain antibody (dAb) and non-immunoglobulin affinity proteins. Examples of a domain antibody include VH single domain, VL single domain, VHH and Nanobody. Examples of non-immunoglobulin affinity proteins include Affibody, Affilin, Anticalin, Atrimer, Avimer, Bycyclic peptide, Cys-knot, DARPin, FN3 (Adnectin), Fynomer, Kunitz domains, OBodies, and such (Simeon, R., et al., In vitro-engineered non-antibody protein therapeutics. Protein Cell 9, 3-14 (2018)).

In the present invention, Fab is the result of removing the Fc regions from a full-length antibody, and is a set of a polypeptide chain comprising a heavy chain variable region and a CH1 domain of a heavy chain constant region (CH1) and a polypeptide chain comprising a light chain variable region and a light chain constant region. Fab can be obtained by treating an antibody with papain to generate antibody fragments, or by constructing a gene encoding an antibody fragment, introducing it into an expression vector, and then expressing the gene in suitable host cells (see for example, Co, M. S., et al., J. Immunol. (1994) 152, 2968-297; Better, M. & Horwitz, A. H., Methods in Enzymology (1989) 178, 476-496; Plueckthun, A. & Skerra, A., Methods in Enzymology (1989) 178, 497-515; Lamoyi, E., Methods in Enzymology (1989) 121, 652-663; Rousseaux, J., et al., Methods in Enzymology (1989) 121, 663-66; Bird, R. E., et al., TIBTECH (1991) 9, 132-137).

scFv can be obtained by linking the heavy-chain variable region and light-chain variable region of an antibody. In scFv, the heavy-chain variable region and light-chain variable region are ligated via a linker, preferably a peptide linker (Huston, J. S. et al., Proc. Natl. Acad. Sci. U.S.A. (1988) 85, 5879-5883). For example, an arbitrary single chain peptide consisting of 12-19 amino acid residues may be used as the peptide linker for ligating the variable regions.

A single-domain antibody is an antibody fragment comprising all or a part of an antibody's heavy-chain variable region or heavy-chain variable domain, or all or a part of the light-chain variable region or light-chain variable domain. In a specific embodiment, the single-domain antibody is a human single-domain antibody (Domantis, Inc., Waltham, MA; see, e.g., U.S. Pat. No. 6,248,516 B1). The single-domain antibody is not limited by its structure as long as the domain can exert antigen binding activity by itself. A general antibody, for example, an IgG antibody, exhibits antigen binding activity by forming a variable region through the pairing of VH and VL, whereas the own domain structure of the single-domain antibody can exert antigen binding activity by itself without pairing with another domain. Usually, the single-domain antibody has a relatively low molecular weight and exists in the form of a monomer.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Examples of the single-domain antibody include, but are not limited to, antigen binding molecules congenitally lacking a light chain, such as VHH of an animal of the family Camelidae and shark V_{NAR}, and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present invention, one single-domain antibody has three CDRs (CDR1, CDR2 and CDR3).

The single-domain antibody can be obtained from an animal capable of producing the single-domain antibody or by the immunization of the animal capable of producing the single-domain antibody. Examples of the animal capable of producing the single-domain antibody include, but are not limited to, animals of the family Camelidae, and transgenic animals harboring a gene capable of raising the single-domain antibody. The animals of the family Camelidae include camels, lamas, alpacas, one-hump camels and guanacos, etc. Examples of the transgenic animals harboring a gene capable of raising the single-domain antibody include, but are not limited to, transgenic animals described in International Publication No. WO2015/143414 and U.S. Patent Publication No. US2011/0123527 A1. The framework sequences of the single-domain antibody obtained from the animal may be converted to human germline sequences or sequences similar thereto to obtain a humanized single-domain antibody. The humanized single-domain antibody (e.g., humanized VHH) is also one embodiment of the single-domain antibody of the present invention.

Alternatively, the single-domain antibody can be obtained by ELISA, panning, or the like from a polypeptide library containing single-domain antibodies. Examples of the polypeptide library containing single-domain antibodies include, but are not limited to, naive antibody libraries obtained from various animals or humans (e.g., Methods in Molecular Biology 2012 911 (65-78); and Biochimica et Biophysica Acta—Proteins and Proteomics 2006 1764: 8 (1307-1319)), antibody libraries obtained by the immunization of various animals (e.g., Journal of Applied Microbiology 2014 117: 2 (528-536)), and synthetic antibody libraries prepared from antibody genes of various animals or humans (e.g., Journal of Biomolecular Screening 2016 21: 1 (35-43); Journal of Biological Chemistry 2016 291:24 (12641-12657); and AIDS 2016 30: 11 (1691-1701)).

In one embodiment of the present invention, albumin is presented as an example of the plasma protein. In this case, a first antigen-binding portion capable of binding to albumin (albumin-binding portion) is used as the first antigen-binding portion capable of specifically binding to a plasma protein. Examples of such a first antigen-binding portion include small molecule compounds having specific binding activity for albumin, peptides having specific binding activity for albumin, albumin-binding protein domains, or fragments thereof, and their origin is not particularly limited. There is no limit to the biological species from which the albumin originates. Examples of albumin in the present invention include human albumin, rabbit albumin, mouse albumin, rat albumin, monkey albumin, and bovine albumin. These albumins can be obtained readily by techniques known to those skilled in the art. Alternatively, they may be purchased from suppliers. A first antigen-binding portion (albumin-binding portion) having specific binding activity toward albumin derived from humans, rabbits, mice, rats, monkeys, bovines, and such may be used for a polypeptide complex of the present invention.

In one embodiment, examples of an albumin-binding portion include small molecule compounds, peptides, protein domains, and fragments thereof as described in Zorzi A, et. al., Non-covalent albumin-binding ligands for extending the circulating half-life of small biotherapeutics. Medchemcomm. 2019 Jun. 6; 10(7):1068-1081. Furthermore, examples of the albumin-binding portion may be antibody fragments such as Fab, scFv, domain antibody (dAb), and non-immunoglobulin affinity proteins. Examples of a domain antibody include VH single domain, VL single domain, VHH and Nanobody. Examples of non-immunoglobulin affinity proteins include Affibody, Affilin, Anticalin, Atrimer, Avimer, Bycyclic peptide, Cys-knot, DARPin, FN3 (Adnectin), Fynomer, Kunitz domains, OBodies, and such (Simeon, R., et al., In vitro-engineered non-antibody protein therapeutics. Protein Cell 9, 3-14 (2018)).

### Albumin-Binding Peptides (ABP)

In the present invention, a peptide having binding activity for albumin (albumin-binding peptides) can also be used as the first antigen-binding portion. Albumin-binding peptides are peptides obtained by the phage display method, which have a maximum length of 20 amino acids and comprise 2 cysteines that bind to albumin. The peptides were obtained by performing a panning operation for albumin using phages with random amino acid occurrence, and selecting the clones that bind to albumin. In particular, albumin-binding peptides comprising the core sequence “DICLPRWGCLW (SEQ ID NO: 38)” have been demonstrated to have high binding affinity against albumins derived from humans, rabbits, rats, and mice (Dennis M S, et al., Albumin binding as a general strategy for improving the pharmacokinetics of proteins. J. Biol. Chem. 2002 Sep. 20; 277(38):35035-43).

In one embodiment, albumin-binding peptides in the present invention are peptides that comprise the amino acid sequence: DICLPRWGCLW (SEQ ID NO: 38) and have 20 or fewer amino acid residues (for example, 19, 18, 17, 16, 15, 14, 13, 12, or 11 residues). Preferably, albumin-binding peptides in the present invention include peptides having the amino acid sequence of SEQ ID NO: 3 or SEQ ID NO: 39. Alternatively, albumin-binding peptides in the present invention are peptides consisting of the amino acid sequence of SEQ ID NO: 3 or SEQ ID NO: 39. The amino acid sequences of SEQ ID NO: 3 and SEQ ID NO: 39 are the same as the amino acid sequences of SA06 and SA21 which are albumin-binding peptides reported in J Biol Chem. 2002 Sep. 20; 277(38):35035-43 mentioned above.

### Albumin Binding Protein Domains or Fragments Thereof

The albumin binding protein domain comprises or is a three-helical protein domain found in various surface proteins expressed by Gram-positive bacteria. The albumin binding protein derived from streptococcal scaffold protein G has 214 amino acids, and contains three albumin-binding domains (ABD1 to 3) used to bind to human serum albumin and evade the host's immune system. Albumin binding domain 3 is a sequence corresponding to 46 amino acids, and has been demonstrated to bind to human serum albumin. Through such studies, a significant number of ABD polypeptides having various properties have been produced.

Albumin-binding proteins are found in other bacteria as well. Examples of naturally-occurring albumin-binding proteins include certain surface proteins derived from Gram-positive bacteria, such as streptococcal M proteins (for example, M1/Emm1, M3/Emm3, M12/Emm12, EmmL55/Emm55, Emm49/EmmL49, and protein H), streptococcal protein G, MAG and ZAG, and PPL and PAB derived from certain strains of Finegoldia magna.

In one embodiment, examples of an albumin-binding portion in the present invention comprise a streptococcal scaffold protein which optionally binds to albumin. In one embodiment, albumin-binding portions in the present invention include albumin-binding domain (ABD) polypeptides that is or is derived from the streptococcal protein G albumin-binding domain. In a further embodiment, an ABD polypeptide is or is derived from the complete streptococcal protein G albumin binding domain 3 or a functional fragment and/or a variant thereof. In a further embodiment, an ABD polypeptide is or is derived from the streptococcal strain G148 protein G. In a further embodiment, the albumin-binding domain may comprise 3 alpha-helices. In one embodiment, the albumin-binding domain in the present invention is G148-ABD3, ALB8-GA, G148-ABD1, G148-ABD2, ALB1-GA, ALB8-uGA, ALB1B-uGA, L3316-GA1, L3315-GA2, L3316-GA3, L3316-GA4, DG12-GA1, DG12-GA2, ZAG-GA, MAG-GA1, MAG-GA2, PSD-1, or ABDstable disclosed in Nilvebrant, J., et al., The albumin-binding domain as a scaffold for protein engineering. Comput. Struct. Biotechnol. J. 2013 Sep. 1. In a further embodiment, the albumin-binding portion in the present invention comprises a non-immunoglobulin affinity protein. Examples of the non-immunoglobulin affinity protein comprise Affibody, Affilin, Anticalin, Atrimer, Avimer, Bycyclic peptide, Cys-knot, DARPin, FN3 (Adnectin), Fynomer, Kunitz domains, OBodies, and such (Simeon, R., et al., In vitro-engineered non-antibody protein therapeutics. Protein Cell 9, 3-14 (2018)).

In a preferred embodiment, albumin-binding portions in the present invention include albumin binding domain 3 (G148-GA3) (SEQ ID NO: 24) of the hemolytic streptococcus strain G148 protein G, or variants thereof. G148-GA3 consisting of 46 amino acid residues that form a stable 3-helix bundle has been reported to bind to albumins of humans, monkeys, mice, and such (Johansson, M. U. et al., (2002) Structure, Specificity, and Mode of Interaction for Bacterial Albumin-Binding Modules, J. Biol. Chem. 277(10):8114-8120).

Examples of the variants of G148-GA3 include an albumin-binding domain having an amino acid sequence represented by the following sequence (M. U. Johansson, I. M. Frick, H. Nilsson, et al., Structure, specificity, and mode of interaction for bacterial albumin-binding modules, J. Biol. Chem., 277 (10) (2002), pp. 8114-8120):

(SEQ ID NO: 69)
L[X2][X3]AKE[X7]AI[X10]ELK[X14][X15]GI[X18]SD
[X21]Y[X23][X24][X25]INKAKTVEGV[X36]ALK[X40]EIL
[X44][X45].

Herein,
[X2] is selected from A, D, K, L, Q, or S;
[X3] is selected from E, K, L, N, Q, or R;
[X7] is selected from A, D, E, K, L, or M;
[X10] is selected from A, I, K, L, N, Q, or R;
[X14] is selected from A, E, K, Q, R, or V;
[X15] is selected from A, K, L, N, or Y;
[X18] is selected from F, I, L, or Y, or does not exist;
[X21] is selected from F, I, L, or Y, or does not exist;
[X23] is selected from F, I, L, K, T, or V;
[X24] is selected from D, K, N, S, or T;
[X25] is selected from A, K, L, N, or Q;
[X36] is selected from E, K, M, N, T, or V;
[X40] is selected from A, D, E, N, or Q;
[X44] is selected from A, E, K, N, Q, or S, or does not exist; and
[X45] is selected from A, Q, or S, or does not exist.

Alternatively, examples of the variants of G148-GA3 include an albumin-binding domain having an amino acid sequence represented by the following sequence (WO2009/016043):

(SEQ ID NO: 70)
LAEAK[X6][[X7]A[X9][[X10]EL[[X13]KYGVSD
[X20]Y[[X23][[X24]I[X26][[X27]A[[X29]TVEGV
[X35]AL[X38][[X39][[X40]ILAALP.

Herein,
[X6] is selected from V or E;
[X7] is selected from L, E, or D;
[X9] is selected from N, L, or I;
[X10] is selected from R or K;
[X13] is selected from D or K;
[X20] is selected from Y or F;
[X23] is selected from N, R, or S;
[X24] is selected from V, I, L, M, F, or Y;
[X26] is selected from N, S, E, or D;
[X27] is selected from R, K, or N;
[X29] is selected from K or R;
[X35] is selected from D, N, Q, E, H, S, R, or K;
[X38] is selected from K, I, or T;
[X39] is selected from A, S, T, G, H, L, or D; and
[X40] is selected from H, E, or D.

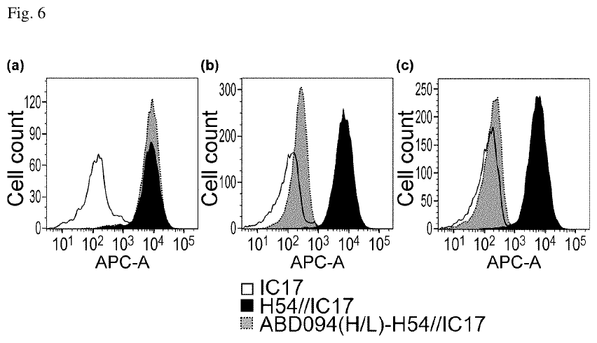

Alternatively, an example of the variant of G148-GA3 is ABD035 (SEQ ID NO: 23) (Jonsson A, et al., Engineering of a femtomolar affinity binding protein to human serum albumin. Protein Eng. Des. Sel. 2008 August; 21(8):515-27). ABD035, a G148-GA3 variant having improved binding activity for albumin, is known to bind strongly to human, rat, mouse, and monkey albumins, and bind also to rabbit and bovine serum albumins (see Table 11 and FIG. 6 of Jonsson, A. et al. mentioned above). In a preferred embodiment, an albumin-binding domain in the present invention comprises the amino acid sequence of SEQ ID NO: 23 (ABD035). Alternatively, an albumin-binding domain in the present invention consists of the amino acid sequence of SEQ ID NO: 23 (ABD035).

Further examples of the variant of G148-GA3 include an albumin-binding domain having an amino acid sequence represented by the following sequence (WO2012/004384):

(SEQ ID NO: 40)
LA[X3]AK[X6][X7]AN[X10]ELD[X14]YGVSDFYKRLI[X26]
KAKTVEGVEALK[X39][X40]IL[X43][X44]LP.

Herein,
[X3] is selected from E, S, Q, or C;
[X6] is selected from E, S, or C;
[X7] is selected from A or S;
[X10] is selected from A, S, or R;
[X14] is selected from A, S, C, or K;
[X26] is selected from D or E;
[X39] is selected from D or E;
[X40] is selected from A or E;
[X43] is selected from A or K;
[X44] is selected from A, S, or E;
L at position 45 is present or absent; and
P at position 46 is present or absent.

Preferred examples of the variants of G148-GA3 include an albumin-binding domain having an amino acid sequence represented by the following sequence:

(SEQ ID NO: 71)
LA[X3]AK[X6][X7]AN[X10]ELD[X14]YGVSDFYKRLI[X26]
KAKTVEGVEALK[X39][X40]IL[X43][X44]LP.

Herein,
[X3] is selected from E, S, or Q;
[X6] is selected from E or S;
[X7] is selected from A or S;
[X10] is selected from A, S, or R;
[X14] is selected from A, S, or K;
[X26] is selected from D or E;
[X39] is selected from D or E;
[X40] is selected from A or E;
[X43] is selected from A or K;
[X44] is selected from A, S, or E;
L at position 45 is present or absent; and
P at position 46 is present or absent.

More preferred examples of the variants of G148-GA3 include an albumin-binding domain having an amino acid sequence represented by the following sequence:

(SEQ ID NO: 72)
LA[X3]AK[X6][X7]AN[X10]ELD[X14]YGVSDFYKRLI[X26]
KAKTVEGVEALK[X39][X40]IL[X43][X44]LP.

Herein,
[X3] is selected from E or S;
[X6] is E;
[X7] is A;
[X10] is selected from A or R;
[X14] is selected from A, S, or K;
[X26] is D;
[X39] is D;
[X40] is A;
[X43] is A;
[X44] is A;
L at position 45 is present or absent; and
P at position 46 is present or absent.

Further examples of the variants of G148-GA3 include an albumin-binding domain having an amino acid sequence represented by the following sequence (WO2013177398):

(SEQ ID NO: 73)
LKEAKEKAIEELKKAGITSD[X21][X22]FDLINKA[X30][X31]
VEGVN[X37]LKD[X41]ILKA.

Herein,
[X21] is selected from A, K, or Y;
[X22] is selected from Y, A, V, or S;
[X30] is selected from K or D;
[X31] is selected from T or A;
[X37] is selected from V, Y, or A; and
[X41] is selected from E or Q.

Alternatively, an albumin-binding domain having an amino acid sequence represented by the following sequence is also an example of domains suitably used in polypeptide complexes of the present invention (WO2014048977):

(SEQ ID NO: 74)
LAEAKEAANAELDSYGVSDFYK[[X23]LIDKAKTVEGVEALKDAILAA
LP.

Herein,
[X23] is selected from K, R, N, and S.

Alternatively, an albumin-binding domain having an amino acid sequence represented by the following sequence is also an example of domains suitably used in polypeptide complexes of the present invention:

(SEQ ID NO: 41)
LA[X3]AKEAANAELD[X14]YGVSDFYKRLIDKAKTVEGVEALKDAILA
ALP.

Herein,
[X3] is E or S; and
[X14] is A, S, C, or K.

In the most preferable embodiment, an albumin-binding domain in the present invention comprises the amino acid sequence of SEQ ID NO: 4 (ABD094). Alternatively, an albumin-binding domain in the present invention consists of the amino acid sequence of SEQ ID NO: 4 (ABD094). ABD094 is a variant of ABD035. ABD035 was “deimmunized” by a modification that removes the T-cell epitope, and this resulted in the production of ABD094 which has reduced immunogenicity while maintaining stability, solubility, and high binding affinity (Frejd, F. (2012) Half-Life Extension by Binding to Albumin through an Albumin Binding Domain In: Kontermann, R., editor. Therapeutic Proteins: Strategies to Modulate Their Plasma Half-Lives. Weinheim: Wiley-VCH Verlag GmbH & Co). The amino acid sequence of ABD094 is the same as the amino acid sequence of PP013 disclosed in WO2012004384.

In one embodiment, albumin-binding domains in the present invention can comprise an amino acid sequence having at least 80%, 85%, or 90% sequence identity with the amino acid sequence of ABD035 (SEQ ID NO: 23). Preferably, albumin-binding domains in the present invention can comprise an amino acid sequence having at least 95%, 96%, 97%, 98%, or 99% sequence identity with the amino acid sequence of ABD035 (SEQ ID NO: 23).

Alternatively, albumin-binding domains in the present invention may be domains having 1, 2, 3, 4, 5, 6, 7, 8, 9, or 10 amino acid modifications (for example, addition(s), insertion(s), deletion(s), and substitution(s)) introduced to the amino acid sequence of ABD035 (SEQ ID NO: 23).

In one embodiment, albumin-binding domains in the present invention can comprise an amino acid sequence having at least 80%, 85%, or 90% sequence identity with the amino acid sequence of ABD094 (SEQ ID NO: 4). Preferably, albumin-binding domains in the present invention can comprise an amino acid sequence having at least 95%, 96%, 97%, 98%, or 99% sequence identity with the amino acid sequence of ABD094 (SEQ ID NO: 4).

Alternatively, albumin-binding domains in the present invention may be domains having 1, 2, 3, 4, 5, 6, 7, 8, 9, or 10 amino acid modifications (for example, addition(s), insertion(s), deletion(s), and substitution(s)) introduced to the amino acid sequence of ABD094 (SEQ ID NO: 4).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Amino-acid addition, deletion, substitution, and/or insertion can be performed by methods known in the art. For example, site-directed mutagenesis methods (Kunkel et al., Proc. Natl. Acad. Sci. USA 82, 488-492(1985)) and overlap extension PCR may be performed on a nucleic acid encoding an amino acid sequence. These methods can be appropriately applied alone or in combination.

Generally, one or multiple (for example, 10 or fewer, 9 or fewer, 8 or fewer, 7 or fewer, 6 or fewer, 5 or fewer, 4 or fewer, or 3 or fewer, or 2, 3, 4, 5, 6, 7, 8, 9, or 10, or more than 10) amino acid modifications (for example, conservative substitution(s), deletion(s), insertion(s), and/or addition(s)) in a protein do not affect its peptide function, or are known to even enhance the function of the original protein. Amino acid residues are classified, for example, into the following groups according to the properties of side chains included in their structures:
(1) hydrophobic: norleucine, methionine (Met), alanine (Ala), valine (Val), leucine (Leu), isoleucine (Ile);
(2) neutral hydrophilic: cysteine (Cys), serine (Ser), threonine (Thr), asparagine (Asn), glutamine (Gln);
(3) acidic: aspartic acid (Asp), glutamic acid (Glu);
(4) basic: histidine (His), lysine (Lys), arginine (Arg);
(5) residues that influence chain orientation: glycine (Gly), proline (Pro); and
(6) aromatic: tryptophan (Trp), tyrosine (Tyr), phenylalanine (Phe).

Exchanging amino acid residues within each of these groups is called conservative substitution; meanwhile, exchanging amino acid residues between different groups is called non-conservative substitution. Domains formed by modifying amino acid(s) contained in the albumin-binding domain having the amino acid sequence of SEQ ID NO: 23 or 4 with different amino acid(s) classified into a group of amino acids having similar properties are also included in the albumin-binding domains of the present invention. Albumin-binding domains in the present invention, however, may contain non-conservative modifications as long as they are functionally equivalent to the protein comprising the amino acid sequence of SEQ ID NO: 23 or 4.

### Second Antigen-Binding Portions (Target Antigen-Binding Portions)

As the second antigen-binding portion (target antigen-binding portion) of the present invention, a domain of any structure can be used as long as it binds to the target antigen of interest. The target antigen-binding portion may be, for example, an antibody fragment such as Fab, scFv, a domain antibody (dAb), and non-immunoglobulin affinity proteins. Examples of a domain antibody include a VH single domain, a VL single domain, VHH and Nanobody. Examples of non-immunoglobulin affinity proteins include Affibody, Affilin, Anticalin, Atrimer, Avimer, Bycyclic peptide, Cys-knot, DARPin, FN3 (Adnectin), Fynomer, Kunitz domains, and OBodies (Simeon, R., et al., In vitro-engineered non-antibody protein therapeutics. Protein Cell 9, 3-14 (2018)).

In one embodiment, the second antigen-binding portion may involve integration of amino acid deletion(s), substitution(s), insertion(s), or addition(s) into the amino acid sequence of an antigen-binding portion to enhance the pH-dependent binding ability of the antibody for the antigen. When the second antigen-binding portion comprises antibody variable region(s) (VH and/or VL), the amino acid mutation may be included in the antibody variable region(s), for example, in one or multiple HVR(s) (for example, CDR(s)). For example, mutations can include substitution of amino acids in one or multiple HVRs (for example, CDR(s)) of the antibody variable region with other amino acids. Alternatively, mutations can include substitution of one or multiple amino acids in at least one HVR (for example, CDR) of the antibody variable region with histidine(s). In one embodiment, “enhanced pH-dependent binding ability” means that the mutated antigen-binding portion shows larger acidic KD/neutral KD ratio or larger acidic kd/neutral kd ratio than the original “parent” antigen-binding portion prior to inducing the mutation (that is, the antigen-binding portion having low pH-dependence). In one embodiment, the mutated antigen-binding portion has an acidic KD/neutral KD ratio of 2 or more. Alternatively, the mutated antigen-binding portion has an acidic kd/neutral kd ratio of 2 or more.

### Target Antigens

In one embodiment, the second antigen-binding portion in the present invention binds to a target antigen different from the antigen of the first antigen-binding portion. That is, the second antigen-binding portion in the present invention binds to an antigen that is not a plasma protein (for example, albumin). The target antigen in the present invention only needs to be an antigen other than a plasma protein (for example, albumin), and its structure is not limited as long as it comprises an epitope to which the second antigen-binding portion (target antigen-binding portion) binds. In other words, the target antigen can be an inorganic or an organic substance. Furthermore, a target antigen in the present invention may be a soluble antigen, or a membrane-type antigen (membrane-bound antigen). Examples of the target antigen include the following molecules: 17-IA, 4-1BB, 4Dc, 6-keto-PGF1a, 8-iso-PGF2a, 8-oxo-dG, A1 adenosine receptor, A33, ACE, ACE-2, activin, activin A, activin AB, activin B, activin C, activin RIA, activin RIA ALK-2, activin RIB ALK-4, activin RIIA, activin RIIB, ADAM, ADAM10, ADAM12, ADAM15, ADAM17/TACE, ADAM8, ADAM9, ADAMTS, ADAMTS4, ADAMTS5, addressin, aFGF, ALCAM, ALK, ALK-1, ALK-7, alpha-1-antitrypsin, alpha-V/beta-1 antagonist, ANG, Ang, APAF-1, APE, APJ, APP, APRIL, AQP4, AR, ARC, ART, artemin, anti-Id, ASPARTIC, atrial natriuretic factor, av/b3 integrin, Axl, b2M, B7-1, B7-2, B7-H, B-lymphocyte stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMP, b-NGF, BOK, bombesin, bone-derived neurotrophic factor, BPDE, BPDE-DNA, BTC, complement factor 3 (C3), C3a, C4, C5, C5a, C10, CA125, CAD-8, calcitonin, cAMP, carcinoembryonic antigen (CEA), cancer associated antigen, cathepsin A, cathepsin B, cathepsin C/DPPI, cathepsin D, cathepsin E, cathepsin H, cathepsin L, cathepsin O, cathepsin S, cathepsin V, cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 protein), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CRTH2 (CD294), CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, PD1, PDL1, LAG3, TIM3, galectin-9, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor associated antigen, DAN, DCC, DcR3, DC-SIGN, complement regulatory factor (Decay accelerating factor), des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EGFRvIII, EMA, EMMPRIN, ENA, endothelin receptor, enkephalinase, eNOS, Eot, eotaxin 1, EpCAM, ephrin B2/EphB4, EphA2, EPO, ERCC, E-selectin, ET-1, factor IIa, factor VII, factor VIIIc, factor IX, fibroblast activation protein (FAP), Fas, FcR1, FEN-1, ferritin, FGF, FGF-19, FGF-2, FGF3, FGF-8, FGFR, FGFR-3, fibrin, FL, FLIP, Flt-3, Flt-4, follicle stimulating hormone, fractalkine, FZD1, FZD2, FZD3, FZD4, FZD5, FZD6, FZD7, FZD8, FZD9, FZD10, G250, Gas6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (myostatin), GDF-9, GDF-15 (MIC-1), GDNF, GFAP, GFRa-1, GFR-alpha 1, GFR-alpha 2, GFR-alpha 3, GITR, glucagon, Glut4, glycoprotein IIb/IIIa (GPIIb/IIIa), GM-CSF, gp130, gp72, GRO, growth hormone-releasing factor, hapten (NP-cap or NIP-cap), HB-EGF, HCC, HCMV gB envelope glycoprotein, HCMV gH envelope glycoprotein, HCMV UL, hematopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, high-molecular-weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, I-309, IAP, ICAM, ICAM-1, ICAM-3, ICE, ICOS, IFNg, Ig, IgA receptor, IgE, IGF, IGF binding protein, IGF-1R, IGFBP, IGF-I, IGF-II, IL, IL-1, IL-1R, IL-2, IL-2R, IL-3, IL-3R, IL-4, IL-4R, IL-5, IL-5R, IL-6, IL-6R, IL-7, IL-7R, IL-8, IL-9, IL-10, IL-11, IL-12, IL-13, IL-15, IL-18, IL-18R, IL-21, IL-23, IL-27, interferon (IFN)-alpha, INF-beta, INF-gamma, inhibin, iNOS, insulin A chain, insulin B chain, insulin-like growth factor 1, integrin alpha 2, integrin alpha 3, integrin alpha 4, integrin alpha 4/beta 1, integrin alpha 4/beta 7, integrin alpha 5 (alpha V), integrin alpha 5/beta 1, integrin alpha 5/beta 3, integrin alpha 6, integrin beta 1, integrin beta 2, interferon gamma, IP-10, I-TAC, JE, kallikrein 2, kallikrein 5, kallikrein 6, kallikrein 11, kallikrein 12, kallikrein 14, kallikrein 15, kallikrein L1, kallikrein L2, kallikrein L3, kallikrein L4, KC, KDR, keratinocyte growth factor (KGF), laminin 5, LAMP, LAP, LAP (TGF-1), latent TGF-1, latent TGF-1 bp1, LBP, LDGF, LECT2, lefty, Lewis-Y antigen, Lewis-Y associated antigen, LFA-1, LFA-3, Lfo, LIF, LIGHT, lipoprotein, LIX, LKN, Lptn, L-selectin, LT-a, LT-b, LTB4, LTBP-1, lung surface, luteinizing hormone, lymphotoxin beta receptor, Mac-1, MAdCAM, MAG, MAP2, MARC, MBP (myelin basic protein), MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MOG (myelin-oligodendrocyte glycoprotein), MMP-8, MMP-9, MPIF, Mpo, MSK, MSP, mucin (Muc1), MUC18, Mullerian-inhibiting substance, Mug, MuSK, NAIP, NAP, NCAD, N-C adherin, NCA 90, NCAM, neprilysin, neurotrophin-3, -4, or -6, neurturin, nerve growth factor (NGF), NGFR, NGF-beta, NMDAR (NMDA-type glutamate receptor), nNOS, NO, NOS, Npn, NRG-3, NT, NTN, OB, OGG1, OPG, OPN, OSM, OSM receptor, OX40L, OX40R, p150, p95, PADPr, parathyroid hormone, PARC, PARP, PBR, PBSF, PCAD, P-cadherin, PCNA, PDGF, PDGF-D, PDK-1, PECAM, PEM, PF4, PGE, PGF, PGI2, PGJ2, PIN, PLA2, placental alkaline phosphatase (PLAP), PlGF, PLP, PP14, proinsulin, prorelaxin, protein C, PS, PSA, PSCA, prostate-specific membrane antigen (PSMA), PTEN, PTHrp, Ptk, PTN, R51, RANK, RANKL, RANTES, relaxin A chain, relaxin B chain, renin, respiratory syncytial virus (RSV) F, RSV Fgp, Ret, rheumatoid factor, RLIP76, RPA2, RSK, S100, SCF/KL, SDF-1, SERINE, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptor (for example, T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta 1, TGF-beta 2, TGF-beta 3, TGF-beta 4, TGF-beta 5, thrombin, thymus Ck-1, thyroid-stimulating hormone, Tie, TIMP, TIQ, tissue factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 ligand, TL2), TNFSF11 (TRANCE/RANK ligand ODF, OPG ligand), TNFSF12 (TWEAK Apo-3 ligand, DR3 ligand), TNFSF13 (APRIL TALL2), TNFSF13B (BAFF BLYS, TALL1, THANK, TNFSF20), TNFSF14 (LIGHT HVEM ligand, LTg), TNFSF15 (TL1A/VEGI), TNFSF18 (GITR ligand AITR ligand, TL6), TNFSF1A (TNF-α Connectin, DIF, TNFSF2), TNFSF1B (TNF-b LTa, TNFSF1), TNFSF3 (LTb TNFC, p33), TNFSF4 (OX40 ligand gp34, TXGP1), TNFSF5 (CD40 ligand CD154, gp39, HIGM1, IMD3, TRAP), TNFSF6 (Fas ligand Apo-1 ligand, APT1 ligand), TNFSF7 (CD27 ligand CD70), TNFSF8 (CD30 ligand CD153), TNFSF9 (4-1BB ligand CD137 ligand), TP-1, t-PA, Tpo, TRAIL, TRAIL R, TRAIL-R1, TRAIL-R2, TRANCE, transferrin receptor, TRF, Trk, TROP-2, TLR (Toll-like receptor)1, TLR2, TLR3, TLR4, TLR5, TLR6, TLR7, TLR8, TLR9, TLR10, TSG, TSLP, tumor-associated antigen CA125, tumor associated antigen expressing Lewis-Y associated carbohydrates, TWEAK, TXB2, Ung, uPAR, uPAR-1, urokinase, VCAM, VCAM-1, VECAD, VE-Cadherin, VE-cadherin-2, VEFGR-1 (flt-1), VEGF, VEGFR, VEGFR-3 (flt-4), VEGI, VIM, virus antigen, VLA, VLA-1, VLA-4, VNR integrin, von Willebrand factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9B, WNT10A, WNT10B, WNT11, WNT16, XCL1, XCL2, XCR1, XEDAR, XIAP, XPD, HMGB1, IgA, A beta, CD81, CD97, CD98, DDR1, DKK1, EREG, Hsp90, IL-17/IL-17R, IL-20/IL-20R, oxidized LDL, PCSK9, prekallikrein, RON, TMEM16F, SOD1, Chromogranin A, Chromogranin B, tau, VAP1, high-molecular-weight kininogen, IL-31, IL-31R, Nav1.1, Nav1.2, Nav1.3, Nav1.4, Nav1.5, Nav1.6, Nav1.7, Nav1.8, Nav1.9, EPCR, C1, C1q, C1r, C1s, C2, C2a, C2b, C3, C3a, C3b, C4, C4a, C4b, C5, C5a, C5b, C6, C7, C8, C9, factor B, factor D, factor H, properdin, sclerostin, fibrinogen, fibrin, prothrombin, thrombin, tissue factor, factor V, factor Va, factor VII, factor VIIa, factor VIII, factor VIIIa, factor IX, factor IXa, factor X, factor Xa, factor XI, factor XIa, factor XII, factor XIIa, factor XIII, factor XIIIa, TFPI, antithrombin III, EPCR, thrombomodulin, TAPI, tPA, plasminogen, plasmin, PAI-1, PAI-2, GPC3, Syndecan-1, Syndecan-2, Syndecan-3, Syndecan-4, Alpha synuclein, LPA, SIP, LGI1, CASPR2, Glycine receptor, GABAA receptor, GABAB receptor, AMPA receptor, mGluR1, mGluR5, DPPX, D2R, IgLON5, Neurexin 3 alpha, Amphiphysin, Septin 5, Synapsin, and receptors for hormones and growth factors.

Examples of particularly preferred target antigens include ADAM10, EGFRvIII, EphA2, CD52, HLA-DR, Alpha synuclein, TNF-alpha, IL-6R, C1q, C3, and CD20.

### Albumin Concentration in Tissues

Albumin is the most abundant protein in blood, binds to various substances such as bilirubins, ions, fatty acids, and foreign ligands (drugs) in the blood, and is involved in the transport of substances in a body. Albumin is synthesized mainly in the liver, 30 to 40% of it circulate in the blood, and it also exists in the interstitial fluid of various organs (www.ncbi.nlm.nih.gov/books/NBK459198/). On the other hand, albumin concentration in the cerebrospinal fluid, skeletal muscles, adipose tissues, skin, ears, and eyes is known to be lower than in the blood. Especially, transport of substances to the brain is limited, and albumin concentration is particularly low in the cerebrospinal fluid and brain tissues. Albumin concentration is approximately 600 micro M in human plasma, and is approximately 3 micro M in human cerebrospinal fluid (CSF) (see, Kay, A. D., et al., CSF and serum concentrations of albumin and IgG in Alzheimer's disease., Neurobiol. Aging. 1987 January-February; 8(1):21-5; and Seyfert, S., What determines the CSF concentrations of albumin and plasma-derived IgG? J. Neurol. Sci. 2004 Apr. 15; 219(1-2):31-3). Furthermore, according to reports, albumin concentration in mouse plasma is more than approximately 160-times the albumin concentration in mouse cerebrospinal fluid (CSF) (Liddelow, Shane A., et al., Cellular specificity of the blood-CSF barrier for albumin transfer across the choroid plexus epithelium. PloS One. 2014; 9(9):e106592).

### Binding Activity Dependent on Plasma Protein Concentration

In one embodiment, in an environment where a polypeptide complex of the present invention has greater binding activity to either one of a plasma protein and a target antigen, the complex shows smaller binding activity to the other one of the two. Plasma protein concentration is high in systemic blood and low in CNS tissues. In an environment where the plasma protein concentration is high, such as in plasma, polypeptide complexes of the present invention show strong binding activity to plasma protein and weak binding activity to target antigen. On the other hand, in an environment where the plasma protein concentration is low, such as in CNS tissues, the complexes show strong binding activity to target antigen and weak or hardly any binding activity to plasma protein.

In the present invention, “binding activity” refers to the strength of the sum total of noncovalent interactions between one or more binding sites of a polypeptide complex and its binding partner (e.g., a target antigen). Herein, “binding activity” is not strictly limited to a 1:1 interaction between members of a binding pair (e.g., polypeptide complex and target antigen). For example, when the members of a binding pair reflect a monovalent 1:1 interaction, the binding activity is particularly called the intrinsic binding affinity (affinity). When a member of a binding pair is capable of both monovalent binding and multivalent binding, the binding activity is the sum of each binding strength. The binding activity of a molecule X for its partner Y can generally be represented by the dissociation constant (KD) or “binding amount of analyte per unit amount of ligand” (hereinbelow, may be referred to as “binding amount”). Those skilled in the art would understand that, generally, lower value of dissociation constant (KD) means higher binding activity, and higher value of “binding amount of analyte per unit amount of ligand” or “binding amount” means higher binding activity. Binding activity can be measured by common methods known in the art, including those described herein. Specific illustrative and exemplary embodiments for measuring binding activity are described in the following.

In one embodiment, polypeptide complexes of the present invention may be polypeptide complexes whose binding activity to a target antigen in the presence of plasma protein is lower than the binding activity to the target antigen in the absence of plasma protein.

In another embodiment, polypeptide complexes of the present invention may be polypeptide complexes whose binding activity to a target antigen in the presence of plasma protein at a first concentration is different from the binding activity to the target antigen in the presence of plasma protein at a second concentration. In one embodiment, polypeptide complexes of the present invention may be polypeptide complexes whose binding activity to a target antigen in the presence of plasma protein at a first concentration is lower than the binding activity to the target antigen in the presence of plasma protein at a second concentration which is a concentration lower than the first concentration. In one embodiment, the term “high concentration” mentioned later can be presented as an example of the first concentration, and the term “low concentration” mentioned later can be presented as an example of the second concentration. In this case, the binding activity to a target antigen in the presence of plasma protein at a first concentration will have a lower value than the binding activity to the target antigen in the presence of plasma protein at a second concentration.

In a certain embodiment, examples of a first concentration and a second concentration are about 20, 25, 30, 35, 40, 45, 50, 55, 60, 65, 70, 75 or 80 mg/mL and about 0.05, 0.10, 0.15, 0.20, 0.25, 0.30, 0.35, 0.40, 0.45 or 0.50 mg/mL, respectively. More preferably, examples of a first concentration and a second concentration are 50 mg/mL and 0.25 mg/mL, respectively. More specifically, polypeptide complexes of the present invention may be polypeptide complexes whose binding activity to a target antigen in the presence of a plasma protein (for example, albumin) at 50 mg/mL is lower than the binding activity to the target antigen in the presence of the plasma protein (for example, albumin) at 0.25 mg/mL.

In another embodiment, polypeptide complexes of the present invention may be polypeptide complexes whose binding activity to a target antigen in a human plasma sample is lower than the binding activity to the target antigen in the absence of human plasma protein.

In a further embodiment, polypeptide complexes of the present invention may be polypeptide complexes whose binding activity to a target antigen in a human plasma sample is lower than the binding activity to the target antigen in a human cerebrospinal fluid (CSF) sample.

In several embodiments, the binding activity to a target antigen of a polypeptide complex of the present invention can be presented by a dissociation constant (KD) value. Alternatively, when the target antigen is a membrane-type molecule, apparent dissociation constant (apparent KD) can be used. The dissociation constant (KD) and apparent dissociation constant (apparent KD) can be determined by methods known to those skilled in the art, for example, using Biacore (GE Healthcare), a Scatchard plot, a flow cytometer, or such. In the present invention, when measuring the binding activity of a polypeptide complex to a target antigen under different plasma protein concentrations, conditions other than the plasma protein concentration are preferably kept the same.

In one embodiment, the binding activity to a target antigen (KD) of a polypeptide complex is most preferably calculated from the amount of the complex formed between the polypeptide complex and the target antigen under a certain plasma protein concentration. As an example of such a method for calculating the binding activity (KD), the case where albumin is the plasma protein is shown below.

First, a polypeptide complex of the present invention is added to a solution or sample containing a certain concentration of albumin (for example, a plasma sample or a cerebrospinal fluid (CSF) sample), and this is incubated at room temperature. A target antigen prepared at stepwise concentrations is added to the solution or sample, and this is incubated at room temperature. The polypeptide complex of the present invention and the target antigen can be added to the solution or sample simultaneously, or the polypeptide complex can be added first. Furthermore, when the target antigen is intrinsically present in the sample (for example, a plasma sample or a CSF sample) at an amount sufficient for the sensitivity of the measurement system, addition of the target antigen is unnecessary. Next, to detect the complex formed by the polypeptide complex and the target antigen, a labeled antibody that specifically binds to the polypeptide complex (secondary antibody) is additionally added to the solution or the sample, and this is incubated at room temperature and then O/N incubated at 4 degrees C. For example, when the polypeptide complex comprises an Fc region lacking the two amino acids, glycine and lysine, from the C terminus of the heavy chain (delta GK), an anti-delta GK antibody (an antibody that specifically binds to the Fc region lacking the two amino acids, glycine and lysine, from the C terminus of the heavy chain) can be used as the secondary antibody. As the label, for example, AF647 can be used. The labeled secondary antibody can be added after dilution in an appropriate buffer, for example, PBS 0.05% Tween 20.

The complexes formed between the polypeptide complex and the target antigen can be measured using Gyrolab xP. For the measurements using Gyrolab xP, Gyrolab Bioaffy 200 (P0004180, Gyros Protein Technologies) or such is used as the measurement disc. First, biotinylated labeled anti-target antigen antibody (“solid-phased antibody”) is added to the measurement disc and solid-phased on the reaction layer in the disc. In this case, the solid-phased antibody can be added after dilution in an appropriate buffer (for example, PBS 0.05% Tween 20) as necessary. Here, the solid-phased antibody binds to the same target antigen as the polypeptide complex, but preferably they bind to different epitopes of the target antigen molecule. When the incubated sample described above (the mixed solution to which the polypeptide complex, the target antigen, and the labeled secondary antibody have been added) is added thereto, the “polypeptide complex-target antigen-labeled secondary antibody” complex becomes captured by the solid-phased antibody. The amount of complex captured is detected by the signal from the label, for example, the fluorescence signal from AF647. The fluorescence signal of AF647 is analyzed by Gyro Evaluator (Gyros Protein Technologies). Then, from the concentration of the added target antigen and the obtained AF647 fluorescence signal, the target antigen concentration that gives ½ the fluorescence signal of 100% binding strength is calculated to provide the KD value of the polypeptide complex for the target antigen. When measuring the binding activity of a polypeptide complex to a target antigen at different albumin concentrations, conditions other than the albumin concentration are preferably kept the same.

When the target antigen is not a soluble antigen (for example a membrane-type antigen), measuring the amount of complex formed by the polypeptide complex and the target antigen may be difficult using Gyrolab xP. In such cases, the ELISA method may be used as the next preferable method for measuring KD. The method for calculating KD using the ELISA method is shown below, using as an example, the case where the plasma protein is albumin.

First, to a solution or sample containing a certain concentration of albumin (for example, a plasma sample or a cerebrospinal fluid (CSF) sample), a polypeptide complex of the present invention prepared at stepwise concentrations is added, and this is incubated at room temperature. The incubated solution is added to an ELISA plate to which the target antigen has been solid-phased or to an ELISA plate coated with target antigen-expressing cells, and this is incubated at room temperature. After removing the polypeptide complex of the present invention that did not bind to the target antigen by a washing operation, a labeled antibody that binds specifically to the polypeptide complex (secondary antibody) is added to the sample to detect the complex formed by the polypeptide complex and the target antigen, and this is incubated at room temperature. For example, when the polypeptide complex comprises an Fc region that lacks the two amino acids, glycine and lysine, from the C terminus of the heavy chain (delta GK), an anti-delta GK antibody (an antibody that specifically binds to the Fc region lacking the two amino acids, glycine and lysine, from the C terminus of the heavy chain) can be used as the secondary antibody. As the label, for example, horseradish peroxidase can be used. The labeled secondary antibody can be added after dilution in an appropriate buffer, for example, PBS 0.05% Tween 20. When using secondary antibodies that are not labeled, detection can be carried out by adding an antibody that recognizes the secondary antibody. Then, from the detected label signal, the target antigen concentration that gives ½ the signal of 100% binding strength is calculated to provide the KD value of the polypeptide complex for the target antigen. When measuring the binding activity of a polypeptide complex to a target antigen at different albumin concentrations, conditions other than the albumin concentration are preferably kept the same.

In another embodiment, the binding activity of the polypeptide complex to the target antigen in mouse plasma or cerebrospinal fluid (CSF) can be calculated from (1) the total polypeptide complex concentration, (2) the concentration of free target antigen (target antigen not bound to the polypeptide complex), and (3) the concentration of bound target antigen (target antigen bound to the polypeptide complex) in the mouse plasma or mouse CSF after administration of the polypeptide complex to mice. Herein, binding activity (KD) calculated in this manner is also called in vivo affinity (KD).

When measuring the target antigen concentration in mouse plasma, a suitable amount of a polypeptide complex of the present invention is administered to mice (for example, 6 to 8-weeks-old male C57BL/6J mice) through the tail vein. Thereafter, an appropriate amount of the target antigen is administered at an appropriate timing, for example, 10 or 20 minutes after administration of the polypeptide complex. When the target antigen is intrinsically present, target antigen administration is not necessary. Thereafter, blood is collected at an appropriate timing, for example, 5 minutes after target antigen administration, and the plasma is obtained by centrifugation (12,000 rpm, 4 degrees C., 5 minutes).

When measuring the target antigen concentration in mouse cerebrospinal fluid (CSF), an appropriate amount of a polypeptide complex of the present invention is administered to the lateral cerebroventricle of mice (for example, 6 to 8-weeks-old male C57BL/6J mice). Then at an appropriate timing, for example, 10 or 20 minutes after polypeptide complex administration, an appropriate amount of the target antigen is administered. When the target antigen is present intrinsically, administration of the target antigen is not necessary. Thereafter, at an appropriate timing, for example, 10 or 90 minutes after target antigen administration, CSF is collected.

The total polypeptide complex concentration in mouse plasma or CSF can be measured by enzyme-linked immunosorbent assay (ELISA). For example, first a capturing antibody (an antibody that binds to the polypeptide complex) is added to the plate. Next, a blocking solution is added to the plate, and then a plasma sample or a CSF sample (which may be diluted as necessary) is added. Then, a biotin-labeled anti-polypeptide complex antibody (detection antibody) and a Streptavidin-labeled secondary detection antibody are added. Finally, a substrate (for example, tetramethylbenzidine) is added, and then the absorbance at 650 nm is measured on an absorptiometer, and the polypeptide complex concentration is calculated based on the absorbance.

The concentration of free target antigen in mouse plasma or CSF can be measured on a fully automated ELISA system (Gyrolab xP workstation). For example, first, solutions for preparing the calibration curve of the target antigen are prepared, and next, a solution containing a labeled (for example, biotin-labeled) anti-target antigen antibody (“capturing-molecule solution”) is prepared. Additionally, a solution containing a labeled (for example, Alexa Fluor (registered trademark) 647-labeled) anti-target antigen antibody (“detecting-molecule solution”) is prepared. The capturing-molecule solution, the plasma sample or CSF sample (which may be diluted as necessary), and the detecting-molecule solution are placed onto a PCR plate in this order, and then the PCR plate is sealed with a plate sealer. The measurement can be carried out after placing the PCR plate and the Bioaffy 200 disc in the Gyrolab xP workstation. Using the data processed by the Gyros Evaluater 3.6.2.30 software and the calibration curve concentrations, the concentration of free sIL6R in plasma is calculated by logistic regression using the XLfit 5.5.0.5 software.

The concentration of bound target antigen in mouse plasma or CSF can be measured using an electrospray ionization mass spectrometer connected to a high-performance liquid chromatography (LC/ESI-MS/MS). For example, first, solutions for preparing the calibration curve of the target antigen are prepared. Next, the calibration curve solutions and a plasma sample are added to magnetic beads to which antibodies that specifically bind to a polypeptide complex (secondary antibodies) are solid-phased, this is shaken, and the shaken magnetic beads are washed. When the polypeptide complex comprises an Fc region lacking the two amino acids, glycine and lysine, from the C terminus of the heavy chain (delta GK), an anti-delta GK antibody (an antibody that specifically binds to the Fc region lacking the two amino acids, glycine and lysine, from the C terminus of the heavy chain) can be used as the secondary antibody. Next, the magnetic beads are suspended in ammonium bicarbonate containing urea, dithiothreitol, and lysozyme (chicken egg white), and then shaken. Subsequently, iodoacetamide is added, and this is shaken. Next, ammonium bicarbonate containing sequencing grade modified trypsin is added, and the sample after this addition is shaken. Trifluoroacetic acid is added to this to stop the reaction. The enzyme-digested sample prepared this way is used in the analysis by LC/ESI-MS/MS. Xevo TQ-S triple quadrupole instrument (Waters) connected to I-class plus UPLC (Waters) can be used for the LC/ESI-MS/MS analysis, and human IL-6R-specific peptide HVVQLR (SEQ ID NO: 75) is detected by selected reaction monitoring (SRM). The concentration of bound sIL6R in plasma is calculated using the analysis software Masslynx Ver.4.1 (Waters).

Using the total polypeptide complex concentration, free target antigen concentration, and bound target antigen concentration in plasma or CSF measured as described above, the in vivo affinity (KD) can be calculated using the following equation,

$$in vivo KD = \frac{( A {b}_{total} - IC ) \times {Ag}_{free}}{IC}$$

Herein, in vivo KD indicates the in vivo affinity to the target antigen, Abₜₒₜₐₗ indicates the total polypeptide complex concentration, Ag_{free} indicates the free target antigen concentration, and IC indicates the bound target antigen concentration.

Herein, regarding in vivo affinity (KD) in plasma, when there are multiple polypeptide complex doses, the larger value among the multiple calculated in vivo affinities (KDs) can be used. Regarding in vivo affinity (KD) in CSF, when there are multiple evaluation timings, the smaller value among the multiple calculated in vivo affinities (KDs) can be used. Thereafter, the in vivo affinity (KD) ratio between plasma and CSF is calculated by dividing in vivo affinity (KD) in CSF by the in vivo affinity (KD) in plasma. When the in vivo affinity (KD) ratio is larger, this means that the difference in binding activities in the plasma and CSF is large, and this suggests that such polypeptide complexes are more preferable as plasma protein switch molecules.

In one embodiment, when comparing the KD value, apparent KD value, or in vivo KD value of a polypeptide complex for a target antigen in the presence of plasma protein, with the KD value, apparent KD value, or in vivo KD value of the polypeptide complex for the target antigen in the absence of plasma protein, the latter value is smaller than the former value.

Alternatively, in another embodiment, when comparing the KD value, apparent KD value, or in vivo KD value of a polypeptide complex for a target antigen in the presence of a plasma protein at a high or higher concentration, with the KD value, apparent KD value, or in vivo KD value of the polypeptide complex for the target antigen in the presence of the plasma protein at low or lower concentration, the latter value is smaller than the former value.

Alternatively, in another embodiment, when comparing the KD value, apparent KD value, or in vivo KD value of a polypeptide complex for a target antigen in a human plasma sample, with the KD value, apparent KD value, or in vivo KD value of the polypeptide complex for the target antigen in the absence of human plasma protein, the latter value is smaller than the former value.

Alternatively, in another embodiment, when comparing the KD value, apparent KD value, or in vivo KD value of a polypeptide complex for a target antigen in a human plasma sample, with the KD value, apparent KD value, or in vivo KD value of the polypeptide complex for the target antigen in a human cerebrospinal fluid (CSF) sample, the latter value is smaller than the former value.

Alternatively, in another embodiment, when comparing the KD value, apparent KD value, or in vivo KD value of a polypeptide complex for a target antigen in the presence of a plasma protein (for example, albumin) at about 20, 25, 30, 35, 40, 45, 50, 55, 60, 65, 70, 75 or 80 mg/mL, with the KD value, apparent KD value, or in vivo KD value of the polypeptide complex for the target antigen in the presence of the plasma protein (for example, albumin) at about 0.05, 0.10, 0.15, 0.20, 0.25, 0.30, 0.35, 0.40, 0.45 or 0.50 mg/mL, the latter value is smaller than the former value. Preferably, when comparing the KD value, apparent KD value, or in vivo KD value of a polypeptide complex for a target antigen in the presence of a plasma protein (for example, albumin) at 50 mg/mL, with the KD value, apparent KD value, or in vivo KD value of the polypeptide complex for the target antigen in the presence of the plasma protein (for example, albumin) at 0.25 mg/mL, the latter value is smaller than the former value.

In one embodiment, the smaller KD value, apparent KD value, or in vivo KD value can be, for example, 9×10⁻⁷ M or less, 8×10⁻⁷ M or less, 7×10⁻⁷ M or less, 6×10⁻⁷ M or less, 5×10⁻⁷ M or less, 4×10⁻⁷ M or less, 3×10⁻⁷ M or less, 2×10⁻⁷ M or less, 1×10⁻⁷ M or less, 9×10⁻⁸ M or less, 8×10⁻⁸ M or less, 7×10⁻⁸ M or less, 6×10⁻⁸ M or less, 5×10⁻⁸ M or less, 4×10⁻⁸ M or less, 3×10⁻⁸ M or less, 2×10⁻⁸ M or less, 1×10⁻⁸ M or less, 9×10⁻⁹ M or less, 8×10⁻⁹ M or less, 7×10⁻⁹ M or less, 6×10⁻⁹ M or less, 5×10⁻⁹ M or less, 4×10⁻⁹ M or less, 3×10⁻⁹ M or less, 2×10⁻⁹ M or less, 1×10⁻⁹ M or less, 9×10⁻¹⁰ M or less, 8×10⁻¹⁰ M or less, 7×10⁻¹⁰ M or less, 6×10⁻¹⁰ M or less, 5×10⁻¹⁰ M or less, 4×10⁻¹⁰ M or less, 3×10⁻¹⁰ M or less, 2×10⁻¹⁰ M or less, 1×10⁻¹⁰ M or less, 9×10⁻¹¹ M or less, 8×10⁻¹¹ M or less, 7×10⁻¹¹ M or less, 6×10⁻¹¹ M or less, 5×10⁻¹¹ M or less, 4×10⁻¹¹ M or less, 3×10⁻¹¹ M or less, 2×10⁻¹¹ M or less, or 1×10⁻¹¹ M or less.

In one embodiment, the larger KD value, apparent KD value, or in vivo KD value can be, for example, 1×10⁻⁹ M or more, 2×10⁻⁹ M or more, 3×10⁻⁹ M or more, 4×10⁻⁹ M or more, 5×10⁻⁹ M or more, 6×10⁻⁹ M or more, 7×10⁻⁹ M or more, 8×10⁻⁹ M or more, 9×10⁻⁹ M or more, 1×10⁻⁸ M or more, 2×10⁻⁸ M or more, 3×10⁻⁸ M or more, 4×10⁻⁸ M or more, 5×10⁻⁸ M or more, 6×10⁻⁸ M or more, 7×10⁻⁸ M or more, 8×10⁻⁸ M or more, 9×10⁻⁸ M or more, 1×10⁻⁷ M or more, 2×10⁻⁷ M or more, 3×10⁻⁷ M or more, 4×10⁻⁷ M or more, 5×10⁻⁷ M or more, 6×10⁻⁷ M or more, 7×10⁻⁷ M or more, 8×10⁻⁷ M or more, 9×10⁻⁷ M or more, 1×10⁻⁶ M or more, 2×10⁻⁶ M or more, 3×10⁻⁶ M or more, 4×10⁻⁶ M or more, 5×10⁻⁶ M or more, 6×10⁻⁶ M or more, 7×10⁻⁶ M or more, 8×10⁻⁶ M or more, or 9×10⁻⁶ M or more.

In one embodiment, the ratio of the KD value, apparent KD value, or in vivo KD value in the presence of plasma protein or in the presence of plasma protein at a high concentration with respect to the KD value, apparent KD value, or in vivo KD value in the absence of plasma protein or in the presence of plasma protein at a low concentration can be, for example, 2-fold or more, 3-fold or more, 4-fold or more, 5-fold or more, 6-fold or more, 7-fold or more, 8-fold or more, 9-fold or more, 10-fold or more, 11-fold or more, 12-fold or more, 13-fold or more, 14-fold or more, 15-fold or more, 16-fold or more, 17-fold or more, 18-fold or more, 19-fold or more, 20-fold or more, 21-fold or more, 22-fold or more, 23-fold or more, 24-fold or more, 25-fold or more, 26-fold or more, 27-fold or more, 28-fold or more, 29-fold or more, 30-fold or more, 31-fold or more, 32-fold or more, 33-fold or more, 34-fold or more, 35-fold or more, 36-fold or more, 37-fold or more, 38-fold or more, 39-fold or more, 40-fold or more, 50-fold or more, 100-fold or more, 200-fold or more, 300-fold or more, 500-fold or more, 1×10³-fold or more, 2×10³-fold or more, 3×10³-fold or more, 5×10³-fold or more, 1×10⁴-fold or more, 2×10⁴-fold or more, 3×10⁴-fold or more, 5×10⁴-fold or more, or 1×10⁵-fold or more.

Furthermore, in one embodiment, in the presence of plasma protein or in the presence of plasma protein at a high concentration, the KD value, apparent KD value, or in vivo KD value of a polypeptide complex of the present invention for a target antigen can be a larger value compared to that of a control polypeptide complex having the same structure as the polypeptide complex of the present invention except that it lacks the first antigen-binding portion, for example, by 2-fold or more, 3-fold or more, 4-fold or more, 5-fold or more, 6-fold or more, 7-fold or more, 8-fold or more, 9-fold or more, 10-fold or more, 11-fold or more, 12-fold or more, 13-fold or more, 14-fold or more, 15-fold or more, 16-fold or more, 17-fold or more, 18-fold or more, 19-fold or more, 20-fold or more, 30-fold or more, 50-fold or more, 100-fold or more, 200-fold or more, 300-fold or more, 500-fold or more, 1×10³-fold or more, 2×10³-fold or more, 3×10³-fold or more, 5×10³-fold or more, 1×10⁴-fold or more, 2×10⁴-fold or more, 3×10⁴-fold or more, 5×10⁴-fold or more, or 1×10⁵-fold or more.

Alternatively, as the value for antigen-binding activity, kd (dissociation rate constant) can be used when the target antigen is a soluble molecule, and apparent kd (apparent dissociation rate constant) can be used when the target antigen is a membrane-type molecule. The kd (dissociation rate constant) and apparent kd (apparent dissociation rate constant) can be measured by methods known to those skilled in the art, and for example, Biacore (GE healthcare), flow cytometer, and such may be used.

Alternatively, as a value for antigen-binding activity, ka (association rate constant) can be used when the target antigen is a soluble molecule, and apparent ka (apparent association rate constant) can be used when the target antigen is a membrane-type molecule. The ka (association rate constant) and apparent ka (apparent association rate constant) can be measured by methods known to those skilled in the art, and for example, Biacore (GE healthcare), flow cytometer, and such may be used for the measurements.

In another embodiment, binding activity of polypeptide complexes of the present invention may be expressed, for example, by the binding amount of target antigen to polypeptide complexes. For example, in surface plasmon resonance assays, binding amount of a polypeptide complex immobilized on a sensor chip and binding amount of a target antigen further bound to this are each measured as resonance unit (RU). Antigen binding activity may be expressed using the binding amount of target antigen obtained therefrom as an indicator, or may alternatively be expressed using the value obtained by dividing the binding amount of target antigen by the binding amount of polypeptide complex (that is, the binding amount of target antigen per unit amount of polypeptide complex) as an indicator.

In several embodiments, when the binding amount of target antigen in the presence of plasma protein and the binding amount of target antigen in the absence of plasma protein are compared, the latter value is larger than the former value.

In another embodiment, when the binding amount of target antigen in the presence of plasma protein at a high concentration or higher concentration and the binding amount of target antigen in the presence of plasma protein at a low concentration or lower concentration are compared, the latter value is larger than the former value.

Alternatively, in another embodiment, when the binding amount of target antigen in a human plasma sample and the binding amount of target antigen in the absence of human plasma protein are compared, the latter value is larger than the former value.

Alternatively, in another embodiment, when the binding amount of target antigen in a human plasma sample and the binding amount of target antigen in a human cerebrospinal fluid (CSF) sample are compared, the latter value is larger than the former value.

Alternatively, in another embodiment, when the binding amount of target antigen in the presence of a plasma protein (for example, albumin) at about 20, 25, 30, 35, 40, 45, 50, 55, 60, 65, 70, 75 or 80 mg/mL and the binding amount of target antigen in the presence of the plasma protein (for example, albumin) at about 0.05, 0.10, 0.15, 0.20, 0.25, 0.30, 0.35, 0.40, 0.45 or 0.50 mg/mL are compared, the latter value is larger than the former value. Preferably, when the binding amount of target antigen in the presence of a plasma protein (for example, albumin) at 50 mg/mL and the binding amount of target antigen in the presence of the plasma protein (for example, albumin) at 0.25 mg/mL are compared, the latter value is larger than the former value.

Furthermore, in one embodiment, the binding activity of a polypeptide complex of the present invention to a target antigen can be expressed by the amount of complex formed between the polypeptide complex and the target antigen under a certain plasma protein concentration. For example, in the measurement using Gyrolab xP, one can calculate the fluorescence signal value of the complex formed by a polypeptide complex of the present invention and a target antigen, when defining the fluorescence signal obtained from the complex formed between the target antigen and a control polypeptide complex as 100, the control polypeptide complex having the same structure as the polypeptide complex of the present invention except that it lacks the first antigen-binding portion. In this case, the fluorescence signal value of the complex formed by a polypeptide complex of the present invention and a target antigen can be, for example, 10% or less, 20% or less, 30% or less, 40% or less, 50% or less, 60% or less, 70% or less, 80% or less, 90% or less, or 95% or less, as compared to the fluorescence signal value obtained from the complex formed between the target antigen and the control polypeptide complex having the same structure as the polypeptide complex of the present invention except that it lacks the first antigen-binding portion, in the presence of plasma protein or in the presence of plasma protein at a high concentration. The amount of the captured complex is detected by the signal of the label, for example, the fluorescence signal of AF647. The fluorescence signal of AF647 is analyzed by Gyro Evaluator (Gyros Protein Technologies). Then, from the concentration of the added target antigen and the obtained AF647 fluorescence signal, the antigen concentration that gives ½ the fluorescence signal of 100% binding strength is calculated to provide the KD value of the evaluated antibody for the bound antigen. When measuring the binding activity of a polypeptide complex to a target antigen at different plasma protein concentrations, conditions other than the plasma protein concentration are preferably kept the same.

Any concentration can be selected for the concentration of plasma protein (for example, albumin) as long as difference is detected in the binding activities of the polypeptide complex. In a certain embodiment, examples of a high concentration or a higher concentration include 100 micro M or higher concentration, 150 micro M or higher concentration, 200 micro M or higher concentration, 250 micro M or higher concentration, 300 micro M or higher concentration, 350 micro M or higher concentration, 400 micro M or higher concentration, 450 micro M or higher concentration, 500 micro M or higher concentration, 550 micro M or higher concentration, 600 micro M or higher concentration, 650 micro M or higher concentration, 700 micro M or higher concentration, 750 micro M or higher concentration, 800 micro M or higher concentration, 850 micro M or higher concentration, 900 micro M or higher concentration, 950 micro M or higher concentration, 1 mM or higher concentration, 2 mM or higher concentration, 3 mM or higher concentration, 4 mM or higher concentration, 5 mM or higher concentration, 6 mM or higher concentration, 7 mM or higher concentration, 8 mM or higher concentration, 9 mM or higher concentration, 10 mM or higher concentration, 30 mM or higher concentration, 100 mM or higher concentration, 300 mM or higher concentration, and 1 M or higher concentration.

In one embodiment, examples of a high concentration or higher concentration include plasma protein concentration in the plasma of humans, rabbits, mice, rats, monkeys, and bovines.

In one embodiment, a sufficient amount of polypeptide complex that can show maximum binding activity for a target antigen can be selected as the high concentration or higher concentration mentioned herein. Furthermore, in one embodiment, plasma protein concentration that shows “a larger KD value, apparent KD value, or in vivo KD value for the target antigen” and/or “a smaller binding amount of the target antigen” as described in the present specification can be selected as the high concentration or higher concentration mentioned herein.

In a certain embodiment, examples of a low concentration or lower concentration include, 100 micro M or lower concentration, 90 micro M or lower concentration, 80 micro M or lower concentration, 70 micro M or lower concentration, 60 micro M or lower concentration, 50 micro M or lower concentration, 40 micro M or lower concentration, 30 micro M or lower concentration, 20 micro M or lower concentration, 10 micro M or lower concentration, 9 micro M or lower concentration, 8 micro M or lower concentration, 7 micro M or lower concentration, 6 micro M or lower concentration, 5 micro M or lower concentration, 4 micro M or lower concentration, 3 micro M or lower concentration, 2 micro M or lower concentration, 1 micro M or lower concentration, 900 nM or lower concentration, 800 nM or lower concentration, 700 nM or lower concentration, 600 nM or lower concentration, 500 nM or lower concentration, 400 nM or lower concentration, 300 nM or lower concentration, 200 nM or lower concentration, 100 nM or lower concentration, 30 nM or lower concentration, 10 nM or lower concentration, 3 nM or lower concentration, 1 nM or lower concentration, 300 pM or lower concentration, 100 pM or lower concentration, 30 pM or lower concentration, 10 pM or lower concentration, 3 pM or lower concentration, and 1 pM or lower concentration.

In one embodiment, examples of a low concentration or lower concentration include plasma protein concentration in the central nervous system (CNS) tissues, such as the cerebrospinal fluid (CSF), of humans, rabbits, mice, rats, monkeys, and bovines.

In one embodiment, sufficient amount of polypeptide complex that can show minimum binding activity for a target antigen can be selected as the low concentration mentioned herein. A case where the concentration is substantially zero (where plasma protein is absent) may be selected as an embodiment of low concentration. Furthermore, in one embodiment, plasma protein concentration that shows “a smaller KD value, apparent KD value, or in vivo KD value for the target antigen” and/or “a larger binding amount of the target antigen” as described in the present specification can be selected as the low concentration or lower concentration herein.

In another embodiment, as an example of the ratio between a low concentration and a high concentration, one can select a value of 3-fold or more, 10-fold or more, 30-fold or more, 50-fold or more, 100-fold or more, 150-fold or more, 160-fold or more, 170-fold or more, 180-fold or more, 190-fold or more, 200-fold or more, 250-fold or more, 300-fold or more, 350-fold or more, 400-fold or more, 450-fold or more, 500-fold or more, 1×10³-fold or more, 3×10³-fold or more, or 1×10⁴-fold or more.

Conditions when measuring target antigen-binding activity other than the concentration of a plasma protein are not particularly limited, and can be selected appropriately by those skilled in the art. For example, it is possible to measure under conditions of HEPES buffer and 37 degrees C. For example, Biacore (GE Healthcare) or such can be used for measurement. When the target antigen is a soluble molecule, the activity of a polypeptide complex to bind to the soluble molecule can be determined by loading the target antigen as an analyte onto a chip immobilized with the polypeptide complex. Alternatively, when the target antigen is a membrane-type molecule, the binding activity towards the membrane-type molecule can be determined by loading the polypeptide complex as an analyte onto a chip immobilized with the target antigen.

To obtain a polypeptide complex whose binding activity to a target antigen varies with the concentration of a plasma protein, the following methods and such may be suitably applied. For example, to confirm that the binding activity of a polypeptide complex to a target antigen in the presence of a plasma protein becomes lower than the binding activity of the polypeptide complex to the target antigen in the absence of the plasma protein, the binding activities of the polypeptide complex to the target antigen in the presence and absence of the plasma protein are compared. In another non-limiting embodiment, for example, to confirm that the binding activity of a polypeptide complex to a target antigen in the presence of a high concentration of a plasma protein becomes lower than the binding activity of the polypeptide complex to the antigen in the presence of a low concentration of the plasma protein, the binding activities of the polypeptide complex to the target antigen in the presence of high and low concentrations of the plasma protein are compared.

In one embodiment, binding activity of a polypeptide complex may be measured by a radiolabeled antigen binding assay (RIA). In one embodiment, an RIA is performed with the polypeptide complex of interest and its target antigen. For example, solution binding affinity of a polypeptide complex for its target antigen is measured by equilibrating Fab with a minimal concentration of (¹²⁵I)-labeled antigen in the presence of a titration series of unlabeled antigen, then capturing bound target antigen with a plate coated with antibodies against the polypeptide complex (see, e.g., Chen et al., J. Mol. Biol. 293:865-881(1999)). To establish conditions for the assay, MICROTITER (registered trademark) multi-well plates (Thermo Scientific) are coated overnight with 5 microgram/ml of a capturing anti-polypeptide complex antibody in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵]-target antigen are mixed with serial dilutions of a polypeptide complex of interest (e.g., consistent with assessment of the anti-VEGF antibody, Fab-12, in Presta et al., Cancer Res. 57:4593-4599 (1997)). The polypeptide complex of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 microliter/well of scintillant (MICROSCINT-20™; Packard) is added, and the plates are counted on a TOPCOUNT™ gamma counter (Packard) for ten minutes. Concentrations of each polypeptide complex that give less than or equal to 20% of maximal binding are chosen for use in competitive binding assays.

In one embodiment, for measuring binding activity of a polypeptide complex, ligand-capturing methods, for example, using BIACORE (registered trademark) T200 or BIACORE (registered trademark) 4000 (GE Healthcare, Uppsala, Sweden), which rely upon surface plasmon resonance analysis methods as the measurement principle, are used. BIACORE (registered trademark) Control Software is used for operation of devices. In one embodiment, amine-coupling kit (GE Healthcare, Uppsala, Sweden) is used according to the manufacturer's instructions to let a molecule for ligand capturing, for example, an anti-tag antibody, an anti-IgG antibody, protein A, etc. fixed onto a sensor chip (GE Healthcare, Uppsala, Sweden) coated with carboxymethyldextran. The ligand-capturing molecule is diluted with a 10 mM sodium acetate solution at an appropriate pH and is injected at an appropriate flow rate and for an appropriate injection time. Binding activity measurements are measured using a 0.05% polysorbate 20 (in other name Tween (registered trademark)-20)-containing buffer as a measurement buffer, at a flow rate of 10-30 microliter/minute, and at a measurement temperature of preferably at 25 degrees C. or 37 degrees C. For the measurement carried out with an antibody captured by the ligand-capturing molecule as a ligand, an antibody is injected to let a target amount of the antibody captured, and then a serial dilution of a target antigen (analyte) prepared using the measurement buffer is injected. For the measurement carried out with a target antigen captured by the ligand-capturing molecule as a ligand, a target antigen is injected to let a target amount thereof captured, and then a serial dilution of an antibody (analyte) prepared using the measurement buffer is injected.

In one embodiment, the measurement results are analyzed using BIACORE (registered trademark) Evaluation Software. Kinetics parameter calculation is carried out by fitting sensorgrams of association and dissociation at the same time using a 1:1 binding model, and an association rate (kon or ka), a dissociation rate (koff or kd), and an equilibrium dissociation constant (KD) may be calculated. For the case of weak binding activity, in particular, for the cases where dissociation is fast and kinetics parameters are difficult to calculate, the Steady state model may be used to calculate the equilibrium dissociation constant (KD). As additional parameters concerning binding activity, “binding amount of analyte per unit ligand amount” may be calculated by dividing a binding amount of analyte (resonance unit: RU) at a specific concentration by an amount of captured ligand.

When the target antigen is a membrane-type molecule, methods for assaying the binding activity of a polypeptide complex, which comprises an antigen-binding portion against the target antigen, towards target antigen-expressing cells include, for example, the methods described in Antibodies: A Laboratory Manual (Ed Harlow, David Lane, Cold Spring Harbor Laboratory (1988) 359-420). Specifically, the assessment can be performed based on the principle of ELISA or fluorescence activated cell sorting (FACS) using target antigen-expressing cells as antigen. Methods for assaying the binding activity of a polypeptide complex that binds to IL-6R towards IL-6R-expressing cells are illustrated below. A polypeptide complex that binds to a target antigen other than IL-6R may be suitably assayed according to the example described below.

In the ELISA format, the binding activity of a polypeptide complex containing an IL-6R antigen-binding portion towards IL-6R-expressing cells can be assessed quantitatively by comparing the levels of signal generated by enzymatic reaction. Specifically, a test polypeptide complex is added to an ELISA plate onto which IL-6R-expressing cells are immobilized. Then, the test polypeptide complex bound to the cells is detected using an enzyme-labeled antibody that recognizes the test polypeptide complex. Alternatively, when FACS is used, a dilution series of a test polypeptide complex is prepared, and the antibody binding titer for IL-6R-expressing cells can be determined to compare the binding activity of the test polypeptide complex towards IL-6R-expressing cells.

The binding of a polypeptide complex towards an antigen expressed on the surface of cells suspended in buffer or the like can be detected using a flow cytometer. Known flow cytometers include, for example, the following devices:
FACSCanto™ II
FACSAria™
FACSArray™
FACSVantage™ SE
FACSCalibur™ (all are trade names of BD Biosciences)
EPICS ALTRA HyPerSort
Cytomics FC 500
EPICS XL-MCL ADC EPICS XL ADC
Cell Lab Quanta/Cell Lab Quanta SC (all are trade names of Beckman Coulter).

Preferable methods for assaying the binding activity of a test polypeptide complex containing an IL-6R antigen-binding portion towards a target antigen include, for example, the following method. First, IL-6R-expressing cells are reacted with a test polypeptide complex, and then this is stained with an FITC-labeled secondary antibody that recognizes the test polypeptide complex. The test polypeptide complex is appropriately diluted with a suitable buffer to prepare the polypeptide complex at a desired concentration. For example, the polypeptide complex can be used at a concentration within the range of 10 micro g/ml to 10 ng/ml. Then, the fluorescence intensity and cell count are determined using FACSCalibur (BD). The fluorescence intensity obtained by analysis using the CELL QUEST Software (BD), i.e., the Geometric Mean value, reflects the quantity of antibody bound to cells. That is, the binding activity of a test polypeptide complex, which is represented by the quantity of the test polypeptide complex bound, can be determined by measuring the Geometric Mean value.

### Structure of Polypeptide Complexes

Polypeptide complexes of the present invention can comprise one or more first antigen-binding portions (plasma protein-binding portions), and one or more second antigen-binding portions (target antigen-binding portions). In one embodiment, polypeptide complexes of the present invention may comprise two or more (for example, 2, 3, or 4) first antigen-binding portions (plasma protein-binding portions) and one or more (for example, 1 or 2) second antigen-binding portions (target antigen-binding portions). In another embodiment, polypeptide complexes of the present invention may comprise 1 or 2 first antigen-binding portions (plasma protein-binding portions) per one second antigen-binding portion that can bind to a target antigen (target antigen-binding portion).

Specifically, examples of a polypeptide complex of the present invention include, but are not limited to:
a polypeptide complex comprising one first antigen-binding portion (plasma protein-binding portion) and one second antigen-binding portion (target antigen-binding portion);
a polypeptide complex comprising two first antigen-binding portions (plasma protein-binding portions) and one second antigen-binding portion (target antigen-binding portion);
a polypeptide complex comprising one first antigen-binding portion (plasma protein-binding portion) and two second antigen-binding portions (target antigen-binding portions);
a polypeptide complex comprising two first antigen-binding portions (plasma protein-binding portions) and two second antigen-binding portions (target antigen-binding portions);
a polypeptide complex comprising three first antigen-binding portions (plasma protein-binding portions) and two second antigen-binding portions (target antigen-binding portions); and
a polypeptide complex comprising four first antigen-binding portions (plasma protein-binding portions) and two second antigen-binding portions (target antigen-binding portions).

When comprising two second antigen-binding portions (target antigen-binding portions), each of the second antigen-binding portions may bind to the same target antigen or may bind to different target antigens. Furthermore, the second antigen-binding portion preferably binds to a protein that is not a plasma protein.

In one embodiment, a polypeptide complex of the present invention may comprise one first antigen-binding portion (plasma protein-binding portion) and one second antigen-binding portion (target antigen-binding portion). In one embodiment, the second antigen-binding portion (target antigen-binding portion) may comprise a Fab, i.e., a set of a polypeptide chain comprising a heavy chain variable region and a CH1 domain of a heavy chain constant region (CH1) and a polypeptide chain comprising a light chain variable region and a light chain constant region. In this case, the polypeptide complex of the present invention may be a polypeptide complex in which an amino acid residue positioned in the heavy chain variable region or the light chain variable region and an amino acid residue of the first antigen-binding portion (plasma protein-binding portion) are linked via a non-cleavable linker consisting of 0 to 4 amino acid residues. For example, the polypeptide complex of the present invention may be a polypeptide complex in which an N-terminal amino acid residue of either the heavy chain variable region or the light chain variable region and a C-terminal amino acid residue of the first antigen-binding portion (plasma protein-binding portion) are linked via a non-cleavable linker consisting of 0 to 4 amino acid residues. The non-cleavable linker preferably consists of 0 to 3 amino acid residues, and more preferably consists of 0 to 1 amino acid residue. The phrase “non-cleavable linker consisting of 0 amino acid residue” means that no linker is included at all.

In one embodiment, such a polypeptide complex may be “a polypeptide complex comprising a first polypeptide chain and a second polypeptide chain, wherein the first polypeptide chain comprises a plasma protein-binding portion, a heavy chain variable region (VH), and a CH1 domain of a heavy chain constant region (CH1) in this order from the N terminus, and wherein the second polypeptide chain comprises a light chain variable region (VL) and a light chain constant region (CL) in this order from the N terminus” or “a polypeptide complex comprising a first polypeptide chain and a second polypeptide chain, wherein the first polypeptide chain comprises a heavy chain variable region (VH) and a CH1 domain of a heavy chain constant region (CH1) in this order from the N terminus, and wherein the second polypeptide chain comprises a plasma protein-binding portion, a light chain variable region (VL), and a light chain constant region (CL) in this order from the N terminus”. The first polypeptide chain and the second polypeptide chain may be linked by a disulfide bond. In one embodiment, the polypeptide complex of the present invention may comprise one or two sets of the first polypeptide chain and the second polypeptide chain. In case that the polypeptide complex comprises two sets of the first polypeptide chain and the second polypeptide chain, the first polypeptide chain of one set and the first polypeptide chain of the other set may be linked by a disulfide bond.

The present inventors elucidated that compared to a polypeptide complex in which the N-terminal amino acid residue of the heavy chain variable region and the C-terminal amino acid residue of the first antigen-binding portion (plasma protein-binding portion) are linked via a non-cleavable linker consisting of 0 to 4 amino acid residues (the first antigen-binding portion (plasma protein-binding portion) is not linked to the light chain variable region), a polypeptide complex in which the N-terminal amino acid residue of the light chain variable region and the C-terminal amino acid residue of the first antigen-binding portion (plasma protein-binding portion) are linked via a non-cleavable linker consisting of 0 to 4 amino acid residues (the first antigen-binding portion (plasma protein-binding portion) is not linked to the heavy chain variable region) can more effectively inhibit binding to the target antigen in the presence of plasma protein. Therefore, the former polypeptide complex can be an example of a preferable polypeptide complex of the present invention.

In one embodiment, a polypeptide complex of the present invention can comprise two first antigen-binding portions (plasma protein-binding portions) and one second antigen-binding portion (target antigen-binding portion). In one embodiment, the second antigen-binding portion (target antigen-binding portion) may comprise a Fab, i.e., a set of a polypeptide chain comprising a heavy chain variable region and a CH1 domain of a heavy chain constant region (CH1) and a polypeptide chain comprising a light chain variable region and a light chain constant region. In this case, the polypeptide complex of the present invention may be a polypeptide complex in which an amino acid residue positioned in the heavy chain variable region and an amino acid residue of one of the first antigen-binding portions (plasma protein-binding portions) are linked via a non-cleavable linker consisting of 0 to 4 amino acid residues, and an amino acid residue positioned in the light chain variable region and an amino acid residue of the other first antigen-binding portion (plasma protein-binding portion) are linked via a non-cleavable linker consisting of 0 to 4 amino acid residues. For example, the polypeptide complex of the present invention may be a polypeptide complex in which the N-terminal amino acid residue of the heavy chain variable region and the C-terminal amino acid residue of one of the first antigen-binding portions (plasma protein-binding portions) are linked via a non-cleavable linker consisting of 0 to 4 amino acid residues, and the N-terminal amino acid residue of the light chain variable region and the C-terminal amino acid residue of the other first antigen-binding portion (plasma protein-binding portion) are linked via a non-cleavable linker consisting of 0 to 4 amino acid residues.

The present inventors elucidated that compared to a polypeptide complex in which one first antigen-binding portion (plasma protein-binding portion) is linked to one second antigen-binding portion (target antigen-binding portion), a polypeptide complex in which two first antigen-binding portions (plasma protein-binding portions) are linked to one second antigen-binding portion (target antigen-binding portion) can more effectively inhibit binding to the target antigen in the presence of plasma protein. Therefore, in the present invention, a polypeptide complex in which two first antigen-binding portions (plasma protein-binding portions) are linked to one second antigen-binding portion (target antigen-binding portion) can be presented as a preferable example.

In one embodiment, such a polypeptide complex may be “a polypeptide complex comprising a first polypeptide chain and a second polypeptide chain, wherein the first polypeptide chain comprises a plasma protein-binding portion, a heavy chain variable region (VH), and a CH1 domain of a heavy chain constant region (CH1) in this order from the N terminus, and wherein the second polypeptide chain comprises a plasma protein-binding portion, a light chain variable region (VL), and a light chain constant region (CL) in this order from the N terminus”.

In the above-mentioned embodiment, the heavy chain variable region (VH), CH1 domain of the heavy chain constant region (CH1), light chain variable region (VL), and light chain constant region (CL) form an antibody Fab. Furthermore, the heavy chain variable region (VH) and the light chain variable region (VL) form the binding portion for the target antigen. In one embodiment, the C-terminal amino acid residue of the plasma protein-binding portion is linked to the N-terminal amino acid residue(s) of the heavy chain variable region (VH) and/or the light chain variable region (VL) via a non-cleavable linker consisting of 0 to 4 amino acid residues. Such a polypeptide complex can comprise an antibody Fc region. In this case the C-terminal amino acid residue of the CH1 domain of the heavy chain constant region (CH1) is linked to the N-terminal amino acid residue of the antibody Fc region.

In one embodiment, a polypeptide complex of the present invention can comprise one first antigen-binding portion (plasma protein-binding portion) and two second antigen-binding portions (target antigen-binding portions). In one embodiment, the second antigen-binding portion (target antigen-binding portion) may comprise a Fab, i.e., a set of a polypeptide chain comprising a heavy chain variable region and a CH1 domain of a heavy chain constant region (CH1) and a polypeptide chain comprising a light chain variable region and a light chain constant region. In this case, the polypeptide complex of the present invention may be a polypeptide complex in which in either one of the two second antigen-binding portions (target antigen-binding portions), an amino acid residue positioned in the heavy chain variable region or the light chain variable region and an amino acid residue of the first antigen-binding portion (plasma protein-binding portion) are linked via a non-cleavable linker consisting of 0 to 4 amino acid residues. For example, the polypeptide complex of the present invention may be a polypeptide complex in which in either one of the two second antigen-binding portions (target antigen-binding portions), the N-terminal amino acid residue of either the heavy chain variable region or the light chain variable region and the C-terminal amino acid residue of the first antigen-binding portion (plasma protein-binding portion) are linked via a non-cleavable linker consisting of 0 to 4 amino acid residues. In one embodiment, the polypeptide complex of the present invention is preferably a polypeptide complex in which the N-terminal amino acid residue of the light chain variable region and the C-terminal amino acid residue of the first antigen-binding portion (plasma protein-binding portion) are linked via a non-cleavable linker consisting of 0 to 4 amino acid residues.

In one embodiment, a polypeptide complex of the present invention can comprise two first antigen-binding portions (plasma protein-binding portions) and two second antigen-binding portions (target antigen-binding portions). In one embodiment, each of the second antigen-binding portion (target antigen-binding portion) may comprise a Fab, i.e., a set of a polypeptide chain comprising a heavy chain variable region and a CH1 domain of a heavy chain constant region (CH1) and a polypeptide chain comprising a light chain variable region and a light chain constant region. In this case, the polypeptide complex of the present invention may be a polypeptide complex in which in one of the second antigen-binding portions (target antigen-binding portions), an amino acid residue positioned in the heavy chain variable region or the light chain variable region and an amino acid residue of one of the first antigen-binding portions (plasma protein-binding portions) are linked via a non-cleavable linker consisting of 0 to 4 amino acid residues, and in the other second antigen-binding portion (target antigen-binding portion), an amino acid residue positioned in the heavy chain variable region or the light chain variable region and an amino acid residue of the other first antigen-binding portion (plasma protein-binding portion) are linked via a non-cleavable linker consisting of 0 to 4 amino acid residues. Alternatively, the polypeptide complex of the present invention may be a polypeptide complex in which in one of the second antigen-binding portions (target antigen-binding portions), an amino acid residue positioned in the heavy chain variable region and an amino acid residue of one of the first antigen-binding portions (plasma protein-binding portions) are linked via a non-cleavable linker consisting of 0 to 4 amino acid residues, and an amino acid residue positioned in the light chain variable region and an amino acid residue of the other first antigen-binding portion (plasma protein-binding portion) are linked via a non-cleavable linker consisting of 0 to 4 amino acid residues, and in the other second antigen-binding portion (target antigen-binding portion), the first antigen-binding portion (plasma protein-binding portion) is not linked.

In a certain embodiment, the polypeptide complex of the present invention may be a polypeptide complex in which in one of the two second antigen-binding portions (target antigen-binding portions), the N-terminal amino acid residue of the heavy chain variable region and the C-terminal amino acid residue of one of the first antigen-binding portions (plasma protein-binding portions) are linked via a non-cleavable linker consisting of 0 to 4 amino acid residues, and in the other one of the two second antigen-binding portions (target antigen-binding portions), the N-terminal amino acid residue of the heavy chain variable region and the C-terminal amino acid residue of the other first antigen-binding portion (plasma protein-binding portion) are linked via a non-cleavable linker consisting of 0 to 4 amino acid residues. Furthermore, in a certain embodiment, the polypeptide complex of the present invention may be a polypeptide complex in which in one of the two second antigen-binding portions (target antigen-binding portions), the N-terminal amino acid residue of the light chain variable region and the C-terminal amino acid residue of one of the first antigen-binding portions (plasma protein-binding portions) are linked via a non-cleavable linker consisting of 0 to 4 amino acid residues, and in the other one of the two second antigen-binding portions (target antigen-binding portions), the N-terminal amino acid residue of the light chain variable region and the C-terminal amino acid residue of the other first antigen-binding portion (plasma protein-binding portion) are linked via a non-cleavable linker consisting of 0 to 4 amino acid residues. Furthermore, in a certain embodiment, the polypeptide complex of the present invention may be a polypeptide complex in which in one of the two second antigen-binding portions (target antigen-binding portions), the N-terminal amino acid residue of the heavy chain variable region and the C-terminal amino acid residue of one of the first antigen-binding portions (plasma protein-binding portions) are linked via a non-cleavable linker consisting of 0 to 4 amino acid residues, and in the other one of the two second antigen-binding portions (target antigen-binding portions), the N-terminal amino acid residue of the light chain variable region and the C-terminal amino acid residue of the other first antigen-binding portion (plasma protein-binding portion) are linked via a non-cleavable linker consisting of 0 to 4 amino acid residues. Alternatively, the polypeptide complex of the present invention may be a polypeptide complex in which in one of the two second antigen-binding portions (target antigen-binding portions), the N-terminal amino acid residue of the heavy chain variable region and the C-terminal amino acid residue of one of the first antigen-binding portions (plasma protein-binding portions) are linked via a non-cleavable linker consisting of 0 to 4 amino acid residues, and the N-terminal amino acid residue of the light chain variable region and the C-terminal amino acid residue of the other first antigen-binding portion (plasma protein-binding portion) are linked via a non-cleavable linker consisting of 0 to 4 amino acid residues, and in the other one of the two second antigen-binding portions (target antigen-binding portions), the first antigen-binding portion (plasma protein-binding portion) is not linked to the N-terminal amino acid residues of both the heavy chain variable region and the light chain variable region.

In one embodiment, a polypeptide complex of the present invention can comprise three first antigen-binding portions (plasma protein-binding portions) and two second antigen-binding portions (target antigen-binding portions). In one embodiment, each of the second antigen-binding portion (target antigen-binding portion) may comprise a Fab, i.e., a set of a polypeptide chain comprising a heavy chain variable region and a CH1 domain of a heavy chain constant region (CH1) and a polypeptide chain comprising a light chain variable region and a light chain constant region. In this case, the polypeptide complex of the present invention may be a polypeptide complex in which in one of the second antigen-binding portions (target antigen-binding portions), amino acid residues positioned in each of the heavy chain variable region and the light chain variable region, and amino acid residues positioned in each of any two first antigen-binding portions (plasma protein-binding portions) out of the three first antigen-binding portions (plasma protein-binding portions) are linked without the mediation of a linker, and in the other second antigen-binding portion (target antigen-binding portion), an amino acid residue positioned in the heavy chain variable region or the light chain variable region and an amino acid residue of the remaining one first antigen-binding portion (plasma protein-binding portion) out of the three first antigen-binding portions (plasma protein-binding portions) are linked without the mediation of a linker. For example, the polypeptide complex of the present invention may be a polypeptide complex in which in either one of the two second antigen-binding portions (target antigen-binding portions), each N-terminal amino acid residue of both the heavy chain variable region and the light chain variable region and the C-terminal amino acid residues of each of any two first antigen-binding portions (plasma protein-binding portions) out of the three first antigen-binding portions (plasma protein-binding portions) are linked via a non-cleavable linker consisting of 0 to 4 amino acid residues, and in the other one of the two second antigen-binding portions (target antigen-binding portions), the N-terminal amino acid residue of either one of the heavy chain variable region and the light chain variable region and the C-terminal amino acid residue of the remaining one first antigen-binding portion (plasma protein-binding portion) out of the three first antigen-binding portions (plasma protein-binding portions) are linked via a non-cleavable linker consisting of 0 to 4 amino acid residues.

In one embodiment, a polypeptide complex of the present invention can comprise four first antigen-binding portions (plasma protein-binding portions) and two second antigen-binding portions (target antigen-binding portions). In one embodiment, each of the second antigen-binding portion (target antigen-binding portion) may comprise a Fab, i.e., a set of a polypeptide chain comprising a heavy chain variable region and a CH1 domain of a heavy chain constant region (CH1) and a polypeptide chain comprising a light chain variable region and a light chain constant region. In this case, the polypeptide complex of the present invention may be a polypeptide complex in which in both of the second antigen-binding portions (target antigen-binding portions), amino acid residues positioned in each of the heavy chain variable region and the light chain variable region, and the respective amino acid residues positioned in each of the first antigen-binding portions (plasma protein-binding portions) are linked via a non-cleavable linker consisting of 0 to 4 amino acid residues. For example, the polypeptide complex of the present invention may be a polypeptide complex in which in both of the two second antigen-binding portions (target antigen-binding portions), each of the N-terminal amino acid residues of both the heavy chain variable region and the light chain variable region and the C-terminal amino acid residue of each of the first antigen-binding portions (plasma protein-binding portions) are linked via a non-cleavable linker consisting of 0 to 4 amino acid residues. In this example, each heavy chain variable region and light chain variable region of the two second antigen-binding portions is linked via a non-cleavable linker consisting of 0 to 4 amino acid residues to one first antigen-binding portion. Alternatively, each heavy chain variable region and light chain variable region of the two second antigen-binding portions is linked to one first antigen-binding portion without a linker.

In the polypeptide complexes described above, a non-cleavable linker consists of preferably 0 to 3 amino acid residues, and more preferably 0 to 1 amino acid residue. The phrase “non-cleavable linker consisting of 0 amino acid residue” means that no linker is included at all.

In one embodiment, the polypeptide complex of the present invention may further comprise an antibody Fc region or a variant Fc region, or a fragment thereof.

The term “Fc region” herein is used to define a C-terminal region of an immunoglobulin heavy chain that contains at least a portion of the constant region. The term includes native sequence Fc regions and variant Fc regions. In one embodiment, the C-terminal lysine (Lys447) or glycine-lysine (Gly446-Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “variant Fc region” as used herein comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fe region of the parent polypeptide. The variant Fc region herein will preferably possess at least about 80% homology with a native sequence Fc region and/or with an Fc region of a parent polypeptide, and most preferably at least about 90% homology therewith, more preferably at least about 95%, 96%, 97%, 98%, or 99% homology therewith.

In another embodiment, the Fc region may be a variant Fc region produced by adding amino acid modification(s) to the Fc region of a native sequence, and preferably the Fc region variant of a native IgG (IgG1, IgG2, IgG3, or IgG4 type) antibody. In a specific embodiment, the variant Fc region has increased or decreased binding activity for at least one Fc gamma receptor selected from the group consisting of Fc gamma RIa, Fc gamma RIIa, Fc gamma RIIb, and Fc gamma RIIIa, as compared to an Fc region of a native sequence.

In one embodiment, the FcRn-binding activity of a variant Fc region is increased under an acidic pH range and neutral pH range, especially under acidic pH range. In one embodiment, a variant Fc region of the present invention contains Ala at position 434; any one of Glu, Arg, Ser, and Lys at position 438; and any one of Glu, Asp, and Gln at position 440, according to EU numbering; and more preferably contains Ala at position 434; either Arg or Lys at position 438; and either Glu or Asp at position 440, according to EU numbering. Preferably, the variant Fc region additionally contains either Ile or Leu at position 428, and/or any one of Ile, Leu, Val, Thr, and Phe at position 436, according to EU numbering. More preferably the variant Fc region contains Len at position 428, and/or either Val or Thr at position 436, according to EU numbering.

Thus, in one embodiment, the variant Fc region may contain a combination of amino acid substitutions selected from the group consisting of (according to EU numbering):
(a) N434A/Q438R/S440E;
(b) N434A/Q438R/S440D;
(c) N434A/Q438K/S440E;
(d) N434A/Q438K/S440D;
(e) N434A/Y436T/Q438R/S440E;
(f) N434A/Y436T/Q438R/S440D;
(g) N434A/Y436T/Q438K/S440E;
(h) N434A/Y436T/Q438K/S440D;
(i) N434A/Y436V/Q438R/S440E;
(j) N434A/Y436V/Q438R/S440D;
(k) N434A/Y436V/Q438K/S440E;
(l) N434A/Y436V/Q438K/S440D;
(m) N434A/R435H/F436T/Q438R/S440E;
(n) N434A/R435H/F436T/Q438R/S440D;
(o) N434A/R435H/F436T/Q438K/S440E;
(p) N434A/R435H/F436T/Q438K/S440D;
(q) N434A/R435H/F436V/Q438R/S440E;
(r) N434A/R435H/F436V/Q438R/S440D;
(s) N434A/R435H/F436V/Q438K/S440E;
(t) N434A/R435H/F436V/Q438K/S440D;
(u) M428L/N434A/Q438R/S440E;
(v) M428L/N434A/Q438R/S440D;
(w) M428L/N434A/Q438K/S440E;
(x) M428L/N434A/Q438K/S440D;
(y) M428L/N434A/Y436T/Q438R/S440E;
(z) M428L/N434A/Y436T/Q438R/S440D;
(aa) M428L/N434A/Y436T/Q438K/S440E;
(ab) M428L/N434A/Y436T/Q438K/S440D;
(ac) M428L/N434A/Y436V/Q438R/S440E;
(ad) M428L/N434A/Y436V/Q438R/S440D;
(ae) M428L/N434A/Y436V/Q438K/S440E;
(af) M428L/N434A/Y436V/Q438K/S440D;
(ag) L235R/G236R/S239K/M428L/N434A/Y436T/Q438R/S440E; and
(ah) L235R/G236R/A327G/A330S/P331S/M428L/N434A/Y436T/Q438R/S440E.

In a preferred embodiment, the variant Fc region may contain a combination of substituted amino acids selected from the group consisting of (according to EU numbering):
(a) N434A/Q438R/S440E;
(b) N434A/Y436T/Q438R/S440E;
(c) N434A/Y436V/Q438R/S440E;
(d) M428L/N434A/Q438R/S440E;
(e) M428L/N434A/Y436T/Q438R/S440E;
(f) M428L/N434A/Y436V/Q438R/S440E;
(g) L235R/G236R/S239K/M428L/N434A/Y436T/Q438R/S440E; and
(h) L235R/G236R/A327G/A330S/P331S/M428L/N434A/Y436T/Q438R/S440E.

In one embodiment, variant Fc regions preferably have increased FcRn-binding activity under acidic pH conditions, when compared to a native sequence Fc region. Increase in FcRn-binding activity of a variant Fc region at a certain pH range may correspond to increase in measured FcRn-binding activity, as compared to the FcRn-binding activity measured for a native sequence Fc region. In this case, KD (native sequence Fc region)/KD (variant Fc region) indicating the difference in binding activities may be at least 1.5-fold, 2-fold, 3-fold, 4-fold, 5-fold, 10-fold, 15-fold, 20-fold, 50-fold, 70-fold, 80-fold, 100-fold, 500-fold, or 1000-fold. Such increase may take place in the acidic pH range and/or neutral pH range, but increase in the acidic pH range may be favorable in some cases.

In one embodiment, when a polypeptide complex of the present invention comprises two second antigen-binding portions (target antigen-binding portions), and each of the second antigen-binding portion is Fab, the two second antigen-binding portions (target antigen-binding portions) may constitute a full-length antibody together with antibody Fc. Therefore, in one embodiment, the polypeptide complex of the present invention may be a polypeptide complex comprising a first antigen-binding portion (plasma protein-binding portion) and an antibody that can bind to a target antigen.

For the polypeptide complexes of the present invention, plasma protein is preferably albumin, and albumin is preferably human albumin. More specifically, the first antigen-binding portion in the present invention is preferably an albumin-binding portion that can bind to albumin, and more preferably a human albumin-binding portion that can bind to human albumin.

When a polypeptide complex of the present invention comprises an antibody as part thereof, the antibody may be a chimeric antibody, humanized antibody, human antibody, or mouse antibody. Furthermore, it may be monoclonal or polyclonal antibodies. Furthermore, it may be multispecific antibodies (for example, bispecific antibodies). In one embodiment, the antibody may be antibody fragments such as Fv, Fab, Fab′, scFv, diabodies, and F(ab′)2. Furthermore, the antibody may be derived from a human, mouse, rat, hamster, rabbit, monkey, or such. In another embodiment, the antibody may be, for example, a complete IgG1 antibody or a complete IgG4 antibody, or a full-length antibody of another antibody class or isotype defined herein.

The terms “full length antibody”, “intact antibody” and “whole antibody” are used herein interchangeably to refer to an antibody having a structure substantially similar to a native antibody structure or having heavy chains that contain an Fc region or variant Fc region as defined herein.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies t may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human HVRs and amino acid residues from human FRs. It may be also referred to as an antibody comprising an amino acid sequence corresponding to the amino acid sequence of an antibody produced by a human or a human cell or of an antibody derived from a non-human source using a human antibody repertoire or other human antibody coding sequence. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

Methods for producing an antibody with desired binding activity are known to those skilled in the art. Below is an example that describes a method for producing an antibody that binds to IL-6R (anti-IL-6R antibody). Antibodies that bind to an antigen other than IL-6R can also be produced according to the example described below.

Anti-IL-6R antibodies can be obtained as polyclonal or monoclonal antibodies using known methods. The anti-IL-6R antibodies preferably produced are monoclonal antibodies derived from mammals. Such mammal-derived monoclonal antibodies include antibodies produced by hybridomas or host cells transformed with an expression vector carrying an antibody gene by genetic engineering techniques. “Humanized antibodies” or “chimeric antibodies” are included in the monoclonal antibodies of the present specification.

Monoclonal antibody-producing hybridomas can be produced using known techniques, for example, as described below. Specifically, mammals are immunized by conventional immunization methods using an IL-6R protein as a sensitizing antigen. Resulting immune cells are fused with known parental cells by conventional cell fusion methods. Then, hybridomas producing an anti-IL-6R antibody can be selected by screening for monoclonal antibody-producing cells using conventional screening methods.

The IL-6R protein can be used as a sensitizing antigen for immunization of mammals. A partial IL-6R peptide may also be used as a sensitizing antigen. In this case, a partial peptide can be prepared by chemical synthesis based on the amino acid sequence of human IL-6R, or by inserting a partial IL-6R gene into an expression vector for expression. Alternatively, a partial peptide can be produced by degrading an IL-6R protein with a protease. The length and region of the partial IL-6R peptide are not limited to particular embodiments. The number of amino acids forming a peptide to be used as a sensitizing antigen is preferably at least 5 or more, 6 or more, or 7 or more. More specifically, a peptide of 8 to 50 residues, more preferably 10 to 30 residues can be used as a sensitizing antigen.

For sensitizing antigen, alternatively it is possible to use a fusion protein prepared by fusing a desired partial polypeptide or peptide of the IL-6R protein with a different polypeptide. For example, antibody Fc fragments and peptide tags are preferably used to produce fusion proteins to be used as sensitizing antigens. Vectors for expression of such fusion proteins can be constructed by fusing in frame genes encoding two or more desired polypeptide fragments and inserting the fusion gene into an expression vector as described above. Methods for producing fusion proteins are described in Molecular Cloning 2nd ed. (Sambrook, J et al., Molecular Cloning 2nd ed., 9.47-9.58 (1989) Cold Spring Harbor Lab. Press). Methods for preparing IL-6R to be used as a sensitizing antigen, and immunization methods using IL-6R are specifically described in WO 2003/000883, WO 2004/022754, WO 2006/006693, and such.

There is no particular limitation on the mammals to be immunized with the sensitizing antigen. However, it is preferable to select the mammals by considering their compatibility with the parent cells to be used for cell fusion. In general, rodents such as mice, rats, and hamsters, rabbits, and monkeys are preferably used.

The above animals are immunized with a sensitizing antigen by known methods. Generally performed immunization methods include, for example, intraperitoneal or subcutaneous injection administration of a sensitizing antigen into mammals. Specifically, a sensitizing antigen is appropriately diluted with PBS (Phosphate-Buffered Saline), physiological saline, or the like. If desired, a conventional adjuvant such as Freund's complete adjuvant is mixed with the antigen, and the mixture is emulsified. Then, the sensitizing antigen is administered to a mammal several times at 4- to 21-day intervals. Appropriate carriers may be used in immunization with the sensitizing antigen. In particular, when a low-molecular-weight partial peptide is used as the sensitizing antigen, it is sometimes desirable to couple the sensitizing antigen peptide to a carrier protein such as albumin or keyhole limpet hemocyanin for immunization.

Alternatively, hybridomas producing a desired antibody can be prepared using DNA immunization as mentioned below. DNA immunization is an immunization method that confers immunostimulation by expressing a sensitizing antigen in an animal immunized as a result of administering a vector DNA constructed to allow expression of an antigen protein-encoding gene in the animal. As compared to conventional immunization methods in which a protein antigen is administered to animals to be immunized, DNA immunization is expected to be superior in that:
immunostimulation can be provided while retaining the structure of a membrane protein such as IL-6R; and
there is no need to purify the antigen for immunization.

In order to prepare a monoclonal antibody using DNA immunization, first, a DNA expressing an IL-6R protein is administered to an animal to be immunized. The IL-6R-encoding DNA can be synthesized by known methods such as PCR. The obtained DNA is inserted into an appropriate expression vector, and then this is administered to an animal to be immunized. Preferably used expression vectors include, for example, commercially-available expression vectors such as pcDNA3.1. Vectors can be administered to an organism using conventional methods. For example, DNA immunization is performed by using a gene gun to introduce expression vector-coated gold particles into cells in the body of an animal to be immunized. Antibodies that recognized IL-6R can also be produced by the methods described in WO 2003/104453.

After immunizing a mammal as described above, an increase in the titer of an IL-6R-binding antibody is confirmed in the serum. Then, immune cells are collected from the mammal, and then subjected to cell fusion. In particular, splenocytes are preferably used as immune cells.

A mammalian myeloma cell is used as a cell to be fused with the above-mentioned immune cells. The myeloma cells preferably comprise a suitable selection marker for screening. A selection marker confers characteristics to cells for their survival (or death) under a specific culture condition. Hypoxanthine-guanine phosphoribosyltransferase deficiency (hereinafter abbreviated as HGPRT deficiency) and thymidine kinase deficiency (hereinafter abbreviated as TK deficiency) are known as selection markers. Cells with HGPRT or TK deficiency have hypoxanthine-aminopterin-thymidine sensitivity (hereinafter abbreviated as HAT sensitivity). HAT-sensitive cells cannot synthesize DNA in a HAT selection medium, and are thus killed. However, when the cells are fused with normal cells, they can continue DNA synthesis using the salvage pathway of the normal cells, and therefore they can grow even in the HAT selection medium.

HGPRT-deficient and TK-deficient cells can be selected in a medium containing 6-thioguanine, 8-azaguanine (hereinafter abbreviated as 8AG), or 5′-bromodeoxyuridine, respectively. Normal cells are killed because they incorporate these pyrimidine analogs into their DNA. Meanwhile, cells that are deficient in these enzymes can survive in the selection medium, since they cannot incorporate these pyrimidine analogs. In addition, a selection marker referred to as G418 resistance provided by the neomycin-resistant gene confers resistance to 2-deoxystreptamine antibiotics (gentamycin analogs). Various types of myeloma cells that are suitable for cell fusion are known.

For example, myeloma cells including the following cells can be preferably used:
P3(P3x63Ag8.653) (J. Immunol. (1979) 123 (4), 1548-1550);
P3x63Ag8U.1 (Current Topics in Microbiology and Immunology (1978)81, 1-7);
NS-1 (C. Eur. J. Immunol. (1976)6 (7), 511-519);
MPC-11 (Cell (1976) 8 (3), 405-415);
SP2/0 (Nature (1978) 276 (5685), 269-270);
FO (J. Immunol. Methods (1980) 35 (1-2), 1-21);
S194/5.XX0.BU.1 (J. Exp. Med. (1978) 148 (1), 313-323);
R210 (Nature (1979) 277 (5692), 131-133), etc.

Cell fusions between the immunocytes and myeloma cells are essentially carried out using known methods, for example, a method by Kohler and Milstein et al. (Methods Enzymol. (1981) 73: 3-46).

More specifically, cell fusion can be carried out, for example, in a conventional culture medium in the presence of a cell fusion-promoting agent. The fusion-promoting agents include, for example, polyethylene glycol (PEG) and Sendai virus (HVJ). If required, an auxiliary substance such as dimethyl sulfoxide is also added to improve fusion efficiency.

The ratio of immune cells to myeloma cells may be determined at one's own discretion, preferably, for example, one myeloma cell for every one to ten immunocytes. Culture media to be used for cell fusions include, for example, media that are suitable for the growth of myeloma cell lines, such as RPMI1640 medium and MEM medium, and other conventional culture medium used for this type of cell culture. In addition, serum supplements such as fetal calf serum (FCS) may be preferably added to the culture medium.

For cell fusion, predetermined amounts of the above immune cells and myeloma cells are mixed well in the above culture medium. Then, a PEG solution (for example, the average molecular weight is about 1,000 to 6,000) prewarmed to about 37 degrees C. is added thereto at a concentration of generally 30% to 60% (w/v). This is gently mixed to produce desired fusion cells (hybridomas). Then, an appropriate culture medium mentioned above is gradually added to the cells, and this is repeatedly centrifuged to remove the supernatant. Thus, cell fusion agents and such which are unfavorable to hybridoma growth can be removed.

The hybridomas thus obtained can be selected by culture using a conventional selective medium, for example, HAT medium (a culture medium containing hypoxanthine, aminopterin, and thymidine). Cells other than the desired hybridomas (non-fused cells) can be killed by continuing culture in the above HAT medium for a sufficient period of time. Typically, the period is several days to several weeks. Then, hybridomas producing the desired antibody are screened and singly cloned by conventional limiting dilution methods.

The hybridomas thus obtained can be selected using a selection medium based on the selection marker possessed by the myeloma used for cell fusion. For example, HGPRT- or TK-deficient cells can be selected by culture using the HAT medium (a culture medium containing hypoxanthine, aminopterin, and thymidine). Specifically, when HAT-sensitive myeloma cells are used for cell fusion, cells successfully fused with normal cells can selectively proliferate in the HAT medium. Cells other than the desired hybridomas (non-fused cells) can be killed by continuing culture in the above HAT medium for a sufficient period of time. Specifically, desired hybridomas can be selected by culture for generally several days to several weeks. Then, hybridomas producing the desired antibody are screened and singly cloned by conventional limiting dilution methods.

Desired antibodies can be preferably selected and singly cloned by screening methods based on known antigen/antibody reaction. For example, an IL-6R-binding monoclonal antibody can bind to IL-6R expressed on the cell surface. Such a monoclonal antibody can be screened by fluorescence activated cell sorting (FACS). FACS is a system that assesses the binding of an antibody to cell surface by analyzing cells contacted with a fluorescent antibody using laser beam, and measuring the fluorescence emitted from individual cells.

To screen for hybridomas that produce a monoclonal antibody by FACS, IL-6R-expressing cells are first prepared. Cells preferably used for screening are mammalian cells in which IL-6R is forcedly expressed. As control, the activity of an antibody to bind to cell-surface IL-6R can be selectively detected using non-transformed mammalian cells as host cells. Specifically, hybridomas producing an anti-IL-6R monoclonal antibody can be isolated by selecting hybridomas that produce an antibody which binds to cells forced to express IL-6R, but not to host cells.

Alternatively, the activity of an antibody to bind to immobilized IL-6R-expressing cells can be assessed based on the principle of ELISA. For example, IL-6R-expressing cells are immobilized to the wells of an ELISA plate. Culture supernatants of hybridomas are contacted with the immobilized cells in the wells, and antibodies that bind to the immobilized cells are detected. When the monoclonal antibodies are derived from mouse, antibodies bound to the cells can be detected using an anti-mouse immunoglobulin antibody. Hybridomas producing a desired antibody having the antigen-binding ability are selected by the above screening, and they can be cloned by a limiting dilution method or the like.

Monoclonal antibody-producing hybridomas thus prepared can be passaged in a conventional culture medium, and stored in liquid nitrogen for a long period.

The above hybridomas are cultured by a conventional method, and desired monoclonal antibodies can be prepared from the culture supernatants. Alternatively, the hybridomas are administered to and grown in compatible mammals, and monoclonal antibodies are prepared from the ascites. The former method is suitable for preparing antibodies with high purity.

Antibodies encoded by antibody genes that are cloned from antibody-producing cells such as the above hybridomas can also be preferably used. A cloned antibody gene is inserted into an appropriate vector, and this is introduced into a host to express the antibody encoded by the gene. Methods for isolating antibody genes, inserting the genes into vectors, and transforming host cells have already been established, for example, by Vandamme et al. (Eur. J. Biochem. (1990) 192(3), 767-775). Methods for producing recombinant antibodies are also known as described below.

For example, a cDNA encoding the variable region (V region) of an anti-IL-6R antibody is prepared from hybridoma cells expressing the anti-IL-6R antibody. For this purpose, total RNA is first extracted from hybridomas. Methods used for extracting mRNAs from cells include, for example:
the guanidine ultracentrifugation method (Biochemistry (1979) 18(24), 5294-5299), and
the AGPC method (Anal. Biochem. (1987) 162(1), 156-159)

Extracted mRNAs can be purified using the mRNA Purification Kit (GE Healthcare Bioscience) or such. Alternatively, kits for extracting total mRNA directly from cells, such as the QuickPrep mRNA Purification Kit (GE Healthcare Bioscience), are also commercially available. mRNAs can be prepared from hybridomas using such kits. cDNAs encoding the antibody variable region can be synthesized from the prepared mRNAs using a reverse transcriptase. cDNAs can be synthesized using the AMV Reverse Transcriptase First-strand cDNA Synthesis Kit (Seikagaku Co.) or such. Furthermore, the SMART RACE cDNA amplification kit (Clontech) and the PCR-based 5′-RACE method (Proc. Natl. Acad. Sci. U.S.A. (1988) 85(23), 8998-9002; Nucleic Acids Res. (1989) 17(8), 2919-2932) can be appropriately used to synthesize and amplify cDNAs. In such a cDNA synthesis process, appropriate restriction enzyme sites described below may be introduced into both ends of a cDNA.

The cDNA fragment of interest is purified from the resulting PCR product, and then this is ligated to a vector DNA. A recombinant vector is thus constructed, and introduced into E. coli or such. After colony selection, the desired recombinant vector can be prepared from the colony-forming E. coli. Then, whether the recombinant vector has the cDNA nucleotide sequence of interest is tested by a known method such as the dideoxy nucleotide chain termination method.

The 5′-RACE method which uses primers to amplify the variable region gene is conveniently used for isolating the gene encoding the variable region. First, a 5′-RACE cDNA library is constructed by cDNA synthesis using RNAs extracted from hybridoma cells as a template. A commercially available kit such as the SMART RACE cDNA amplification kit is appropriately used to synthesize the 5′-RACE cDNA library.

The antibody gene is amplified by PCR using the prepared 5′-RACE cDNA library as a template. Primers for amplifying the mouse antibody gene can be designed based on known antibody gene sequences. The nucleotide sequences of the primers vary depending on the immunoglobulin subclass. Therefore, it is preferable that the subclass is determined in advance using a commercially available kit such as the Iso Strip mouse monoclonal antibody isotyping kit (Roche Diagnostics).

Specifically, for example, primers that allow amplification of genes encoding gamma 1, gamma 2a, gamma 2b, and gamma 3 heavy chains and kappa and lambda light chains are used to isolate mouse IgG-encoding genes. In general, a primer that anneals to a constant region site close to the variable region is used as a 3′-side primer to amplify an IgG variable region gene. Meanwhile, a primer attached to a 5′ RACE cDNA library construction kit is used as a 5′-side primer.

PCR products thus amplified are used to reshape immunoglobulins composed of a combination of heavy and light chains. A desired antibody can be selected using the IL-6R-binding activity of a reshaped immunoglobulin as an indicator. For example, when the objective is to isolate an antibody against IL-6R, it is more preferred that the binding of the antibody to IL-6R is specific. An IL-6R-binding antibody can be screened, for example, by the following steps:
(1) contacting an IL-6R-expressing cell with an antibody comprising the variable region encoded by a cDNA isolated from a hybridoma;
(2) detecting the binding of the antibody to the IL-6R-expressing cell; and
(3) selecting an antibody that binds to the IL-6R-expressing cell.

Methods for detecting the binding of an antibody to IL-6R-expressing cells are known. Specifically, the binding of an antibody to IL-6R-expressing cells can be detected by the above-described techniques such as FACS. Immobilized samples of IL-6R-expressing cells are appropriately used to assess the binding activity of an antibody.

Preferred antibody screening methods that use the binding activity as an indicator also include panning methods using phage vectors. Screening methods using phage vectors are advantageous when the antibody genes are isolated from heavy-chain and light-chain subclass libraries from a polyclonal antibody-expressing cell population. Genes encoding the heavy-chain and light-chain variable regions can be linked by an appropriate linker sequence to form a single-chain Fv (scFv). Phages presenting scFv on their surface can be produced by inserting a gene encoding scFv into a phage vector. The phages are contacted with an antigen of interest. Then, a DNA encoding scFv having the binding activity of interest can be isolated by collecting phages bound to the antigen. This process can be repeated as necessary to enrich scFv having the binding activity of interest.

After isolation of the cDNA encoding the variable region of the anti-IL-6R antibody of interest, the cDNA is digested with restriction enzymes that recognize the restriction sites introduced into both ends of the cDNA. Preferred restriction enzymes recognize and cleave a nucleotide sequence that occurs in the nucleotide sequence of the antibody gene at a low frequency. Furthermore, a restriction site for an enzyme that produces a sticky end is preferably introduced into a vector to insert a single-copy digested fragment in the correct orientation. The cDNA encoding the variable region of the anti-IL-6R antibody is digested as described above, and this is inserted into an appropriate expression vector to construct an antibody expression vector. In this case, if a gene encoding the antibody constant region (C region) and a gene encoding the above variable region are fused in-frame, a chimeric antibody is obtained. Herein, “chimeric antibody” means that the origin of the constant region is different from that of the variable region. Thus, in addition to mouse/human heterochimeric antibodies, human/human allochimeric antibodies are included in the chimeric antibodies of the present invention. A chimeric antibody expression vector can be constructed by inserting the above variable region gene into an expression vector that already has the constant region. Specifically, for example, a recognition sequence for a restriction enzyme that excises the above variable region gene can be appropriately placed on the 5′ side of an expression vector carrying a DNA encoding a desired antibody constant region. A chimeric antibody expression vector is constructed by fusing in frame the two genes digested with the same combination of restriction enzymes.

To produce an anti-IL-6R monoclonal antibody, antibody genes are inserted into an expression vector so that the genes are expressed under the control of an expression regulatory region. The expression regulatory region for antibody expression includes, for example, enhancers and promoters. Furthermore, an appropriate signal sequence may be attached to the amino terminus so that the expressed antibody is secreted to the outside of cells. The expressed polypeptide is cleaved at the carboxyl terminus of the above sequence, and the resulting polypeptide is secreted to the outside of cells as a mature polypeptide. Then, appropriate host cells are transformed with the expression vector, and recombinant cells expressing the anti-IL-6R antibody-encoding DNA are obtained.

DNAs encoding the antibody heavy chain (H chain) and light chain (L chain) are separately inserted into different expression vectors to express the antibody gene. An antibody molecule having the H and L chains can be expressed by co-transfecting the same host cell with vectors into which the H-chain and L-chain genes are respectively inserted. Alternatively, host cells can be transformed with a single expression vector into which DNAs encoding the H and L chains are inserted (see WO 1994/011523).

There are various known host cell/expression vector combinations for antibody preparation by introducing isolated antibody genes into appropriate hosts. All of these expression systems are applicable for isolation of the antigen-binding portions in the present invention. Appropriate eukaryotic cells used as host cells include animal cells, plant cells, and fungal cells. Specifically, the animal cells include, for example, the following cells.
(1) mammalian cells: CHO (Chinese hamster ovary cell line), COS (Monkey kidney cell line), myeloma (Sp2/0, NS0, etc.), BHK (baby hamster kidney cell line), HeLa, Vero, HEK293 (human embryonic kidney cell line with sheared adenovirus (Ad)5 DNA), PER.C6 cell (human embryonic retinal cell line transformed with the Adenovirus Type 5 (Ad5) E1A and E1B genes) and such (Current Protocols in Protein Science (May, 2001, Unit 5.9, Table 5.9.1));
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells:
yeasts: the Saccharomyces genus such as Saccharomyces cerevisiae, and the Pichia genus such as Pichia pastoris; and
filamentous fungi: the Aspergillus genus such as Aspergillus niger.

Furthermore, antibody gene expression systems that utilize prokaryotic cells are also known. For example, when using bacterial cells, E. coli cells, Bacillus subtilis cells, and such can suitably be utilized in the present invention. Expression vectors carrying the antibody genes of interest are introduced into these cells by transfection. The transfected cells are cultured in vitro, and the desired antibody can be prepared from the culture of transformed cells.

In addition to the above-described host cells, transgenic animals can also be used to produce a recombinant antibody. That is, the antibody can be obtained from an animal into which the gene encoding the antibody of interest is introduced. For example, the antibody gene can be constructed as a fusion gene by inserting in frame into a gene that encodes a protein produced specifically in milk. Goat beta-casein or such can be used, for example, as the protein secreted in milk. DNA fragments containing the fused gene inserted with the antibody gene is injected into a goat embryo, and then this embryo is introduced into a female goat. Desired antibodies can be obtained as a protein fused with the milk protein from milk produced by the transgenic goat born from the embryo-recipient goat (or progeny thereof). In addition, to increase the volume of milk containing the desired antibody produced by the transgenic goat, hormones can be administered to the transgenic goat as necessary (Ebert, K. M. et al., Bio/Technology (1994) 12 (7), 699-702).

When a polypeptide complex described herein is administered to human, an antigen-binding portion derived from a genetically recombinant antibody that has been artificially altered to reduce the heterologous antigenicity against human and such, can be appropriately used as the antigen-binding portion of the polypeptide complex. Such genetically recombinant antibodies include, for example, humanized antibodies. These altered antibodies are appropriately produced by known methods.

An antibody variable region used to produce the antigen-binding portion of a polypeptide complex described herein is generally formed by three complementarity-determining regions (CDRs) that are separated by four framework regions (FRs). CDR is a region that substantially determines the binding specificity of an antibody. The amino acid sequences of CDRs are highly diverse. On the other hand, the FR-forming amino acid sequences often have high identity even among antibodies with different binding specificities. Therefore, generally, the binding specificity of a certain antibody can be introduced to another antibody by CDR grafting.

A humanized antibody is also called a reshaped human antibody. Specifically, humanized antibodies prepared by grafting the CDR of a non-human animal antibody such as a mouse antibody to a human antibody and such are known. Common genetic engineering techniques for obtaining humanized antibodies are also known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the FR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, complementary strand synthesis reaction is conducted to anneal the overlapping CDR regions of the products synthesized using a human antibody gene as template. Human FRs are ligated via the mouse CDR sequences by this reaction.

The full length variable region gene, in which three CDRs and four FRs are ultimately ligated, is amplified using primers that anneal to its 5′- or 3′-end, which are added with suitable restriction enzyme recognition sequences. An expression vector for humanized antibody can be produced by inserting the DNA obtained as described above and a DNA that encodes a human antibody constant region into an expression vector so that they will ligate in frame. After the recombinant vector is transfected into a host to establish recombinant cells, the recombinant cells are cultured, and the DNA encoding the humanized antibody is expressed to produce the humanized antibody in the cell culture (see, European Patent Publication No. EP 239400 and International Patent Publication No. WO 1996/002576).

By qualitatively or quantitatively measuring and evaluating the antigen-binding activity of the humanized antibody produced as described above, one can suitably select human antibody FRs that allow CDRs to form a favorable antigen-binding site when ligated through the CDRs. Amino acid residues in FRs may be substituted as necessary, so that the CDRs of a reshaped human antibody form an appropriate antigen-binding site. For example, amino acid sequence mutations can be introduced into FRs by applying the PCR method used for grafting a mouse CDR into a human FR. More specifically, partial nucleotide sequence mutations can be introduced into primers that anneal to the FR. Nucleotide sequence mutations are introduced into the FRs synthesized by using such primers. Mutant FR sequences having the desired characteristics can be selected by measuring and evaluating the activity of the amino acid-substituted mutant antibody to bind to the antigen by the above-mentioned method (Cancer Res. (1993) 53: 851-856).

Alternatively, desired human antibodies can be obtained by immunizing transgenic animals having the entire repertoire of human antibody genes (see WO 1993/012227; WO 1992/003918; WO 1994/002602; WO 1994/025585; WO 1996/034096; WO 1996/033735) by DNA immunization.

Furthermore, techniques for preparing human antibodies by panning using human antibody libraries are also known. For example, the variable region of a human antibody is expressed as a single-chain antibody (scFv) on phage surface by the phage display method. Phages expressing an scFv that binds to the antigen can be selected. The DNA sequence encoding the human antibody variable region that binds to the antigen can be determined by analyzing the genes of selected phages. The DNA sequence of the scFv that binds to the antigen is determined. An expression vector is prepared by fusing the variable region sequence in frame with the constant region sequence of a desired human antibody, and inserting this into an appropriate expression vector. The expression vector is introduced into cells appropriate for expression such as those described above. The human antibody can be produced by expressing the human antibody-encoding gene in the cells. These methods are already known (see WO 1992/001047; WO 1992/020791; WO 1993/006213; WO 1993/011236; WO 1993/019172; WO 1995/001438; WO 1995/015388).

In addition to the techniques described above, techniques of B cell cloning (identification of each antibody-encoding sequence, cloning and its isolation; use in constructing expression vector in order to prepare each antibody (IgG1, IgG2, IgG3, or IgG4 in particular); and such) such as described in Bernasconi et al. (Science (2002) 298: 2199-2202) or in WO 2008/081008 can be appropriately used to isolate antibody genes.

Furthermore, in one embodiment, a polypeptide complex of the present invention may further comprise a substance that can bind to the human transferrin receptor. Polypeptide complexes of the present invention have suppressed target antigen-binding activity in systemic blood where plasma protein concentration is high, and exert target antigen-binding activity in CNS and such where plasma protein concentration is low. Meanwhile, polypeptide complexes of the present invention must pass through the blood-brain barrier (BBB) to access the CNS. Inflow of molecules such as proteins and small molecules into brain tissues is highly regulated by the BBB. Therefore, polypeptide complexes of the present invention are preferably used in combination with a technique for facilitating transport across the BBB.

Techniques for facilitating transport across the BBB include techniques that target receptors expressed at the BBB, such as transferrin receptor, insulin receptor, enkephalin receptor, and glutathione receptor. Integration of substances that target such receptors into polypeptide complexes of the present invention is expected to promote uptake of the polypeptide complexes of the present invention into the CNS. As molecules that target receptors expressed at the BBB, LRP-1 affinity peptide, anti-insulin receptor antibody, glutathione-PEG-liposome, melanotransferrin or melanotransferrin peptide, anti-transferrin receptor antibody, and such are known. In one embodiment, a polypeptide complex of the present invention can comprise a substance that binds to a transferrin receptor. Furthermore, in one embodiment, the substance that can bind to the human transferrin receptor may be an antibody that can bind to the human transferrin receptor. Furthermore, in one embodiment, the antibody may be a minibody (low molecular weight antibody) such as scFv, Fv, Fab, or VHH. Blood-brain barrier shuttles disclosed in WO2014033074A1 and in WO2015101588A1 can be presented as examples of such an antibody and minibody. These are anti-transferrin receptor antibodies and can facilitate transport of pharmaceutical agents across the BBB by transcytosis mediated by transferrin receptors on the cerebrovascular endothelial cells. Polypeptide complexes of the present invention may comprise a substance that can bind to the human transferrin receptor that is widely known to those skilled in the art.

In one embodiment, a polypeptide complex of the present invention may have a first antigen-binding portion comprising an amino acid sequence selected from the group consisting of SEQ ID NOs: 3, 4, 23, 24, 38 to 41, and 69 to 74.

Furthermore, in one embodiment, a polypeptide complex of the present invention may have an antibody heavy chain variable region comprising the amino acid sequence of SEQ ID NO: 1. In another embodiment, a polypeptide complex of the present invention may have an antibody heavy chain constant region comprising the amino acid sequence of SEQ ID NO: 5 or 6. Furthermore, in one embodiment, a polypeptide complex of the present invention may have an antibody light chain variable region comprising the amino acid sequence of SEQ ID NO: 2. In another embodiment, a polypeptide complex of the present invention may have an antibody light chain constant region comprising the amino acid sequence of SEQ ID NO: 7 or 8.

Furthermore, in one embodiment, a polypeptide complex of the present invention may have a fusion polypeptide of a first antigen-binding portion and an antibody heavy chain, which comprises an amino acid sequence selected from the group consisting of SEQ ID NOs: 10, 13, 19, 25, 27, 36, 57, and 63-65, or an antibody heavy chain comprising the amino acid sequence of SEQ ID NO: 12. Furthermore, in one embodiment, a polypeptide complex of the present invention may have a fusion polypeptide of a first antigen-binding portion and an antibody light chain, which comprises an amino acid sequence selected from the group consisting of SEQ ID NOs: 15, 18, 20, 26, 28, 37, 58, and 66-68, or an antibody light chain comprising the amino acid sequence of SEQ ID NO: 14 or 17.

In another embodiment, a polypeptide complex of the present invention may have an antibody heavy chain variable region comprising the amino acid sequence of SEQ ID NO: 47. Furthermore, in one embodiment, a polypeptide complex of the present invention may have an antibody heavy chain constant region comprising the amino acid sequence of SEQ ID NO: 6. Furthermore, in one embodiment, a polypeptide complex of the present invention may have an antibody light chain variable region comprising the amino acid sequence of SEQ ID NO: 48. Furthermore, in one embodiment, a polypeptide complex of the present invention may have an antibody light chain constant region comprising the amino acid sequence of SEQ ID NO: 8.

Furthermore, in one embodiment, a polypeptide complex of the present invention may have a fusion polypeptide of a first antigen-binding portion and an antibody heavy chain, which comprises the amino acid sequences of SEQ ID NOs: 51, 53, 55, 76 or 78. In one embodiment, a polypeptide complex of the present invention may have a fusion polypeptide of a first antigen-binding portion and an antibody light chain, which comprises the amino acid sequences of SEQ ID NOs: 52, 54, 56, 77 or 79.

### Pharmaceutical Compositions

In one embodiment, the present invention relates to pharmaceutical compositions comprising a polypeptide complex as described herein. A polypeptide complex comprised in the pharmaceutical compositions of the present invention show binding activity (or higher binding activity) to a target antigen in the absence of plasma protein or in an environment where the plasma protein concentration is low, and do not show binding activity (or show weaker binding activity) to a target antigen in the presence of plasma protein or in an environment where the plasma protein concentration is high. Therefore, pharmaceutical compositions of the present invention are expected to have potent therapeutic effects on diseases of the central nervous system and such where the plasma protein concentration is low, while avoiding side-effects caused by cytotoxicity, neutralizing actions, and such on tissues with high plasma protein concentration.

Pharmaceutical compositions of the present invention may comprise a pharmaceutically acceptable carrier. “A pharmaceutically acceptable carrier” refers to components other than the active ingredient in a pharmaceutical preparation which are nontoxic to the subject. Pharmaceutically acceptable carriers include, but are not limited to, buffers, vehicles, stabilizers, or preservatives.

The pharmaceutical compositions of the present invention can be formulated by methods known to those skilled in the art. For example, they can be used parenterally, in the form of injections of sterile solutions or suspensions including water or other pharmaceutically acceptable liquid. For example, such compositions can be formulated by mixing in the form of unit dose required in the generally approved medicine manufacturing practice, by appropriately combining with pharmacologically acceptable carriers or media, specifically with sterile water, physiological saline, vegetable oil, emulsifier, suspension, surfactant, stabilizer, flavoring agent, excipient, vehicle, preservative, binder, or such. In such formulations, the amount of active ingredient is adjusted to obtain an appropriate amount in a pre-determined range.

In a pharmaceutical composition or pharmaceutical formulation, “effective amount” refers to the amount that is effective for achieving the desired therapeutic or preventive result at a required dose and over a required period.

Sterile compositions for injection can be formulated using vehicles such as distilled water for injection, according to standard formulation practice. Aqueous solutions for injection include, for example, physiological saline and isotonic solutions containing dextrose or other adjuvants (for example, D-sorbitol, D-mannose, D-mannitol, and sodium chloride). It is also possible to use in combination appropriate solubilizers, for example, alcohols (ethanol and such), polyalcohols (propylene glycol, polyethylene glycol, and such), non-ionic surfactants (Polysorbate 80™, HCO-50, and such).

Oils include sesame oil and soybean oils. Benzyl benzoate and/or benzyl alcohol can be used in combination as solubilizers. It is also possible to combine buffers (for example, phosphate buffer and sodium acetate buffer), soothing agents (for example, procaine hydrochloride), stabilizers (for example, benzyl alcohol and phenol), and/or antioxidants. Appropriate ampules are filled with the prepared injections.

The pharmaceutical compositions of the present invention are preferably administered parenterally. For example, the compositions in the dosage form for injections, transnasal administration, transpulmonary administration, or transdermal administration are administered. For example, they can be administered systemically or locally by intravenous injection, intramuscular injection, intraperitoneal injection, subcutaneous injection, or such.

Administration methods can be appropriately selected in consideration of the patient's age and symptoms. The dose of a pharmaceutical composition containing a polypeptide complex of the present invention can be, for example, from 0.0001 to 1,000 mg/kg for each administration. Alternatively, the dose can be, for example, from 0.001 to 100,000 mg per patient. However, the present invention is not limited by the numeric values described above. The doses and administration methods vary depending on the patient's weight, age, symptoms, and such. Those skilled in the art can set appropriate doses and administration methods in consideration of the factors described above.

### Nucleic Acids, Vectors, and Host Cells

The nucleic acids or polynucleotides of the present invention refer to one or a plurality of nucleic acid molecules encoding the polypeptides constituting the polypeptide complexes of the present invention. The nucleic acids or polynucleotides of the present invention include at least one or a plurality of nucleic acid molecules that encode a first antigen-binding portion capable of specifically binding to a plasma protein and a second antigen-binding portion capable of binding to a target antigen, and include nucleic acid molecules on a single vector or separate vectors, and nucleic acid molecules present at a single position or multiple positions in host cells.

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

Nucleic acids of the present invention can be used, for example, in the production of polypeptide complexes of the present invention. When producing the polypeptide complexes of the present invention, ordinarily, nucleic acids encoding the polypeptide complexes of the present invention are inserted into suitable expression vectors, the vectors are introduced into appropriate cells, the transformed cells are cultured, and the expressed polypeptide complexes are isolated and purified. The polypeptide complexes can be expressed as fusion proteins with other proteins with the objective of facilitating purification. One can use, for example, the method of preparation as fusion proteins with a maltose-binding protein (vectors marketed from New England BioLabs, USA; pMAL series) using Escherichia coli as the host, the method of preparation as fusion proteins with glutathione-S-transferase (GST) (vectors marketed from Amersham Pharmacia Biotech; pGEX series), and the method of preparation by adding histidine tags (pET series from Novagen).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

In one embodiment of the present invention, nucleic acids of the present invention may be those inserted into vectors. For example, when E. coli is the host, for large scale amplification and large-scale preparation of the vector in E. coli (for example, JM109, DH5 alpha, HB101, and XL1Blue), examples of the vectors include but are not limited to vectors having “ori” for amplification in E. coli, and also having a selection gene for the transformed E. coli (for example, a drug-resistance gene discernible by a pharmaceutical (ampicillin or tetracycline, kanamycin, chloramphenicol, or such)). Examples of such vectors include the M13 vector, pUC vector, pBR322, pBluescript, and pCR-Script. Furthermore, when the objective is for subcloning and cutting out cDNA, examples of the vectors include pGEM-T, pDIRECT, and pT7 in addition to the above-mentioned vectors. When using vectors for the objective of producing polypeptide complexes, expression vectors are particularly useful. For example, when the objective is expression in E. coli, in addition to having the above-mentioned features that allow vector amplification in E. coli, if the host is E. coli such as JM109, DH5 alpha, HB101, or XL1-Blue, the expression vectors must have a promoter that enables efficient expression in E. coli, such as the lacZ promoter (Ward, et al., Nature (1989) 341, 544-546; FASEB J. (1992) 6, 2422-2427), the araB promoter (Better, et al., Science (1988) 240, 1041-1043), or the T7 promoter. In addition to the vectors mentioned above, examples of such vectors include pGEX-5X-1 (manufactured by Pharmacia), “QIAexpress system” (manufactured by QIAGEN), pEGFP, and pET.

Furthermore, the vectors may comprise a signal sequence for secreting the polypeptide. In case of production in the periplasm of E. coli, the pelB signal sequence (Lei, S. P. et al., J. Bacteriol. (1987) 169, 4379) can be used as the signal sequence for polypeptide secretion. The vectors can be introduced into a host cell using, for example, the calcium chloride method or electroporation. Furthermore, examples of vectors for expression in the plant body include pMH1, pMH2, and pCAMBIA.

In addition to E. coli, examples of vectors for producing the polypeptide complexes include expression vectors derived from mammals (for example, pcDNA3 (manufactured by Invitrogen), pEGF-BOS (Nucleic Acids. Res. 1990, 18(17), p5322), pEF, and pCDM8), expression vectors derived from insect cells (for example, “Bac-to-BAC baculovirus expression system” (manufactured by Gibco BRL) and pBacPAK8), expression vectors derived from plants (for example, pMH1 and pMH2), expression vectors derived from animal viruses (for example, pHSV, pMV, and pAdexLcw), expression vectors derived from retroviruses (for example, pZIPneo), expression vectors derived from yeast (for example, “Pichia Expression Kit” (manufactured by Invitrogen), pNV11, and SP-Q01), and expression vectors derived from Bacillus subtilis (for example, pPL608 and pKTH50).

When the objective is expression in animal cells such as CHO cells, COS cells, and NIH3T3 cells, the vector must carry promoters necessary for the expression within the cells, for example, the SV40 promoter (Mulligan et al., Nature (1979) 277, 108), the MMLV-LTR promoter, the EF1 alpha promoter (Mizushima et al., Nucleic Acids Res. (1990) 18, 5322), or the CMV promoter, and more preferably it has a gene for selecting transformation into the cells (for example, a drug-resistant gene discernible by pharmaceutical agents (neomycin, G418, and such)). Vectors having such properties include, for example, pMAM, pDR2, pBK-RSV, pBK-CMV, pOPRSV, and pOP13.

The host cells are not particularly limited as long as they are cells suitable for recombinant protein expression, and in addition to E. coli mentioned above, yeast, various animal and plant cells, insect cells, and such may be used. Various methods known to those skilled in the art can be used to introduce the vector into a host cell. For example, for introduction into E. coli, introduction methods that use calcium ions (Mandel, M., Higa, A. (1970) Journal of Molecular Biology, 53, 158-162; and Hanahan, D. (1983) Journal of Molecular Biology, 166, 557-580) can be used. Modified polypeptide complexes expressed in host cells can be purified and recovered by methods known to those skilled in the art from the host cells, or their cell culture or culture supernatant. When the polypeptide complexes are expressed as fusion proteins formed with the above-mentioned maltose-binding protein and such, they can be easily affinity-purified.

Suitable host cells for cloning or expression of vectors encoding the polypeptide complex of the present invention include prokaryotic or eukaryotic cells described herein. For example, polypeptide complexes may be produced in bacteria, in particular, when glycosylation and Fc effector function are not needed. For expression of a polypeptide complex and fragments thereof in bacteria, see for example, U.S. Pat. Nos. 5,648,237, 5,789,199, and 5,840,523 (See also Charlton, Methods in Molecular Biology, Vol. 248 (B. K. C. Lo, ed., Humana Press, Totowa, NJ, 2003), pp. 245-254, for expression of fragments in E. coli). After expression, the polypeptide complex may be isolated from the bacterial cell paste in a soluble fraction and can be further purified.

In addition to prokaryotes, eukaryotic microbes such as filamentous fungi or yeast are suitable hosts for cloning or expression of vectors encoding a polypeptide complex, including fungi and yeast strains whose glycosylation pathways have been “humanized”, which enable production of polypeptide complexes with a partially or fully human glycosylation pattern. See Gerngross, Nat. Biotech. 22:1409-1414 (2004), and Li et al., Nat. Biotech. 24:210-215 (2006).

Suitable host cells for the expression of a glycosylated polypeptide complex are also derived from multicellular organisms (invertebrates and vertebrates). Examples of invertebrate cells include plant and insect cells. Numerous baculovirus strains used in conjunction with insect cells, in particular, for transfection of Spodoptera frugiperda cells have been identified.

Plant cell cultures can also be utilized as hosts. See, for example, U.S. Pat. Nos. 5,959,177, 6,040,498, 6,420,548, 7,125,978, and 6,417,429 (describing PLANTIBODIES (trademark) technology for producing polypeptide complexes in transgenic plants).

Vertebrate cells may also be used as hosts. For example, mammalian cell lines that are adapted to grow in suspension are useful. Other examples of useful mammalian host cells are monkey kidney CV1 line transformed by SV40 (COS-7); human embryonic kidney line (293 or 293 cells as described in Graham et al., J. Gen Virol. 36:59 (1977)); baby hamster kidney cells (BHK); mouse sertoli cells (TM4 cells as described in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells (e.g., as described in Mather et al., Annals N.Y. Acad. Sci. 383:44-68 (1982)); MRC5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp20. In one embodiment, for a review of specific mammalian host cell lines suitable for production of an antibody constituting a polypeptide complex, see for example, Yazaki and Wu, Methods in Molecular Biology, Vol. 248 (B. K. C. Lo, ed., Humana Press, Totowa, NJ), pp. 255-268 (2003).

The terms “host cell”, “host cell line”, and “host cell culture” are used interchangeably and refer to cells into which exogenous nucleic acid has been introduced (including the progeny of such cells). Host cells include “transformants” and “transformed cells”, which include the primary transformed cell and progeny derived therefrom without regard to the number of passages. Progeny may not be completely identical in nucleic acid content to a parent cell, and may contain mutations. Mutant progenies that have the same function or biological activity as that used when screening or selecting the originally transformed cell are included in the “host cell” in the present specification.

Polypeptide complexes of the present invention do not bind (or have low binding activity) to target antigens in the presence of plasma protein (or in the presence of plasma protein at a high concentration), but can bind (or have high binding activity) to target antigens in the absence of plasma protein (or in the presence of plasma protein at a low concentration). Therefore, polypeptide complexes of the present invention can be used to detect and/or target antigen proteins in the absence of plasma protein or in the presence of plasma protein at a low concentration, such as in the central nervous system (CNS). More specifically, in one embodiment, the present invention relates to a composition comprising the polypeptide complex of the present invention, which is for detecting and/or targeting an antigen in the central nervous system (CNS) of a subject. Furthermore, the present invention relates to a method for detecting and/or targeting an antigen in the central nervous system (CNS), which comprises administering the polypeptide complex of the present invention to a subject. Furthermore, the present invention relates to a polypeptide complex for use in the detection and/or targeting of an antigen in the central nervous system (CNS) of a subject. Furthermore, the present invention relates to use of a polypeptide complex in producing a composition for detecting and/or targeting an antigen in the central nervous system (CNS) of a subject.

In one embodiment, targeting antigens refers to inhibiting or promoting the function and/or activity of the antigens, or inhibiting or promoting the binding between ligands and receptors through binding of the polypeptide complexes of the present invention to the antigens. Targeting antigens in the central nervous system (CNS) of subjects includes passing of the polypeptide complexes of the present invention through the blood-brain barrier (BBB) of the subjects to reach the CNS, and then binding to the antigens present in the CNS to cause actions such as those described above.

Route of administration of the polypeptide complexes of the present invention is not particularly limited, but parenteral administration is preferred. For example, polypeptide complexes of the present invention may be administered in the form of injections, transnasal administration, pulmonary administration, or subcutaneous administration. Alternatively, polypeptide complexes of the present invention may be administered systemically or locally by intravenous injection, intramuscular injection, intraperitoneal injection, subcutaneous injection, and such.

### Methods for Producing Polypeptide Complexes

In one embodiment, the present invention provides a method for producing a polypeptide complex, which comprises culturing, under conditions suitable for expression of a polypeptide complex of the present invention, a host cell comprising a nucleic acid encoding the polypeptide complex, and optionally, recovering the polypeptide complex from the host cell or its culture medium.

When producing polypeptide complexes of the present invention, ordinarily, nucleic acids encoding the polypeptide complexes of the present invention are inserted into suitable expression vectors, the vectors are introduced into suitable cells, the transformed cells are cultured, and the expressed polypeptide complexes are isolated and purified.

While expression vectors and host cells known to those skilled in the art may be used, in one embodiment, those described herein may also be used. Regarding culturing, when animal cells are used as hosts, the culture media that may be used include DMEM, MEM, RPMI1640, and IMDM, and these may be appropriately used in combination with serum complements such as FBS or fetal calf serum (FCS). Furthermore, cells may be cultured by serum-free culture.

A polypeptide complex produced by culturing host cells comprising nucleic acids that encode the polypeptide complex of the present invention under conditions that are suitable for its expression may be isolated from inside or outside of the host cells (media, milk, etc.), and purified as a substantially pure and homogeneous polypeptide complex. Isolation and purification methods that are generally used to purify polypeptides can be appropriately used to isolate and purify the polypeptide complex. For example, column chromatography, filters, ultrafiltration, salting out, solvent precipitation, solvent extraction, distillation, immunoprecipitation, SDS-polyacrylamide gel electrophoresis, isoelectric focusing, dialysis, and recrystallization are suitably selected and combined, but are not limited thereto. Chromatography includes, but is not limited to, affinity chromatography, ion exchange chromatography, hydrophobic chromatography, gel filtration chromatography, reverse phase chromatography, and adsorption chromatography. Such chromatography can be performed using liquid chromatography, for example, HPLC and FPLC. Columns for use in affinity chromatography include, but are not limited to, Protein A column and Protein G column. Protein A columns include, but are not limited to, Hyper D, POROS, Sepharose F. F. (Pharmacia), and so on.

In one embodiment, the present invention relates to a method for producing a polypeptide complex whose binding activity to a target antigen varies with the concentration of a plasma protein, which comprises a step of culturing a cell comprising a polynucleotide encoding the polypeptide complex,
wherein the polypeptide complex
(a) comprises a first antigen-binding portion capable of specifically binding to a plasma protein and a second antigen-binding portion capable of binding to a target antigen, and the first antigen-binding portion and the second antigen-binding portion are linked via a non-cleavable linker consisting of 0 to 4 amino acid residues, and
(b) has binding activity to the target antigen that is lower in the presence of the plasma protein than in the absence of the plasma protein.

Such polypeptide complexes can be obtained, for example, by measuring the binding activities of polypeptide complexes to the target antigen both in the presence and absence of the plasma protein, and selecting the polypeptide complexes whose binding activity to the target antigen is lower in the presence of the plasma protein than in the absence of the plasma protein.

Therefore, in one embodiment, the present invention relates to a method for producing a polypeptide complex whose binding activity to a target antigen varies with the concentration of a plasma protein, which comprises the following steps:
(a) measuring the binding activity of a polypeptide complex to the target antigen both in the presence and absence of a plasma protein;
(b) selecting a polypeptide complex whose binding activity to the target antigen is lower in the presence of the plasma protein than in the absence of the plasma protein;
(c) obtaining a polynucleotide encoding the polypeptide complex selected in (b); and
(d) culturing a cell comprising the polynucleotide obtained in (c).

Herein, the polypeptide complex comprises a first antigen-binding portion capable of specifically binding to the plasma protein and a second antigen-binding portion capable of binding to the target antigen. The first antigen-binding portion and the second antigen-binding portion are linked via a non-cleavable linker consisting of 0 to 4 amino acid residues.

In a method of the present invention, a step of contacting the target antigen with the polypeptide complex both in the presence and absence of the plasma protein may be included before step (a).

In one embodiment, the present invention relates to a method for producing a polypeptide complex whose binding activity to a target antigen varies with the concentration of a plasma protein, which comprises a step of culturing a cell comprising a polynucleotide encoding the polypeptide complex,
wherein the polypeptide complex
(a) comprises a first antigen-binding portion capable of specifically binding to a plasma protein and a second antigen-binding portion capable of binding to a target antigen, and the first antigen-binding portion and the second antigen-binding portion are linked via a non-cleavable linker consisting of 0 to 4 amino acid residues, and
(b) has binding activity to the target antigen that is different between in the presence of a first concentration of the plasma protein and in the presence of a second concentration of the plasma protein.

Here, the phrases “first concentration” and “second concentration” are as described herein.

Such polypeptide complexes can be obtained, for example, by measuring the binding activities of polypeptide complexes to the target antigen both in the presence of a first concentration of the plasma protein and in the presence of a second concentration of the plasma protein, and selecting polypeptide complexes whose binding activity to the target antigen is different between in the presence of the first concentration of the plasma protein and in the presence of the second concentration of the plasma protein.

Therefore, in one embodiment, the present invention relates to a method for producing a polypeptide complex whose binding activity to a target antigen varies with the concentration of a plasma protein, which comprises the following steps:
(a) measuring the binding activity of a polypeptide complex to the target antigen both in the presence of a first concentration of the plasma protein and in the presence of a second concentration of the plasma protein;
(b) selecting a polypeptide complex whose binding activity to the target antigen is different between in the presence of the first concentration of the plasma protein and in the presence of the second concentration of the plasma protein;
(c) obtaining a polynucleotide encoding the polypeptide complex selected in (b); and
(d) culturing a cell comprising the polynucleotide obtained in (c).

Herein, the polypeptide complex comprises a first antigen-binding portion capable of specifically binding to the plasma protein and a second antigen-binding portion capable of binding to the target antigen. The first antigen-binding portion and the second antigen-binding portion are linked via a non-cleavable linker consisting of 0 to 4 amino acid residues.

In a method of the present invention, a step of contacting the target antigen with the polypeptide complex both in the presence of the first concentration of the plasma protein and in the presence of the second concentration of the plasma protein may be included before step (a).

In one embodiment, the present invention relates to a method for producing a polypeptide complex whose binding activity to a target antigen varies with the concentration of a plasma protein, which comprises a step of culturing a cell comprising a polynucleotide encoding the polypeptide complex,
wherein the polypeptide complex
(a) comprises a first antigen-binding portion capable of specifically binding to the plasma protein and a second antigen-binding portion capable of binding to the target antigen, and the first antigen-binding portion and the second antigen-binding portion are linked via a non-cleavable linker consisting of 0 to 4 amino acid residues, and
(b) has binding activity to the target antigen that is lower in a human plasma sample than in the absence of the plasma protein.

Such polypeptide complexes can be obtained, for example, by measuring the binding activity of polypeptide complexes to the target antigen both in human plasma samples and in the absence of human plasma protein, and selecting polypeptide complexes whose binding activity to the target antigen is lower in human plasma samples than in the absence of the plasma protein.

Therefore, in one embodiment, the present invention relates to a method for producing a polypeptide complex whose binding activity to a target antigen varies with the concentration of a plasma protein, which comprises the following steps:
(a) measuring the binding activity of a polypeptide complex to the target antigen both in a human plasma sample and in the absence of a human plasma protein;
(b) selecting a polypeptide complex whose binding activity to the target antigen is lower in the human plasma sample than in the absence of the plasma protein;
(c) obtaining a polynucleotide encoding the polypeptide complex selected in (b); and
(d) culturing a cell comprising the polynucleotide obtained in (c).

Herein, the polypeptide complex comprises a first antigen-binding portion capable of specifically binding to the plasma protein and a second antigen-binding portion capable of binding to the target antigen. The first antigen-binding portion and the second antigen-binding portion are linked via a non-cleavable linker consisting of 0 to 4 amino acid residues.

In a method of the present invention, a step of contacting the target antigen with the polypeptide complex both in the human plasma sample and in the absence of the human plasma protein may be included before step (a).

In one embodiment, the present invention relates to a method for producing a polypeptide complex whose binding activity to a target antigen varies with the concentration of a plasma protein, which comprises a step of culturing a cell comprising a polynucleotide encoding the polypeptide complex,
wherein the polypeptide complex
(a) comprises a first antigen-binding portion capable of specifically binding to the plasma protein and a second antigen-binding portion capable of binding to the target antigen, and the first antigen-binding portion and the second antigen-binding portion are linked via a non-cleavable linker consisting of 0 to 4 amino acid residues, and
(b) has binding activity to the target antigen that is lower in a human plasma sample than in a human cerebrospinal fluid (CSF) sample.

Such polypeptide complexes can be obtained, for example, by measuring the binding activity of polypeptide complexes to the target antigen both in human plasma samples and in human cerebrospinal fluid (CSF) samples, and selecting polypeptide complexes whose binding activity to the target antigen is lower in human plasma samples than in human cerebrospinal fluid (CSF) samples.

Therefore, in one embodiment, the present invention relates to a method for producing a polypeptide complex whose binding activity to a target antigen varies with the concentration of a plasma protein, which comprises the following steps:
(a) measuring the binding activity of a polypeptide complex to the target antigen both in a human plasma sample and in a human cerebrospinal fluid (CSF) sample;
(b) selecting a polypeptide complex whose binding activity to the target antigen is lower in the human plasma sample than in the human cerebrospinal fluid (CSF) sample;
(c) obtaining a polynucleotide encoding the polypeptide complex selected in (b); and
(d) culturing a cell comprising the polynucleotide obtained in (c).

Herein, the polypeptide complex comprises a first antigen-binding portion capable of specifically binding to the plasma protein and a second antigen-binding portion capable of binding to the target antigen. The first antigen-binding portion and the second antigen-binding portion are linked via a non-cleavable linker consisting of 0 to 4 amino acid residues.

In a method of the present invention, a step of contacting the target antigen with the polypeptide complex both in the human plasma sample and in the human cerebrospinal fluid (CSF) sample may be included before step (a).

In one embodiment, the present invention relates to a method for producing a polypeptide complex whose binding activity to a target antigen varies with the concentration of a plasma protein, which comprises a step of culturing a cell comprising a polynucleotide encoding the polypeptide complex,
wherein the polypeptide complex
(a) comprises a first antigen-binding portion capable of specifically binding to the plasma protein and a second antigen-binding portion capable of binding to the target antigen, and the first antigen-binding portion and the second antigen-binding portion are linked via a non-cleavable linker consisting of 0 to 4 amino acid residues, and
(b) has binding activity to the target antigen that is lower in the presence of the plasma protein at about 20, 25, 30, 35, 40, 45, 50, 55, 60, 65, 70, 75 or 80 mg/mL than in the presence of the plasma protein at about 0.05, 0.10, 0.15, 0.20, 0.25, 0.30, 0.35, 0.40, 0.45 or 0.50 mg/mL.

Preferably, the polypeptide complex has binding activity to the target antigen that is lower in the presence of the plasma protein at 50 mg/mL than in the presence of the plasma protein at 0.25 mg/mL.

Such polypeptide complexes can be obtained, for example, by measuring the binding activity of polypeptide complexes to the target antigen both in the presence of plasma protein at about 20, 25, 30, 35, 40, 45, 50, 55, 60, 65, 70, 75 or 80 mg/mL and at about 0.05, 0.10, 0.15, 0.20, 0.25, 0.30, 0.35, 0.40, 0.45 or 0.50 mg/mL, and selecting the polypeptide complexes whose binding activity to the target antigen is lower in the presence of the plasma protein at 50 mg/mL than in the presence of the plasma protein at 0.25 mg/mL. Preferably, such polypeptide complex can be obtained, for example, by measuring the binding activity of polypeptide complexes to the target antigen both in the presence of plasma protein at 50 mg/mL and at 0.25 mg/mL, and selecting the polypeptide complexes whose binding activity to the target antigen is lower in the presence of the plasma protein at 50 mg/mL than in the presence of the plasma protein at 0.25 mg/mL.

Therefore, in one embodiment, the present invention relates to a method for producing a polypeptide complex whose binding activity to a target antigen varies with the concentration of a plasma protein, which comprises the following steps:
(a) measuring the binding activity of a polypeptide complex to the target antigen both in the presence of about 20, 25, 30, 35, 40, 45, 50, 55, 60, 65, 70, 75 or 80 mg/mL, preferably 50 mg/mL of the plasma protein and in the presence of about 0.05, 0.10, 0.15, 0.20, 0.25, 0.30, 0.35, 0.40, 0.45 or 0.50 mg/mL, preferably 0.25 mg/mL of the plasma protein;
(b) selecting a polypeptide complex whose binding activity to the target antigen is lower in the presence of about 20, 25, 30, 35, 40, 45, 50, 55, 60, 65, 70, 75 or 80 mg/mL, preferably 50 mg/mL of the plasma protein than in the presence of about 0.05, 0.10, 0.15, 0.20, 0.25, 0.30, 0.35, 0.40, 0.45 or 0.50 mg/mL, preferably 0.25 mg/mL of the plasma protein;
(c) obtaining a polynucleotide encoding the polypeptide complex selected in (b); and
(d) culturing a cell comprising the polynucleotide obtained in (c).

Herein, the polypeptide complex comprises a first antigen-binding portion capable of specifically binding to the plasma protein and a second antigen-binding portion capable of binding to the target antigen. The first antigen-binding portion and the second antigen-binding portion are linked via a non-cleavable linker consisting of 0 to 4 amino acid residues.

In a method of the present invention, a step of contacting the target antigen with the polypeptide complex both in the presence of about 20, 25, 30, 35, 40, 45, 50, 55, 60, 65, 70, 75 or 80 mg/mL of the plasma protein and in the presence of about 0.05, 0.10, 0.15, 0.20, 0.25, 0.30, 0.35, 0.40, 0.45 or 0.50 mg/mL of the plasma protein may be included before step (a).

In a method of the present invention, a step of contacting the target antigen with the polypeptide complex both in the presence of 50 mg/mL of the plasma protein and in the presence of 0.25 mg/mL of the plasma protein may be included before step (a).

In one embodiment, the present invention relates to a method for producing a polypeptide complex whose binding activity to a target antigen varies with the concentration of a plasma protein, which comprises a step of culturing a cell comprising a polynucleotide encoding the polypeptide complex,
wherein the polypeptide complex
(a) comprises a first antigen-binding portion capable of specifically binding to the plasma protein and a second antigen-binding portion capable of binding to the target antigen, and the first antigen-binding portion and the second antigen-binding portion are linked via a non-cleavable linker consisting of 0 to 4 amino acid residues, and
(b) has a KD value for the target antigen in the presence of the plasma protein at about 20, 25, 30, 35, 40, 45, 50, 55, 60, 65, 70, 75 or 80 mg/mL, preferably 50 mg/mL that is 5-fold or more, 10-fold or more, 15-fold or more, 20-fold or more, 25-fold or more, 30-fold or more, or 35-fold or more with respect to the KD value of the complex for the target antigen in the presence of the plasma protein at about 0.05, 0.10, 0.15, 0.20, 0.25, 0.30, 0.35, 0.40, 0.45 or 0.50 mg/mL, preferably 0.25 mg/mL.

Such polypeptide complexes can be obtained, for example, by measuring the binding activity of polypeptide complexes to the target antigen both in the presence of the plasma protein at about 20, 25, 30, 35, 40, 45, 50, 55, 60, 65, 70, 75 or 80 mg/mL, preferably 50 mg/mL and at about 0.05, 0.10, 0.15, 0.20, 0.25, 0.30, 0.35, 0.40, 0.45 or 0.50 mg/mL, preferably 0.25 mg/mL, and selecting the polypeptide complexes having KD values for the target antigen in the presence of plasma protein at about 20, 25, 30, 35, 40, 45, 50, 55, 60, 65, 70, 75 or 80 mg/mL, preferably 50 mg/mL that are 5-fold or more, 10-fold or more, 15-fold or more, 20-fold or more, 25-fold or more, 30-fold or more, or 35-fold or more with respect to the KD values of the complexes for the target antigen in the presence of the plasma protein at about 0.05, 0.10, 0.15, 0.20, 0.25, 0.30, 0.35, 0.40, 0.45 or 0.50 mg/mL, preferably 0.25 mg/mL.

Therefore, in one embodiment, the present invention relates to a method for producing a polypeptide complex whose binding activity to a target antigen varies with the concentration of a plasma protein, which comprises the following steps:
(a) measuring the binding activity of a polypeptide complex to the target antigen both in the presence of about 20, 25, 30, 35, 40, 45, 50, 55, 60, 65, 70, 75 or 80 mg/mL, preferably 50 mg/mL of the plasma protein and in the presence of about 0.05, 0.10, 0.15, 0.20, 0.25, 0.30, 0.35, 0.40, 0.45 or 0.50 mg/mL, preferably 0.25 mg/mL of the plasma protein;
(b) selecting a polypeptide complex whose KD value for the target antigen in the presence of about 20, 25, 30, 35, 40, 45, 50, 55, 60, 65, 70, 75 or 80 mg/mL, preferably 50 mg/mL of the plasma protein is 5-fold or more, 10-fold or more, 15-fold or more, 20-fold or more, 25-fold or more, 30-fold or more, or 35-fold or more with respect to the KD value of the complex for the target antigen in the presence of about 0.05, 0.10, 0.15, 0.20, 0.25, 0.30, 0.35, 0.40, 0.45 or 0.50 mg/mL, preferably 0.25 mg/mL of the plasma protein;
(c) obtaining a polynucleotide encoding the polypeptide complex selected in (b); and
(d) culturing a cell comprising the polynucleotide obtained in (c).

Herein, the polypeptide complex comprises a first antigen-binding portion capable of specifically binding to the plasma protein and a second antigen-binding portion capable of binding to the target antigen. The first antigen-binding portion and the second antigen-binding portion are linked via a non-cleavable linker consisting of 0 to 4 amino acid residues.

In a method of the present invention, a step of contacting the target antigen with the polypeptide complex both in the presence of about 20, 25, 30, 35, 40, 45, 50, 55, 60, 65, 70, 75 or 80 mg/mL of the plasma protein and in the presence of about 0.05, 0.10, 0.15, 0.20, 0.25, 0.30, 0.35, 0.40, 0.45 or 0.50 mg/mL of the plasma protein may be included before step (a).

In a method of the present invention, a step of contacting the target antigen with the polypeptide complex both in the presence of 50 mg/mL of the plasma protein and in the presence of 0.25 mg/mL of the plasma protein may be included before step (a).

In the above methods, the non-cleavable linker preferably consists of 0 to 3 amino acid residues, and more preferably consists of 0 to 1 amino acid residue. The phrase “non-cleavable linker consisting of 0 amino acid residue” means that no linker is included at all, namely, that the first antigen-binding portion and the second antigen-binding portion are linked without the mediation of a linker.

In the production methods of the present invention, the plasma protein is preferably albumin, and albumin is preferably human albumin. Furthermore, the target antigen is preferably a protein that is not a plasma protein. Specific binding activities of polypeptide complexes for a plasma protein and binding activities for a target antigen can be measured by methods known to those skilled in the art, and in one embodiment, measurements can be performed by methods described in this specification. Furthermore, polynucleotides encoding the polypeptide complexes can be obtained through nucleotide sequence determination by methods well known to those skilled in the art.

### Methods of Screening for Polypeptide Complexes

The present invention also relates to methods of screening for polypeptide complexes whose binding activity to a target antigen varies with the concentration of a plasma protein. The polypeptide complex of the present invention comprises a first antigen-binding portion capable of specifically binding to a plasma protein and a second antigen-binding portion capable of binding to a target antigen, and the first antigen-binding portion and the second antigen-binding portion are linked via a non-cleavable linker consisting of 0 to 4 amino acid residues. Herein the non-cleavable linker preferably consists of 0 to 3 amino acid residues, and more preferably consists of 0 to 1 amino acid residue. The phrase “non-cleavable linker consisting of 0 amino acid residue” means that no linker is included at all, namely, that the first antigen-binding portion and the second antigen-binding portion are linked without the mediation of a linker.

In one embodiment, a screening method of the present invention comprises the steps of:
(a) contacting a target antigen with a polypeptide complex both in the presence and absence of a plasma protein;
(b) measuring the binding activity of the polypeptide complex to the target antigen both in the presence and absence of the plasma protein; and
(c) selecting a polypeptide complex whose binding activity to the target antigen is lower in the presence of the plasma protein than in the absence of the plasma protein.

Furthermore, in one embodiment, a screening method of the present invention comprises the steps of:
(a) contacting a target antigen with a polypeptide complex both in the presence of a plasma protein at a first concentration and at a second concentration;
(b) measuring the binding activity of the polypeptide complex to the target antigen both in the presence of the first concentration of the plasma protein and in the presence of the second concentration of the plasma protein; and
(c) selecting a polypeptide complex whose binding activity to the target antigen is different between in the presence of the first concentration of the plasma protein and in the presence of the second concentration of the plasma protein.

Here, the terms “first concentration” and “second concentration” are as described in this specification.

Furthermore, in one embodiment, a screening method of the present invention comprises the steps of:
(a) contacting a target antigen with a polypeptide complex both in a human plasma sample and in the absence of a human plasma protein;
(b) measuring the binding activity of the polypeptide complex to the target antigen both in the human plasma sample and in the absence of the human plasma protein; and
(c) selecting a polypeptide complex whose binding activity to the target antigen is lower in the human plasma sample than in the absence of the plasma protein.

Furthermore, in one embodiment, a screening method of the present invention comprises the steps of:
(a) contacting a target antigen with a polypeptide complex both in a human plasma sample and in a human cerebrospinal fluid (CSF) sample;
(b) measuring the binding activity of the polypeptide complex to the target antigen both in the human plasma sample and in the human cerebrospinal fluid (CSF) sample; and
(c) selecting a polypeptide complex whose binding activity to the target antigen is lower in the human plasma sample than in the human cerebrospinal fluid (CSF) sample.

Furthermore, in one embodiment, a screening method of the present invention comprises the steps of:
(a) contacting a target antigen with a polypeptide complex both in the presence of a plasma protein at about 20, 25, 30, 35, 40, 45, 50, 55, 60, 65, 70, 75 or 80 mg/mL, preferably 50 mg/mL and at about 0.05, 0.10, 0.15, 0.20, 0.25, 0.30, 0.35, 0.40, 0.45 or 0.50 mg/mL, preferably 0.25 mg/mL;
(b) measuring the binding activity of the polypeptide complex to the target antigen both in the presence of about 20, 25, 30, 35, 40, 45, 50, 55, 60, 65, 70, 75 or 80 mg/mL, preferably 50 mg/mL of the plasma protein and in the presence of about 0.05, 0.10, 0.15, 0.20, 0.25, 0.30, 0.35, 0.40, 0.45 or 0.50 mg/mL, preferably 0.25 mg/mL of the plasma protein; and
(c) selecting a polypeptide complex whose binding activity to the target antigen is lower in the presence of about 20, 25, 30, 35, 40, 45, 50, 55, 60, 65, 70, 75 or 80 mg/mL, preferably 50 mg/mL of the plasma protein than in the presence of about 0.05, 0.10, 0.15, 0.20, 0.25, 0.30, 0.35, 0.40, 0.45 or 0.50 mg/mL, preferably 0.25 mg/mL of the plasma protein.

Furthermore, in one embodiment, a screening method of the present invention comprises the steps of:
(a) contacting a target antigen with a polypeptide complex both in the presence of a plasma protein at about 20, 25, 30, 35, 40, 45, 50, 55, 60, 65, 70, 75 or 80 mg/mL, preferably 50 mg/mL and at about 0.05, 0.10, 0.15, 0.20, 0.25, 0.30, 0.35, 0.40, 0.45 or 0.50 mg/mL, preferably 0.25 mg/mL;
(b) measuring the binding activity of the polypeptide complex to the target antigen both in the presence of about 20, 25, 30, 35, 40, 45, 50, 55, 60, 65, 70, 75 or 80 mg/mL, preferably 50 mg/mL of the plasma protein and in the presence of about 0.05, 0.10, 0.15, 0.20, 0.25, 0.30, 0.35, 0.40, 0.45 or 0.50 mg/mL, preferably 0.25 mg/mL of the plasma protein; and
(c) selecting a polypeptide complex whose KD value for the target antigen in the presence of about 20, 25, 30, 35, 40, 45, 50, 55, 60, 65, 70, 75 or 80 mg/mL, preferably 50 mg/mL of the plasma protein is 5-fold or more, 10-fold or more, 15-fold or more, 20-fold or more, 25-fold or more, 30-fold or more, or 35-fold or more with respect to the KD value of the complex for the target antigen in the presence of about 0.05, 0.10, 0.15, 0.20, 0.25, 0.30, 0.35, 0.40, 0.45 or 0.50 mg/mL, preferably 0.25 mg/mL of the plasma protein.

Screening methods of the present invention comprise a step of contacting a target antigen with a polypeptide complex. The contact can be carried out, for example, by adding a target antigen and a polypeptide complex into a sample that comprises a plasma protein at a certain concentration (for example, a sample comprising a plasma protein at a concentration described in this specification), a sample that does not comprise a plasma protein, a plasma sample derived from various animals such as humans, mice, rats, hamsters, rabbits, and monkeys, or a cerebrospinal fluid (CSF) sample. Alternatively, to such samples comprising either one of a target antigen or a polypeptide complex, the other one of a target antigen or a polypeptide complex may be added.

In the screening methods of the present invention, the plasma protein is preferably albumin, and albumin is preferably human albumin. Furthermore, the target antigen is preferably a protein that is not a plasma protein.

The screening methods of the present invention may further comprise after step (c) a step of (d) selecting a polypeptide complex that shows specific binding activity for the plasma protein.

Specific binding activity for a plasma protein and binding activity for a target antigen can be measured by methods well known to those skilled in the art. Examples include, but are not limited to Gyrolab xP (Gyros Protein Technologies), ELISA, Biacore (GE healthcare), Scatchard plot, and flow cytometer methods.

In the screening methods of the present invention, each of steps (a) to (d) may be repeated two or more times. Alternatively, the cycle of steps (a) to (c) and the cycle of steps (a) to (d) may be repeated two or more times.

The first antigen-binding portion capable of specifically binding to a plasma protein and the second antigen-binding portion capable of binding to a target antigen, which constitute the polypeptide complex to be screened by the screening method of the present invention, are not particularly limited. They may be those having naturally-occurring sequences. Alternatively, their amino acid sequences may have substitutions.

All prior art documents cited in this specification are incorporated herein by reference.

### EXAMPLES

### Example 1: Concept of Controlling Antigen Binding Using Plasma Protein-Binding Portions

Antigen-binding molecules, including antibodies, are known to have low rate of migration to the brain, which is 0.1% or lower (St-Amour, I., et al., Brain bioavailability of human intravenous immunoglobulin and its transport through the murine blood-brain barrier. J. Cereb. Blood Flow Metab. 2013 December; 33(12):1983-92). Therefore, antigen-binding molecules administered to a subject are present at high concentration in the plasma and at low concentration in the central nervous system (CNS) which includes the brain. To attain concentrations necessary for drug efficacy in the CNS, large amounts of antigen-binding molecules must be administered to the subject. As a result, concentration of the antigen-binding molecules in the plasma and the whole body becomes excessive, and binding with target antigens takes place in tissues other than the CNS which results in side effects. For example, although the TNF inhibitor infliximab is used in the treatment of neuro-Behcet disease, since it suppresses the immune response systemically, tuberculosis may relapse and lead to discontinuation of its administration (Horiguchi, N., et al., A 10-year follow-up of infliximab monotherapy for refractory uveitis in Behcet's syndrome. Sci. Rep. 10, 22227 (2020)).

If one can create a molecule that binds to a target antigen in the CNS but has decreased binding with the target antigen in tissues other than the CNS (non-CNS), actions in non-CNS can be decreased. An exemplary idea is a molecule that binds with an antigen in answer to the environmental difference between the CNS and non-CNS, but such a molecule had not existed.

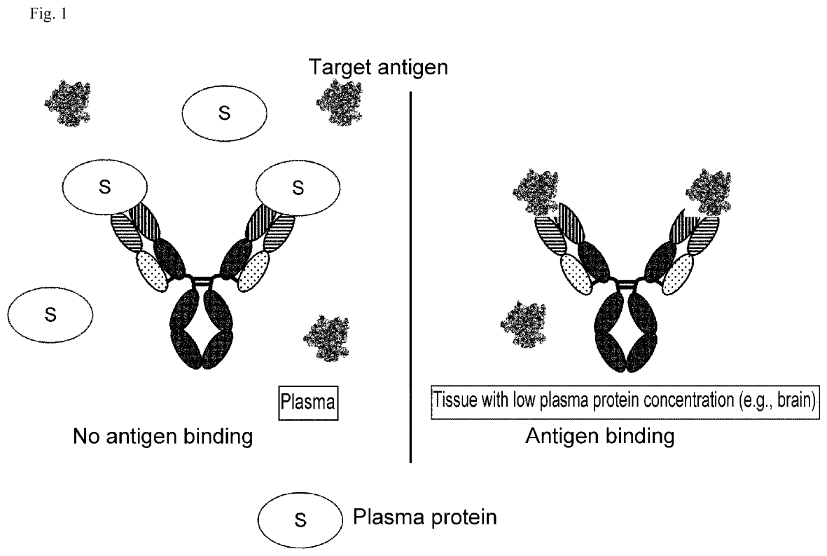

Plasma proteins (for example, albumin) are known as proteins present at the highest concentration in blood, systemically, including cerebral blood. Meanwhile, molecules must pass through a barrier known as the blood brain barrier (BBB) to access the CNS. Inflow of molecules such as proteins and small molecules are highly regulated by the BBB. As a result, plasma protein (for example, albumin) concentrations are known to be decreased in the CNS. According to a report, albumin concentration is approximately 600 micro M in the plasma, and approximately 3 micro M in the cerebrospinal fluid (CSF) (Kay, A. D., et al., CSF and serum concentrations of albumin and IgG in Alzheimer's disease. Neurobiol. Aging. 1987 January-February; 8(1):21-5). If antigen binding can be altered according to the concentration of plasma proteins (for example, albumin), one would be able to create a molecule that binds to a target antigen in the CNS but shows decreased binding with the target antigen (relative to that in the CNS) in tissues other than the CNS (FIG. 1).

When compared to systemic blood, tissues other than the CNS in which the concentrations of plasma proteins (for example, albumin) are known to be decreased include the ears (Thomsen, J., et., al. Quantitated Determination of Proteins in Perilymph in Patients with Acoustic Neuromas. ORL 1982; 44:61-65), the eyes (Stur, M., et., al. The effect of timolol on the concentrations of albumin and IgG in the aqueous humor of the human eye. Am. J. Ophthalmol. 1983 December; 96(6):726-9), the muscles (Ellmerer, M., et., al. Measurement of interstitial albumin in human skeletal muscle and adipose tissue by open-flow microperfusion. Am. J. Physiol. Endocrinol. Metab. 2000 February; 278(2):E352-6), and such. Plasma protein switch molecules of the present invention may also be applicable as therapeutic molecules targeting these tissues.

Protease-dependent antigen-binding molecules have been reported as examples of molecules that act disease-specifically. A protease-dependent antigen-binding molecule is composed of a peptide that inhibits an antigen-binding site, a protease cleavage site, and a target antigen-binding site. Treatment of such a molecule with a protease causes dissociation of the peptide that inhibits antigen-binding, leading to exposure of the target antigen-binding site, and allows the antibody to bind to the target antigen (for example, WO2019222282 and WO2013192546). However, since these molecules carry a protease cleavage site as their essential component, if protease cleavage does not take place, the peptide that inhibits antigen-binding would not dissociate, and therefore inhibition of binding to the target antigen would not be cancelled. Furthermore, since cleavage by protease is irreversible, when molecules after protease cleavage are redistributed throughout the body, they may create side effects by acting on normal tissues.

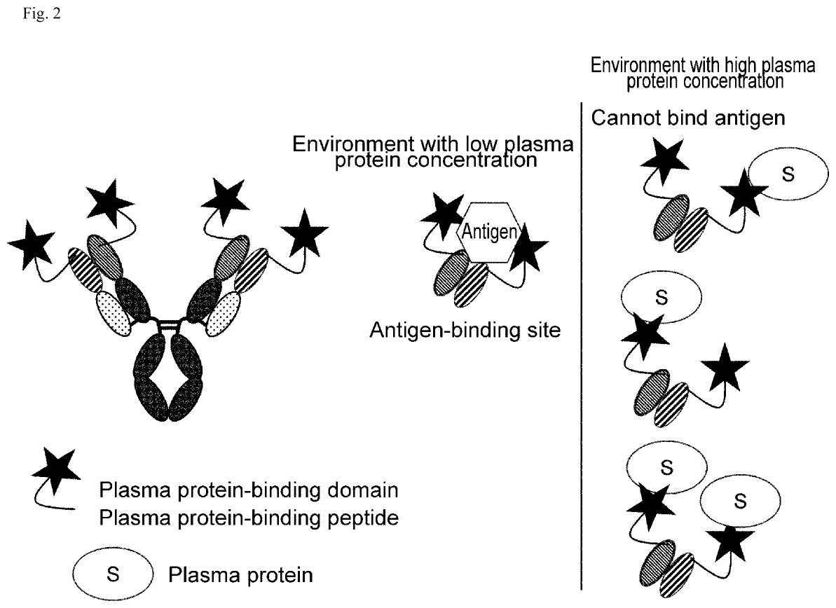

In contrast, a plasma protein switch molecule that does not use a protease cleavage site is valuable, firstly, because it can be used for diseases for which use of tissue-specific or disease-specific proteases are difficult. Secondly, when a plasma protein switch molecule that does not use a protease cleavage site is redistributed throughout the body from the disease site (CNS), the plasma protein can bind again to the plasma protein-binding site. Therefore, side effects caused by binding of this molecule to the target antigens in normal tissues can be avoided. The “reduced re-binding to the target antigen” is extremely important, considering the phenomena where various molecules are excreted by the cerebral tissue interstitial fluid and the cerebrospinal fluid (CSF). In CNS disease therapy, even if the therapeutic molecules reach the disease site (CNS) in one instance, their redistribution throughout the body along with excretion of the brain tissue interstitial fluid cannot be avoided; therefore, as the therapeutic molecules migrate from the CNS to the non-CNS, binding to the target antigen has to be decreased again. The present invention provides molecules whose binding activity for the target antigen changes according to the plasma protein concentration (plasma protein switch molecules). In one example, a plasma protein switch molecule has one or more plasma protein-binding portion linked to a single target antigen-binding site (FIG. 2). The present invention provides methods that enable selective binding in the brain of a molecule (plasma protein switch molecule) whose binding activity for the target antigen changes according to the plasma protein concentration.

### Example 2: Production of Molecules Whose Binding Changes by Albumin (Albumin Switch Molecules)

First, the heavy chain variable region (VH) (SEQ ID NO: 1) and the light chain variable region (VL) (SEQ ID NO: 2) of an anti-human IL-6R antibody (H54) were designed.

The VH and VL were designed so that an albumin-binding portion is attached to the N-terminal amino acid residue of VH and/or the N-terminal amino acid residue of VL of the anti-human IL-6R antibody. Specifically, a gene encoding the albumin-binding peptide ABP (SEQ ID NO: 3) or the albumin-binding domain ABD094 (SEQ ID NO: 4) was linked upstream of the gene(s) encoding the VH and/or VL of the anti-human IL-6R antibody. For some antibodies, N02 (SEQ ID NO: 35), N10, N11, N12, or N13 (SEQ ID NO: 62) was placed as a linker between the VH and/or VL of the anti-human IL-6R antibody and the albumin-binding portion. IC17 is a molecule that does not bind to the target antigen (human IL-6R) (WO2018043734A1). Aside from ABD094, ABD035 (SEQ ID NO: 23) or G418 (SEQ ID NO: 24) was used for the albumin-binding domain.

Genes encoding VH and VL that were designed this way, were synthesized and linked to the genes encoding the heavy chain constant region of SEQ ID NO: 5 (SG181) or SEQ ID NO: 6 (both heavy chain constant regions lack the C-terminal glycine and lysine) and the light chain constant region of SEQ ID NO: 7 (SK1) or SEQ ID NO: 8, respectively. Each of the linked genes were cloned into expression vectors.

Using the genes produced as described above, the antibodies listed in Table 1 were expressed transiently in mammalian cells by a method known to those skilled in the art, and purified by a method known to those skilled in the art when necessary.

| Name | Explanation | Albumin-binding portion |
|---|---|---|
| H54 | Anti-human IL-6R antibody | None |
| | (2 IL-6R-binding sites) | |
| ABP(H).N13-H54 | Anti-human IL-6R antibody | Albumin-binding peptide (SEQ ID NO: 3) |
| | (2 IL-6R-binding sites) | is linked at the VH N-terminus via |
| | | a linker (N13). |
| ABP(H/L).N13- | Anti-human IL-6R antibody | Albumin-binding peptide (SEQ ID NO: 3) |
| H54 | (2 IL-6R-binding sites) | is linked at the VH and VL N- |
| | | termini via a linker (N13). |
| IC17 | Anti-KLH antibody | None |
| | (no IL-6R-binding site) | |
| H54//IC17 | Anti-human IL-6R/KLH bispecific | None |
| | antibody (1 IL-6R-binding site) | |
| ABP(H/L).N13- | Anti-human IL-6R/KLH bispecific | Albumin-binding peptide (SEQ ID NO: 3) |
| H54//IC17 | antibody (1 IL-6R-binding site) | is linked at the VH and VL N-termini of |
| | | human IL-6R-binding arm via a linker (N13). |
| ABD094(H)- | Anti-human IL-6R/KLH bispecific | Albumin-binding domain (SEQ ID NO: 4) |
| H54//IC17 | antibody (1 IL-6R-binding site) | at the VH N-terminus of human |
| | | IL-6R-binding arm. No linker. |
| ABD094(L)- | Anti-human IL-6R/KLH bispecific | Albumin-binding domain (SEQ ID NO: 4) |
| H54//IC17 | antibody (1 IL-6R-binding site) | at the VL N-terminus of human |
| | | IL-6R-binding arm. No linker. |
| ABD094(H/L)- | Anti-human IL-6R/KLH bispecific | Albumin-binding domain (SEQ ID NO: 4) |
| H54//IC17 | antibody (1 IL-6R-binding site) | at the VH and VL N-termini of |
| | | human IL-6R-binding arm. No linker. |
| ABD094(H/L).N02- | Anti-human IL-6R/KLH bispecific | Albumin-binding domain (SEQ ID NO: 4) |
| H54//IC17 | antibody (1 IL-6R-binding site) | is linked at the VH and VL N-termini of |
| | | human IL-6R-binding arm via a linker (N02). |
| ABD094(H/L).N10- | Anti-human IL-6R/KLH bispecific | Albumin-binding domain (SEQ ID NO: 4) |
| H54//IC17 | antibody (1 IL-6R-binding site) | is linked at the VH and VL N-termini of |
| | | human IL-6R-binding arm via a linker (N10). |
| ABD094(H/L).N11- | Anti-human IL-6R/KLH bispecific | Albumin-binding domain (SEQ ID NO: 4) |
| H54//IC17 | antibody (1 IL-6R-binding site) | is linked at the VH and VL N-termini of |
| | | human IL-6R-binding arm via a linker (N11). |
| ABD094(H/L).N12- | Anti-human IL-6R/KLH bispecific | Albumin-binding domain (SEQ ID NO: 4) |
| H54//IC17 | antibody (1 IL-6R-binding site) | is linked at the VH and VL N-termini of |
| | | human IL-6R-binding arm via a linker (N12). |
| ABD094(H/L).N13- | Anti-human IL-6R/KLH bispecific | Albumin-binding domain (SEQ ID NO: 4) |
| H54//IC17 | antibody (1 IL-6R-binding site) | is linked at the VH and VL N-termini of |
| | | human IL-6R-binding arm via a linker (N13). |
| ABD035(H)- | Anti-human IL-6R/KLH bispecific | Albumin-binding domain (SEQ ID NO: 23) |
| H54//IC17 | antibody (1 IL-6R-binding site) | at the VH N-terminus of human |
| | | IL-6R-binding arm. No linker. |
| ABD035(L)- | Anti-human IL-6R/KLH bispecific | Albumin-binding domain (SEQ ID NO: 23) |
| H54//IC17 | antibody (1 IL-6R-binding site) | at the VL N-terminus of human |
| | | IL-6R-binding arm. No linker. |
| ABD035(H/L)- | Anti-human IL-6R/KLH bispecific | Albumin-binding domain (SEQ ID NO: 23) |
| H54//IC17 | antibody (1 IL-6R-binding site) | at the VH and VL N-termini of |
| | | human IL-6R-binding arm. No linker. |
| G418(H)- | Anti-human IL-6R/KLH bispecific | Albumin-binding domain (SEQ ID NO: 24) |
| H54//IC17 | antibody (1 IL-6R-binding site) | at the VH N-terminus of human |
| | | IL-6R-binding arm. No linker. |
| G418(L)- | Anti-human IL-6R/KLH bispecific | Albumin-binding domain (SEQ ID NO: 24) |
| H54//IC17 | antibody (1 IL-6R-binding site) | at the VL N-terminus of human |
| | | IL-6R-binding arm. No linker. |
| G418(H/L)- | Anti-human IL-6R/KLH bispecific | Albumin-binding domain (SEQ ID NO: 24) |
| H54//IC17 | antibody (1 IL-6R-binding site) | at the VH and VL N-termini of |
| | | human IL-6R-binding arm. No linker. |

The combinations of amino acid sequences for H54, ABP(H).N13-H54, ABP(H/L).N13-H54, and IC17 are shown in Table 2.

TABLE 2

| | | | | | | H chain full length | L chain full length |
|---|---|---|---|---|---|---|---|
| | Albumin-binding | | | | | (including albumin- | (including albumin- |
| Name | portion | VH | CH | VL | CL | binding portion) | binding portion) |
| H54 | None | SEQ ID NO: 1 | SEQ ID NO: 5 | SEQ ID NO: 2 | SEQ ID NO: 7 | SEQ ID NO: 9 | SEQ ID NO: 14 |
| ABP(H).N13- | SEQ ID NO: 3 + | SEQ ID NO: 1 | SEQ ID NO: 5 | SEQ ID NO: 2 | SEQ ID NO: 7 | SEQ ID NO: 10 | SEQ ID NO: 14 |
| H54 | linker (SEQ ID | | | | | | |
| | NO: 62) | | | | | | |
| ABP(H/L).N13- | SEQ ID NO: 3 + | SEQ ID NO: 1 | SEQ ID NO: 5 | SEQ ID NO: 2 | SEQ ID NO: 7 | SEQ ID NO: 10 | SEQ ID NO: 15 |
| H54 | linker (SEQ ID | | | | | | |
| | NO: 62) | | | | | | |
| IC17 | None | — | SEQ ID NO: 5 | — | SEQ ID NO: 7 | SEQ ID NO: 11 | SEQ ID NO: 16 |

The combinations of amino acid sequences for H54//IC17, ABP(H/L)-H54//IC17, ABD094(H)-H54//IC17, ABD094(L)-H54//IC17, ABD094(H/L)-H54//IC17, and ABD094(H/L).N02-H54//IC17 are shown in Tables 3-1 and 3-2.

TABLE 3-1

| | Human IL-6R-binding arm | | | | | | |
|---|---|---|---|---|---|---|---|
| | | | | | | H chain full length | L chain full length |
| | Albumin-binding | | | | | (including albumin- | (including albumin- |
| Name | portion | VH | CH | VL | CL | binding portion) | binding portion) |
| H54//IC17 | None | SEQ ID NO: 1 | SEQ ID NO: 6 | SEQ ID NO: 2 | SEQ ID NO: 8 | SEQ ID NO: 12 | SEQ ID NO: 17 |
| ABP(H/L).N13- | SEQ ID NO: 3 + | SEQ ID NO: 1 | SEQ ID NO: 6 | SEQ ID NO: 2 | SEQ ID NO: 8 | SEQ ID NO: 13 | SEQ ID NO: 18 |
| H54//IC17 | linker (SEQ ID | | | | | | |
| | NO: 62) | | | | | | |
| ABD094(H)- | SEQ ID NO: 4 | SEQ ID NO: 1 | SEQ ID NO: 6 | SEQ ID NO: 2 | SEQ ID NO: 8 | SEQ ID NO: 19 | SEQ ID NO: 17 |
| H54//IC17 | | | | | | | |
| ABD094(L)- | SEQ ID NO: 4 | SEQ ID NO: 1 | SEQ ID NO: 6 | SEQ ID NO: 2 | SEQ ID NO: 8 | SEQ ID NO: 12 | SEQ ID NO: 20 |
| H54//IC17 | | | | | | | |
| ABD094(H/L)- | SEQ ID NO: 4 | SEQ ID NO: 1 | SEQ ID NO: 6 | SEQ ID NO: 2 | SEQ ID NO: 8 | SEQ ID NO: 19 | SEQ ID NO: 20 |
| H54//IC17 | | | | | | | |
| ABD094(H/L).N02- | SEQ ID NO: 4 + | SEQ ID NO: 1 | SEQ ID NO: 6 | SEQ ID NO: 2 | SEQ ID NO: 8 | SEQ ID NO: 36 | SEQ ID NO: 37 |
| H54//IC17 | linker (SEQ ID | | | | | | |
| | NO: 35) | | | | | | |
| ABD094(H/L).N10- | SEQ ID NO: 4 + | SEQ ID NO: 1 | SEQ ID NO: 6 | SEQ ID NO: 2 | SEQ ID NO: 8 | SEQ ID NO: 57 | SEQ ID NO: 58 |
| H54//IC17 | linker | | | | | | |
| ABD094(H/L).N11- | SEQ ID NO: 4 + | SEQ ID NO: 1 | SEQ ID NO: 6 | SEQ ID NO: 2 | SEQ ID NO: 8 | SEQ ID NO: 63 | SEQ ID NO: 66 |
| H54//IC17 | linker | | | | | | |
| ABD094(H/L).N12- | SEQ ID NO: 4 + | SEQ ID NO: 1 | SEQ ID NO: 6 | SEQ ID NO: 2 | SEQ ID NO: 8 | SEQ ID NO: 64 | SEQ ID NO: 67 |
| H54//IC17 | linker | | | | | | |
| ABD094(H/L).N13- | SEQ ID NO: 4 + | SEQ ID NO: 1 | SEQ ID NO: 6 | SEQ ID NO: 2 | SEQ ID NO: 8 | SEQ ID NO: 65 | SEQ ID NO: 68 |
| H54//IC17 | linker (SEQ ID | | | | | | |
| | NO: 62) | | | | | | |
| ABD035(H)- | SEQ ID NO: 23 | SEQ ID NO: 1 | SEQ ID NO: 6 | SEQ ID NO: 2 | SEQ ID NO: 8 | SEQ ID NO: 25 | SEQ ID NO: 17 |
| H54//IC17 | | | | | | | |
| ABD035(L)- | SEQ ID NO: 23 | SEQ ID NO: 1 | SEQ ID NO: 6 | SEQ ID NO: 2 | SEQ ID NO: 8 | SEQ ID NO: 12 | SEQ ID NO: 26 |
| H54//IC17 | | | | | | | |
| ABD035(H/L)- | SEQ ID NO: 23 | SEQ ID NO: 1 | SEQ ID NO: 6 | SEQ ID NO: 2 | SEQ ID NO: 8 | SEQ ID NO: 25 | SEQ ID NO: 26 |
| H54//IC17 | | | | | | | |
| G418(H)- | SEQ ID NO: 24 | SEQ ID NO: 1 | SEQ ID NO: 6 | SEQ ID NO: 2 | SEQ ID NO: 8 | SEQ ID NO: 27 | SEQ ID NO: 17 |
| H54//IC17 | | | | | | | |
| G418(L)- | SEQ ID NO: 24 | SEQ ID NO: 1 | SEQ ID NO: 6 | SEQ ID NO: 2 | SEQ ID NO: 8 | SEQ ID NO: 12 | SEQ ID NO: 28 |
| H54//IC17 | | | | | | | |
| G418(H/L)- | SEQ ID NO: 24 | SEQ ID NO: 1 | SEQ ID NO: 6 | SEQ ID NO: 2 | SEQ ID NO: 8 | SEQ ID NO: 27 | SEQ ID NO: 28 |
| H54//IC17 | | | | | | | |

TABLE 3-2

| | KLH-binding arm | | |
|---|---|---|---|
| | Albumin- | H chain | L chain |
| | binding | full | full |
| Name | portion | length | length |
| H54//IC17 | None | SEQ ID | SEQ ID |
| ABP(H/L).N13-H54//IC17 | | NO: 21 | NO: 22 |
| ABD094(H)-H54//IC17 | | | |
| ABD094(L)-H54//IC17 | | | |
| ABD094(H/L)-H54//IC17 | | | |
| ABD094(H/L).N02-H54//IC17 | | | |
| ABD094(H/L).N10-H54//IC17 | | | |
| ABD094(H/L).N11-H54//IC17 | | | |
| ABD094(H/L).N12-H54//IC17 | | | |
| ABD094(H/L).N13-H54//IC17 | | | |
| ABD035(H)-H54//IC17 | | | |
| ABD035(L)-H54//IC17 | | | |
| ABD035(H/L)-H54//IC17 | | | |
| G418(H)-H54//IC17 | | | |
| G418(L)-H54//IC17 | | | |
| G418(H/L)-H54//IC17 | | | |

### Example 3: Confirmation of Antigen Binding of Albumin Switch Molecules (ABP and ABD094)

### Example 3-1: Evaluation of Binding to Human IL-6R

Binding activities of the produced albumin switch molecules against human IL-6R were evaluated using Biacore T200 (Cytiva). As the running buffer, 50 mM Na Phosphate (pH 7.4), 150 mM NaCl, 0.05% Tween20 was used, and the evaluation was performed at 37 degrees C. First, Protein G (CALBIOCHEM) was used as a ligand-capturing molecule. Protein G (CALBIOCHEM) was solid-phased on a Series S Sensor Chip CM4 (Cytiva) using an Amine Coupling Kit (Cytiva). Antibody solution prepared using the running buffer was allowed to interact with this CM4 sensor chip to capture approximately 200 RU of the antibody. Human IL-6R used in the present measurement was prepared by an approach described in WO2018097307 (Example 3-2). Human IL-6R was diluted to 0, 100, and 400 nM with the running buffer, and allowed to bind to the captured antibody. The chip was regenerated using 3 M MgCl₂, and measurements were performed by repeatedly capturing the antibodies. Dissociation constant (KD) of each antibody for human IL-6R was calculated using Biacore T200 Evaluation Software 2.0. Specifically, the association rate constant ka (L/mol/s) and the dissociation rate constant kd (1/s) were calculated by global fitting of the sensorgrams obtained by measurement with the 1:1 Langmuir binding model, and the dissociation constant KD (mol/L) was calculated from these values. Table 4 shows the results of these measurements.

TABLE 4

| | Affinity against hIL-6R | | |
|---|---|---|---|
| Antibody Name | ka (1/Ms) | kd (1/s) | KD (M) |
| H54//IC17 | 4.12E+05 | 1.41E−03 | 3.41E−09 |
| ABP(H/L).N13-H54//IC17 | 4.71E+05 | 9.51E−04 | 2.02E−09 |
| ABD094(H)-H54//IC17 | 2.55E+05 | 1.33E−03 | 5.21E−09 |
| ABD094(L)-H54//IC17 | 4.45E+05 | 1.18E−03 | 2.65E−09 |
| ABD094(H/L)-H54//IC17 | 2.10E+05 | 7.79E−04 | 3.70E−09 |

Regardless of the presence or absence, or the number of albumin-binding portions, all of the above anti-human IL-6R/KLH bispecific antibodies were confirmed to have comparable level of binding activity against human IL-6R in the absence of albumin.

In each of ABD094(H)-H54//IC17, ABD094(L)-H54//IC17, and ABD094(H/L)-H54//IC17, the target antigen-binding portion and the albumin-binding portion are directly linked without being mediated by a linker. The fact that even without the use of a linker, the binding activities of these molecules for the target antigen (human IL-6R) are comparable to that of H54//IC17, which does not have an albumin-binding portion, was an unexpected finding. ABP(H/L).N13-H54//IC17 has the target antigen-binding portion and the albumin-binding portion linked via a linker (Gly-Gly-Gly-Ser (SEQ ID NO: 42)). Even when such a short linker was used, the binding activity of the molecule for the target antigen (human IL6R) was found to be comparable to that of H54//IC17 which does not have an albumin-binding portion.

### Example 3-2: Evaluation of Binding to Mouse Albumin

Binding activities of the produced antibodies against mouse albumin were evaluated using Biacore T200 (Cytiva).

As the running buffer, 50 mM Na Phosphate (pH 7.4), 150 mM NaCl, 0.05% Tween20 was used, and the evaluation was performed at 25 degrees C. First, anti-human Fc antibody (Cytiva) was used as a ligand-capturing molecule. Anti-human Fc antibody (Cytiva) was solid-phased on a Series S Sensor Chip CM4 (Cytiva) using an Amine Coupling Kit (Cytiva). Antibody solution prepared using the running buffer was allowed to interact with this CM4 sensor chip to capture approximately 120 RU of the antibody. Mouse albumin (Bioscience) was diluted to 0 and 300 nM with the running buffer and allowed to bind to the captured antibody. The chip was regenerated using 3 M MgCl₂, and measurements were performed by repeatedly capturing the antibodies. Dissociation constant (KD) of each antibody for mouse albumin was calculated using Biacore T200 Evaluation Software 2.0. Specifically, the association rate constant ka (L/mol/s) and the dissociation rate constant kd (1/s) were calculated by global fitting of the sensorgrams obtained by measurement with the 1:1 Langmuir binding model, and the dissociation constant KD (mol/L) was calculated from these values. Table 5 shows the results of these measurements.

TABLE 5

| | Affinity against recombinant mouse Albumin | | |
|---|---|---|---|
| Antibody Name | ka (1/Ms) | kd (1/s) | KD (M) |
| H54//IC17 | N.D. | N.D. | N.D. |
| ABP(H/L).N13-H54//IC17 | 4.35E+04 | 4.43E−04 | 1.02E−08 |
| ABD094(H)-H54//IC17 | 7.18E+04 | 1.03E−03 | 1.43E−08 |
| ABD094(L)-H54//IC17 | 1.13E+05 | 1.31E−03 | 1.16E−08 |
| ABD094(H/L)-H54//IC17 | 5.76E+04 | 1.12E−03 | 1.94E−08 |

Regardless of the position and the number of albumin-binding portions, all of the above anti-human IL-6R/KLH bispecific antibodies having the albumin-binding portion were confirmed to have comparable level of binding activity against mouse albumin.

### Example 4-1: Assessment of Antigen Binding of Anti-Human IL-6R Albumin Switch Molecules in Mouse Plasma (ABP and ABD094)

To evaluate whether the antigen binding of anti-human IL-6R albumin switch molecules changes by albumin (mouse albumin), antibody-human soluble IL-6R complexes in mouse plasma were measured. The measurements were performed using Gyrolab xP (Gyros Protein Technologies).

First, to bind the albumin-binding portion to albumin in the plasma, the anti-human IL-6R antibodies (albumin switch molecules) indicated in Example 2 were diluted to 0.6 micro g/mL using C57BL/6J mouse plasma, and then incubated at room temperature for one hour. Next, for binding of the anti-human IL-6R antibody to the target antigen, one equivalent of human soluble IL-6R diluted to 0.6 micro g/mL in mouse plasma was added to the antibody solutions, and then the solutions were incubated at room temperature for one hour.

To detect the antigen-antibody complexes, first, the anti-delta GK antibody (AF647-anti-delta GK) was labeled using an Alexa flour 647 labeling kit (A20186, Thermo Fisher Scientific) according to the attached protocols. The labeled anti-delta GK antibody (an antibody that binds specifically to the Fc region lacking two amino acids, glycine and lysine at the heavy chain C terminus) was diluted to 20 micro g/mL with PBS 0.05% Tween 20 (P3563, sigma), one equivalent of this solution was added to a mixed solution of anti-human IL-6R antibody and human soluble IL-6R, and this was incubated at room temperature for three hours and then O/N incubated at 4 degrees C. (the final concentrations were 0.15 micro g/mL for the antibody, 0.15 micro g/mL for human soluble IL-6R, and 10 micro g/mL for AF647-anti-delta GK).

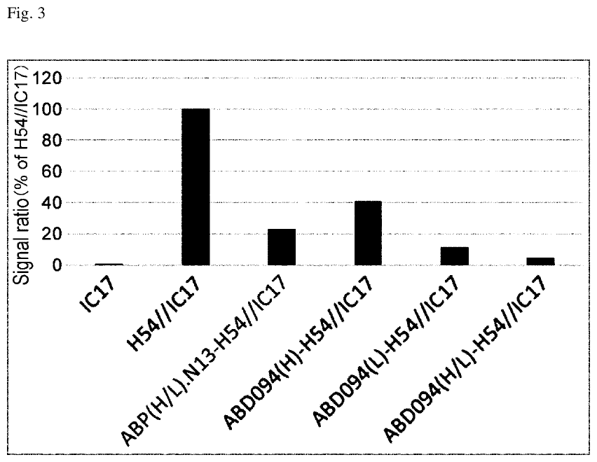

Measurement of antigen-antibody complexes was performed using Gyrolab xP. For the measurements using Gyrolab xP, Gyrolab Bioaffy 200 (P0004180, Gyros Protein Technologies) was used as the measurement disc. Biotinylated anti-human IL-6R antibody (R&D Systems, BAF227) diluted to 10 micro g/mL with PBS 0.05% Tween 20 was added to the measurement disc, and then solid-phased on the reaction layer in the disc. A mixed solution of the labeled anti-delta GK antibody, anti-human IL-6R antibody (albumin switch molecule), and human soluble IL-6R, incubated as described above, was added to the disc, and the anti-human IL-6R antibody-human soluble IL-6R-AF647-anti-delta GK complex was captured by the solid-phased antibody. The amount of the captured complex was detected by the fluorescence signal of AF647 (Detect PMT 25%). The fluorescence signal of AF647 was analyzed by Gyro Evaluator (version 3.6.2.30, Gyros Protein Technologies). The results of the analysis, which are signal values of each of the samples when the signal value obtained with H54//IC17 was set at 100%, are shown in FIG. 3.

Compared to the anti-human IL-6R/KLH bispecific antibody H54//IC17, not carrying an albumin-binding portion, ABP(H/L).N13-H54//IC17, carrying an added albumin-binding peptide (ABP), showed 77% decrease in the signal derived from the complex formed with human soluble IL-6R. Furthermore, regarding antibodies carrying the albumin-binding domain (ABD094), the signal decreased in the order of ABD094(H)-H54//IC17, ABD094(L)-H54//IC17, and ABD094(H/L)-H54//IC17. Regarding ABD094 (H/L)-H54//IC17 whose signal decreased most remarkably, the signal decreased 95% compared to that of H54//IC17.

In view of the above, it was shown that by adding an albumin-binding portion to an antibody, binding to a target antigen in the presence of albumin can be reduced without substantially changing the binding to the target antigen in the absence of albumin. Moreover, surprisingly, it was shown that compared to when one albumin-binding portion is added to one target antigen-binding portion (one arm of the antibody), when two or more albumin-binding portions are added, binding to the target antigen in the presence of albumin can be inhibited more effectively.

Antibodies ABD094(H)-H54//IC17 and ABD094(L)-H54//IC17, produced by adding one albumin-binding portion to one target antigen-binding portion (one arm of the antibody), were compared. Surprisingly, the signal derived from the complex formed with soluble human IL-6R was greatly decreased for ABD094(L)-H54//IC17 having the albumin-binding portion added to the N terminus of the L-chain variable region, when compared to that of ABD094(H)-H54//IC17 having the albumin-binding portion added to the N terminus of the H-chain variable region (FIG. 3). Accordingly, it was shown that when adding an albumin-binding portion to the H54 antibody (anti-human IL-6R antibody), binding to the target antigen in the presence of albumin can be inhibited more effectively by adding the albumin-binding portion to the N terminus of the L-chain variable region.

### Example 4-2: Assessment of Antigen Binding of Anti-IL-6R Albumin Switch Molecules in Human, Rat, and Monkey Plasma (ABP and ABD094)

To evaluate whether the antigen binding of anti-IL-6R albumin switch molecules in human, rat, and monkey plasma changes by albumin (human, rat, or monkey albumin), antibody-human soluble IL-6R complexes in human or rat plasma and antibody-monkey soluble IL-6R complexes in monkey plasma were measured. The measurements were performed using Gyrolab xP (Gyros Protein Technologies).

In the measurements of antibody-human soluble IL-6R complexes in human and rat plasma, first, to bind the albumin-binding portion to albumin in the plasma, the anti-human IL-6R antibodies (albumin switch molecules) indicated in Example 2 were diluted to 4 nM using human plasma (HUMANPLNHUNN, BIOIVT) or SD rat plasma, and then incubated at room temperature for 45 minutes. Next, for binding of the anti-human IL-6R antibody to the target antigen, one equivalent of human soluble IL-6R diluted to 16 nM in human or rat plasma was added to the antibody solutions, and then the solutions were incubated at room temperature for one hour.

In the measurement of antibody-monkey soluble IL-6R complexes in monkey plasma, first, to bind the albumin-binding portion to albumin in the plasma, the anti-human IL-6R antibodies (albumin switch molecules) indicated in Example 2 were diluted to 4 nM using cynomolgus monkey plasma, and then incubated at room temperature for 45 minutes. Next, for binding of the anti-human IL-6R antibody to the target antigen, one equivalent of monkey soluble IL-6R diluted to 16 nM in monkey plasma was added to the antibody solutions, and then the solutions were incubated at room temperature for one hour. It is noted that the anti-human IL-6R antibody (H54) indicated in Example 2 has been confirmed to have cross-reactivity to monkey soluble IL-6R.

To detect the antigen-antibody complexes, first, the anti-delta GK antibody (AF647-anti-delta GK) was labeled using an Alexa flour 647 labeling kit (A20186, Thermo Fisher Scientific) according to the attached protocols. The labeled anti-delta GK antibody was diluted to 133 nM with PBS 0.05% Tween 20 (P3563, sigma), one equivalent of this solution was added to a mixed solution of anti-human IL-6R antibody and human soluble IL-6R and to a mixed solution of anti-human IL-6R antibody and monkey soluble IL-6R, and these were incubated at room temperature for 45 minutes, and then O/N incubated at 4 degrees C. (the final concentrations were 1 nM for the antibody, 4 nM for human soluble IL-6R and monkey soluble IL-6R, and 67 nM for AF647-anti-delta GK).

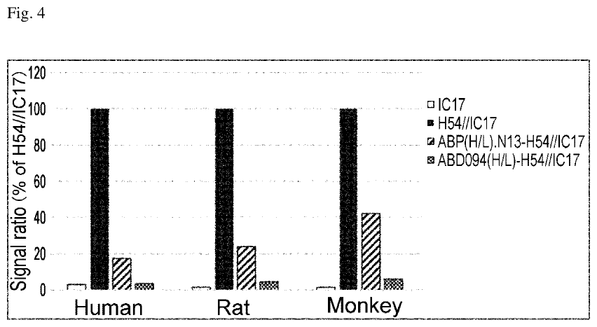

Measurement of antigen-antibody complexes was performed using Gyrolab xP. For the measurements using Gyrolab xP, Gyrolab Bioaffy 200 (P0004180, Gyros Protein Technologies) was used as the measurement disc. Biotinylated anti-human IL-6R antibody (R&D Systems, BAF227) diluted to 10 micro g/mL with PBS 0.05% Tween 20 was added to the measurement disc, and then solid-phased on the reaction layer in the disc. A mixed solution of the labeled anti-delta GK antibody, anti-human IL-6R antibody (albumin switch molecule), and human soluble IL-6R or monkey IL-6R, incubated as described above, was added to the disc, and the anti-human IL-6R antibody-human soluble IL-6R-AF647-anti-delta GK complex or the anti-human IL-6R antibody-monkey soluble IL-6R-AF647-anti-delta GK complex was captured by the solid-phased antibody. The amount of the captured complex was detected by the fluorescence signal of AF647 (Detect PMT 25%). The fluorescence signal of AF647 was analyzed by Gyro Evaluator (version 3.6.2.30, Gyros Protein Technologies). The signal from H54//IC17 was set at 100%, and the analysis results which are signal values of each of the samples, are shown in FIG. 4.

Compared to the anti-human IL-6R/KLH bispecific antibody H54//IC17, not carrying an albumin-binding portion, ABP(H/L).N13-H54//IC17, carrying an added albumin-binding peptide (ABP), showed 82% and 76% decrease in the signal derived from the complex formed with human soluble IL-6R in human and rat plasma, respectively. Furthermore, regarding ABD094(H/L)-H54//IC17, carrying an added albumin-binding domain (ABD094), the signal derived from the complex formed with human soluble IL-6R decreased 97% and 96% in human and rat plasma, respectively.

In monkey plasma, compared to H54//IC17, the antibodies carrying an added ABP or ABD094 showed 58% and 94% decrease in the signal derived from the complex formed with monkey soluble IL-6R, respectively.

In view of the above results, it was shown that binding to the target antigen in the presence of human, rat, or monkey albumin can be decreased by adding ABP or ABD094 to the antibody, similarly to that in the presence of mouse albumin (see Example 4-1). At the same time, ABP and ABD094 were shown to be able to bind to any one of mouse, human, rat, and monkey albumins.

### Example 5: Assessment of Antigen Binding of Anti-Human IL-6R Albumin Switch Molecules in Mouse Plasma (ABD035 and G418)

The measurements were performed using Gyrolab xP (Gyros Protein Technologies).

First, to bind the albumin-binding domain to albumin in the plasma, the anti-human IL-6R antibodies (albumin switch molecules) indicated in Example 2 were diluted to 4 nM using C57BL/6J mouse plasma, and then incubated at room temperature for two hours. Next, for binding of the anti-human IL-6R antibody to the target antigen, one equivalent of human soluble IL-6R diluted to 16 nM in mouse plasma was added to the antibody solutions, and then the solutions were incubated at room temperature for 1 hour 40 minutes.

To detect the antigen-antibody complexes, first, the anti-delta GK antibody (AF647-anti-delta GK) was labeled using an Alexa flour 647 labeling kit (A20186, Thermo Fisher Scientific) according to the attached protocols. The labeled anti-delta GK antibody was diluted to 133 nM with PBS 0.05% Tween 20 (P3563, sigma), one equivalent of this solution was added to a mixed solution of anti-human IL-6R antibody and human soluble IL-6R, and this was incubated at room temperature for 30 minutes and then O/N incubated at 4 degrees C. (the final concentrations were 1 nM for the antibody, 4 nM for human soluble IL-6R, and 67 nM for AF647-anti-delta GK).

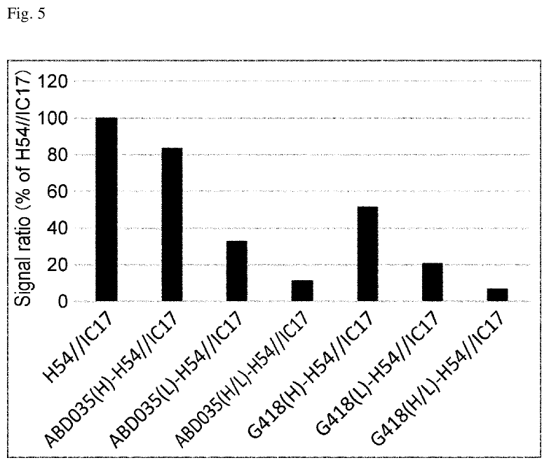

Measurement of antigen-antibody complexes was performed using Gyrolab xP. For the measurements using Gyrolab xP, Gyrolab Bioaffy 200 (P0004180, Gyros Protein Technologies) was used as the measurement disc. Biotinylated anti-human IL-6R antibody (R&D Systems, BAF227) diluted to 10 micro g/mL with PBS 0.05% Tween 20 was added to the measurement disc, and then solid-phased on the reaction layer in the disc. A mixed solution of the labeled anti-delta GK antibody, anti-human IL-6R antibody (albumin switch molecule), and human soluble IL-6R, incubated as described above, was added to the disc, and the anti-human IL-6R antibody-human soluble IL-6R-AF647-anti-delta GK complex was captured by the solid-phased antibody. The amount of the captured complex was detected by the fluorescence signal of AF647 (Detect PMT 25%). The fluorescence signal of AF647 was analyzed by Gyro Evaluator (version 3.6.2.30, Gyros Protein Technologies). The analysis results are shown in FIG. 5.

Compared to the anti-human IL-6R/KLH bispecific antibody H54//IC17, not carrying an albumin-binding site, among the antibodies carrying an albumin-binding domain (ABD035), the signal decreased in the order of ABD035(H)-H54//IC17, ABD035(L)-H54//IC17, and ABD035(H/L)-H54//IC17. Regarding ABD035 (H/L)-H54//IC17 whose signal decreased most remarkably, the signal decreased 89% compared to that of H54//IC17. Similarly, compared to H54//IC17, among the antibodies carrying an albumin-binding domain (G418), the signal decreased in the order of G418(H)-H54//IC17, G418(L)-H54//IC17, and G418(H/L)-H54//IC17. Regarding G418 (H/L)-H54//IC17 whose signal decreased most remarkably, the signal decreased 93% compared to that of H54//IC17.

Accordingly, similarly to ABP and ABD094 indicated in Example 4, it was shown that by adding other albumin-binding portions such as ABD035 and G418 to the antibody, binding to a target antigen in the presence of albumin can also be reduced without substantially changing the binding to the target antigen in the absence of albumin. Moreover, as in Example 4, it was shown that compared to when one albumin-binding site is added to one target antigen-binding portion (one arm of the antibody), binding to the target antigen in the presence of albumin can be inhibited more effectively when two or more albumin-binding sites are added.

Antibodies ABD035(H)-H54//IC17 and ABD035(L)-H54//IC17, produced by adding one albumin-binding portion to one target antigen-binding portion (one arm of the antibody), were compared. Surprisingly, the signal derived from the complex formed with soluble human IL-6R was greatly decreased for ABD035(L)-H54//IC17 having the albumin-binding portion added to the N terminus of the L-chain variable region, when compared to that of ABD035(H)-H54//IC17 having the albumin-binding portion added to the N terminus of the H-chain variable region (FIG. 5). Similarly, when G418(H)-H54//IC17 and G418(L)-H54//IC17 were compared, the signal derived from the complex formed with soluble human IL-6R was greatly decreased for G418(L)-H54//IC17 having the albumin-binding site added to the N terminus of the L-chain variable region, when compared to that of G418(H)-H54//IC17 having the albumin-binding site added to the N terminus of the H-chain variable region (FIG. 5). Accordingly, it was also shown that when adding an albumin-binding portion such as ABD035 or G418 to the H54 antibody (anti-human IL-6R antibody), binding to the target antigen in the presence of albumin can be inhibited more effectively by adding the albumin-binding site to the N terminus of the L-chain variable region, as in Example 4.

### Example 6: Assessment of Binding of Anti-IL6R Albumin Switch Molecules in CHO Cells Forced to Express IL6R (IL6R-CHO Cells)

Binding ability of albumin switch molecules against the membrane-type antigens were verified. Specifically, the antibodies (10 micro g/mL) indicated below were incubated with IL6R-CHO cells (Chiome Bioscience, 2×10⁵ cells/well) in mouse serum albumin (MSA)-free CHO-S-SFM II medium (Thermo Fisher Scientific), 50 mg/mL Recombinant MSA (Albumin Bioscience)-containing medium, or mouse plasma (6-week-old male C57BL/6J mouse) at 4 degrees C. for 60 minutes. Antibodies used for the reaction were IC17, H54//IC17, and ABD094(H/L)-H54//IC17. After the reaction, the cells were washed three times with PBS(−). Addition of Alexa Fluor 647-labeled anti-dGK antibody (10 micro g/mL) was followed by incubation at 4 degrees C. for 30 minutes. Lastly, the cells were suspended in PBS(−) and analyzed by FACSCantoII (BD).

Binding histograms of the antibodies in IL6R-CHO cells are shown in FIG. 6. IC17 showed low fluorescence intensity under MSA-free medium (FIG. 6(a)), MSA-containing medium (FIG. 6(b)), and mouse plasma (FIG. 6(c)) conditions, and showed similar histograms even under different conditions. This suggests that IC17 does not bind to IL6R-CHO cells regardless of the presence or absence of albumin.

In MSA-free medium, H54//IC17, not containing an albumin-binding site, showed higher fluorescence intensity than IC17 (FIG. 6(a)). Furthermore, H54//IC17 showed higher fluorescence intensities than IC17 in MSA-containing medium (FIG. 6(b)) and mouse plasma (FIG. 6(c)). This suggests that H54//IC17 binds IL6R on the cell membrane at comparable levels regardless of the presence or absence of albumin.

ABD094(H/L)-H54//IC17, which is an albumin switch molecule, showed comparable level of fluorescence intensity as that of H54//IC17 in the MSA-free medium (FIG. 6(a)). On the other hand, it showed fluorescence intensities lower than those of H54//IC17 and close to those of IC17 in the MSA-containing medium (FIG. 6(b)) and mouse plasma (FIG. 6(c)). This suggests that in the absence of albumin, binding of an albumin switch molecule to IL6R on the cell membrane is comparable to that of conventional antibodies (antibodies not containing an albumin-binding site), but in the presence of albumin its binding to membrane-type IL6R is inhibited.

### Example 7-1: Plasma Kinetics of Albumin Switch Molecules and Target Antigen (Albumin-Binding Peptide)

To examine the pharmacokinetics (PK) of albumin switch molecules and target antigen in plasma, the following anti-human IL-6R antibodies prepared in Example 2 were administered at a dose of 1 mg/kg to C57BL/6J mice (6 to 8-weeks old, male) through the tail vein. Twenty minutes after antibody administration, human soluble IL-6R (sIL6R) was administered at 0.05 mg/kg. The administered antibodies were IC17, H54, ABP(H).N13-H54, and ABP(H/L).N13-H54. Blood was collected over time from sIL6R administration to 7 days after the administration, and plasma was obtained by centrifugation (12,000 rpm, 4 degrees C., 5 minutes).

Plasma antibody concentration (total antibody concentration) was measured by enzyme-linked immunosorbent assay (ELISA). First, as a capturing antibody, anti-human IgG heavy and light chain antibody (Bethyl Laboratories) was added to a plate. To this plate, phosphate buffered saline with Tween20 (PBST, Sigma-Aldrich) containing 1% bovine serum albumin (BSA, Sigma-Aldrich) was added as a blocking solution, and then a diluted plasma sample was added. Then, biotin-labeled anti-dGK antibody was added as a detection antibody, and streptavidin-labeled polyHRP80 (Stereospecific Detection Technologies) was added as a secondary detection antibody. Finally, tetramethylbenzidine (TMB, Surmodics) was added as a substrate, and then the absorbance at 650 nm was measured on a CLARIOstar absorptiometer (BMG Labotech).

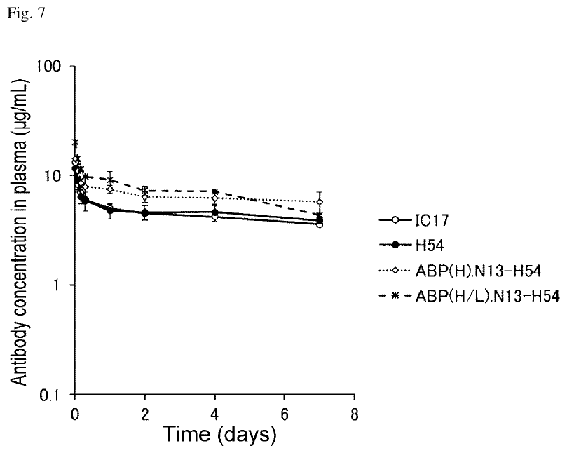

The plasma antibody concentration vs time curves are shown in FIG. 7. At 1 mg/kg, all antibodies showed similar PK profiles. Furthermore, PK parameters of the antibodies which were calculated using Phoenix WinNonlin (Certara) are shown in Table 6. All antibodies had comparable half-lives.

TABLE 6

| | AUC | CL | Vss | T½ |
|---|---|---|---|---|
| Antibody | μg*day/mL | mL/day/kg | mL/kg | day |
| IC17 | 112 | 9.29 | 196 | 15.5 |
| H54 | 120 | 8.66 | 186 | 15.9 |
| ABP(H).N13-H54 | 164 | 6.49 | 128 | 14.1 |
| ABP(H/L).N13-H54 | 86.7 | 11.5 | 97 | 5.97 |

The concentration of sIL6R in plasma (total sIL6R concentration) was measured by electrochemiluminescence immunoassay (ECLIA). Biotin-labeled anti-human IL6R polyclonal antibody (R&D), SULFO-Tag-labeled anti-human IL6R monoclonal antibody (R&D), and a diluted plasma sample were mixed, and allowed to react overnight. Subsequently, the mixed solution was added to a streptavidin immobilized ECL plate (Meso Scale Discovery). Finally, after addition of Read buffer (Meso Scale Discovery), the signals were measured on SECTOR Imager (Meso Scale Discovery).

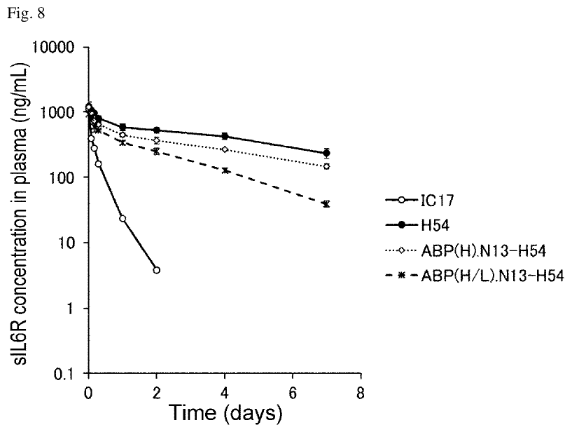

The plasma antigen concentration-time curves are shown in FIG. 8. Furthermore, the PK parameters of the antigen (sIL6R) calculated using Phoenix WinNonlin (Certara) are shown in Table 7.

TABLE 7

| | AUC | CL | Vss | T½ |
|---|---|---|---|---|
| Antibody | ng*day/mL | mL/day/kg | mL/kg | day |
| IC17 | 178 | 281 | 92.7 | 0.377 |
| H54 | 4640 | 10.9 | 62.1 | 4.03 |
| ABP(H).N13-H54 | 3060 | 16.4 | 84 | 3.71 |
| ABP(H/L).N13-H54 | 1490 | 33.7 | 87.4 | 1.82 |

In the group subjected to H54 administration, sIL6R disappeared gradually, and the clearance was 10.9 mL/day/kg. On the other hand, in the group subjected to IC17 administration, sIL6R disappeared quickly, and the clearance was 281 mL/day/kg. Generally, IgG antibodies have long plasma half-lives, and their clearance is small. Therefore, such results suggest that in the group subjected to anti-human IL-6R antibody administration, the target antigen (sIL6R) binds to the antibody and disappears gradually, whereas in the group subjected to administration of an antibody that does not bind to human IL6R, sIL6R does not bind to the antibody and disappears quickly.

In the group subjected to administration of the albumin switch molecule ABP(H).N13-H54, sIL6R showed a PK profile similar to that of the group subjected to H54 administration. On the other hand, in the group subjected to administration of ABP(H/L).N13-H54, sIL6R disappeared more quickly and clearance was larger than in the H54-administered group. This showed that, by adding two or more albumin-binding sites to one target antigen-binding site (one arm of the antibody), binding to the target antigen in plasma can be inhibited more effectively.

### Example 7-2: Plasma Kinetics of Albumin Switch Molecules and Target Antigen (Albumin-Binding Domain and Albumin-Binding Peptide)

To examine the PK of albumin switch molecules and target antigen in plasma, the following anti-human IL-6R antibodies were administered at a dose of 0.2 or 1 mg/kg to C57BL/6J mice (6 to 8-weeks old, male) through the tail vein. Twenty minutes after antibody administration, human soluble IL-6R (sIL6R) was administered at 0.05 mg/kg. The administered antibodies were IC17, H54//IC17, ABP(H/L).N13-H54//IC17, ABD094(H)-H54//IC17, ABD094(L)-H54//IC17, and ABD094(H/L)-H54//IC17. Blood was collected over time from antigen administration to 7 days after the administration, and plasma was obtained by centrifugation (12,000 rpm, 4 degrees C., 5 minutes).

Plasma antibody concentration (total antibody concentration) was measured by enzyme-linked immunosorbent assay (ELISA). First, as a capturing antibody, anti-human IgG heavy and light chain antibody (Bethyl Laboratories) was added to a plate. To this plate, phosphate buffered saline with Tween20 (PBST, Sigma-Aldrich) containing 1% bovine serum albumin (BSA, Sigma-Aldrich) was added as a blocking solution, and then a diluted plasma sample was added. Then, biotin-labeled anti-dGK antibody was added as a detection antibody, and streptavidin-labeled polyHRP80 (Stereospecific Detection Technologies) was added as a secondary detection antibody. Finally, tetramethylbenzidine (TMB, Surmodics) was added as a substrate, and the absorbance at 650 nm was measured on a CLARIOstar absorptiometer (BMG Labotech).

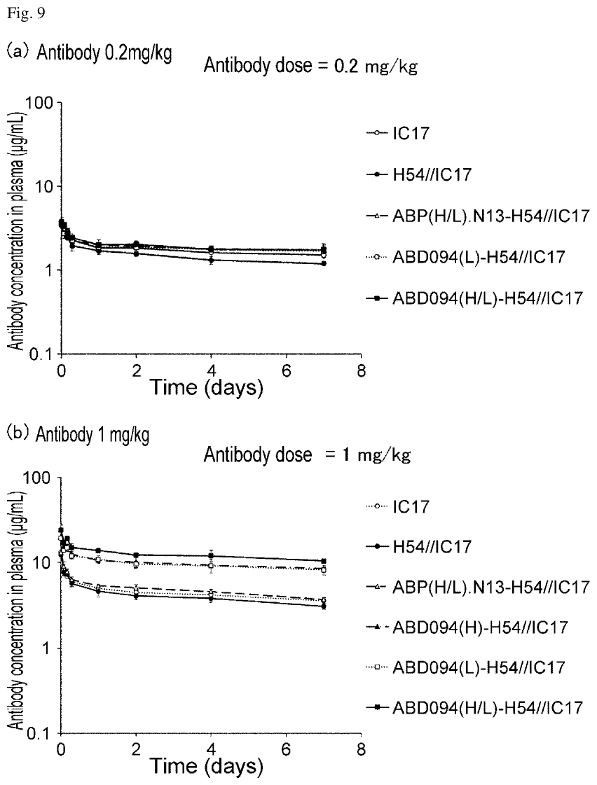

The plasma antibody concentration-time curves are shown in FIG. 9. At each dose, all antibodies showed similar PK profiles. Furthermore, PK parameters of the antibodies which were calculated using Phoenix WinNonlin (Certara) are shown in Table 8. The half-lives of all antibodies were of comparable levels.

TABLE 8

| | Dose | AUC | CL | Vss | T½ |
|---|---|---|---|---|---|
| Antibody | mg/kg | μg*day/mL | mL/day/kg | mL/kg | day |
| IC17 | 1 | 112 | 9.29 | 196 | 15.5 |
| H54//IC17 | 1 | 97.7 | 11.7 | 212 | 15.2 |
| ABP(H/L).N13- | 1 | 92.4 | 10.8 | 170 | 11.0 |
| H54//IC17 | | | | | |
| ABD094(H)- | 1 | 249 | 4.19 | 84.6 | 14.6 |
| H54//IC17 | | | | | |
| ABD094(L)- | 1 | 233 | 4.35 | 88.3 | 14.2 |
| H54//IC17 | | | | | |
| ABD094(H/L)- | 1 | 366 | 2.82 | 72.5 | 18.8 |
| H54//IC17 | | | | | |
| IC17 | 0.2 | 59.4 | 3.59 | 103 | 21.4 |
| H54//IC17 | 0.2 | 30.6 | 6.59 | 111 | 11.8 |
| ABP(H/L).N13- | 0.2 | 51.2 | 3.91 | 88.3 | 15.5 |
| H54//IC17 | | | | | |
| ABD094(L)- | 0.2 | 176 | 3.24 | 95.4 | 63.5 |
| H54//IC17 | | | | | |
| ABD094(H/L)- | 0.2 | 79.2 | 2.92 | 92.4 | 25.8 |
| H54//IC17 | | | | | |

The concentration of sIL6R in plasma (total sIL6R concentration) was measured by electrochemiluminescence immunoassay (ECLIA). Biotin-labeled anti-human IL6R polyclonal antibody (R&D), SULFO-Tag-labeled anti-human IL6R monoclonal antibody (R&D), and a diluted plasma sample were mixed, and allowed to react overnight. Subsequently, the mixed solution was added to a streptavidin immobilized ECL plate (Meso Scale Discovery). Finally, after addition of Read buffer (Meso Scale Discovery), the signals were measured on SECTOR Imager (Meso Scale Discovery).

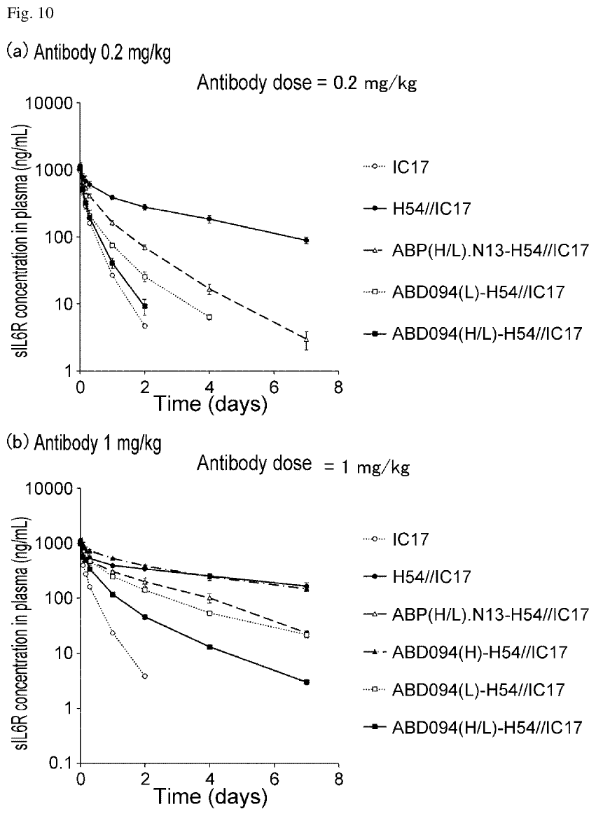

The plasma antigen concentration-time curves are shown in FIG. 10. Furthermore, the PK parameters of the antigen (sIL6R) calculated using Phoenix WinNonlin (Certara) are shown in Table 9.

TABLE 9

| | Dose | AUC | CL | Vss | T½ |
|---|---|---|---|---|---|
| Antibody | mg/kg | ng*day/mL | mL/day/kg | mL/kg | day |
| IC17 | 1 | 178 | 281 | 92.7 | 0.377 |
| H54//IC17 | 1 | 3322 | 15.4 | 103 | 4.90 |
| ABP(H/L).N13- | 1 | 1222 | 41.2 | 91.3 | 1.54 |
| H54//IC17 | | | | | |
| ABD094(H)- | 1 | 3120 | 16.0 | 77.9 | 3.56 |
| H54//IC17 | | | | | |
| ABD094(L)- | 1 | 983 | 51.0 | 114 | 2.12 |
| H54//IC17 | | | | | |
| ABD094(H/L)- | 1 | 463 | 108 | 122 | 1.40 |
| H54//IC17 | | | | | |
| IC17 | 0.2 | 186 | 270 | 94.1 | 0.396 |
| H54//IC17 | 0.2 | 2120 | 23.7 | 94.1 | 2.97 |
| ABP(H/L).N13- | 0.2 | 582 | 86.2 | 97.4 | 1.18 |
| H54//IC17 | | | | | |
| ABD094(L)- | 0.2 | 313 | 160 | 132 | 0.969 |
| H54//IC17 | | | | | |
| ABD094(H/L)- | 0.2 | 224 | 226 | 96.9 | 0.452 |
| H54//IC17 | | | | | |

Comparison of each of the groups subjected to antibody dose of 0.2 mg/kg shows that in the group subjected to H54//IC17 administration, sIL6R disappeared gradually, and the clearance was 23.7 mL/day/kg. On the other hand, in the group subjected to IC17 administration, sIL6R disappeared quickly, and the clearance was 270 mL/day/kg. As in the Examples described above, such results suggest that in the group subjected to anti-human IL-6R antibody administration, the target antigen (sIL6R) binds to the antibody and disappears gradually, whereas in the group subjected to administration of an antibody that does not bind to human IL6R, sIL6R does not bind to the antibody and disappears quickly.

In the group subjected to administration of an albumin switch molecule, ABP(H/L).N13-H54//IC17, ABD094(L)-H54//IC17, or ABD094(H/L)-H54//IC17, sIL6R disappeared more quickly and clearance was greater than in the H54//IC17-administered group. This showed that, albumin switch molecules have decreased binding to the target antigen in plasma.

Comparison of each of the groups subjected to antibody dose of 1 mg/kg shows that in the group subjected to H54//IC17 administration, sIL6R disappeared gradually, whereas in the group subjected to IC17 administration, sIL6R disappeared quickly. In the group subjected to ABD094(H/L)-H54//IC17 administration, sIL6R disappeared more quickly than in the groups subjected to administration of ABP(H/L).N13-H54//IC17, ABD094(H)-H54//IC17, or ABD094(L)-H54//IC17. These results show that albumin switch molecules, particularly ABD094(H/L)-H54//IC17, has greatly decreased binding to the target antigen (sIL6R) in plasma, and therefore, sIL6R does not bind to the antibody and disappears quickly.

In the ABD094(H/L)-H54//IC17-administered group, sIL6R disappeared more quickly than in the groups subjected to administration of ABD094(L)-H54//IC17 or ABD094(H)-H54//IC17. This showed that, by adding two albumin-binding sites to one target antigen-binding site (one arm of the antibody), binding to the target antigen in plasma can be inhibited more effectively. Furthermore, in the ABD094(L)-H54//IC17-administered group, sIL6R disappeared more quickly than in the ABD094(H)-H54//IC17-administered group. Accordingly, this showed that when adding an albumin-binding portion to the H54 antibody (anti-human IL-6R antibody), binding to the target antigen in the presence of albumin can be inhibited more effectively by adding an albumin-binding portion to the N-terminus of the L chain variable region.

### Example 7-3: Further Analysis of Plasma Kinetics of the Target Antigen when Albumin Switch Molecules are Administered

To further analyze the plasma kinetics of albumin switch molecules and target antigen, plasma samples collected in Example 7-2 were used to quantify the total sIL6R in plasma, the antibody-bound sIL6R (bound sIL6R), and sIL6R not bound to an antibody (free sIL6R), respectively.

(1) Concentration of Total sIL6R in Plasma

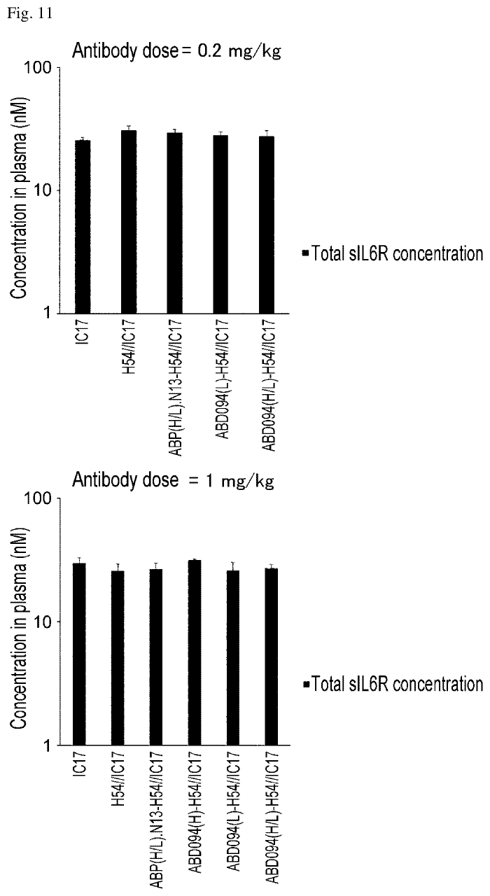

The total sIL6R concentration in plasma at five minutes after sIL6R administration was measured by the method of Example 7-2. The measurement results are shown in FIG. 11. In both the groups subjected to 0.2 mg/kg administration and 1 mg/kg administration, the total sIL6R concentrations were at comparable levels for all antibodies.

(2) Concentration of Free sIL6R in Plasma

Next, free sIL6R in plasma was measured. The concentration of free sIL6R in plasma was measured on a fully automated ELISA system (Gyrolab xP workstation). First, sIL6R prepared at 23.4, 46.9, 93.8, 188, 375, 750, and 1500 ng/mL using a Protein-Free (PBS) Blocking Buffer (Thermo Scientific) was used as solutions for preparing the calibration curve. Mouse plasma samples were used in the measurements without diluting them. A solution of biotin-labeled H54 diluted to 10 micro g/mL using a Protein-Free (PBS) Blocking Buffer was used as a solution of the sIL6R-capturing molecule. Alexa Fluor (registered trademark) 647-labeled anti-human IL-6R antibody (R&D Systems) diluted to 1 micro g/mL using a Protein-Free (PBS) Blocking Buffer was used as a solution of the human IL-6R-detection molecule. PBS (pH 7.4) containing 0.05% TWEEN was used as a washing solution. The calibration curve solutions, mouse plasma samples, sIL6R-capturing molecule solution, sIL6R-detection molecule solution, and the washing solution were placed onto a PCR plate, and then the PCR plate was sealed with a plate sealer. After the PCR plate and the Bioaffy 200 disc were placed in the Gyrolab xP workstation, measurements were taken. Using the data processed by the Gyros Evaluater 3.6.2.30 software and the calibration curve concentrations, the concentration of free sIL6R in plasma was calculated by logistic regression using the XLfit 5.5.0.5 software.

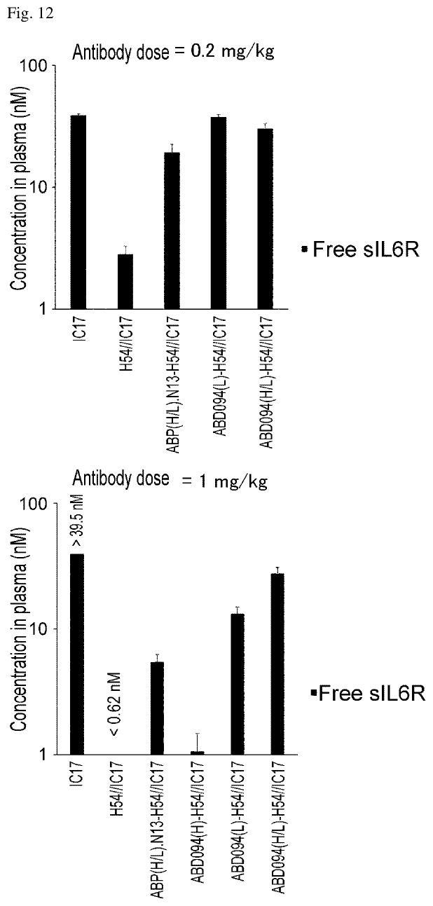

The concentrations of free sIL6R in plasma at five minutes after sIL6R administration are shown in FIG. 12. In the groups subjected to IC17 administration, the concentrations of free sIL6R were comparable to those of the total sIL6R in both the 0.2 mg/kg and the 1 mg/kg-administered groups. On the other hand, in the groups subjected to H54//IC17 administration, the concentrations of free sIL6R were low. In the groups subjected to albumin switch molecule administration, particularly the ABD094(L)-H54//IC17-administered groups and the ABD094(H/L)-H54//IC17-administered groups showed high concentrations of free sIL6R in plasma which were almost equal to those of the IC17-administered groups. These results suggest that binding of an albumin switch molecule, particularly ABD094(L)-H54//IC17 or ABD094(H/L)-H54//IC17, to sIL6R was decreased in plasma. Furthermore, in the groups subjected to ABD094(H/L)-H54//IC17 administration, the concentrations of free sIL6R in plasma were higher than in the groups subjected to ABD094(L)-H54//IC17 or ABD094(H)-H54//IC17 administration. This indicates that by adding two albumin-binding sites to one target antigen-binding site (one arm of an antibody), binding to the target antigen in plasma can be inhibited more effectively.

(3) Concentration of Bound sIL6R in Plasma

Additionally, bound sIL6R in plasma was measured. The concentration of bound sIL6R in plasma was measured on an electrospray ionization mass spectrometer connected to a high-performance liquid chromatography (LC/ESI-MS/MS). First, H54//IC17 and sIL6R were mixed, and solutions for preparing the calibration curve of bound sIL6R were prepared at theoretical plasma concentrations of 0.0468, 0.467, 4.60, and 20.1 nmol/mL. The solutions for preparing the calibration curve and 1 or 2 micro L of a plasma sample were added to 75 or 150 micro g of magnetic beads (Magnosphere MS300/Carboxyl, JSR) to which anti-delta GK antibodies were solid-phased, and this was shaken at 25 degrees C. for one hour. The magnetic beads after shaking were washed three times with 0.1 or 0.2 mL of PBS containing 0.05% Tween 20, and then washed with 0.1 or 0.2 mL of PBS. The magnetic beads were suspended in 12.5 or 25 micro L of 50 mmol/L ammonium bicarbonate containing 7.5 mol/L urea, 8 mmol/L dithiothreitol, and 0.1 micro g/mL lysozyme (chicken egg white), and then shaken at 56 degrees C. for 45 minutes. Next, 1 or 2 micro L of 500 mmol/L iodoacetamide was added, and this was shaken at 37 degrees C. for 30 minutes under protection from light. Next, 80 or 160 micro L of 50 mmol/L ammonium bicarbonate containing 0.625 micro g/mL sequencing grade modified trypsin (Promega) was added. After the addition, the sample was shaken overnight at 37 degrees C., and then 2.5 or 5 micro L of 10% trifluoroacetic acid was added to stop the reaction. 40 or 80 micro L of the enzyme-digested sample prepared this way was used for the analysis by LC/ESI-MS/MS. Xevo TQ-S triple quadrupole instrument (Waters) connected to I-class plus UPLC (Waters) was used for the LC/ESI-MS/MS analysis. The human IL-6R-specific peptide HVVQLR was detected by selected reaction monitoring (SRM). In the SRM transition, the parent ion of human IL-6R was [M+2H]2+ (m/z 376.2) and its daughter ion was the y4 ion (m/z 515.3). The internal calibration standard curve was produced by linear regression using the concentration and peak area with 1/x2 weighting. The concentration of bound sIL6R in plasma was calculated using the analysis software Masslynx Ver.4.1 (Waters).

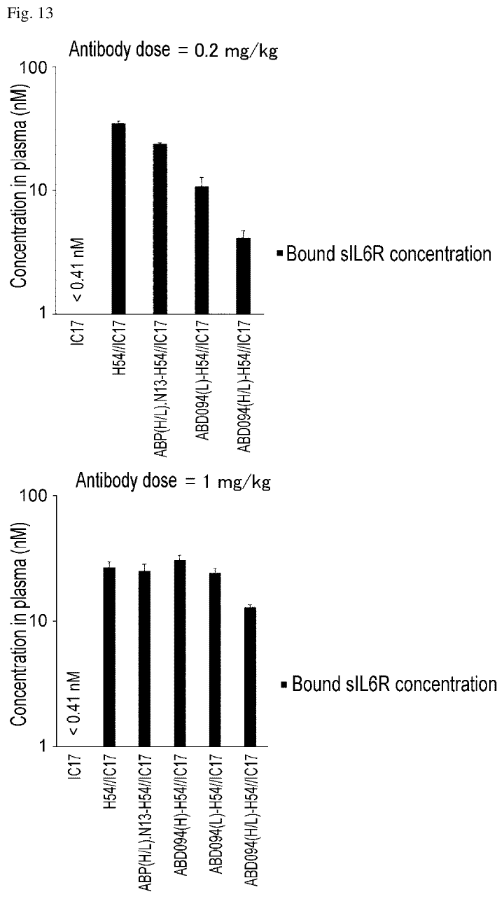

The concentrations of bound sIL6R at five minutes after sIL6R administration are shown in FIG. 13. In the groups subjected to IC17 administration, concentrations of bound sIL6R were lower than the detection limit in both the 0.2 mg/kg and the 1 mg/kg-administered groups. On the other hand, in the groups subjected to H54//IC17 administration, concentrations of bound sIL6R were almost equal to the concentrations of total sIL6R. In the groups subjected to albumin switch molecule administration, particularly the ABD094(L)-H54//IC17-administered groups and the ABD094(H/L)-H54//IC17-administered groups, concentrations of bound sIL6R in plasma were low compared to the groups subjected to H54//IC17 administration. These results suggest that binding of an albumin switch molecule, particularly ABD094(L)-H54//IC17 or ABD094(H/L)-H54//IC17, to sIL6R was decreased in plasma. Furthermore, in the groups subjected to ABD094(H/L)-H54//IC17 administration, concentrations of bound sIL6R in plasma were lower than in the groups subjected to administration of ABD094(L)-H54//IC17 or ABD094(H)-H54//IC17. This indicates that by adding two albumin-binding sites to one target antigen-binding site (one arm of an antibody), binding to the target antigen in plasma can be inhibited more effectively.

### Example 7-4: In Vivo Affinity of Albumin Switch Molecules to the Target Antigen in Plasma

Using the measurement results of Examples 7-2 and 7-3, the in vivo affinities (dissociation constant, KD) of the albumin switch molecules to the target antigen (sIL6R) in plasma were calculated. The concentrations of total antibody, free sIL6R, and bound sIL6R at five minutes after sIL6R administration were used for the calculation, and in vivo KD was calculated using Formula 1 shown below. Here, in vivo KDsIL6R indicates in vivo affinity to sIL6R, Abtotal indicates the total antibody concentration, Agfree indicates the free sIL6R concentration, and IC indicates the bound sIL6R concentration.

$$in vivo {KD}_{SIL 6 R} = \frac{( {Ab}_{total} - IC ) \times {Ag}_{free}}{IC} \qquad ( Formula 1 )$$

The calculated in vivo affinities to plasma sIL6R (KD (in vivo KDsIL6R)) are shown in Table 10.

TABLE 10

| | in vivo KD_{sIL6R} | in vivo KD_{sIL6R} |
|---|---|---|
| | nM | nM |
| Antibody | (Antibody 0.2 mg/kg) | (Antibody 1 mg/kg) |
| IC17 | NA | NA |
| H54//IC17 | NA | 1.3 |
| ABP(H/L).N13-H54//IC17 | 2.0 | 13.4 |
| ABD094(H)-H54//IC17 | NA | 3.6 |
| ABD094(L)-H54//IC17 | 49.1 | 57.3 |
| ABD094(H/L)-H54//IC17 | 140.9 | 306.4 |

Compared to H54//IC17, ABD094(L)-H54//IC17 and ABD094(H/L)-H54//IC17 showed larger in vivo KDs. This suggests that in plasma, binding of ABD094(L)-H54//IC17 and ABD094(H/L)-H54//IC17 to the target antigen (sIL6R) is inhibited. Furthermore, ABD094(H/L)-H54//IC17 showed larger in vivo KD than ABD094(L)-H54//IC17 or ABD094(H)-H54//IC17. This indicates that by adding two albumin-binding sites to one target antigen-binding site (one arm of an antibody), binding to the target antigen in plasma can be inhibited more effectively.

### Example 8-1: Kinetics of Albumin Switch Molecules and Target Antigen in the Cerebrospinal Fluid (CSF) of Mice

To examine the PK of albumin switch molecules and target antigen in CSF, the following anti-human IL-6R antibodies were administered at a dose of 0.25 mg/kg to the lateral cerebroventricle of C57BL/6J mice (6 to 8-weeks old, male). Ten minutes after antibody administration, sIL6R (human soluble IL-6R) was administered at 0.0125 mg/kg. The administered antibodies were IC17, H54//IC17, ABP(H/L).N13-H54//IC17, ABD094(L)-H54//IC17, and ABD094(H/L)-H54//IC17. CSF was collected at 10 minutes and 90 minutes after sIL6R administration.

### (1) Concentration of Antibody in CSF

Concentration of antibody in CSF (total antibody concentration) was measured by enzyme-linked immunosorbent assay (ELISA). As a capturing antibody, anti-human IgG heavy and light chain antibody (Bethyl Laboratories) was added to a plate. To this plate, phosphate buffered saline with Tween20 (PBST, Sigma-Aldrich) containing 1% bovine serum albumin (BSA, Sigma-Aldrich) was added as a blocking solution, and then a diluted CSF sample was added. Then, biotin-labeled anti-dGK antibody was added as a detection antibody, and streptavidin-labeled polyHRP80 (Stereospecific Detection Technologies) was added as a secondary detection antibody. Finally, tetramethylbenzidine (TMB, Surmodics) was added as a substrate, and then the absorbance at 650 nm was measured on a CLARIOstar absorptiometer (BMG Labotech).

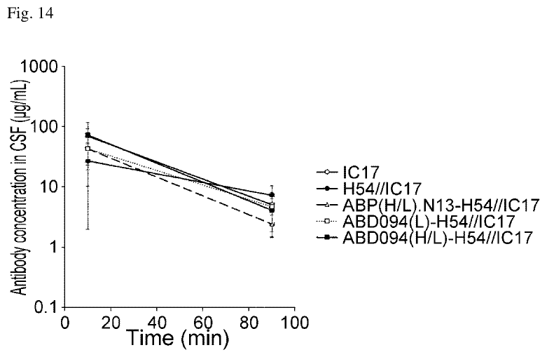

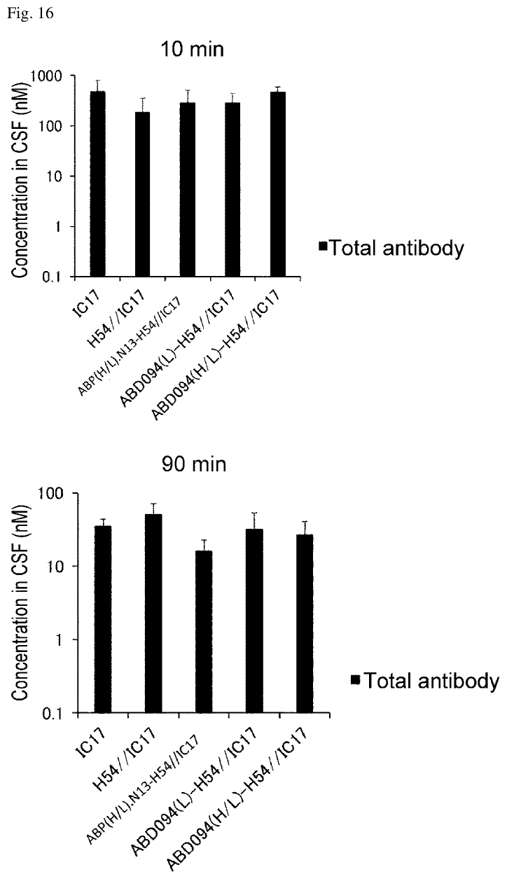

The CSF antibody concentration-time curves are shown in FIG. 14. In all groups, the administered antibodies showed similar PK profiles. Furthermore, the total antibody concentrations at 10 minutes or 90 minutes after sIL6R administration are shown in FIG. 16. In all groups, the total antibody concentrations were comparable.

(2) Concentration of Total sIL6R in CSF

The concentration of sIL6R in CSF (total sIL6R concentration) was measured by electrochemiluminescence immunoassay (ECLIA). Biotin-labeled anti-human IL6R polyclonal antibody (R&D), SULFO-Tag-labeled anti-human IL6R monoclonal antibody (R&D), and a diluted CSF sample were mixed, and allowed to react overnight. Subsequently, the mixed solution was added to a streptavidin immobilized ECL plate (Meso Scale Discovery). Finally, after addition of Read buffer (Meso Scale Discovery), the signals were measured on SECTOR Imager (Meso Scale Discovery).

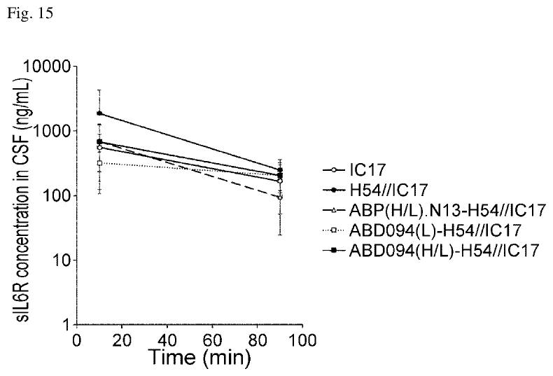

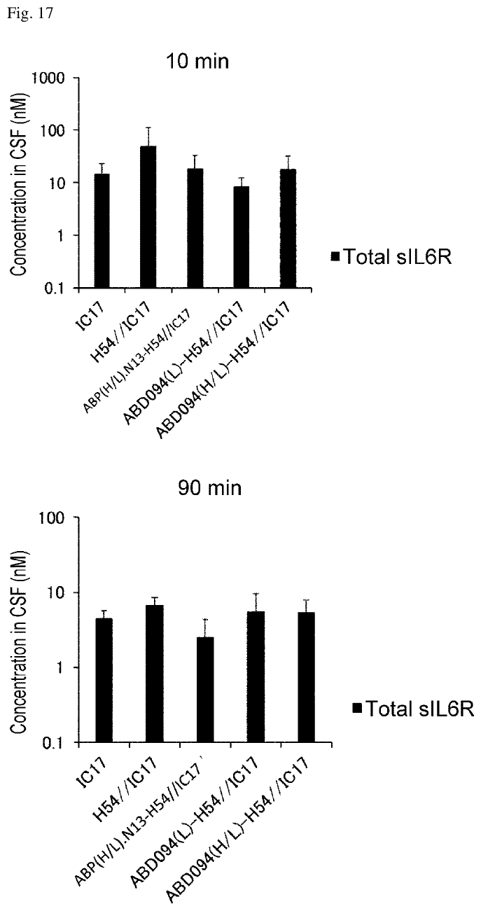

The CSF antigen concentration-time curves are shown in FIG. 15. All of the antibody-administered groups showed similar target antigen PK profiles. Furthermore, the total sIL6R concentrations at 10 minutes or 90 minutes after sIL6R administration are shown in FIG. 17. All antibody-administered groups showed similar total sIL6R concentrations. This shows that in CSF where the albumin concentration is low, binding of the antibody to the target antigen does not change according to the presence or absence of an albumin-binding site.

(3) Concentration of Free sIL6R in CSF

The concentration of free sIL6R (concentration of sIL6R not bound to antibodies) in CSF was measured on a fully automated ELISA system (Gyrolab xP workstation). Human IL-6R prepared at 15.6, 31.3, 62.5, 125, 250, 500, 1000, and 2000 ng/mL using a Protein-Free (PBS) Blocking Buffer (Thermo Scientific) was used as solutions for preparing the calibration curve. Mouse CSF samples diluted two-fold with a Protein-Free (PBS) Blocking Buffer were used in the measurements. Biotin-labeled H54 diluted to 10 micro g/mL using a Protein-Free (PBS) Blocking Buffer was used as a solution of the human IL-6R-capturing molecule. Alexa Fluor (registered trademark) 647-labeled anti-human IL-6R antibody (R&D Systems) diluted to 1 micro g/mL in a Protein-Free (PBS) Blocking Buffer was used as a solution of the human IL-6R-detection molecule. PBS (pH 7.4) containing 0.05% TWEEN was used as a washing solution. The calibration curve solutions, mouse CSF samples, human IL-6R-capturing molecule solution, human IL-6R-detection molecule solution, and the washing solution were placed onto a PCR plate, and then the PCR plate was sealed with a plate sealer. After the PCR plate and the Bioaffy 200 disc were placed in the Gyrolab xP workstation, measurements were taken. Using the data processed by the Gyros Evaluater 3.6.2.30 software and the calibration curve concentrations, the concentration of free human IL-6R in CSF was calculated by logistic regression using the XLfit 5.5.0.5 software.

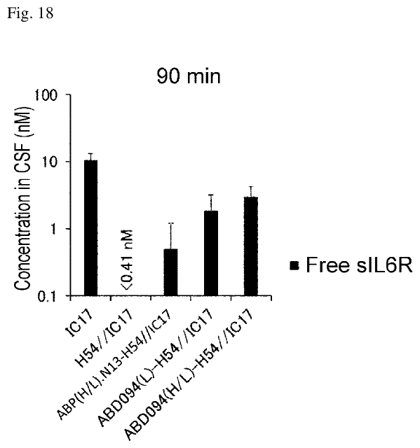

The concentrations of free sIL6R at 90 minutes after sIL6R administration are shown in FIG. 18. Compared to IC17, albumin switch molecules, particularly ABD094(L)-H54//IC17 and ABD094(H/L)-H54//IC17, showed lower free sIL6R concentrations. These results indicate that in CSF where albumin concentration is low, binding of albumin switch molecules to the target antigen (sIL6R) is not inhibited by the albumin-binding site, and albumin switch molecules bind to sIL6R in CSF.

(4) Concentration of Bound sIL6R in CSF

The concentration of bound sIL6R (concentration of sIL6R bound to antibodies) in CSF was measured on an electrospray ionization mass spectrometer connected to a high-performance liquid chromatography (LC/ESI-MS/MS). The sIL6R-bound H54//IC17 antibody was diluted with blank CSF to produce solutions for preparing the calibration curve at 0.41, 1.23, 3.70, 11.1, 33.3, 100, and 300 nmol/mL. The solutions for preparing the calibration curve and 1 micro L of a plasma sample were added to 75 micro g of magnetic beads (Magnosphere MS300/Carboxyl, JSR) to which anti-delta GK antibodies were solid-phased, and this was shaken at 25 degrees C. for one hour. After shaking, the magnetic beads were washed three times with 0.1 mL of PBS containing 0.05% Tween 20, and then washed with 0.1 mL of PBS. The magnetic beads were suspended in 12.5 micro L of 50 mmol/L ammonium bicarbonate containing 7.5 mol/L urea, 8 mmol/L dithiothreitol, and 0.1 micro g/mL lysozyme (chicken egg white), and then shaken at 56 degrees C. for 45 minutes. Next, 1 micro L of 500 mmol/L iodoacetamide was added, and then shaken at 37 degrees C. for 30 minutes under protection from light. Next, 80 micro L of 50 mmol/L ammonium bicarbonate containing 0.625 micro g/mL sequencing grade modified trypsin (Promega) was added. After the addition, the sample was shaken overnight at 37 degrees C., and then 2.5 micro L of 10% trifluoroacetic acid was added to stop the reaction. 40 micro L of the enzyme-digested sample prepared this way was used for the analysis by LC/ESI-MS/MS. Xevo TQ-S triple quadrupole instrument (Waters) connected to I-class plus UPLC (Waters) was used for the LC/ESI-MS/MS analysis. The human IL-6R-specific peptide HVVQLR was detected by selected reaction monitoring (SRM). In the SRM transition, the parent ion of human IL-6R was [M+2H]2+(m/z 376.2) and the daughter ion was the y4 ion (m/z 515.3). The internal calibration standard curve was produced by linear regression using the concentration and peak area with 1/x2 weighting. The concentration of bound sIL6R CSF was calculated using the analysis software Masslynx Ver.4.1 (Waters).

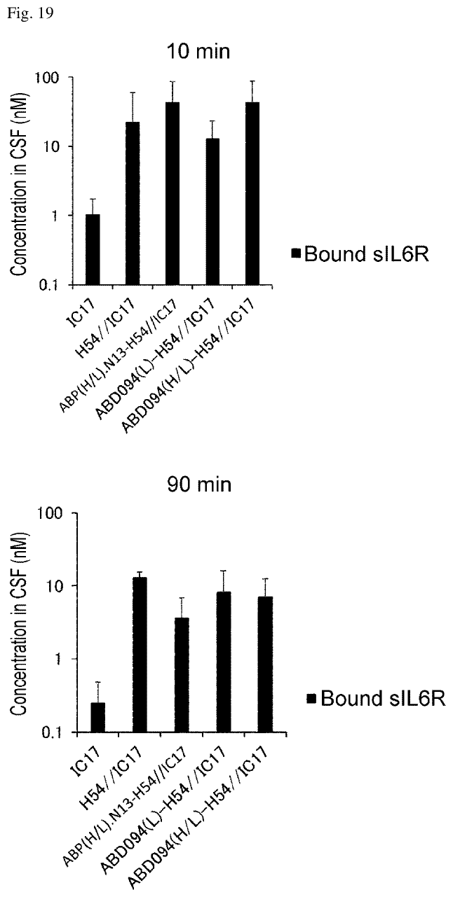

The concentrations of bound sIL6R at 10 minutes or 90 minutes after sIL6R administration are shown in FIG. 19. Compared to IC17, albumin switch molecules ABP(H/L).N13-H54//IC17, ABD094(L)-H54//IC17, and ABD094(H/L)-H54//IC17 showed higher concentrations of bound sIL6R, and they showed bound sIL6R concentrations which were almost equal to that of H54//IC17. These results indicate that in CSF where albumin concentration is low, binding of albumin switch molecules to the target antigen (sIL6R) is not inhibited by the albumin-binding site, and albumin switch molecules bind to sIL6R in CSF.

### Example 8-2: In Vivo Affinity of Albumin Switch Molecules to the Target Antigen in CSF

Using the measurement results of Example 8-1, the in vivo affinities (dissociation constant, KD) of the albumin switch molecules to the target antigen (sIL6R) in CSF were calculated. The total antibody concentration, free sIL6R concentration, and bound sIL6R concentration at 90 minutes after sIL6R administration were used for the calculation, and in vivo KD was calculated using Formula 1.

The calculated in vivo affinities to sIL6R in CSF (KD (in vivo KDsIL6R)) are shown in Table 11.

TABLE 11

| | in vivo KD_{sIL6R} |
|---|---|
| Antibody | (Calculation with concentration at 90 min) |
| IC17 | NA |
| H54//IC17 | 1.2 |
| ABP(H/L).N13-H54//IC17 | 1.7 |
| ABD094(L)-H54//IC17 | 5.2 |
| ABD094(H/L)-H54//IC17 | 8.1 |

Compared to IC17 not carrying a human IL-6R-binding arm, H54//IC17 showed very low apparent KD. Albumin switch molecules ABP(H/L).N13-H54//IC17, ABD094(L)-H54//IC17, and ABD094(H/L)-H54//IC17 showed values close to that of H54//IC17. This indicates that in CSF where albumin concentration is low, these antibodies bind to sIL6R at an equivalent level to that of H54//IC17 which does not carry an albumin-binding site. In addition, as described in Example 7-4, the apparent KDs of these albumin switch molecules in plasma were significantly larger than the apparent KDs in CSF. These results suggest that albumin switch molecules bind to the target antigen in CSF at an equivalent level to that of the molecule not containing an albumin-binding site, but in the plasma, binding of these molecules to the target antigen is inhibited by the albumin-binding site.

### Example 9: Examination of Albumin Switch Molecules Using Linkers

### Example 9-1: Assessment of Human IL6R Binding in Mouse Plasma by Albumin Switch Molecules that Use Linkers

Whether binding of an anti-human IL6R albumin switch molecule to the target antigen in the presence of albumin changes by placing a linker between the albumin-binding portion and the target antigen-binding portion was examined. Specifically, antibody-human soluble IL-6 complexes in mouse plasma were measured for ABD094(H/L).N02-H54//IC17 formed by addition of ABD094 via a linker (N02) to the N termini of the H and L chains of the anti-human IL6R-binding arm, and ABD094(H/L).NTO-H54//IC17 formed by addition of ABD094 via a linker (N10), for ABD094(H/L)-H54//IC17 formed by addition of ABD094 without a linker, and for anti-human IL-6R/KLH bispecific antibody H54//IC17 not carrying an albumin-binding site. The measurements were performed using Gyrolab xP (Gyros Protein Technologies).

First, to bind the albumin-binding domain to albumin in the plasma, various anti-human IL-6R antibodies (albumin switch molecules) including the three types of antibodies mentioned above and anti-human IL-6R/KLH bispecific antibody H54//IC17 which does not carry an albumin-binding site were diluted to 4 nM using C57BL/6J mouse plasma, and then incubated at room temperature for 30 minutes. Next, for binding of the anti-human IL6R antibody to the target antigen, one equivalent of human soluble IL-6R diluted to 16 nM in mouse plasma was added to the antibody solutions, and then the solutions were incubated at room temperature for 30 minutes.

To detect the antigen-antibody complexes, first, the anti-delta GK antibody (AF647-anti-delta GK) was labeled using an Alexa flour 647 labeling kit (A20186, Thermo Fisher Scientific) according to the attached protocols. The labeled anti-delta GK antibody was diluted to 133 nM with PBS 0.05% Tween 20 (P3563, sigma), one equivalent of this solution was added to a mixed solution of anti-human IL6R antibody and human soluble IL-6R, and this was incubated at room temperature for 30 minutes, and then O/N incubated at 4 degrees C. (the final concentrations were 1 nM for the antibody, 4 nM for human soluble IL-6R, and 67 nM for AF647-anti-delta GK).

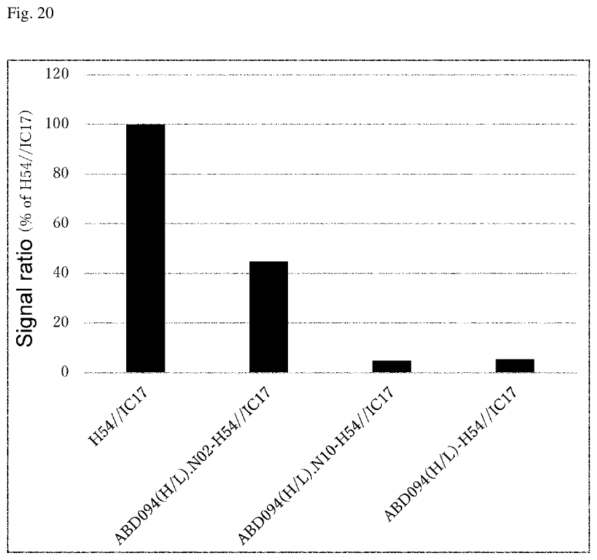

Measurement of antigen-antibody complexes were performed using Gyrolab xP. For the measurements using Gyrolab xP, Gyrolab Bioaffy 200 (P0004180, Gyros Protein Technologies) was used as the measurement disc. Biotinylated anti-human IL-6R antibody (R&D Systems, BAF227) diluted to 10 micro g/mL with PBS 0.05% Tween 20 was added to the measurement disc, and then solid-phased on the reaction layer in the disc. A mixed solution of the labeled anti-delta GK antibody, anti-human IL-6R antibody (albumin switch molecule), and human soluble IL-6R, incubated as described above, was added to the disc, and the anti-human IL6R antibody-human soluble IL-6R-AF647-anti-delta GK complex was captured by the solid-phased antibody. The amount of the captured complex was detected by the fluorescence signal of AF647 (Detect PMT 25%). The fluorescence signal of AF647 was analyzed by Gyro Evaluator (version 3.6.2.30, Gyros Protein Technologies). The analysis results are shown in FIG. 20.

Compared to the anti-human IL-6R/KLH bispecific antibody H54//IC17, not carrying an albumin-binding site, ABD094(H/L).N02-H54//IC17 carrying albumin-binding domains (ABD094) added via the N02 linker to the H and L chains, showed 55% decrease in the signal derived from the complex formed with human soluble IL-6R. Meanwhile, regarding ABD094(H/L).N10-H54//IC17 and ABD094(H/L)-H54//IC17 in which albumin-binding domains (ABD094) are bound to the H and L chains via the N10 linker or without the mediation of a linker, respectively, both showed 95% decrease in the signal compared to that of H54//IC17. That is, binding to the target antigen in the presence of albumin was more decreased for ABD094(H/L).N10-H54//IC17 and ABD094 (H/L)-H54//IC17. This shows that compared to when placing a linker having the length of N02 between the albumin-binding portion and the target antigen-binding portion, placing a shorter N10 linker or not placing a linker enables more effective inhibition of binding to the target antigen in the presence of albumin.

### Example 9-2: Plasma Kinetics of the Target Antigen and Albumin Switch Molecules that Use Linkers

To examine the pharmacokinetics (PK) of the target antigen in plasma after administration of albumin switch molecules that use linkers, the following anti-human IL-6R antibodies were administered at a dose of 1 mg/kg to C57BL/6J mice (6 to 8-weeks old, male) through the tail vein. Twenty minutes after antibody administration, human soluble IL6R (sIL6R) was administered at 0.05 mg/kg. The administered antibodies were IC17, H54//IC17, ABD094(H/L).N02-H54//IC17, and ABD094(H/L)-H54//IC17. Blood was collected over time from sIL6R administration to 7 days after the administration, and plasma was obtained by centrifugation (12,000 rpm, 4 degrees C., 5 minutes).

Concentration of sIL6R in plasma (total sIL6R concentration) was measured by electrochemiluminescence immunoassay (ECLIA). Biotin-labeled anti-human IL6R polyclonal antibody (R&D), SULFO-Tag-labeled anti-human IL6R monoclonal antibody (R&D), and a diluted plasma sample were mixed, and allowed to react overnight. Subsequently, the mixed solution was added to streptavidin immobilized ECL plates (Meso Scale Discovery). Finally, after addition of Read buffer (Meso Scale Discovery), the signals were measured on SECTOR Imager (Meso Scale Discovery).

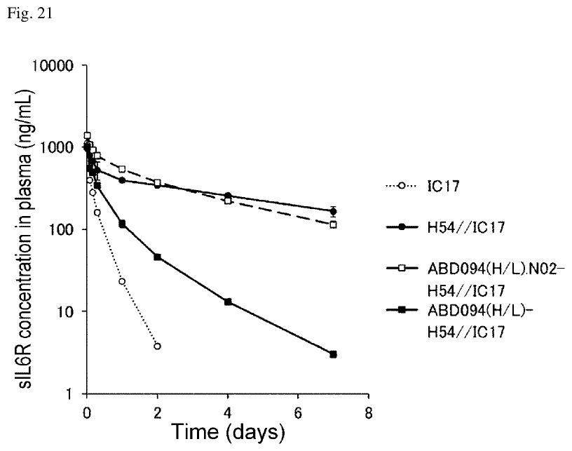

The plasma antigen concentration-time curves are shown in FIG. 21. Furthermore, the PK parameters of the antigen calculated using Phoenix WinNonlin (Certara) are shown in Table 12.

TABLE 12

| | Dose | AUC | CL | Vss | T½ |
|---|---|---|---|---|---|
| Antibody | mg/kg | ng*day/mL | mL/day/kg | mL/kg | day |
| IC17 | 1 | 178 | 281 | 92.7 | 0.377 |
| H54//IC17 | 1 | 3322 | 15.4 | 103 | 4.90 |
| ABD094(H/L).N02- | 1 | 2780 | 18 | 71.2 | 3.03 |
| H54//IC17 | | | | | |
| ABD094(H/L)- | 1 | 463 | 108 | 122 | 1.4 |
| H54//IC17 | | | | | |

In the group subjected to H54//IC17 administration, sIL6R disappeared gradually; whereas, in the group subjected to IC17 administration, sIL6R disappeared quickly. Generally, IgG antibodies have long plasma half-lives, and their clearance is small. Therefore, such results suggest that in the group subjected to anti-human IL-6R antibody administration, the target antigen (sIL6R) binds to the antibody and disappears gradually; whereas in the group subjected to administration of an antibody that does not bind to human IL-6R, sIL6R does not bind to the antibody and disappears quickly.

In the group subjected to administration of ABD094(H/L).N02-H54//IC17 which is an albumin switch molecule that uses the N02 linker consisting of 5 amino acid residues (GGGGS (SEQ ID NO: 35)), sIL6R showed a PK profile similar to that of the group subjected to H54//IC17 administration. This shows that ABD094(H/L).N02-H54//IC17 binds to sIL6R in plasma at a comparable level to that of H54//IC17. On the other hand, in the group subjected to administration of ABD094(H/L)-H54//IC17, an albumin switch molecule that does not use a linker, sIL6R disappeared more quickly compared to H54//IC17 and clearance was larger. More specifically, binding activity of ABD094(H/L)-H54//IC17 towards sIL6R was shown to be decreased in plasma.

The above results indicate that when the target antigen-binding portion and the albumin-binding portion are linked through a 5-amino-acid-residue linker, binding to the target antigen in plasma does not decrease sufficiently. Moreover, surprisingly, direct linking of the target antigen-binding portion and the albumin-binding portion without the mediation of a linker was shown to enable effective inhibition of binding to the target antigen in plasma.

### Example 9-3: Effects of Difference in Linker Lengths and Difference in the Albumin-Binding Domains on Antigen-Binding Inhibition

To examine the pharmacokinetics (PK) of the target antigen in plasma after administration of the albumin switch molecules carrying linkers of different lengths and the albumin switch molecules carrying different albumin-binding domains, the following anti-human IL6R antibodies were administered at a dose of 0.2 mg/kg to C57BL/6J mice (6 to 8-weeks old, male) through the tail vein. Twenty minutes after antibody administration, human soluble IL6R (sIL6R) was administered at 0.05 mg/kg. The administered antibodies were IC17, H54//IC17, ABD094(H/L)-H54//IC17, ABD094(H/L).N10-H54//IC17, ABD094(H/L).N11-H54//IC17, ABD094(H/L).N12-H54//IC17, ABD094(H/L).N13-H54//IC17, ABD035(H/L)-H54//IC17, and G418.ABD3(H/L)-H54//IC17. Blood was collected over time from sIL6R administration to 7 days after the administration, and plasma was obtained by centrifugation (12,000 rpm, 4 degrees C., 5 minutes).

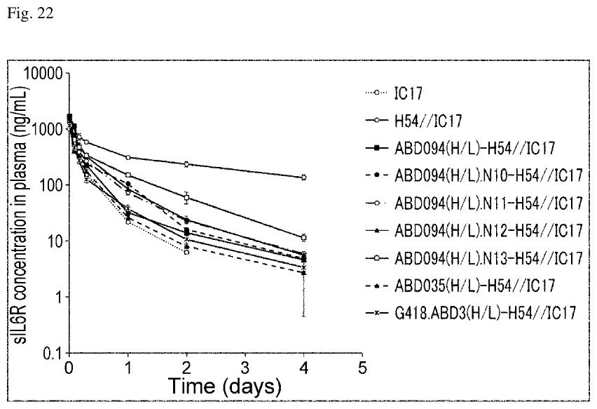

Concentration of sIL6R in plasma (total sIL6R concentration) was measured by electrochemiluminescence immunoassay (ECLIA). Biotin-labeled anti-human IL6R polyclonal antibody (R&D), SULFO-Tag-labeled anti-human IL6R monoclonal antibody (R&D), and a diluted plasma sample were mixed, and allowed to react overnight. Subsequently, the mixed solution was added to streptavidin immobilized ECL plates (Meso Scale Discovery). Finally, after addition of Read buffer (Meso Scale Discovery), the signals were measured on SECTOR Imager (Meso Scale Discovery). The plasma antigen concentration-time curves up to day 4 are shown in FIG. 22. Furthermore, the PK parameters of the antigen calculated using Phoenix WinNonlin (Certara) are shown in Table 13.

TABLE 13

| | AUC | CL | Vss | T½ |
|---|---|---|---|---|
| Antibody | ng*day/mL | mL/day/kg | mL/kg | day |
| IC17 | 208 | 242 | 82.4 | 0.538 |
| H54//IC17 | 1933 | 26.3 | 112 | 3.47 |
| ABD094(H/L)-H54//IC17 | 338 | 148 | 156 | 3.25 |
| ABD094(H/L).N10- | 393 | 127 | 101 | 1.97 |
| H54//IC17 | | | | |
| ABD094(H/L).N11- | 328 | 153 | 119 | 0.974 |
| H54//IC17 | | | | |
| ABD094(H/L).N12- | 397 | 126 | 86.5 | 0.931 |
| H54//IC17 | | | | |
| ABD094(H/L).N13- | 527 | 95.4 | 113 | 1.68 |
| H54//IC17 | | | | |
| ABD035(H/L)-H54//IC17 | 219 | 228 | 316 | 4.07 |
| G418.ABD3(H/L)- | 212 | 237 | 163 | 1.17 |
| H54//IC17 | | | | |

In the group subjected to H54//IC17 administration, sIL6R disappeared gradually; whereas, in the group subjected to IC17 administration, sIL6R disappeared quickly. Generally, IgG antibodies have long plasma half-lives, and their clearance is small. Therefore, such results suggest that in the group subjected to anti-human IL6R antibody administration, the target antigen (sIL6R) binds to the antibody and disappears gradually; whereas in the group subjected to administration of an antibody that does not bind to human IL6R, sIL6R does not bind to the antibody and disappears quickly. In the group subjected to administration of ABD094(H/L)-H54//IC17, an albumin switch molecule that does not use a linker, sIL6R disappeared significantly more quickly compared to H54//IC17 and clearance was larger. More specifically, binding activity of ABD094(H/L)-H54//IC17 towards sIL6R was shown to be decreased in plasma.

The group subjected to administration of ABD094(H/L).NO-H54//IC17 which is an albumin switch molecule that uses the N10 linker consisting of one amino acid residue (G), sIL6R showed a PK profile close to that of the group subjected to ABD094(H/L)-H54//IC17 administration. This shows that binding of ABD094(H/L).NTO-H54//IC17 to sIL6R in plasma is inhibited at a comparable level to that of ABD094(H/L)-H54//IC17.

In the group subjected to administration of ABD094(H/L).NT1-H54//IC17 which is an albumin switch molecule that uses the N11 linker consisting of 2 amino acid residues (GS) and the group subjected to administration of ABD094(H/L).N12-H54//IC17 which is an albumin switch molecule that uses the N12 linker consisting of 3 amino acid residues (GGS), sIL6R disappeared more gradually compared to ABD094(H/L)-H54//IC17 and ABD094(H/L).N10-H54//IC17, but disappeared more quickly compared to H54//IC17. This suggests that binding of ABD094(H/L).N11-H54//IC17 and ABD094(H/L).N12-H54//IC17 to sIL6R is also inhibited in plasma.

In the group subjected to administration of ABD094(H/L).N13-H54//IC17 which is an albumin switch molecule that uses the N13 linker consisting of 4 amino acid residues (GGGS SEQ ID NO: 42), sIL6R disappeared more gradually compared to ABD094(H/L).N11-H54//IC17 and ABD094(H/L).N12-H54//IC17, but disappeared more quickly compared to H54//IC17. This suggests that binding of ABD094(H/L).N13-H54//IC17 to sIL6R is also inhibited in plasma.

In the groups subjected to administration of ABD035(H/L)-H54//IC17 and G418.ABD3(H/L)-H54//IC17, which are albumin switch molecules having an albumin domain different from ABD094, sIL6R showed PK profiles close to that of the group subjected to ABD094(H/L)-H54//IC17 administration. This shows that binding to sIL6R in plasma is inhibited for ABD035(H/L)-H54//IC17 and G418.ABD3(H/L)-H54//IC17 at the same level as for ABD094(H/L)-H54//IC17. These results show that even when the target antigen-binding portion and the albumin-binding portion are linked through a linker, if the linker length is 4 amino acid residues or shorter, binding to the target antigen in plasma can be suppressed. Furthermore, even when the albumin domain is ABD035 or G418.ABD3, and is different from ABD094, binding to the target antigen in plasma can be suppressed.

### Example 10: Production of Molecules Whose Binding Changes by Albumin (Albumin Switch Molecules) (Myelin Oligodendrocyte Protein-Binding Molecule)

As the anti-Myelin Oligodendrocyte Glycoprotein (MOG) antibody heavy chain variable region (VH) and light chain variable region (VL), MOG307VH (SEQ ID NO: 47) and MOG307VL (SEQ ID NO: 48) were designed, respectively. MOG is an antigen expressed in the central nervous system (CNS) including the brain.

The design involved attachment of an albumin-binding domain to the N-terminal amino acid residue of VH and/or the N-terminal amino acid residue of VL of the anti-MOG antibody. Specifically, a gene encoding the albumin-binding domain ABD094 (SEQ ID NO: 4) was linked upstream of the gene(s) encoding the VH and/or VL of the anti-MOG antibody.

Genes encoding VH and VL, which were designed this way, were synthesized and linked to the gene encoding the heavy chain constant region (SEQ ID NO: 6) or the light chain constant region (SEQ ID NO: 8), respectively. Each of the linked genes was cloned into an expression vector.

Using the genes produced as described above, the antibodies listed in Table 14 were expressed transiently in mammalian cells by a method known to those skilled in the art, and purified by a method known to those skilled in the art when necessary.

TABLE 14

| Name | Explanation | Albumin-binding site |
|---|---|---|
| MOG//IC17 | Anti-MOG/KLH bispecific | None |
| | antibody (1 MOG-binding | |
| | site) | |
| ABD094(H/L)- | Anti-MOG/KLH bispecific | Albumin-binding domain (SEQ ID NO: 4) |
| MOG//IC17 | antibody (1 MOG-binding | at the VH and VL N-termini of MOG- |
| | site) | binding arm. No linker. |
| ABD094(H/L).N02- | Anti-MOG/KLH bispecific | Albumin-binding domain (SEQ ID NO: 4) |
| MOG//IC17 | antibody (1 MOG-binding | is linked at the VH and VL N-termini of |
| | site) | MOG-binding arm via a linker (N02). |
| ABD094(H/L).N10- | Anti-MOG/KLH bispecific | Albumin-binding domain (SEQ ID NO: 4) |
| MOG//IC17 | antibody (1 MOG-binding | is linked at the VH and VL N-termini of |
| | site) | MOG-binding arm via a linker (N10). |

The combinations of amino acid sequences for MOG//IC17, ABD094(H/L)-MOG//IC17, ABD094(H/L).N02-MOG//IC17, and ABD094(H/L).N10-MOG//IC17 are shown in Tables 15-1 and 15-2.

TABLE 15-1

| | MOG-binding arm | | | | | | |
|---|---|---|---|---|---|---|---|
| | | | | | | H chain full | L chain full |
| | | | | | | length (including | length (including |
| | Albumin- | | | | | albumin-binding | albumin-binding |
| Name | binding site | VH | CH | VL | CL | site) | site) |
| MOG//IC17 | None | SEQ ID NO: 47 | SEQ ID NO: 6 | SEQ ID NO: 48 | SEQ ID NO: 8 | SEQ ID NO: 49 | SEQ ID NO: 50 |
| ABD094(H/L)- | SEQ ID NO: 4 | SEQ ID NO: 47 | SEQ ID NO: 6 | SEQ ID NO: 48 | SEQ ID NO: 8 | SEQ ID NO: 51 | SEQ ID NO: 52 |
| MOG//IC17 | | | | | | | |
| ABD094(H/L).N02- | SEQ ID NO: 4 | SEQ ID NO: 47 | SEQ ID NO: 6 | SEQ ID NO: 48 | SEQ ID NO: 8 | SEQ ID NO: 53 | SEQ ID NO: 54 |
| MOG//IC17 | | | | | | | |
| ABD094(H/L).N10- | SEQ ID NO: 4 | SEQ ID NO: 47 | SEQ ID NO: 6 | SEQ ID NO: 48 | SEQ ID NO: 8 | SEQ ID NO: 55 | SEQ ID NO: 56 |
| MOG//IC17 | | | | | | | |

TABLE 15-2

KLH-binding arm

| | Albumin- | | |
|---|---|---|---|
| | binding | H chain full | L chain full |
| Name | portion | length | length |
| MOG//IC17 | None | SEQ ID NO: | SEQ ID NO: |
| ABD094(H/L)- | | 21 | 22 |
| MOG//IC17 | | | |
| ABD094(H/L).N02- | | | |
| MOG//IC17 | | | |
| ABD094(H/L).N10- | | | |
| MOG//IC17 | | | |

### Example 11: Assessment of Binding of Albumin Switch Molecules to Mouse MOG

Binding activities of the antibodies produced in Example 10 against mouse MOG were assessed using Biacore T200 (Cytiva).

As the running buffer, 50 mM Na Phosphate (pH 7.4), 150 mM NaCl, 0.05% Tween 20, and 0.005% NaN₃ was used, and the assessment was performed at 25 degrees C. First, anti-human Fc antibody (Cytiva) was used as a ligand-capturing molecule. Anti-human Fc antibody (Cytiva) was solid-phased on a Series S Sensor Chip CM4 (Cytiva) using an Amine Coupling Kit (Cytiva). Antibody solutions prepared using the running buffer was allowed to interact with this CM4 sensor chip to capture approximately 480 RU of the antibody. Mouse MOG (R&D system) was diluted to 0, 10, 40, 160, and 640 nM with the running buffer, and then bound to the captured antibody. The chip was regenerated using 3 M MgCl₂, and measurements were performed by repeatedly capturing antibodies. Dissociation constant (KD) of the respective antibodies for mouse MOG was calculated using Biacore T200 Evaluation Software 2.0. Specifically, the association rate constant ka (L/mol/s) and the dissociation rate constant kd (1/s) were calculated by global fitting of the sensorgrams obtained by measurement with the 1:1 Langmuir binding model, and the dissociation constant KD (mol/L) was calculated from these values. Table 16 shows the results of these measurements.

TABLE 16

| | Affinity against mouse MOG | | |
|---|---|---|---|
| Antibody Name | ka (1/Ms) | kd (1/s) | KD (M) |
| MOG//IC17 | 1.55E+05 | 6.99E−05 | 4.52E−10 |
| ABD094(H/L)-MOG//IC17 | 7.26E+03 | 1.39E−05 | 1.91E−09 |
| ABD094.N02(H/L)-MOG//IC17 | 5.70E+03 | 3.46E−07 | 6.07E−11 |
| ABD094.N10(H/L)-MOG//IC17 | 1.71E+03 | 9.46E−06 | 5.53E−09 |

Regardless of the presence or absence of albumin-binding sites, all of the anti-mouse MOG/IC17 bispecific antibodies were confirmed to have comparable levels of binding activity against mouse MOG in the absence of albumin.

### Example 12: Assessment of Antigen Binding in Mouse Plasma by Albumin Switch Molecules that Use Linkers

Whether binding of an anti-mouse MOG albumin switch molecule to the target antigen in the presence of albumin changes by placing a linker between the albumin-binding portion and the target antigen-binding portion was examined using the antibodies produced in Example 10. Specifically, antibody-mouse soluble MOG complexes in mouse plasma were measured for ABD094(H/L).N02-MOG//IC17 and ABD094(H/L).N10-MOG//IC17 formed by linker (N02 or N10)-mediated addition of ABD094, ABD094(H/L)-MOG//IC17 formed by adding ABD094 without linker mediation, and MOG//IC17 which does not carry an albumin-binding site. The measurements were performed using Gyrolab xP (Gyros Protein Technologies).

First, to bind the albumin-binding domain to albumin in plasma, each of the anti-mouse MOG antibodies mentioned above were diluted to 8 nM using C57BL/6J mouse plasma, and then incubated at room temperature for 40 minutes. Next, for binding of the anti-mouse MOG antibody to the target antigen, one equivalent of His-tagged mouse MOG (8536-MO, R&D Systems) diluted to 38 nM in mouse plasma was added to the antibody solutions, and then the solutions were incubated at room temperature for 1 hour 25 minutes.

To detect the antibody-antigen complexes, first, the anti-delta GK antibody (AF647-anti-delta GK) was labeled using an Alexa flour 647 labeling kit (A20186, Thermo Fisher Scientific) according to the attached protocols. The labeled anti-delta GK antibody was diluted to 133 nM with PBS 0.05% Tween 20 (P3563, sigma), one equivalent of this solution was added to a mixed solution of anti-mouse MOG antibody and mouse soluble MOG, and this was incubated at room temperature for one hour, and then O/N incubated at 4 degrees C. (the final concentrations were 2 nM for the antibody, 9 nM for His-tagged mouse MOG, and 67 nM for AF647-anti-delta GK).

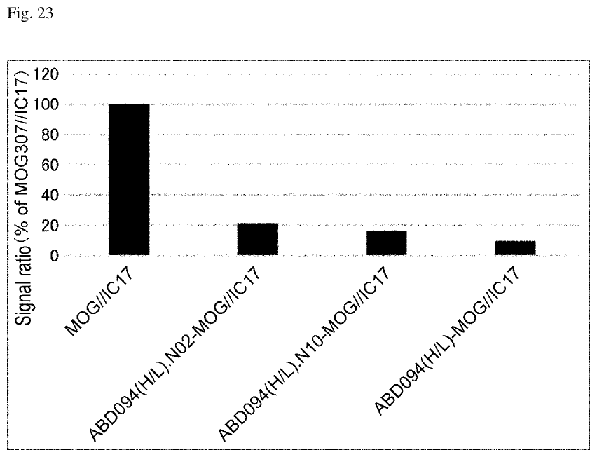

Measurement of antigen-antibody complexes were performed using Gyrolab xP. For the measurements using Gyrolab xP, Gyrolab Bioaffy 200 (P0004180, Gyros Protein Technologies) was used as the measurement disc. Biotinylated anti-His-tagged antibody (BAM050, R&D Systems) diluted to 10 micro g/mL with PBS 0.05% Tween 20 was added to the measurement disc, and then solid-phased on the reaction layer in the disc. A mixed solution of the labeled anti-delta GK antibody, anti-mouse MOG antibody (albumin switch molecule), and mouse MOG, incubated as described above, was added to the disc, and the anti-mouse MOG antibody-mouse MOG-AF647-anti-delta GK complex was captured by the solid-phased antibody. The amount of the captured complex was detected by the fluorescence signal of AF647 (Detect PMT 25%). The fluorescence signal of AF647 was analyzed by Gyro Evaluator (version 3.6.2.30, Gyros Protein Technologies). The analysis results are shown in FIG. 23.

Compared to MOG//IC17 which does not carry an albumin-binding site, ABD094(H/L).N02-MOG//IC17 carrying an albumin-binding domain (ABD094) added via the N02 linker, showed 79% decrease in the signal derived from the complex formed with mouse soluble MOG. Meanwhile, ABD094(H/L).N10-MOG//IC17, in which an albumin-binding domain (ABD094) is linked via the N10 linker, and ABD094 (H/L)-MOG//IC17, in which the albumin-binding domain is linked without linker mediation, showed 83% and 90% decrease in the signals, respectively, compared to that of MOG//IG17. That is, binding to the target antigen in the presence of albumin was more decreased for ABD094(H/L).N10-MOG//IC17 and ABD094 (H/L)-MOG//IC17. This shows that compared to when placing a linker having the length of N02 between the albumin-binding portion and the target antigen-binding portion, placing a shorter N10 linker or not placing a linker enables more effective inhibition of binding to the target antigen in the presence of albumin.

### Example 13: Assessment of Binding of Anti-MOG Albumin Switch Molecules in Oligodendrocytes

The binding ability of albumin switch molecules against the membrane-type antigens were examined. Specifically, the antibodies (10 micro g/mL) indicated below were incubated with mouse brain cell suspension (2×10⁵ cells/well), in mouse serum albumin (MSA)-free DPBS (Thermo Fisher Scientific), DPBS containing Recombinant MSA (Albumin Bioscience) at 50 mg/mL, or mouse plasma (6 weeks old, male C57BL/6J mouse) at 4 degrees C. for 60 minutes. The mouse brain cell suspension was prepared from mouse brain using Adult Brain Dissociation Kit (Miltenyi Biotec) and gentleMACS (Miltenyi Biotec). Antibodies used for the reaction were IC17, MOG//IC17, and ABD094(H/L)-MOG//IC17. After the reaction, the cells were washed once with DPBS. Addition of Alexa Fluor 647-labeled anti-delta GK antibody (10 micro g/mL), PE-labeled anti-O4 antibody (Miltenyi Biotec, 50 fold dilution), and VioBlue-labeled anti-CD45 antibody (Miltenyi Biotec, 50 fold dilution) was followed by incubation at 4 degrees C. for 30 minutes. Lastly, the cells were suspended in DPBS and analyzed by FACSCantoII (BD). The fluorescence intensities of Alexa Fluor 647 in the MOG-expressing oligodendrocyte O4-positive/CD45-negative fractions were measured (Schroeter, C. B. et al., One Brain-All Cells: A Comprehensive Protocol to Isolate All Principal CNS-Resident Cell Types from Brain and Spinal Cord of Adult Healthy and EAE Mice. Cells 2021, 10(3), 651).

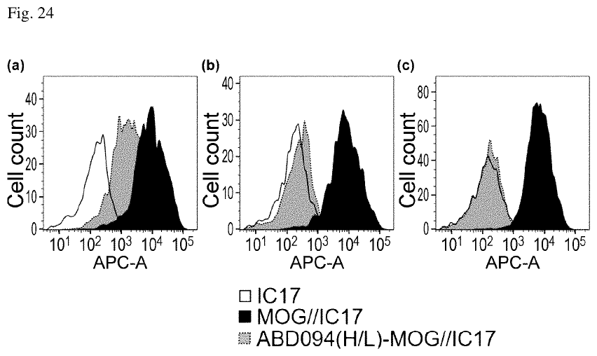

Binding histogram of the antibodies in oligodendrocytes is shown in FIG. 24. IC17 had similar, low fluorescence intensities under MSA-free DPBS (FIG. 24 (a)), MSA-containing DPBS (FIG. 24 (b)), and mouse plasma (FIG. 24 (c)) conditions. This suggests that IC17 does not bind to oligodendrocytes regardless of the presence or absence of albumin. On the other hand, MOG//IC17 showed similar, high fluorescence intensities in MSA-free DPBS (FIG. 24 (a)), MSA-containing DPBS (FIG. 24 (b)), and mouse plasma (FIG. 24 (c)), which were higher than those of IC17. This suggests that MOG//IC17 binds to MOG on the cell membrane at comparable levels regardless of the presence or absence of albumin.

ABD094(H/L)-MOG//IC17, which is an albumin switch molecule, showed fluorescence intensity close to those of MOG//IC17 and higher than those of IC17 in the MSA-free DPBS (FIG. 24 (a)). On the other hand, it showed fluorescence intensities lower than those of MOG//IC17 and at comparable levels as those of IC17 in the MSA-containing DPBS (FIG. 24 (b)) and mouse plasma (FIG. 24 (c)). This suggests that in the absence of albumin, an albumin switch molecule binds to MOG on the cell membrane, but in the presence of albumin their binding to MOG on the cell membrane is inhibited.

### Example 14: Pharmacokinetics of Anti-MOG Albumin Switch Molecules and MOG Antigen in Plasma

To examine the pharmacokinetics (PK) of albumin switch molecules and target antigen in plasma, the following anti-MOG antibodies were administered at 0.2 mg/kg to C57BL/6J mice (6 to 8-weeks old, male) through the tail vein. Twenty minutes after antibody administration, mouse soluble MOG (MOG) was administered at 0.021 mg/kg. The administered antibodies were IC17, MOG//IC17, and ABD094(H/L)-MOG//IC17. Blood was collected over time from MOG administration to 60 minutes after the administration, and plasma was obtained by centrifugation (12,000 rpm, 4 degrees C., 5 minutes).

Concentration of antibody in plasma (total antibody concentration) was measured by enzyme-linked immunosorbent assay (ELISA). As a capturing antibody, anti-human IgG heavy and light chain antibody (Bethyl Laboratories) was added to a plate. To this plate, phosphate buffered saline with Tween 20 (PBST, Sigma-Aldrich) containing 1% bovine serum albumin (BSA, Sigma-Aldrich) was added as a blocking solution, and then a diluted plasma sample was added. Then, biotin-labeled anti-dGK antibody was added as a detection antibody, and streptavidin-labeled polyHRP80 (Stereospecific Detection Technologies) was added as a secondary detection antibody. Finally, tetramethylbenzidine (TMB, Surmodics) was added as a substrate, and the absorbance at 650 nm was measured on a CLARIOstar absorptiometer (BMG Labotech).

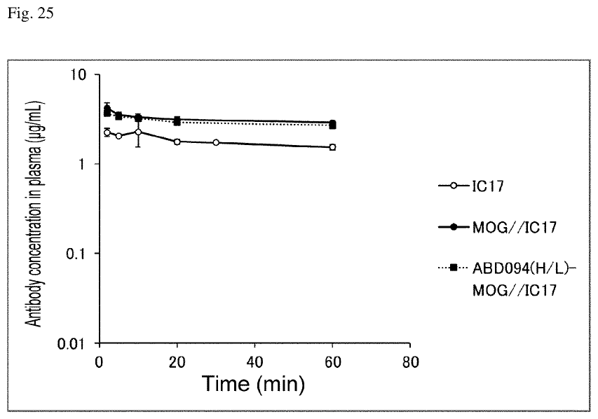

The plasma antibody concentration-time curves are shown in FIG. 25. All antibodies showed similar PK profiles.

Concentration of MOG in plasma (total MOG concentration) was measured by enzyme-linked immunosorbent assay (ELISA). As a capturing antibody, anti-mouse MOG polyclonal antibody (R&D) was added to a plate. To this plate, phosphate buffered saline with Tween 20 (PBST, Sigma-Aldrich) containing 1% bovine serum albumin (BSA, Sigma-Aldrich) was added as a blocking solution, and then a diluted plasma sample was added. Then, biotin-labeled anti-human/mouse MOG monoclonal antibody (R&D) was added as a detection antibody, and streptavidin-labeled polyHRP80 (Stereospecific Detection Technologies) was added as a secondary detection antibody. Finally, tetramethylbenzidine (TMB, Surmodics) was added as a substrate, and the absorbance at 650 nm was measured on a CLARIOstar absorptiometer (BMG Labotech).

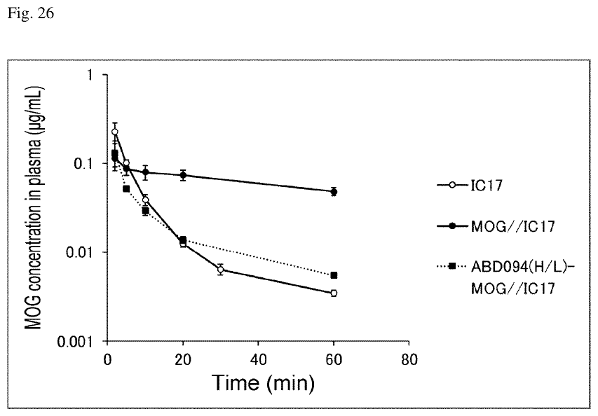

The plasma antigen concentration-time curves are shown in FIG. 26. Furthermore, the PK parameters calculated by the non-compartment model analysis using Phoenix WinNonlin (Certara) are shown in Table 17. In the group subjected to MOG//IC17 administration, MOG disappeared gradually, and the clearance was 2.35 mL/min/kg. On the other hand, in the group subjected to IC17 administration, MOG disappeared quickly, and the clearance was 10.6 mL/min/kg. Generally, IgG antibodies have long plasma half-lives, and their clearance is small. Therefore, such results suggest that in the group subjected to anti-MOG antibody administration, the target antigen (MOG) binds to the antibody and disappears gradually, whereas in the group subjected to administration of an antibody that does not bind to MOG, MOG does not bind to the antibody and disappears quickly.

In the group subjected to administration of the albumin switch molecule ABD094(H/L)-MOG//IC17, MOG disappeared more quickly compared to the MOG//IC17-administered group and clearance was larger (13.4 mL/min/kg. This shows that binding of the albumin switch molecule to the target antigen is decreased in plasma.

TABLE 17

| | AUC | CL | Vss | T½ |
|---|---|---|---|---|
| Antibody | min*/μg/mL | mL/min/kg | mL/kg | min |
| KLH | 2.01 | 10.6 | 166 | 28.7 |
| MOG//IC17 | 8.98 | 2.35 | 233 | 69.9 |
| ABD094(H/L)-MOG//IC17 | 1.60 | 13.4 | 342 | 27.0 |

### Example 15: Pharmacokinetics of Anti-MOG Albumin Switch Molecules in the Brain

To examine the pharmacokinetics (PK) of albumin switch molecules in the brain, the following anti-MOG antibodies were administered at 5 mg/kg to C57BL/6J mice (6 to 8-weeks old, male) through the tail vein. The administered antibodies were IC17, MOG//IC17, and ABD094(H/L)-MOG//IC17. Blood was collected on day 1, day 3, and day 7 after antibody administration, and plasma was obtained by centrifugation (12,000 rpm, 4 degrees C., 5 minutes). After blood collection, a catheter was inserted into the heart for perfusion with PBS. After the blood was removed, the brain was sampled. PBS containing NP-40 alternative detergent (Millipore) and Complete Mini Protease Inhibitor Cocktail (Roche), and 5-mm stainless beads (Qiagen) were added to the fragmented brain tissue sections, and this was homogenized using a homogenizer (Qiagen). The homogenate was rotated at 4 degrees C. for 60 minutes, and then centrifuged (15,000 rpm, 4 degrees C., 20 minutes) to collect the homogenate supernatant.

Plasma antibody concentration was measured by enzyme-linked immunosorbent assay (ELISA). As a capturing antibody, anti-human IgG heavy and light chain antibody (Bethyl Laboratories) was added to a plate. To this plate, phosphate buffered saline with Tween 20 (PBST, Sigma-Aldrich) containing 1% bovine serum albumin (BSA, Sigma-Aldrich) was added as a blocking solution, and then a diluted plasma sample was added. Then, biotin-labeled anti-dGK antibody was added as a detection antibody, and streptavidin-labeled polyHRP80 (Stereospecific Detection Technologies) was added as a secondary detection antibody. Finally, tetramethylbenzidine (TMB, Surmodics) was added as a substrate, and then the absorbance at 650 nm was measured on a CLARIOstar absorptiometer (BMG Labotech).

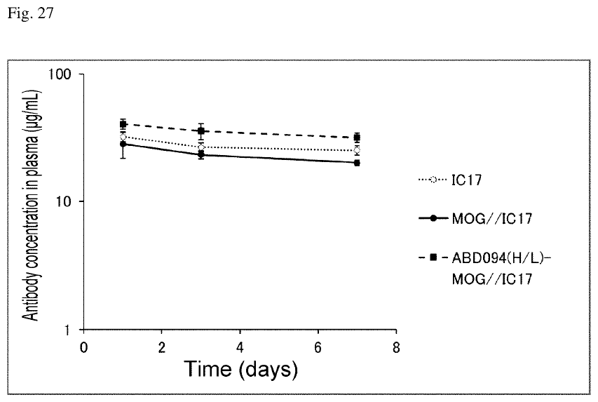

The plasma antibody concentration-time curves are shown in FIG. 27. All antibodies showed similar PK profiles.

The brain antibody concentration was measured by enzyme-linked immunosorbent assay (ELISA). As a capturing antibody, anti-human IgG heavy and light chain antibody (Bethyl Laboratories) was added to a plate. To this plate, phosphate buffered saline with Tween 20 (PBST, Sigma-Aldrich) containing 1% bovine serum albumin (BSA, Sigma-Aldrich) was added as a blocking solution, and then a brain homogenate sample diluted with LowCross-Buffer (CANDOR Bioscience) was added. Then, biotin-labeled anti-dGK antibody was added as a detection antibody, and streptavidin-labeled polyHRP80 (Stereospecific Detection Technologies) was added as a secondary detection antibody. Finally, tetramethylbenzidine (TMB, Surmodics) was added as a substrate, and then the absorbance at 650 nm was measured on a CLARIOstar absorptiometer (BMG Labotech).

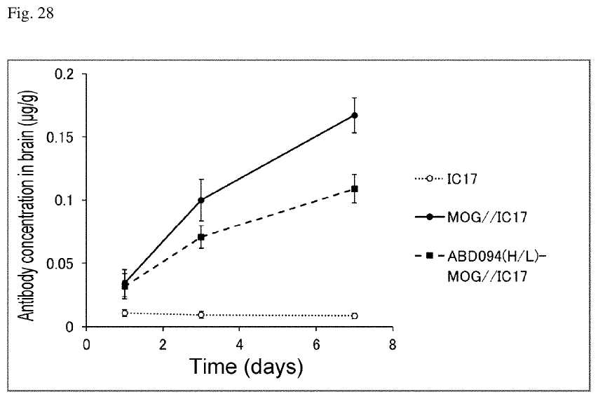

The brain antibody concentration-time curves are shown in FIG. 28. From day 1 to day 7 after IC17 administration, the brain antibody concentration did not change and showed roughly constant values. This suggests that IC17 does not bind to MOG expressed by cells in the brain. On the other hand, from day 1 to day 7, the brain antibody concentration of MOG//IC17 increased over time. This suggests that the anti-MOG antibody accumulates in the brain as a result of binding to the target antigen (MOG) expressed in the brain.

The brain antibody concentration of albumin switch molecule ABD094(H/L)-MOG//IC17 increased over time from day 1 to day 7 after administration. This suggests that the albumin switch molecule accumulates in the brain as a result of binding to the target antigen (MOG) expressed in the brain. This shows that, the intravenously administered albumin switch molecules migrate into the brain where the albumin concentration is lower than in the blood, and then binds to the target antigen expressed by cells in the brain. The results of this Example and Examples 7, 8, and 14 show that while binding of albumin switch molecules to the target antigen in plasma is inhibited by albumin, binding to the target antigen does take place after migration into the brain.

### Example 16: Effect of Difference in Linker Lengths on Antigen-Binding Inhibition

As the anti-Myelin Oligodendrocyte Glycoprotein (MOG) antibody heavy chain variable region (VH) and light chain variable region (VL), MOG307VH (SEQ ID NO: 47) and MOG307VL (SEQ ID NO: 48) were designed, respectively. MOG is an antigen expressed in the central nervous system (CNS) including the brain.

The VH and VL were designed so that an albumin-binding portion is attached to the N-terminal amino acid residue of VH and/or the N-terminal amino acid residue of VL of the anti-MOG antibody. Specifically, a gene encoding the albumin-binding domain ABD094 (SEQ ID NO: 4) was linked upstream of the gene(s) encoding the VH and/or VL of the anti-MOG antibody. For some antibodies, N10, N11 or N12 was placed as a linker between the VH and/or VL of the anti-MOG. IC17 is a molecule that does not bind to the target antigen (MOG) (WO2018043734A1).

Genes encoding VH and VL that were designed this way, were synthesized and linked to the genes encoding the heavy chain constant region of SEQ ID NO: 5 (SG181) or SEQ ID NO: 6 (both heavy chain constant regions lack the C-terminal glycine and lysine) and the light chain constant region of SEQ ID NO: 7 (SK1) or SEQ ID NO: 8, respectively. Each of the linked genes were cloned into expression vectors.

Using the genes produced as described above, the antibodies listed in Table 18 were expressed transiently in mammalian cells by a method known to those skilled in the art, and purified by a method known to those skilled in the art when necessary.

TABLE 18

| Name | Explanation | Albumin-binding site |
|---|---|---|
| MOG307//IC17 | Anti-MOG/KLH bispecific | None |
| | antibody (1 MOG-binding | |
| | site) | |
| ABD094(H/L)- | Anti-MOG/KLH bispecific | Albumin-binding domain (SEQ ID NO: 4) |
| MOG307//IC17 | antibody (1 MOG-binding | at the VH and VL N-termini of MOG- |
| | site) | binding arm. No linker. |
| ABD094(H/L).N10- | Anti-MOG/KLH bispecific | Albumin-binding domain (SEQ ID NO: 4) |
| MOG307//IC17 | antibody (1 MOG-binding | is linked at the VH and VL N-termini of |
| | site) | MOG-binding arm via a linker (N10). |
| ABD094(H/L).N11- | Anti-MOG/KLH bispecific | Albumin-binding domain (SEQ ID NO: 4) |
| MOG307//IC17 | antibody (1 MOG-binding | is linked at the VH and VL N-termini of |
| | site) | MOG-binding arm via a linker (N11). |
| ABD094(H/L).N12- | Anti-MOG/KLH bispecific | Albumin-binding domain (SEQ ID NO: 4) |
| MOG307//IC17 | antibody (1 MOG-binding | is linked at the VH and VL N-termini of |
| | site) | MOG-binding arm via a linker (N12). |

The combinations of amino acid sequences for MOG307//IC17, ABD094(H/L)-MOG307//IC17, ABD094(H/L).N10-MOG307//IC17, ABD094(H/L).N11-MOG307//IC17, and ABD094(H/L).N12-MOG307//IC17 are shown in Table 19 and Table 20.

TABLE 19
MOG-binding arm

| | Albumin-binding | | | | | H chain full | L chain full |
|---|---|---|---|---|---|---|---|
| Name | portion | VH | CH | VL | CL | sequence | sequence |
| MOG307//IC17 | None | SEQ ID NO: 47 | SEQ ID NO: 6 | SEQ ID NO: 48 | SEQ ID NO: 8 | SEQ ID NO: 49 | SEQ ID NO: 50 |
| ABD094(H/L)- | SEQ ID NO: 4 | SEQ ID NO: 47 | SEQ ID NO: 6 | SEQ ID NO: 48 | SEQ ID NO: 8 | SEQ ID NO: 51 | SEQ ID NO: 52 |
| MOG307//IC17 | | | | | | | |
| ABD094(H/L).N10- | SEQ ID NO: 4 + | SEQ ID NO: 47 | SEQ ID NO: 6 | SEQ ID NO: 48 | SEQ ID NO: 8 | SEQ ID NO: 55 | SEQ ID NO: 56 |
| MOG307//IC17 | Linker | | | | | | |
| ABD094(H/L).N11- | SEQ ID NO: 4 + | SEQ ID NO: 47 | SEQ ID NO: 6 | SEQ ID NO: 48 | SEQ ID NO: 8 | SEQ ID NO: 76 | SEQ ID NO: 77 |
| MOG307//IC17 | Linker | | | | | | |
| ABD094(H/L).N12- | SEQ ID NO: 4 + | SEQ ID NO: 47 | SEQ ID NO: 6 | SEQ ID NO: 48 | SEQ ID NO: 8 | SEQ ID NO: 78 | SEQ ID NO: 79 |
| MOG307//IC17 | Linker | | | | | | |

TABLE 20

| | KLH-binding arm | | |
|---|---|---|---|
| | Albumin- | | |
| | binding | H chain full | L chain full |
| Name | portion | sequence | sequence |
| MOG307//IC17 | None | SEQ ID NO:21 | SEQ ID NO: 22 |
| ABD094(H/L)- | | | |
| MOG307//IC17 | | | |
| ABD094(H/L).N10- | | | |
| MOG307//IC17 | | | |
| ABD094(H/L).N11- | | | |
| MOG307//IC17 | | | |
| ABD094(H/L).N12- | | | |
| MOG307//IC17 | | | |

To examine the pharmacokinetics (PK) of the target antigen in plasma after administration of the albumin switch molecules carrying linkers of different lengths, the following anti-MOG antibodies were administered at a dose of 0.2 mg/kg to C57BL/6J mice (6 to 8-weeks old, male) through the tail vein. Twenty minutes after antibody administration, soluble MOG (sMOG) was administered at 0.021 mg/kg. The administered antibodies were IC17, MOG307//IC17, ABD094(H/L)-MOG307//IC17, ABD094(H/L).N10-MOG307//IC17, ABD094(H/L).N11-MOG307//IC17, and ABD094(H/L).N12-MOG307//IC17. Blood was collected over time from sMOG administration to 60 minutes after the administration, and plasma was obtained by centrifugation (12,000 rpm, 4 degrees C., 5 minutes).

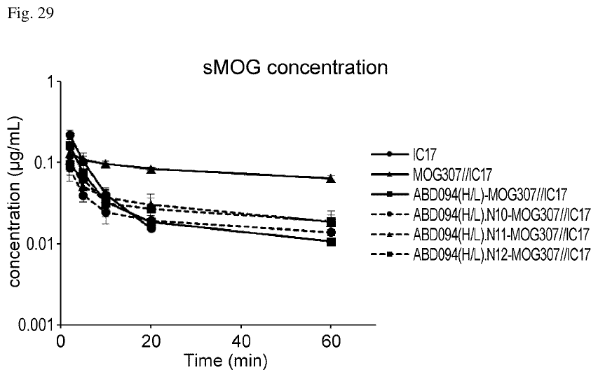

As a capture antibody, an anti-MOG polyclonal antibody (R&D) was applied in the 384-well plate. After PBS with Tween 20 (PBST, Sigma-Aldrich) containing 1% bovine serum albumin (Sigma-Aldrich) was added as blocking solution, diluted plasma were applied. As a detection antibody, a biotin-labeled anti-MOG monoclonal antibody (R&D) was added. Finally, a streptavidin labeled with poly-HRP (Stereospecific Detection Technologies) was added, and signals were detected. The plasma antigen concentration-time curves up to 60 min are shown in FIG. 29. Furthermore, the PK parameters of the antigen calculated using Phoenix WinNonlin (Certara) are shown in Table 21.

TABLE 21

| | AUC | CL | Vss | T½ |
|---|---|---|---|---|
| Antibody | min*ug/mL | mL/min/kg | mL/kg | min |
| IC17 | 1.709 | 12.59 | 74.27 | 5.317 |
| IC17//MOG307 | 13.03 | 1.637 | 202.7 | 87.47 |
| ABD094(H/L)- | 2.137 | 10.70 | 263.9 | 28.46 |
| MOG307//IC17 | | | | |
| ABD094(H/L).N10- | 2.871 | 7.517 | 671.6 | 70.80 |
| MOG307//IC17 | | | | |
| ABD094(H/L).N11- | 3.824 | 5.879 | 449.2 | 65.09 |
| MOG307//IC17 | | | | |
| ABD094(H/L).N12- | 4.305 | 5.678 | 535.6 | 83.58 |
| MOG307//IC17 | | | | |

In the group subjected to MOG307//IC17 administration, sMOG disappeared gradually; whereas, in the group subjected to IC17 administration, sMOG disappeared rapidly. Generally, IgG antibodies have long plasma half-lives, and their clearance is small. Therefore, such results suggest that in the group subjected to anti-MOG antibody administration, the target antigen (sMOG) binds to the antibody and disappears gradually; whereas in the group subjected to administration of an antibody that does not bind to MOG, sMOG does not bind to the antibody and disappears rapidly. In the group subjected to administration of ABD094(H/L)-MOG307//IC17, an albumin switch molecule that does not have a linker, sMOG disappeared significantly more rapidly compared to MOG307//IC17 and clearance was larger. More specifically, binding activity of ABD094(H/L)-MOG307//IC17 towards sMOG was suggested to be decreased in plasma.

The group subjected to administration of ABD094(H/L).N10-MOG307//IC17 which is an albumin switch molecule that uses the N10 linker consisting of one amino acid residue (G), sMOG showed a PK profile close to that of the group subjected to ABD094(H/L)-MOG307//IC17 administration. This shows that binding of ABD094(H/L).N10-MOG307//IC17 to sMOG in plasma is inhibited at a comparable level to that of ABD094(H/L)-MOG307//IC17.

In the group subjected to administration of ABD094(H/L).N11-MOG307//IC17 which is an albumin switch molecule that uses the N11 linker consisting of 2 amino acid residues (GS) and the group subjected to administration of ABD094(H/L).N12-MOG307//IC17 which is an albumin switch molecule that uses the N12 linker consisting of 3 amino acid residues (GGS), sMOG disappeared more gradually compared to ABD094(H/L)-MOG307//IC17 and ABD094(H/L).N10-MOG307//IC17, but disappeared more rapidly compared to MOG307//IC17. This suggests that binding of ABD094(H/L).N11-MOG307//IC17 and ABD094(H/L).N12-MOG307//IC17 to sMOG is also inhibited in plasma.

The results of this Example show that the linker length between the albumin-binding portion and the antigen-binding portion affects the binding of the albumin switch molecule to the target antigen in plasma. A short linker or no linker may be effective in inhibiting binding of albumin switch molecule to the target antigen in plasma.

### INDUSTRIAL APPLICABILITY

The present invention provides a polypeptide complex whose binding activity to a target antigen varies with the concentration of albumin. The polypeptide complex of the present invention does not bind to the target antigen in systemic blood where albumin concentration is high and can strongly bind to the target antigen in CNS where albumin concentration is low. Accordingly, the polypeptide complex of the present invention is expected to be a therapeutic agent and such for a CNS disease having decreased side effects accompanying binding to the target antigen in non-CNS tissues with high albumin concentration.

1. A polypeptide complex comprising a first antigen-binding portion capable of specifically binding to a plasma protein and a second antigen-binding portion capable of binding to a target antigen,
wherein the target antigen is not a plasma protein, and
wherein the first antigen-binding portion is linked with the second antigen-binding portion without a linker.

2. A polypeptide complex comprising a first antigen-binding portion capable of specifically binding to a plasma protein and a second antigen-binding portion capable of binding to a target antigen,
wherein the target antigen is not a plasma protein, and
wherein the first antigen-binding portion is linked with the second antigen-binding portion via a non-cleavable linker.

3. The polypeptide complex of claim 2, wherein the non-cleavable linker is a peptide of 4 amino acid residues or less.

4. The polypeptide complex of any one of claims 1 to 3, which does not bind to the target antigen when bound to the plasma protein.

5. The polypeptide complex of any one of claims 1 to 3, wherein the binding activity of the complex to the target antigen in the presence of the plasma protein is lower than the binding activity of the complex to the target antigen in the absence of the plasma protein.

6. The polypeptide complex of any one of claims 1 to 5, wherein the plasma protein is albumin, and preferably is human albumin.

7. The polypeptide complex of any one of claims 1 to 6, wherein the second antigen-binding portion comprises a Fab or a scFv.

8. A polypeptide complex comprising a first polypeptide chain and a second polypeptide chain,
wherein the first polypeptide chain comprises, in order from the N-terminus, a plasma protein-binding portion, a heavy chain variable region (VH), and a heavy chain constant region CH1 domain (CH1), and
the second polypeptide chain comprises, in order from the N-terminus, a plasma protein-binding portion, a light chain variable region (VL), and a light chain constant region (CL),
wherein the VH and the VL form a binding portion to a target antigen,
wherein the target antigen is not a plasma protein.

9. The polypeptide complex of claim 8, wherein the N-terminal amino acid of the VH and/or the VL is linked with the C-terminal amino acid of the plasma protein-binding portion without a linker.

10. The polypeptide complex of claim 8, wherein the N-terminal amino acid of the VH and/or the VL is linked with the C-terminal amino acid of the plasma protein-binding portion via a non-cleavable linker.

11. A pharmaceutical composition comprising the polypeptide complex of any one of claims 1 to 10, and a pharmaceutically acceptable carrier.

12. A pharmaceutical composition comprising the polypeptide complex of any one of claims 1 to 10, which is for targeting an antigen in a central nervous system (CNS), and which is for systemic administration to a subject.

13. The pharmaceutical composition of claim 12, wherein the systemic administration is intravenous administration or subcutaneous administration.

14. A method for producing a polypeptide complex whose binding activity to a target antigen varies with the concentration of a plasma protein, comprising:
(a) measuring the binding activity of a polypeptide complex to the target antigen both in the presence and absence of the plasma protein,
(b) selecting a polypeptide complex whose binding activity to the target antigen is lower in the presence of the plasma protein than in the absence of the plasma protein,
(c) obtaining a polynucleotide encoding the polypeptide complex selected in (b), and
(d) culturing a cell comprising the polynucleotide obtained in (c),
wherein the polypeptide complex comprises a first antigen-binding portion capable of specifically binding to the plasma protein and a second antigen-binding portion capable of binding to the target antigen.

15. A method for producing a polypeptide complex whose binding activity to a target antigen varies with the concentration of a plasma protein, comprising:
culturing a cell comprising a polynucleotide encoding the polypeptide complex, wherein:
(a) the polypeptide complex comprises a first antigen-binding portion capable of specifically binding to the plasma protein and a second antigen-binding portion capable of binding to the target antigen, and
(b) the binding activity of the polypeptide complex to the target antigen is lower in the presence of the plasma protein than in the absence of the plasma protein.


SEQUENCE LISTING (79 sequences)

SEQ ID NO: 1 - protein, 119 aa, synthetic construct
  source 1..119: note = H54
QVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSS

SEQ ID NO: 2 - protein, 107 aa, synthetic construct
  source 1..107: note = L28
DIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIE

SEQ ID NO: 3 - protein, 20 aa, synthetic construct
  source 1..20: note = QRLMEDICLPRWGCLWEDDF
QRLMEDICLPRWGCLWEDDF

SEQ ID NO: 4 - protein, 46 aa, synthetic construct
  source 1..46: note = LAEAKEAANAELDSYGVSDFYKRLIDKAKTVEGVEALKDAILAALP
LAEAKEAANAELDSYGVSDFYKRLIDKAKTVEGVEALKDAILAALP

SEQ ID NO: 5 - protein, 328 aa, synthetic construct
  source 1..328: note = SG181
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 6 - protein, 328 aa, synthetic construct
  source 1..328: note = SG181009v11h
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 7 - protein, 107 aa, synthetic construct [sequence omitted]

SEQ ID NO: 8 - protein, 107 aa, synthetic construct
  source 1..107: note = RKRohuK1
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 447 aa, synthetic construct
  source 1..447: note = H54-SG181
QVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 10 - protein, 471 aa, synthetic construct
  source 1..471: note = AlbH54-SG181
QRLMEDICLPRWGCLWEDDFGGGSQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 11 - protein, 444 aa, synthetic construct
  source 1..444: note = IC17HdK-SG181
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 12 - protein, 447 aa, synthetic construct
  source 1..447: note = H54-SG181009v11h
QVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 13 - protein, 471 aa, synthetic construct
  source 1..471: note = AlbH54-SG181009v11h
QRLMEDICLPRWGCLWEDDFGGGSQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 14 - protein, 214 aa, synthetic construct
  source 1..214: note = L28-SK1
DIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 238 aa, synthetic construct
  source 1..238: note = AlbL28-SK1
QRLMEDICLPRWGCLWEDDFGGGSDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 214 aa, synthetic construct
  source 1..214: note = IC17L-SK1
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 214 aa, synthetic construct
  source 1..214: note = L28-RKRohuK1
DIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 238 aa, synthetic construct
  source 1..238: note = AlbL28-RKRohuK1
QRLMEDICLPRWGCLWEDDFGGGSDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 493 aa, synthetic construct
  source 1..493: note = ABD094.N00.H54-SG181009v11h
LAEAKEAANAELDSYGVSDFYKRLIDKAKTVEGVEALKDAILAALPQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 20 - protein, 260 aa, synthetic construct
  source 1..260: note = ABD094.N00.L28-RKRohuK1
LAEAKEAANAELDSYGVSDFYKRLIDKAKTVEGVEALKDAILAALPDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 437 aa, synthetic construct
  source 1..437: note = IC17L-xSG181v11k
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 22 - protein, 223 aa, synthetic construct
  source 1..223: note = IC17HdK-xSK1
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 46 aa, synthetic construct
  source 1..46: note = ABD035
LAEAKVLANRELDKYGVSDFYKRLINKAKTVEGVEALKLHILAALP

SEQ ID NO: 24 - protein, 46 aa, synthetic construct
  source 1..46: note = G418
LAEAKVLANRELDKYGVSDYYKNLINNAKTVEGVKALIDEILAALP

SEQ ID NO: 25 - protein, 493 aa, synthetic construct
  source 1..493: note = ABD035.N00.H54-SG181009v11h
LAEAKVLANRELDKYGVSDFYKRLINKAKTVEGVEALKLHILAALPQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 26 - protein, 260 aa, synthetic construct
  source 1..260: note = ABD035.N00.L28-RKRohuK1
LAEAKVLANRELDKYGVSDFYKRLINKAKTVEGVEALKLHILAALPDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 493 aa, synthetic construct
  source 1..493: note = G148.N00.H54-SG181009v11h
LAEAKVLANRELDKYGVSDYYKNLINNAKTVEGVKALIDEILAALPQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 28 - protein, 260 aa, synthetic construct
  source 1..260: note = G148.N00.L28-RKRohuK1
LAEAKVLANRELDKYGVSDYYKNLINNAKTVEGVKALIDEILAALPDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29-34 - skipped (6 entries)

SEQ ID NO: 35 - protein, 5 aa, synthetic construct
  source 1..5: note = N02
GGGGS

SEQ ID NO: 36 - protein, 498 aa, synthetic construct
  source 1..498: note = ABD094.N02.H54-SG181009v11h
LAEAKEAANAELDSYGVSDFYKRLIDKAKTVEGVEALKDAILAALPGGGGSQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 37 - protein, 265 aa, synthetic construct
  source 1..265: note = ABD094.N02.L28-RKRohuK1
LAEAKEAANAELDSYGVSDFYKRLIDKAKTVEGVEALKDAILAALPGGGGSDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 11 aa, synthetic construct
  source 1..11: note = core seq of albumin binding peptides
DICLPRWGCLW

SEQ ID NO: 39 - protein, 18 aa, synthetic construct
  source 1..18: note = SA21
RLIEDICLPRWGCLWEDD

SEQ ID NO: 40 - protein, 46 aa, synthetic construct
  source 1..46: note = ABD variants
  VARIANT 3: note = X is E, S, Q or C
  VARIANT 6: note = X is E, S or C
  VARIANT 7: note = X is A or S
  VARIANT 10: note = X is A, S or R
  VARIANT 14: note = X is A, S, C or K
  VARIANT 26: note = X is D or E
  VARIANT 39: note = X is D or E
  VARIANT 40: note = X is A or E
  VARIANT 43: note = X is A or K
  VARIANT 44: note = X is A, S or E
  VARIANT 45: note = L may be absent
  VARIANT 46: note = P may be absent
LAXAKXXANXELDXYGVSDFYKRLIXKAKTVEGVEALKXXILXXLP

SEQ ID NO: 41 - protein, 46 aa, synthetic construct
  source 1..46: note = ABD variants
  VARIANT 3: note = X is E or S
  VARIANT 14: note = X is A, S, C or K
LAXAKEAANAELDXYGVSDFYKRLIDKAKTVEGVEALKDAILAALP

SEQ ID NO: 42 - protein, 4 aa, synthetic construct
  source 1..4: note = GGGS
GGGS

SEQ ID NO: 43 - protein, 4 aa, synthetic construct
  source 1..4: note = GGSG
GGSG

SEQ ID NO: 44 - protein, 4 aa, synthetic construct
  source 1..4: note = GSGG
GSGG

SEQ ID NO: 45 - protein, 4 aa, synthetic construct
  source 1..4: note = SGGG
SGGG

SEQ ID NO: 46 - protein, 4 aa, synthetic construct
  source 1..4: note = GSSG
GSSG

SEQ ID NO: 47 - protein, 127 aa, synthetic construct
  source 1..127: note = MOG307VH
RVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSS

SEQ ID NO: 48 - protein, 107 aa, synthetic construct
  source 1..107: note = MOG307VL
EIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 49 - protein, 455 aa, synthetic construct
  source 1..455: note = MOG307VH-SG181009v11h
RVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 50 - protein, 214 aa, synthetic construct
  source 1..214: note = MOG307VL-RKRohuK1
EIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 501 aa, synthetic construct
  source 1..501: note = ABD094.N00.MOG307VH-SG181009v11h
LAEAKEAANAELDSYGVSDFYKRLIDKAKTVEGVEALKDAILAALPRVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 52 - protein, 260 aa, synthetic construct
  source 1..260: note = ABD094.N00.MOG307VL-RKRohuK1
LAEAKEAANAELDSYGVSDFYKRLIDKAKTVEGVEALKDAILAALPEIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 506 aa, synthetic construct
  source 1..506: note = ABD094.N02.MOG307VH-SG181009v11h
LAEAKEAANAELDSYGVSDFYKRLIDKAKTVEGVEALKDAILAALPGGGGSRVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 54 - protein, 265 aa, synthetic construct
  source 1..265: note = ABD094.N02.MOG307VL-RKRohuK1
LAEAKEAANAELDSYGVSDFYKRLIDKAKTVEGVEALKDAILAALPGGGGSEIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 502 aa, synthetic construct
  source 1..502: note = ABD094.N10.MOG307VH-SG181009v11h
LAEAKEAANAELDSYGVSDFYKRLIDKAKTVEGVEALKDAILAALPGRVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 56 - protein, 261 aa, synthetic construct
  source 1..261: note = ABD094.N10.MOG307VL-RKRohuK1
LAEAKEAANAELDSYGVSDFYKRLIDKAKTVEGVEALKDAILAALPGEIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 494 aa, synthetic construct
  source 1..494: note = ABD094.N10.H54-SG181009v11h
LAEAKEAANAELDSYGVSDFYKRLIDKAKTVEGVEALKDAILAALPGQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 58 - protein, 261 aa, synthetic construct
  source 1..261: note = ABD094.N10.L28-RKRohuK1
LAEAKEAANAELDSYGVSDFYKRLIDKAKTVEGVEALKDAILAALPGDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59-61 - skipped (3 entries)

SEQ ID NO: 62 - protein, 4 aa, synthetic construct
  source 1..4: note = N13
GGGS

SEQ ID NO: 63 - protein, 495 aa, synthetic construct
  source 1..495: note = ABD094.N11.H54-SG181009v11h
LAEAKEAANAELDSYGVSDFYKRLIDKAKTVEGVEALKDAILAALPGSQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 64 - protein, 496 aa, synthetic construct
  source 1..496: note = ABD094.N12.H54-SG181009v11h
LAEAKEAANAELDSYGVSDFYKRLIDKAKTVEGVEALKDAILAALPGGSQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 65 - protein, 497 aa, synthetic construct
  source 1..497: note = ABD094.N13.H54-SG181009v11h
LAEAKEAANAELDSYGVSDFYKRLIDKAKTVEGVEALKDAILAALPGGGSQVQLQESGPGLVKPSETLSLTCAVSGYSISDDQAWSWVRQPPGEGLEWIGYISYSGITNYNPSLKGRVTISRDTSKNQFSLKLSSVTAADTAAYYCARSLARTTAMDYWGEGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 66 - protein, 262 aa, synthetic construct
  source 1..262: note = ABD094.N11.L28-RKRohuK1
LAEAKEAANAELDSYGVSDFYKRLIDKAKTVEGVEALKDAILAALPGSDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 263 aa, synthetic construct
  source 1..263: note = ABD094.N12.L28-RKRohuK1
LAEAKEAANAELDSYGVSDFYKRLIDKAKTVEGVEALKDAILAALPGGSDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 264 aa, synthetic construct
  source 1..264: note = ABD094.N13.L28-RKRohuK1
LAEAKEAANAELDSYGVSDFYKRLIDKAKTVEGVEALKDAILAALPGGGSDIQMTQSPSSLSASVGDSVTITCQASQDISSYLNWYQQKPGKAPELLIYYGSELHSGVPSRFSGSGSGTDFTFTISSLEAEDAATYYCQQGNSLPYTFGQGTKVEIERTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 45 aa, synthetic construct
  VARIANT 2: note = X is A, D, K, L, Q or S
  VARIANT 3: note = X is E, K, L, N, Q or R
  VARIANT 7: note = X is A, D, E, K, L or M
  VARIANT 10: note = X is A, I, K, L, N, Q or R
  VARIANT 14: note = X is A, E, K, Q, R or V
  VARIANT 15: note = X is A, K, L, N or Y
  VARIANT 18: note = X is F, I, L, Y or absent
  VARIANT 21: note = X is F, I, L, Y or absent
  VARIANT 23: note = X is F, I, L, K, T or V
  VARIANT 24: note = X is D, K, N, S or T
  VARIANT 25: note = X is A, K, L, N or Q
  VARIANT 36: note = X is E, K, M, N, T or V
  VARIANT 40: note = X is A, D, E, N or Q
  VARIANT 44: note = X is A, E, K, N, Q, S or absent
  VARIANT 45: note = X is A, Q, S or absent
LXXAKEXAIXELKXXGIXSDXYXXXINKAKTVEGVXALKXEILXX

SEQ ID NO: 70 - protein, 46 aa, synthetic construct
  VARIANT 6: note = X is V or E
  VARIANT 7: note = X is L, E or D
  VARIANT 9: note = X is N, L or I
  VARIANT 10: note = X is R or K
  VARIANT 13: note = X is D or K
  VARIANT 20: note = X is Y or F
  VARIANT 23: note = X is N, R or S
  VARIANT 24: note = X is V, I, L, M, F or Y
  VARIANT 26: note = X is N, S, E or D
  VARIANT 27: note = X is R, K or N
  VARIANT 29: note = X is K or R
  VARIANT 35: note = X is D, N, Q, E, H, S, R or K
  VARIANT 38: note = X is K, I or T
  VARIANT 39: note = X is A, S, T, G, H, L or D
  VARIANT 40: note = X is H, E or D
LAEAKXXAXXELXKYGVSDXYKXXIXXAXTVEGVXALXXXILAALP

SEQ ID NO: 71 - protein, 46 aa, synthetic construct; identical to SEQ ID NO: 40
  VARIANT 3: note = X is E, S or Q
  VARIANT 6: note = X is E or S
  VARIANT 7: note = X is A or S
  VARIANT 10: note = X is A, S or R
  VARIANT 14: note = X is A, S or K
  VARIANT 26: note = X is D or E
  VARIANT 39: note = X is D or E
  VARIANT 40: note = X is A or E
  VARIANT 43: note = X is A or K
  VARIANT 44: note = X is A, S or E
  VARIANT 45: note = L may be absent
  VARIANT 46: note = P may be absent

SEQ ID NO: 72 - protein, 46 aa, synthetic construct; identical to SEQ ID NO: 40
  VARIANT 3: note = X is E or S
  VARIANT 6: note = X is E
  VARIANT 7: note = X is A
  VARIANT 10: note = X is A or R
  VARIANT 14: note = X is A, S or K
  VARIANT 26: note = X is D
  VARIANT 39: note = X is D
  VARIANT 40: note = X is A
  VARIANT 43: note = X is A
  VARIANT 44: note = X is A
  VARIANT 45: note = L may be absent
  VARIANT 46: note = P may be absent

SEQ ID NO: 73 - protein, 45 aa, synthetic construct
  VARIANT 21: note = X is A, K or Y
  VARIANT 22: note = X is Y, A, V or S
  VARIANT 30: note = X is K or D
  VARIANT 31: note = X is T or A
  VARIANT 37: note = X is V, Y or A
  VARIANT 41: note = X is E or Q
LKEAKEKAIEELKKAGITSDXXFDLINKAXXVEGVNXLKDXILKA

SEQ ID NO: 74 - protein, 46 aa, synthetic construct
  VARIANT 23: note = X is K, R, N or S
LAEAKEAANAELDSYGVSDFYKXLIDKAKTVEGVEALKDAILAALP

SEQ ID NO: 75 - protein, 6 aa, synthetic construct
HVVQLR

SEQ ID NO: 76 - protein, 503 aa, synthetic construct
  source 1..503: note = ABD094.N11.MOG307VH-SG181009v11h
LAEAKEAANAELDSYGVSDFYKRLIDKAKTVEGVEALKDAILAALPGSRVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 77 - protein, 262 aa, synthetic construct
  source 1..262: note = ABD094.N11.MOG307VL-RKRohuK1
LAEAKEAANAELDSYGVSDFYKRLIDKAKTVEGVEALKDAILAALPGSEIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 504 aa, synthetic construct
  source 1..504: note = ABD094.N12.MOG307VH-SG181009v11h
LAEAKEAANAELDSYGVSDFYKRLIDKAKTVEGVEALKDAILAALPGGSRVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 79 - protein, 263 aa, synthetic construct
  source 1..263: note = ABD094.N12.MOG307VL-RKRohuK1
LAEAKEAANAELDSYGVSDFYKRLIDKAKTVEGVEALKDAILAALPGGSEIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC